(19) United States
(12) Patent Application Publication
(10) Pub. No.: US 2024/0139320 A1
(43) Pub. Date: May 2, 2024

(54) CHIMERIC ANTIGEN RECEPTOR (CAR) SIGNALING MOLECULES FOR CONTROLLED AND SPECIFIC CAR T CELL ACTIVITY

(71) Applicant: The Board of Trustees of the Leland Stanford Junior University, Stanford, CA (US)

(72) Inventors: Robbie G. Majzner, Palo Alto, CA (US); Aidan Tousley, Stanford, CA (US); Louai Labanieh, Stanford, CA (US); Crystal L. Mackall, Stanford, CA (US); Maria Caterina Rotiroti, Stanford, CA (US)

(21) Appl. No.: 18/547,652

(22) PCT Filed: Feb. 23, 2022
(86) PCT No.: PCT/US2022/017544

§ 371 Date: Aug. 23, 2023

Related U.S. Application Data
(60) Provisional application No. 63/153,033, filed on Feb. 24, 2021.

(51) Int. Cl. A61K 39/00; A61P 35/00; C07K 14/705; C07K 14/725; C07K 14/73; C07K 16/32; C12N 5/0783; C12N 9/12; C12N 9/16

(52) U.S. Cl. CPC A61K 39/4631; A61K 39/4611; A61K 39/4644; A61P 35/00; C07K 14/7051; C07K 14/70514; C07K 14/70517; C07K 14/70521; C07K 16/32; C12N 5/0636; C12N 9/12; C12N 9/16; C12Y 207/10002; C12Y 301/04011; C07K 2317/31; C07K 2317/35; C07K 2319/02; C07K 2319/03

(57) ABSTRACT

The present disclosure generally relates to, inter alia, chimeric antigen receptors (CARs) that contain an intracellular signaling domain without an immune receptor tyrosine based activation motif (ITAM). The disclosure also provides compositions and methods useful for producing such molecules, as well as methods for the detection and treatment of diseases, such as cancer.

### RELATED APPLICATIONS

This application claims priority to and benefit of U.S. Provisional Application No. 63/153,033, filed on Feb. 24, 2021; the contents of which are hereby incorporated in its entirety.

### STATEMENT REGARDING FEDERALLY SPONSORED R&D

The invention was made with government support under grant no. 1P01CA217959-01 (P01) and 1DP2CA272092-01 (DP2) awarded by National Institutes of Health. The government has certain rights in the present invention.

### INCORPORATION BY REFERENCE

This application contains a Sequence Listing, which is hereby incorporated herein by reference in its entirety. The accompanying Sequence Listing text file, named “078430-524001WO_SequenceListing_ST25.txt,” was created on Feb. 18, 2022 and is 893,877 bytes.

### FIELD

The present disclosure relates generally to the fields of oncology and immuno-therapeutics, and particularly relates to compositions of polypeptides, e.g., chimeric antigen receptors (CARs) that have enhanced efficacy and/or can be used for logic gating to target and eliminate specific cells (e.g., cancer cells) when multiple antigens are present. The disclosure also provides compositions and methods useful for producing such molecules, as well as methods for the detection and treatment of conditions, such as diseases (e.g., cancer or autoimmune diseases).

### BACKGROUND

Chimeric antigen receptor (CAR) T cells have revolutionized the care of patients with relapsed and refractory B cell malignancies, but have not yet demonstrated substantial therapeutic benefit in patients with solid tumors. Complete response rates of up to 90% for CD19 CAR T cells in patients with leukemia illustrate the enormous potential of these therapeutics to revolutionize care of solid tumors if they could be unleashed safely. However, the lack of truly tumor-specific cell surface antigens has hampered development of CARs for solid tumors due to concerns about on-target killing of normal tissues that share the target antigen (known as “on-target, off tumor toxicity”), as has been observed in early phase clinical trials and in preclinical models. CAR T cells cannot discriminate between cancer and normal tissue if both express the target antigen. To date, no system developed can effectively overcome this intractable problem, greatly limiting the number of potential therapeutic targets and diseases that can be treated. Consequently, there remains a need for more potent and specific CARs to overcome these obstacles to extend the reach of these therapeutics to more diseases and to treat more patients.

### SUMMARY

The present disclosure relates generally to the development of immuno-therapeutics, including recombinant polypeptides such as chimeric antigen receptors (CARs), either alone or in combination as Boolean logic AND gates, as well as pharmaceutical compositions containing the same for use in treating various conditions, such as diseases (e.g., cancer). As described in greater detail below, various modifications of the intracellular signaling domain (a.k.a. cytosolic signaling region) have been found to have dramatic effects on the CAR's potency and specificity to antigens. In some embodiments, the intracellular signaling domains of these CAR molecules do not have an immune receptor tyrosine based activation motif (ITAM), such as CD3zeta (CD3ζ). Furthermore, experimental results described herein have demonstrated that combinations of these CARs similar to Boolean logic gates (e.g. AND) integrate signals based on the presence of multiple antigens, which would drastically increase their safety. Domain swap and mutations have been used to engineer CAR molecules or CAR molecule combinations with enhanced specificity and/or potency.

In one aspect, provided herein is a chimeric antigen receptor (CAR) polypeptide including:
a) an extracellular ligand-binding domain having a binding affinity for a ligand;
b) a transmembrane domain; and
c) an intracellular signaling domain,
wherein binding of the ligand to the extracellular ligand-binding domain activates the intracellular signaling domain,
and wherein the intracellular signaling domain does not have an immune receptor tyrosine based activation motif (ITAM).

Non-limiting exemplary embodiments of the disclosed chimeric antigen receptor (CAR) polypeptide of the disclosure include one or more of the following features. In some embodiments, the CAR polypeptide has the multiple domains described in a) to c) above, in N-terminal to C-terminal direction. In some embodiments, the intracellular signaling domain has a full-length or biologically active fragment of a protein kinase, a G protein, a GTP-binding protein, an adaptor signaling protein, or a scaffold protein capable of inducing cell activation. In some embodiments, the intracellular signaling domain does not have a CD3ζ domain. In some embodiments, the intracellular signaling domain contains ZAP70, PLCG1, PKC, ITK, NCK, VAV1, GRB2, GADS, SOS1, ADAP, SYK, LYN, PI3K, BLNK, or a biologically active fragment, mutant, or variant thereof. In some embodiments, a CAR molecule disclosed herein contains more than one intracellular signaling domain selected from the group consisting of ZAP70, PLCG1, PKC, ITK, NCK, VAV1, GRB2, GADS, SOS1, ADAP, SYK, LYN, PI3K, BLNK, and a biologically active fragment, mutant, or variant thereof.

In some embodiments, the intracellular signaling domain contains ZAP70 or PLCG1, or a biologically active fragment, mutant, or variant thereof. In some embodiments, the biologically active fragment, mutant, or variant thereof is a fragment containing the full-length or a fragment of Interdomain B and the kinase domain from ZAP70, or a mutant or variant thereof. In some embodiments, the biologically active fragment, mutant, or variant thereof contains the full-length or a fragment of a ZAP70²⁵⁵⁻⁶⁰⁰ fragment. In some embodiments, the biologically active fragment, mutant, or variant thereof contains:
i) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment;
ii) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further containing at least one of the mutations at the position of Y292, Y492, K544, Y597, Y598, V314, D327, R360, and K362;
iii) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further containing at least one of the mutations of Y292F, Y492F, K544R, Y597F, Y598F, V314A, D327P, R360P, and K362E;
iv) a ZAP70^{255-600 Y292F} fragment;
v) a ZAP70^{255-600 Y492F} fragment;
vi) a ZAP70^{255-600 K544R} fragment;
vii) a ZAP70^{255-600 Y597F Y598F} fragment;
viii) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further containing at least one costimulatory domain;
ix) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further containing a 4-1BB costimulatory domain;
x) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further containing a CD28 costimulatory domain;
xi) a ZAP70^{255-600 V314A} fragment;
xii) a ZAP70^{255-600 D327P} fragment;
xiii) a ZAP70^{255-600 R360P} fragment; and/or
xiv) a ZAP70^{255-600 K362E} fragment.

In some embodiments, the intracellular signaling domain described herein has an amino acid sequence having at least 20%, 25%, 30%, 35%, 40%, 45%, 50%, 55%, 60%, 65%, 70%, 75%, 80%, 85%, 90%, 95%, 99%, or more identity to any one of SEQ ID NOs: 11-26, 103-110, 163, 165, 167, 169, 177, 179, 185, 188, 191, 193, and 195. In some embodiments, the intracellular signaling domain described herein has an amino acid sequence having at least 50%, 60%, 70%, 75%, 80%, 90%, 95%, 99%, or more identity to any one of SEQ ID NOs: 11-26 and 103-110, 163, 165, 167, 169, 177, 179, 185, 188, 191, 193, and 195. In some embodiments, the intracellular signaling domain has an amino acid sequence of any one of SEQ ID NOs: 11-26, 103-110, 163, 165, 167, 169, 177, 179, 185, 188, 191, 193, and 195. In some embodiments, the intracellular signaling domain consists of an amino acid sequence of any one of SEQ ID NOs: 11-26, 103-110, 163, 165, 167, 169, 177, 179, 185, 188, 191, 193, and 195.

In some embodiments, the extracellular ligand-binding domain (a.k.a., extracellular antigen-binding domain) has a ligand-binding domain of an antibody, an antigen-binding fragment, an antibody mimetic, a receptor, a peptide specifically binding to a ligand or antigen, or a ligand for a targeted receptor. In some embodiments, the antibody or the antigen-binding fragment is selected from the group consisting of a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a VH domain, a VL domain, an Fv fragment, a VNAR domain, and a VHH domain. In some embodiments, the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof. In some embodiments, the receptor is NKG2D or a biologically active fragment thereof. In some embodiments, the ligand for a targeted receptor is an IL-13 polypeptide, an IL-13 mutein, chlorotoxin, or a biologically active fragment thereof. In some embodiments, the extracellular ligand-binding domain is multivalent (e.g., bivalent). In some embodiments, the extracellular ligand-binding domain is multispecific (e.g., bispecific).

In some embodiments, the ligand or antigen recognized by the extracellular ligand-binding domain localizes on the surface of a cell. In some embodiments, the ligand or antigen is an adaptor molecule. In some embodiments, the adaptor is specifically recognized by a cell (e.g., a cancer cell, or a cell correlated to a disease or disorder described herein, such as proliferative diseases (e.g., cancers), hematological malignancies, solid tumors, autoimmune diseases, inflammations, allergic diseases, infections, senescence/aging, etc.). Non-limiting examples of suitable ligand types include cell surface receptors, adhesion proteins, carbohydrates, lipids, glycolipids, lipoproteins, and lipopolysaccharides that are surface-bound, integrins, mucins, and lectins. In some embodiments, the ligand is a protein. In some embodiments, the ligand is a carbohydrate. In some embodiments, the ligand is at least one selected from the group consisting of: CD19, HER2, ROR1, B7-H3 (CD276), influenza hemagglutinin (HA), CD22, IL13Ra2, CD2, CD5, CD6, FcγR1, integrins, gangliosides, and glycopeptides. In some embodiments, the ligand is at least one selected from the group consisting of: CD1a, CD1b, CD1c, CD2, CD3, CD4, CD5, CD6, CD7, CD8, CD9, CD10, CD11a, CD11b, CD11c, CD12, CD13, CD14, CD15 (SSEA-1), CD16 (FcγRIII), CD17, CD18, CD19, CD20, CD21, CD22, CD23, CD24, CD25, CD26, CD27, CD28, CD29, CD30, CD31, CD32 (FcγRII), CD33, CD34, CD35, CD36, CD37, CD38, CD39, CD40, CD41, CD43, CD44, CD44V6, CD45, CD45R/B220, CD45RO, CD49b, CD49d, CD49f, CD52, CD53, CD54, CD56 (NCAM), CD57, CD61 (integrin β3), CD62L, CD63, CD64, CD66b, CD68, CD69, CD70, CD73, CD74, CD79a (Igα), CD79b (Igβ), CD80, CD83, CD85k (ILT3), CD86, CD88, CD93 (C1Rqp), CD94, CD95, CD99, CD103, CD105 (Endoglin), CD107a, CD107b, CD114 (G-CSFR), CD115, CD117, CD122, CD123, CD129, CD133, CD134, CD138 (Syndecan-1), CD141, CD146, CD152 (CTLA-4), CD158 (Kir), CD161 (NK-1.1), CD163, CD183, CD191, CD193 (CCR3), CD194 (CCR4), CD195 (CCR5), CD197 (CCR7), CD203c, CD205 (DEC-205), CD207 (Langerin), CD209 (DC-SIGN), CD223, CD235, CD244 (2B4), CD252 (OX40L), CD267, CD268 (BAFF-R), CD273 (B7-DC, PD-L2), CD276 (B7-H3), CD279 (PD1), CD282 (TLR2), CD284 (TLR4), CD294, CD304 (Neuropilin-1), CD305, CD314 (NKG2D), CD319 (CRACC), CD326, CD328 (Siglec-7), CD335 (NKp46), fetal acetylcholine receptor (AChR), ADGRE2, alpha-fetoprotein (AFP), ALK, BCMA, BDCA3, C3AR, Lewis A (CA19.9), carbonic anhydrase IX (CA1X), calretinin, cancer antigen-125 (CA-125), CCR1, CCR4, CDS, carcinoembryonic antigen (CEA), chromogranin, CLEC12A, an antigen of a cytomegalovirus (CMV) infected cell (e.g., a cell surface antigen), CS-1, CSPG4, cytokeratin, desmin, DLK1, DLL3, EGFRvIII (epidermal growth factor variant III), EGFR and isovariants thereof, epithelial cell adhesion molecule (EpCAM), epithelial glycoprotein2 (EGP 2), epithelial glycoprotein-40 (EGP-40), epithelial membrane protein (EMA), ERBB, epithelial tumor antigen (ETA), FAP, folate-binding protein (FBP), FcγR1, FcεRIα, FITC, FLT3, FOLR1, FOLR3, galactin, ganlgiosides, gross cystic disease fluid protein (GCDFP-15), GD2 (ganglioside G2), GD3, GM2, GM3, glial fibrillary acidic protein (GFAP), gpA33, glycopeptides, Glypican 2 (GPC2), oncofetal antigen (h5T4), influenza hemagglutinin (HA), human epidermal growth factor receptor 2 (Her2/neu), HLA-DR, HM1.24, HMB-45 antigen, HPV E6, HPV E7, ICAM-1, IgG, IgD, IgE, IgM, IL-13-receptor alpha 1, integrins, Integrin B7, Interleukin-13 receptor subunit alpha-2 (IL-13Rα2), Kappa light chain, kinase insert domain receptor (KDR), Lamba light chain, LILRB2, Lewis Y (LeY), LGR5, Ly49, Ly108, L1 cell adhesion molecule (L1-CAM), melanoma-associated antigen (MAGE), melanoma antigen family A 1 (MAGE-A1), protein melan-A (melanoma antigen recognized by T lymphocytes; MART-1), MCSP, c-Met, MICA/B, mesothelin, muscle-specific actin (MSA), Mesothelin (MSLN), the dimeric form of the pyruvate kinase isoenzyme type M2 (tumor M2-PK), Mucin 1 (Muc-1), Mucin 16 (Muc-16), myo-D1, Necl-2, neurofilament, NKCSI, NKG2D, neuron-specific enolase (NSE), NY-ESO, cancer-testis antigen NY-ESO-1, an abnormal p53 protein, PAP (prostatic acid phosphatase), PAMA, P-cadherin, placental alkaline phosphatase, PRAIVIE, prostein, prostate stem cell antigen (PSCA), prostate-specific membrane antigen (PSMA), Ral-B, K-Ras (V-Ki-ras2 Kirsten rat sarcoma viral oncogene), an abnormal ras protein, ROR1, SLAMF7/CS1, receptor tyrosine-protein kinases erb-B2,3,4, sperm protein 17 (Sp17), STEAP1 (six-transmembrane epithelial antigen of the prostate 1), synaptophysin, tumor-associated glycoprotein 72 (TAG-72), TALLA-1, TARP (T cell receptor gamma alternate reading frame protein), TEM-8, human telomerase reverse transcriptase (hTERT), TIM-3, TLR4, TRBC1, TRBC2, Trp-p8, thyroglobulin, thyroid transcription factor-1, TYRP1, tyrosinase, vascular endothelial growth factor R2 (VEGF-R2), Vα24, Wilms tumor protein (WT-1), and various pathogen antigen known in the art.

In some embodiments, binding of the ligand or antigen leads to activation of the intracellular signaling domain of a CAR molecule described herein. In some embodiments, activation of the intracellular signaling domain leads to activation of a cell (e.g., a T cell) expressing the CAR molecule described herein. In some embodiments, activation of a cell expressing a CAR molecule described herein (e.g., a T cell activation) promotes cellular functions, such as functions to regulate a target cell. In some embodiments, activation of a T cell expressing a CAR molecule described herein promotes T cell functions, such as inhibiting and/or killing a target cell expressing at least one ligand or antigen specifically recognized by the CAR molecule, as well as a target cell recognizable by the CAR molecule through an adaptor molecule. In some embodiments, the target cell described herein is a cell in a microenvironment of a biological sample. In some embodiments, the target cell is a cell in a microenvironment of a disease or disorder (e.g., cancer). In some embodiments, the target cell is correlated to a disease or disorder. Exemplary diseases or disorders may include, e.g., cancers, hematological malignancies, solid tumors, autoimmune diseases, inflammations, allergic diseases, infections, senescence/aging, etc. In some embodiments, the target cell is a cancer cell (e.g., a tumor cell, a solid tumor cell, etc.).

In some embodiments, the transmembrane (TM) domain described herein is derived from a transmembrane domain of CD4, CD8, CD28, PD-1, OX40, 4-1BB, CTLA-4, or CD2. In some embodiments, the transmembrane (TM) domain described herein has a transmembrane domain of CD4, CD8, or CD28. In some embodiments, the transmembrane (TM) domain described herein is derived from a transmembrane domain of CD28, CD8, CD4, CD3, CTLA-4, OX40, 4-1BB, CD2, PD-1, CD3D, CD3E, CD3G, CD3zeta, CD8a, CD8b, CD16, CD25, CD27, CD40, CD79A, CD79B, CD80, CD84, CD86, CD95, CD150 (SLAMF1), CD166, CD200R, CD223 (AG3), CD270 (HVEM), CD272 (BTLA), CD273 (PD-L2), CD274 (PD-L1), CD278 (ICOS), CD300, CD357 (GITR), A2aR, ICAM-1, 2B4, BTLA, DAP10, FcRα, FcRβ, Fyn, GAL9, IL7, IL12, IL15, KIR, KIR2DL4, KIR2DS1, LAG-3, Lck, LAT, LPA5, LRP, NKp30, NKp44, NKp46, NKG2C, NKG2D, NOTCH1, NOTCH2, NOTCH3, NOTCH4, PTCH2, ROR2, Ryk, SLP-76, SIRPα, pTα, T cell receptor polypeptides (e.g., TCRα and TCRβ), TIM3, TRIM, and ZAP70.

In some embodiments, the CAR polypeptide described herein further has a hinge domain. In some embodiments, the hinge domain is derived from a hinge domain of CD8, CD28, CD4, IgG (e.g., IgG4), PD-1, CTLA-4, or CD2. In some embodiments, the hinge domain is a hinge domain of CD8, CD28, CD4, or IgG (e.g., IgG4). In some embodiments, the hinge domain described herein is derived from a CD8 hinge domain, a CD28 hinge domain, a CD4 hinge domain, a PD-1 hinge domain, a CD2 hinge domain, a CTLA4 hinge domain, an IgG4 hinge domain, a human CD8α (a.k.a. CD8a), LFA-1 (CD11a/CD18), CD5, CD27 (TNFRSF7), CD70, 4-1BB, OX40 (CD134), ICOS (CD278), IgG1 Fc region, IgG2 Fc region, IgG3 Fc region, IgG4 Fc region, IgE Fc region, IgM Fc region, IgA Fc region, or a combination thereof.

In some embodiments, the CAR polypeptide described herein further has a costimulatory domain. In some embodiments, the costimulatory domain is derived from a costimulatory domain of CD28, ICOS (CD278), CD27, 4-1BB (CD137), OX40 (CD134), CD2, CD4, CD5, CD7, CD8, CD8α, CD8β, CD11a, CD11b, CD11c, CD11d, CD18, CD19, CD19a, CD29, CD30, CD30L, CD40, CD40L (CD154), CD48, CD49a, CD49D, CD49f, CD58, CD53, ICAM-1 (CD54), CD69, CD70, CD80 (B7-1), CD82, CD83, CD84, CD86 (B7-2), CD90, CD96, CD100, CD103, CD122, CD132, CD150 (SLAMF1), CD160 (BY55), CD162 (DNAM1), CD223 (LAG3), CD226, CD229, CD244, CD270 (HVEM), CD273 (PD-L2), CD274 (PD-L1), CD278, LAT, lymphocyte function-associated antigen-1 (LFA-1), LIGHT, NKG2C, NKG2D, NKp30, NKp44, NKp46, NKp80 (KLRF1), B7-H2, B7-H3, CD83 ligand, PD-1, SLP-76, Toll-like receptors (TLRs, such as TLR2), DAP10, DAP12, LAG-3, 2B4, CARD1, CTLA-4 (CD152), TRIM, ZAP70, FcERIγ, 4-1BBL, BAFF, GADS, GITR, GITR-L, BAFF-R, HVEM, CD27L, OX40L, TAC1, BLAME, CRACC, CD2F-10, NTB-A, integrin α4, integrin α4β1, integrin α4β7, IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LTBR, PAG/Cbp, PSGL1, SLAMF6 (NTB-A, Ly108), SLAMF7, SLP-76, TNFR2, TRANCE/RANKL, VLA1, VLA-6, BTLA, ikaros, LAG-3, LMIR, CEACAM1, CRTAM, TCL1A, DAP12, TIM-1, Dectin-1, PDCD6, PD-1, TIM-4, TSLP, EphB6, TSLP-R, HLA-DR, or any combination thereof. In some embodiments, the costimulatory domain is a costimulatory domain of CD28, ICOS, CD27, 4-1BB, OX40, or CD40L.

In some embodiments, the CAR polypeptide described herein further has at least one mutation from a wild type (e.g., naturally occurring) sequence in at least one of domains described herein. In some embodiments, of the at least one mutation is selected from the mutations listed in the below section titled “Mutations to chimeric antigen receptors (CARs)”.

In some embodiments, the CAR polypeptide described herein has an amino acid sequence having at least 20%, 25%, 30%, 35%, 40%, 45%, 50%, 55%, 60%, 65%, 70%, 75%, 80%, 85%, 90%, 95%, 99%, or more identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, the CAR polypeptide described herein has an amino acid sequence having at least 50%, 60%, 70%, 75%, 80%, 90%, 95%, 99%, or more identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, the CAR polypeptide described herein has an amino acid sequence of any one of SEQ ID NOs: 27-49, 111-123, 148-151, 162, 164, 166, 168, 170-176, 178, 180-184, 186, 187, 189, 190, 192, 194, 196, and 198-204. In some embodiments, the CAR polypeptide described herein consists of an amino acid sequence of any one of SEQ ID NOs: 27-49, 111-123, 148-151, 162, 164, 166, 168, 170-176, 178, 180-184, 186, 187, 189, 190, 192, 194, 196, and 198-204.

In some embodiments, the CAR polypeptide described herein is capable of activating a cell expressing the CAR polypeptide. In some embodiments, the cell is an immune cell. In some embodiments, the immune cell is a T cell, a regulatory T cell (Treg), a natural killer (NK) cell, a macrophage, a monocyte, a stem cell, a natural killer T (NKT) cell, a gamma delta T cell, or an induced pluripotent stem cell (iPSC)-derived T cell. In some embodiments, the cell is a non-immune cell.

In some embodiments, activation of the CAR polypeptide increases cytokine production in a T cell expressing the CAR polypeptide. In some embodiments, the cytokine includes IL-2, TNF-α, and/or IFN-γ. For example, cytokine production may be increased by at least 10%, 20%, 30%, 40%, 50%, 60%, 70%, 80%, 90%, 100%, 150%, 200%, 250%, 3-fold, 5-fold, 10-fold, 20-fold, 50-fold, 100-fold, 200-fold, 500-fold, 1000-fold, or more that a control T cell not expressing the CAR polypeptide or a control T cell expressing a CAR molecule containing CD3zeta.

In some embodiments, the CAR polypeptide described herein is capable of reducing T cell exhaustion, compared to a CAR polypeptide containing CD3zeta. Such reduction may be at most 90%, 80%, 70%, 60%, 50%, 40%, 30%, 20%, 10%, 5%, 1%, 0.5%, 0.1%, or less that the T cell exhaustion in a control T cell expressing a CAR molecule containing CD3zeta. In some embodiments, the CAR polypeptide described herein is capable of reducing T cell exhaustion while further maintaining its efficacy/potency. In some embodiments, the reduction of T cell exhaustion is in comparison to the level of T cell exhaustion when the T cell expresses a traditional CAR polypeptide having a different intracellular signaling domain (e.g., CD3zeta). For example, T cell exhaustion may be reduced to 90%, 80%, 70%, 60%, 50%, 40%, 30%, 20%, 10%, 5%, 1%, or less than a control T cell not expressing the CAR polypeptide described herein. The levels of T cell exhaustion may be represented by any methods known in the art, such as expression of surface markers, the levels of cytokine production by the T cell in the absence of tumor or target cells and/or the cytotoxicity of the T cell. In some embodiment, reducing T cell exhaustion by the CAR polypeptide described herein results in an increase of cytokine production by the T cell and/or the cytotoxicity of the T cell, and/or a prolonged timeframe for the T cell to maintain certain levels of cytokine production and/or cytotoxicity.

In another aspect, provided herein is a polynucleotide encoding the chimeric antigen receptor (CAR) polypeptide described herein.

In another aspect, provided herein is an expression vector containing the polynucleotide described herein. In some embodiments, the polynucleotide is a recombinant and/or isolated polynucleotide.

In another aspect, provided herein is a cell expressing the CAR polypeptide described herein. In some embodiments, the cell is an immune cell. In some embodiments, the immune cell is a T cell, a regulatory T cell (Treg), a natural killer (NK) cell, a macrophage, a monocyte, a stem cell, a natural killer T (NKT) cell, a gamma-delta T cell, an induced pluripotent stem cell (iPSC)-derived NK cell, or an induced pluripotent stem cell (iPSC)-derived T cell. In some embodiments, the cell is a non-immune cell.

In another aspect, provided herein is a composition containing the CAR polypeptide, a polynucleotide encoding the CAR polypeptide, an expression vector, and/or a cell, as described herein. In some embodiments, the cell is a regulatory T cell (Treg).

In another aspect, provided herein is a pharmaceutical composition containing the CAR polypeptide, a polynucleotide encoding the CAR polypeptide, an expression vector, a cell, and/or a composition as described herein, plus a pharmaceutically acceptable carrier.

In another aspect, provided herein is a method of preparing or producing the CAR polypeptide described herein. For example, the method may include introducing a polynucleotide or an expression vector described herein into a cultured cell and inducing expression of the CAR polypeptide under a condition. In some embodiments, the method further includes purifying and/or separating the produced CAR polypeptide from the cultured cell. In some embodiments, the cultured cell is a regulatory T cell (Treg).

In another aspect, provided herein is a method for selectively activating a cell including contacting the cell with a ligand, wherein the cell expresses the CAR polypeptide, as described herein, wherein the binding of the ligand to the extracellular ligand-binding domain activates the intracellular signaling domain of the CAR polypeptide, thereby activating the cell. In some embodiments, the cell is a regulatory T cell (Treg).

In another aspect, provided herein is a method of antagonizing or killing a target cell including contacting the target cell with a cell expressing the CAR polypeptide described herein, wherein the target cell expresses or specifically recognizes the ligand recognized by the CAR polypeptide, wherein binding of the ligand to the extracellular ligand-binding domain of the CAR polypeptide activates the cell expressing the CAR polypeptide to antagonize or kill the target cell. In some embodiments, the target cell is a cancer cell. In some embodiments, the target cell is a cell correlated to a disease or disorder, such as proliferative diseases (e.g., cancers), hematological malignancies, solid tumors, autoimmune diseases, inflammations, allergic diseases, infections, senescence/aging, etc. In some embodiments, the cell is a regulatory T cell (Treg).

In another aspect, provided herein is a method of treating a subject having a disease or disorder, including administering to the subject a pharmaceutically effective amount of cells expressing a CAR polypeptide described herein, wherein a target cell correlated to the disease or disorder in the subject express the ligand on the surface, wherein the binding of the ligand to the extracellular ligand-binding domain of the CAR polypeptide activates the cell expressing the CAR polypeptide to antagonize or kill the target cell. In some embodiments, the target cell is a cancer cell. In some embodiments, the target cell is a cell correlated to a disease or disorder, such as proliferative diseases (e.g., cancers), hematological malignancies, solid tumors, autoimmune diseases, inflammations, allergic diseases, infections, senescence/aging, etc. In some embodiments, the cell expressing the CAR polypeptide is a regulatory T cell (Treg).

In another aspect, provided herein is a composition containing at least two CAR polypeptides described herein. In some embodiments, the at least two CAR polypeptides collaborate to transfer signaling from binding to extracellular antigen(s) to activating the intracellular downstream signaling cascade. For example, the at least two CAR polypeptides may bind to different antigens and/or have different intracellular signaling domains. In some embodiments, a cell expressing the at least two CAR polypeptides is activated only when the extracellular ligand-binding domain of each of the at least two CAR polypeptides binds to its specific ligand (e.g., Boolean logic AND gates). Binding of the extracellular ligand-binding domain of one CAR polypeptide, in a CAR combination containing two CAR polypeptides (e.g., an AND gate), to its specific ligand may or may not activate the CAR polypeptide itself. Only binding of both extracellular ligand-binding domains of the two CAR polypeptides to their specific ligands, synergically activates a cell expressing the two CAR polypeptides.

In another aspect, provided herein is a composition containing
i) a first chimeric antigen receptor (CAR) polypeptide having:
   a) a first extracellular ligand-binding domain having a binding affinity for a first ligand;
   b) a first transmembrane domain; and
   c) a first intracellular signaling domain,
      and
ii) a second chimeric antigen receptor (CAR) polypeptide having:
   a) a second extracellular ligand-binding domain having a binding affinity for a second ligand different from the first ligand;
   b) a second transmembrane domain; and
   c) a second intracellular signaling domain,
wherein a cell expressing both CAR polypeptides is activated only when the first extracellular ligand-binding domain binds to the first ligand and the second extracellular ligand-binding domain binds to the second ligand, and
wherein neither of the first and the second intracellular signaling domain has an ITAM, such as CD3zeta.

Non-limiting exemplary embodiments of the disclosed composition of chimeric antigen receptor (CAR) polypeptides of the disclosure include one or more of the following features. In some embodiments, at least one of the first and the second intracellular signaling domains has a full-length or biologically active fragment of a protein kinase, a G protein, a GTP-binding protein, an adaptor signaling protein, or a scaffold protein capable of inducing cell activation. In some embodiments, neither of the first and the second intracellular signaling domain is a CD3ζ domain. In some embodiments, at least one of the first and the second intracellular signaling domains is selected from the group consisting of: LAT, SLP-76, CD28, CD2, 4-1BB, CD6, and a biologically active fragment, mutant or variant thereof. In some embodiments, at least one of the first and the second intracellular signaling domains is LAT or SLP-76, or a biologically active fragment, mutant or variant thereof. In some embodiments, the first intracellular signaling domain is LAT or a biologically active fragment, mutant or variant thereof, and the second intracellular signaling domain is SLP-76 or a biologically active fragment, mutant or variant thereof. In some embodiments, the first intracellular signaling domain is LAT or a biologically active fragment, mutant or variant thereof, and the second intracellular signaling domain is CD28 or a biologically active fragment, mutant or variant thereof. In some embodiments, the biologically active fragment, mutant, or variant thereof is a mutant of LAT, SLP-76, CD28, CD2, 4-1BB, or CD6, wherein the mutant has at least one mutation or deletion to the corresponding wild-type sequence. In some embodiments, the at least one mutation
i) enhances the potency of the composition;
ii) reduces the background activation levels of the cell when only one of the first and the second intracellular signaling domains is activated;
iii) reduces aggregation of the first and the second CAR polypeptides in absence of the ligand;
iv) reduces ubiquitination and/or degradation of the first and/or the second CAR polypeptides; and/or
v) reduces the ability of at least one of the CAR polypeptides to bind to GADS and/or GRB2. In some embodiments, the at least one mutation contains
i) a mutation of G160D, Y200F, Y220F, Y252F, Y200F/Y220F, or Y200F/Y220F/Y252F, a deletion of amino acid residues at the C terminus (e.g., positions 200-262), a deletion of amino acid residues at positions 28-90, a deletion of amino acid residues at positions 28-130, deletions of amino acid residues at positions 28-90 and at positions 200-262, or deletions of amino acid residues at positions 28-130 and at positions 200-262, corresponding to the wild-type LAT sequence;
ii) a mutation of K30R, a deletion of amino acid residues (e.g., positions 224-244), a deletion of amino acid residues at positions 1-81, a deletion of amino acid residues at positions 224-265, a deletion of amino acid residues at positions 224-300, or deletions of amino acid residues at positions 1-81 and at positions 224-244, 224-265, or 224-300, corresponding to the wild-type SLP-76 sequence; and/or
iii) at least one mutation in a region on the first and/or the second CAR polypeptide (or each of the CAR polypeptides in an “AND” gate combination) capable of binding to GADS and/or GRB2.

In some embodiments, at least one of the first and the second intracellular signaling domains described herein has an amino acid sequence having at least 20%, 25%, 30%, 35%, 40%, 45%, 50%, 55%, 60%, 65%, 70%, 75%, 80%, 85%, 90%, 95%, 99%, or more identity to any one of SEQ ID NOs: 11-26, 103-110, 163, 165, 167, 169, 177, 179, 185, 188, 191, 193, and 195. In some embodiments, at least one of the first and the second intracellular signaling domains has an amino acid sequence having at least 50%, 60%, 70%, 75%, 80%, 90%, 95%, 99%, or more identity to any one of SEQ ID NOs: 11-26, 103-110, 163, 165, 167, 169, 177, 179, 185, 188, 191, 193, and 195. In some embodiments, at least one of the first and the second intracellular signaling domains has an amino acid sequence of any one of SEQ ID NOs: 11-26, 103-110, 163, 165, 167, 169, 177, 179, 185, 188, 191, 193, and 195. In some embodiments, at least one of the first and the second intracellular signaling domains consists of an amino acid sequence of any one of SEQ ID NOs: 11-26, 103-110, 163, 165, 167, 169, 177, 179, 185, 188, 191, 193, and 195.

In some embodiments, at least one of the first and the second extracellular ligand-binding domains is a ligand-binding domain of an antibody, an antigen-binding fragment, an antibody mimetic, a receptor, a peptide specifically binding to a ligand or antigen, or a ligand for a targeted receptor. In some embodiments, the antibody or the antigen-binding fragment is selected from the group consisting of a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a VH domain, a VL domain, an Fv fragment, a VNAR domain, and a VHH domain. In some embodiments, the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof. In some embodiments, the receptor described herein is NKG2D, or a biologically active fragment thereof. In some embodiments, the ligand for a targeted receptor is an IL-13 polypeptide, an IL-13 mutein, chlorotoxin, or a biologically active fragment thereof. In some embodiments, at least one of the first and the second extracellular ligand-binding domains is multivalent (e.g., bivalent). In some embodiments, at least one of the first and the second extracellular ligand-binding domains is multispecific (e.g., bispecific).

In some embodiments, at least one of the first and the second ligands (or antigens) localizes on the surface of a target cell. In some embodiments, the target cell is a cell correlated to a disease or disorder, such as proliferative diseases (e.g., cancers), hematological malignancies, solid tumors, autoimmune diseases, inflammations, allergic diseases, infections, senescence/aging, etc. In some embodiments, the cell is a cancer cell. In some embodiments, at least one of the first and the second ligands (or antigens) is an adaptor molecule. In some embodiments, the adaptor is specifically recognized by a target cell described herein (e.g., a cancer cell). Non-limiting examples of suitable types for at least one of the first and the second ligands include cell surface receptors, adhesion proteins, carbohydrates, lipids, glycolipids, lipoproteins, and lipopolysaccharides that are surface-bound, integrins, mucins, and lectins. In some embodiments, at least one of the first and the second ligands (or antigens) is a protein. In some embodiments, at least one of the first and the second ligands (or antigens) is a carbohydrate. In some embodiments, at least one of the first and the second ligands (or antigens) is selected from the group consisting of: CD19, HER2, ROR1, B7-H3 (CD276), influenza hemagglutinin (HA), CD22, CD2, CD5, CD6, 4-1BB, FcγR1, and integrins. In some embodiments, at least one of the first and the second ligands (or antigens) is selected from the group consisting of: CD1a, CD1b, CD1c, CD2, CD3, CD4, CD5, CD6, CD7, CD8, CD9, CD10, CD11a, CD11b, CD11c, CD12, CD13, CD14, CD15 (SSEA-1), CD16 (FcγRIII), CD17, CD18, CD19, CD20, CD21, CD22, CD23, CD24, CD25, CD26, CD27, CD28, CD29, CD30, CD31, CD32 (FcγRII), CD33, CD34, CD35, CD36, CD37, CD38, CD39, CD40, CD41, CD43, CD44, CD44V6, CD45, CD45R/B220, CD45RO, CD49b, CD49d, CD49f, CD52, CD53, CD54, CD56 (NCAM), CD57, CD61 (integrin β3), CD62L, CD63, CD64, CD66b, CD68, CD69, CD70, CD73, CD74, CD79a (Igα), CD79b (Igβ), CD80, CD83, CD85k (ILT3), CD86, CD88, CD93 (C1Rqp), CD94, CD95, CD99, CD103, CD105 (Endoglin), CD107a, CD107b, CD114 (G-CSFR), CD115, CD117, CD122, CD123, CD129, CD133, CD134, CD138 (Syndecan-1), CD141, CD146, CD152 (CTLA-4), CD158 (Kir), CD161 (NK-1.1), CD163, CD183, CD191, CD193 (CCR3), CD194 (CCR4), CD195 (CCR5), CD197 (CCR7), CD203c, CD205 (DEC-205), CD207 (Langerin), CD209 (DC-SIGN), CD223, CD235, CD244 (2B4), CD252 (OX40L), CD267, CD268 (BAFF-R), CD273 (B7-DC, PD-L2), CD276 (B7-H3), CD279 (PD1), CD282 (TLR2), CD284 (TLR4), CD294, CD304 (Neuropilin-1), CD305, CD314 (NKG2D), CD319 (CRACC), CD326, CD328 (Siglec-7), CD335 (NKp46), fetal acetylcholine receptor (AChR), ADGRE2, alpha-fetoprotein (AFP), ALK, BCMA, BDCA3, C3AR, Lewis A (CA19.9), carbonic anhydrase IX (CA1X), calretinin, cancer antigen-125 (CA-125), CCR1, CCR4, CDS, carcinoembryonic antigen (CEA), chromogranin, CLEC12A, an antigen of a cytomegalovirus (CMV) infected cell (e.g., a cell surface antigen), CS-1, CSPG4, cytokeratin, desmin, DLK1, DLL3, EGFRvIII (epidermal growth factor variant III), EGFR and isovariants thereof, epithelial cell adhesion molecule (EpCAM), epithelial glycoprotein2 (EGP 2), epithelial glycoprotein-40 (EGP-40), epithelial membrane protein (EMA), ERBB, epithelial tumor antigen (ETA), FAP, folate-binding protein (FBP), FcγR1, FcεRIα, FITC, FLT3, FOLR1, FOLR3, galactin, gangliosides, gross cystic disease fluid protein (GCDFP-15), GD2 (ganglioside G2), GD3, GM2, GM3, glial fibrillary acidic protein (GFAP), gpA33, glycopeptides, Glypican 2 (GPC2), oncofetal antigen (h5T4), influenza hemagglutinin (HA), human epidermal growth factor receptor 2 (Her2/neu), HLA-DR, HM1.24, HMB-45 antigen, HPV E6, HPV E7, ICAM-1, IgG, IgD, IgE, IgM, IL-13-receptor alpha 1, integrins, Integrin B7, Interleukin-13 receptor subunit alpha-2 (IL-13Rα2), Kappa light chain, kinase insert domain receptor (KDR), Lamba light chain, LILRB2, Lewis Y (LeY), LGR5, Ly49, Ly108, L1 cell adhesion molecule (L1-CAM), melanoma-associated antigen (MAGE), melanoma antigen family A 1 (MAGE-A1), protein melan-A (melanoma antigen recognized by T lymphocytes; MART-1), MCSP, c-Met, MICA/B, mesothelin, muscle-specific actin (MSA), Mesothelin (MSLN), the dimeric form of the pyruvate kinase isoenzyme type M2 (tumor M2-PK), Mucin 1 (Muc-1), Mucin 16 (Muc-16), myo-D1, Ned-2, neurofilament, NKCSI, NKG2D, neuron-specific enolase (NSE), NY-ESO, cancer-testis antigen NY-ESO-1, an abnormal p53 protein, PAP (prostatic acid phosphatase), PAMA, P-cadherin, placental alkaline phosphatase, PRAIVIE, prostein, prostate stem cell antigen (PSCA), prostate-specific membrane antigen (PSMA), Ral-B, K-Ras (V-Ki-ras2 Kirsten rat sarcoma viral oncogene), an abnormal ras protein, ROR1, SLAMF7/CS1, receptor tyrosine-protein kinases erb-B2,3,4, sperm protein 17 (Sp17), STEAP1 (six-transmembrane epithelial antigen of the prostate 1), synaptophysin, tumor-associated glycoprotein 72 (TAG-72), TALLA-1, TARP (T cell receptor gamma alternate reading frame protein), TEM-8, human telomerase reverse transcriptase (hTERT), TIM-3, TLR4, TRBC1, TRBC2, Trp-p8, thyroglobulin, thyroid transcription factor-1, TYRP1, tyrosinase, vascular endothelial growth factor R2 (VEGF-R2), Vα24, Wilms tumor protein (WT-1). In some embodiments, activation of both of the first and the second intracellular signaling domains, or binding of both of the first and the second extracellular ligand-binding domains to their specific ligands, promotes repression and/or killing of a target cell (e.g., a cancer cell) expressing the first and the second ligands, as described herein. In some embodiments, the target cell described herein is a cell in a microenvironment of a biological sample. In some embodiments, the target cell is a cell in a microenvironment of a disease or disorder (e.g., cancer). In some embodiments, the target cell is a cell correlated to a disease or disorder, such as proliferative diseases (e.g., cancers), hematological malignancies, solid tumors, autoimmune diseases, inflammations, allergic diseases, infections, senescence/aging, etc. In some embodiments, the target cell is a cancer cell (e.g., a tumor cell, a solid tumor cell, etc.).

In some embodiments, at least one of the first and the second transmembrane (TM) domains described herein is derived from a transmembrane domain of: CD4, CD8, CD28, PD-1, OX40, 4-1BB, CTLA-4, or CD2. In some embodiments, at least one of the first and the second transmembrane (TM) domains described herein is a transmembrane domain of: CD4, CD8, CD28, PD-1, OX40, 4-1BB, CTLA-4, or CD2. In some embodiments, at least one of the first and the second transmembrane (TM) domains described herein is or is derived from a TMD of CD3D, CD3E, CD3G, CD3zeta, CD8a, CD8b, CD16, CD25, CD27, CD40, CD79A, CD79B, CD80, CD84, CD86, CD95, CD150 (SLAMF1), CD166, CD200R, CD223 (LAG3), CD270 (HVEM), CD272 (BTLA), CD273 (PD-L2), CD274 (PD-L1), CD278 (ICOS), CD300, CD357 (GITR), A2aR, ICAM-1, 2B4, BTLA, DAP10, FcRα, FcRβ, Fyn, GAL9, IL7, IL12, IL15, KIR, KIR2DL4, KIR2DS1, LAG-3, Lck, LAT, LPA5, LRP, NKp30, NKp44, NKp46, NKG2C, NKG2D, NOTCH1, NOTCH2, NOTCH3, NOTCH4, PTCH2, ROR2, Ryk, SLP-76, SIRPα, pTα, T cell receptor polypeptides (e.g., TCRα and TCRβ), TIM3, TRIM, or ZAP70.

In some embodiments, the first transmembrane domain and the second transmembrane domain are different. In some embodiments, using different transmembrane domains for the first and the second CAR polypeptides reduces background activation levels of the cell when only one of the first and the second intracellular signaling domains is activated. In some embodiments, using different transmembrane domains for the first and the second CAR polypeptides reduces background activation levels of the cell when only one of the first and the second intracellular signaling domains is activated. In some embodiments, the first transmembrane domain and the second transmembrane domain are the same. In some embodiments, the transmembrane domains are mutated to decrease homodimerization or heterodimerization with other CAR receptors on the same cell. In some embodiments, the transmembrane domains are mutated at any Cysteines (e.g., to Alanine or another amino acid) to decrease homodimerization or heterodimerization with other CAR receptors on the same cell. In some embodiments, a pair of the first transmembrane domain and the second transmembrane domain, if different, is selected from the group consisting of: CD4 TM and CD28 TM, CD8 TM and CD28 TM, CD4 TM and CD8 TM, and other combinations of TM domains described herein. In some embodiments, the first CAR polypeptide has a CD4 transmembrane domain and the second CAR polypeptide has a CD8 or CD28 hinge/transmembrane domain. In some embodiments, a pair of the first and the second CAR polypeptides contains a CAR polypeptide having LAT as its intracellular signaling domain and a CAR polypeptide having SLP-76 as its intracellular signaling domain. LAT and/or SLP-76 in these CAR polypeptides may have wild-type sequence(s) or at least one variation (e.g., mutation, deletion, etc.) to the corresponding wild-type sequence(s), as described herein. The LAT CAR and/or the SLP-76 CAR polypeptide(s) may further have at least one additional intracellular signaling domain, as described herein, in the polypeptide(s). The LAT CAR and/or the SLP-76 CAR polypeptide(s) may have at least one ECD, hinge domain, TMD, and/or extracellular spacer domain, as described herein in various sections.

In some embodiments, at least one of the first and the second CAR polypeptides described herein further contains a hinge domain. In some embodiments, the hinge domain is derived from a hinge domain of CD8, CD28, CD4, IgG (e.g., IgG4), PD-1, CTLA-4, or CD2. In some embodiments, the hinge domain is a hinge domain of CD8, CD28, CD4, IgG (e.g., IgG4), PD-1, CTLA-4, or CD2. In some embodiments, the hinge domain can include regions derived from or being a human CD8α (a.k.a. CD8a), LFA-1 (CD11a/CD18), CD5, CD27 (TNFRSF7), CD70, 4-1BB, OX40 (CD134), ICOS (CD278), IgG1 Fc region, IgG2 Fc region, IgG3 Fc region, IgG4 Fc region, IgE Fc region, IgM Fc region, IgA Fc region, or a combination thereof. In some embodiments, the first and the second CAR polypeptides further have a same hinge domain. In some embodiments, the first and the second CAR polypeptides further have different hinge domains. For example, the first and the second CAR polypeptides may each have a hinge domain, as described herein, which is different from each other, or only one of the first and the second CAR polypeptides further has a hinge domain. In some embodiments, a pair of the hinge domains of the first and the second CAR polypeptides, if different, is selected from the group consisting of: CD8 hinge domain and CD28 hinge domain, CD4 hinge domain and IgG4 hinge domain, CD8 hinge domain and IgG4 hinge domain, and CD28 hinge domain and IgG4 hinge domain. In some embodiments, using different hinge domains for the first and the second CAR polypeptides reduces background activation levels of the cell when only one of the first and the second intracellular signaling domains is activated, or only one of the first and the second extracellular ligand-binding domains binds to its specific ligand. In some embodiments, at least one of the first and the second CAR polypeptides described herein has a mutation in the hinge/transmembrane domain. In some embodiments, the mutation reduces aggregation of the first and the second CAR polypeptides in absence of at least one of the ligands for the first and the second CAR polypeptides. In some embodiments, the hinge domains are mutated to decrease homodimerization or heterodimerization with other CAR receptors on the same cell. In some embodiments, the hinge domains are mutated at any Cysteines (e.g., to Alanine or another amino acid) to decrease homodimerization or heterodimerization with other CAR receptors on the same cell. In some embodiments, a pair of the hinge and transmembrane (H/TM) domains of the first and the second CAR polypeptides, if different, contains at least two H/TM domains as described herein, e.g., in Tables 1-2 and sequence listing.

In some embodiments, at least one of the first and the second CAR polypeptides described herein further contains a co-stimulatory domain. In some embodiments, the co-stimulatory domain is derived from a co-stimulatory domain of CD28, ICOS, CD27, 4-1BB, OX40, or CD40L. In some embodiments, the co-stimulatory domain is a costimulatory domain of CD28, ICOS, CD27, 4-1BB, OX40, or CD40L. In some embodiments, the costimulatory domain is or is derived from a co-stimulatory domain of CD28, ICOS (CD278), CD27, 4-1BB (CD137), OX40 (CD134), CD2, CD4, CD5, CD7, CD8, CD8α, CD8β, CD11a, CD11b, CD11c, CD11d, CD18, CD19, CD19a, CD29, CD30, CD30L, CD40, CD40L (CD154), CD48, CD49a, CD49D, CD49f, CD58, CD53, ICAM-1 (CD54), CD69, CD70, CD80 (B7-1), CD82, CD83, CD84, CD86 (B7-2), CD90, CD96, CD100, CD103, CD122, CD132, CD150 (SLAMF1), CD160 (BY55), CD162 (DNAM1), CD223 (LAG3), CD226, CD229, CD244, CD270 (HVEM), CD273 (PD-L2), CD274 (PD-L1), CD278, LAT, lymphocyte function-associated antigen-1 (LFA-1), LIGHT, NKG2C, NKG2D, NKp30, NKp44, NKp46, NKp80 (KLRF1), B7-H2, B7-H3, CD83 ligand, PD-1, SLP-76, Toll-like receptors (TLRs, such as TLR2), DAP10, DAP12, LAG-3, 2B4, CARD1, CTLA-4 (CD152), TRIM, ZAP70, FcERIγ, 4-1BBL, BAFF, GADS, GITR, GITR-L, BAFF-R, HVEM, CD27L, OX40L, TAC1, BLAME, CRACC, CD2F-10, NTB-A, integrin α4, integrin α4β1, integrin α4β7, IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LTBR, PAG/Cbp, PSGL1, SLAMF6 (NTB-A, Ly108), SLAMF7, SLP-76, TNFR2, TRANCE/RANKL, VLA1, VLA-6, BTLA, ikaros, LAG-3, LMIR, CEACAM1, CRTAM, TCL1A, DAP12, TIM-1, Dectin-1, PDCD6, PD-1, TIM-4, TSLP, EphB6, TSLP-R, HLA-DR, or any combination thereof. In some embodiments, the first and the second CAR polypeptides further have a same co-stimulatory domain. In some embodiments, the first and the second CAR polypeptides further have different co-stimulatory domains. For example, the first and the second CAR polypeptides may each have a co-stimulatory domain, as described herein, which is different from each other, or only one of the first and the second CAR polypeptides further has a co-stimulatory domain.

In some embodiments, the composition described herein further has a third CAR polypeptide. In some embodiments, the composition described herein further has a third CAR polypeptide having:
a) a third extracellular ligand-binding domain having a binding affinity for a third ligand different from the first ligand and the second ligand;
b) a third transmembrane domain; and
c) a third intracellular signaling domain,
wherein a cell expressing all three CAR polypeptides is activated only when the first intracellular signaling domain is activated and at least one of the second and the third intracellular signaling domains is activated, or the first extracellular ligand-binding domain binds to the first ligand and at least one of the second and the third extracellular ligand-binding domains binds to the second and/or the third ligand (an “AND AND/OR” gate).

In some embodiments, at least one of three CAR polypeptides in a combination (e.g., an “AND AND/OR” gate) has an intracellular signaling domain as described herein, such as ZAP70, PLCG1, PKC, ITK, NCK, VAV1, GRB2, GADS, SOS1, ADAP, SYK, LYN, PI3K, BLNK, or a biologically active fragment, mutant, or variant thereof. In some embodiments, the second and the third intracellular signaling domains are the same.

In some embodiments, the extracellular ligand-binding domain (a.k.a., extracellular antigen-binding domain) of at least one of three CAR polypeptides in a combination has a ligand-binding domain of an antibody, an antigen-binding fragment, an antibody mimetic, a receptor, a peptide specifically binding to a ligand or antigen, or a ligand for a targeted receptor, as described herein, such as a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a VH domain, a VL domain, an Fv fragment, a VNAR domain, and a VHH domain. In some embodiments, the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof. In some embodiments, the extracellular ligand-binding domain is multivalent (e.g., bivalent). In some embodiments, the extracellular ligand-binding domain is multispecific (e.g., bispecific).

In some embodiments, the ligand or antigen recognized by the extracellular ligand-binding domain of at least one of three CAR polypeptides in a combination localizes on the surface of a target cell. In some embodiments, such ligand or antigen is an adaptor molecule. In some embodiments, the adaptor is specifically recognized by a target cell (e.g., a cancer cell). Non-limiting examples of suitable ligand types include cell surface receptors, adhesion proteins, carbohydrates, lipids, glycolipids, lipoproteins, and lipopolysaccharides that are surface-bound, integrins, mucins, and lectins. In some embodiments, the ligand is a protein. In some embodiments, the ligand is a carbohydrate. In some embodiments, the ligand is at least one selected from the group consisting of: CD19, HER2, ROR1, B7-H3 (CD276), influenza hemagglutinin (HA), CD22, IL13Ra2, CD2, CD5, CD6, FcγR1, integrins, gangliosides, and glycopeptides. In some embodiments, the ligand is at least one selected from the group consisting of: CD1a, CD1b, CD1c, CD2, CD3, CD4, CD5, CD6, CD7, CD8, CD9, CD10, CD11a, CD11b, CD11c, CD12, CD13, CD14, CD15 (SSEA-1), CD16 (FcγRIII), CD17, CD18, CD19, CD20, CD21, CD22, CD23, CD24, CD25, CD26, CD27, CD28, CD29, CD30, CD31, CD32 (FcγRII), CD33, CD34, CD35, CD36, CD37, CD38, CD39, CD40, CD41, CD43, CD44, CD44V6, CD45, CD45R/B220, CD45RO, CD49b, CD49d, CD49f, CD52, CD53, CD54, CD56 (NCAM), CD57, CD61 (integrin β3), CD62L, CD63, CD64, CD66b, CD68, CD69, CD70, CD73, CD74, CD79a (Igα), CD79b (Igβ), CD80, CD83, CD85k (ILT3), CD86, CD88, CD93 (C1Rqp), CD94, CD95, CD99, CD103, CD105 (Endoglin), CD107a, CD107b, CD114 (G-CSFR), CD115, CD117, CD122, CD123, CD129, CD133, CD134, CD138 (Syndecan-1), CD141, CD146, CD152 (CTLA-4), CD158 (Kir), CD161 (NK-1.1), CD163, CD183, CD191, CD193 (CCR3), CD194 (CCR4), CD195 (CCR5), CD197 (CCR7), CD203c, CD205 (DEC-205), CD207 (Langerin), CD209 (DC-SIGN), CD223, CD235, CD244 (2B4), CD252 (OX40L), CD267, CD268 (BAFF-R), CD273 (B7-DC, PD-L2), CD276 (B7-H3), CD279 (PD1), CD282 (TLR2), CD284 (TLR4), CD294, CD304 (Neuropilin-1), CD305, CD314 (NKG2D), CD319 (CRACC), CD326, CD328 (Siglec-7), CD335 (NKp46), fetal acetylcholine receptor (AChR), ADGRE2, alpha-fetoprotein (AFP), ALK, BCMA, BDCA3, C3AR, Lewis A (CA19.9), carbonic anhydrase IX (CA1X), calretinin, cancer antigen-125 (CA-125), CCR1, CCR4, CDS, carcinoembryonic antigen (CEA), chromogranin, CLEC12A, an antigen of a cytomegalovirus (CMV) infected cell (e.g., a cell surface antigen), CS-1, CSPG4, cytokeratin, desmin, DLK1, DLL3, EGFRvIII (epidermal growth factor variant III), EGFR and isovariants thereof, epithelial cell adhesion molecule (EpCAM), epithelial glycoprotein2 (EGP 2), epithelial glycoprotein-40 (EGP-40), epithelial membrane protein (EMA), ERBB, epithelial tumor antigen (ETA), FAP, folate-binding protein (FBP), FcγR1, FcεRIα, FITC, FLT3, FOLR1, FOLR3, galactin, ganlgiosides, gross cystic disease fluid protein (GCDFP-15), GD2 (ganglioside G2), GD3, GM2, GM3, glial fibrillary acidic protein (GFAP), gpA33, glycopeptides, Glypican 2 (GPC2), oncofetal antigen (h5T4), influenza hemagglutinin (HA), human epidermal growth factor receptor 2 (Her2/neu), HLA-DR, HM1.24, HMB-45 antigen, HPV E6, HPV E7, ICAM-1, IgG, IgD, IgE, IgM, IL-13-receptor alpha 1, integrins, Integrin B7, Interleukin-13 receptor subunit alpha-2 (IL-13Rα2), Kappa light chain, kinase insert domain receptor (KDR), Lamba light chain, LILRB2, Lewis Y (LeY), LGR5, Ly49, Ly108, L1 cell adhesion molecule (L1-CAM), melanoma-associated antigen (MAGE), melanoma antigen family A 1 (MAGE-A1), protein melan-A (melanoma antigen recognized by T lymphocytes; MART-1), MCSP, c-Met, MICA/B, mesothelin, muscle-specific actin (MSA), Mesothelin (MSLN), the dimeric form of the pyruvate kinase isoenzyme type M2 (tumor M2-PK), Mucin 1 (Muc-1), Mucin 16 (Muc-16), myo-D1, Necl-2, neurofilament, NKCSI, NKG2D, neuron-specific enolase (NSE), NY-ESO, cancer-testis antigen NY-ESO-1, an abnormal p53 protein, PAP (prostatic acid phosphatase), PAMA, P-cadherin, placental alkaline phosphatase, PRAIVIE, prostein, prostate stem cell antigen (PSCA), prostate-specific membrane antigen (PSMA), Ral-B, K-Ras (V-Ki-ras2 Kirsten rat sarcoma viral oncogene), an abnormal ras protein, ROR1, SLAMF7/CS1, receptor tyrosine-protein kinases erb-B2,3,4, sperm protein 17 (Sp17), STEAP1 (six-transmembrane epithelial antigen of the prostate 1), synaptophysin, tumor-associated glycoprotein 72 (TAG-72), TALLA-1, TARP (T cell receptor gamma alternate reading frame protein), TEM-8, human telomerase reverse transcriptase (hTERT), TIM-3, TLR4, TRBC1, TRBC2, Trp-p8, thyroglobulin, thyroid transcription factor-1, TYRP1, tyrosinase, vascular endothelial growth factor R2 (VEGF-R2), Vα24, Wilms tumor protein (WT-1), and various pathogen antigen known in the art.

In some embodiments, the transmembrane (TM) domain of at least one of three CAR polypeptides in a combination is derived from a transmembrane domain described herein, such as a TMD of CD4, CD8, CD28, PD-1, OX40, 4-1BB, CTLA-4, or CD2. In some embodiments, the transmembrane (TM) domain described herein has a transmembrane domain of CD4, CD8, or CD28.

In some embodiments, at least one of three CAR polypeptides in a combination as described herein further has a hinge domain. In some embodiments, the hinge domain is derived from a hinge domain of CD8, CD28, CD4, IgG (e.g., IgG4), PD-1, CTLA-4, or CD2. In some embodiments, the hinge domain is a hinge domain of CD8, CD28, CD4, or IgG (e.g., IgG4).

In some embodiments, at least one of three CAR polypeptides in a combination described herein further has a costimulatory domain. In some embodiments, the co-stimulatory domain is derived from a co-stimulatory domain of CD28, ICOS (CD278), CD27, 4-1BB (CD137), OX40 (CD134), CD2, CD4, CD5, CD7, CD8, CD8α, CD8β, CD11a, CD11b, CD11c, CD11d, CD18, CD19, CD19a, CD29, CD30, CD30L, CD40, CD40L (CD154), CD48, CD49a, CD49D, CD49f, CD58, CD53, ICAM-1 (CD54), CD69, CD70, CD80 (B7-1), CD82, CD83, CD84, CD86 (B7-2), CD90, CD96, CD100, CD103, CD122, CD132, CD150 (SLAMF1), CD160 (BY55), CD162 (DNAM1), CD223 (LAG3), CD226, CD229, CD244, CD270 (HVEM), CD273 (PD-L2), CD274 (PD-L1), CD278, LAT, lymphocyte function-associated antigen-1 (LFA-1), LIGHT, NKG2C, NKG2D, NKp30, NKp44, NKp46, NKp80 (KLRF1), B7-H2, B7-H3, CD83 ligand, PD-1, SLP-76, Toll-like receptors (TLRs, such as TLR2), DAP10, DAP12, LAG-3, 2B4, CARD1, CTLA-4 (CD152), TRIM, ZAP70, FcERIγ, 4-1BBL, BAFF, GADS, GITR, GITR-L, BAFF-R, HVEM, CD27L, OX40L, TAC1, BLAME, CRACC, CD2F-10, NTB-A, integrin α4, integrin α4β1, integrin α4β7, IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LTBR, PAG/Cbp, PSGL1, SLAMF6 (NTB-A, Ly108), SLAMF7, SLP-76, TNFR2, TRANCE/RANKL, VLA1, VLA-6, BTLA, ikaros, LAG-3, LMIR, CEACAM1, CRTAM, TCL1A, DAP12, TIM-1, Dectin-1, PDCD6, PD-1, TIM-4, TSLP, EphB6, TSLP-R, HLA-DR, or any combination thereof. In some embodiments, the co-stimulatory domain is a co-stimulatory domain of CD28, ICOS, CD27, 4-1BB, OX40, or CD40L.

In some embodiments, at least one of three CAR polypeptides in a combination further has at least one mutation from a wild-type (e.g., naturally occurring) sequence in at least one of domains described herein. In some embodiments, of the at least one mutation is selected from the mutations listed in the below section titled “Mutations to chimeric antigen receptors (CARs)”.

In some embodiments, at least one of CAR polypeptides in the composition described herein has an amino acid sequence having at least 20%, 25%, 30%, 35%, 40%, 45%, 50%, 55%, 60%, 65%, 70%, 75%, 80%, 85%, 90%, 95%, 99%, or more identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, at least one of CAR polypeptides in the composition described herein has an amino acid sequence having at least 50%, 60%, 70%, 75%, 80%, 90%, 95%, 99%, or more identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, at least one of CAR polypeptides in the composition described herein has an amino acid sequence of any one of SEQ ID NOs: 27-49, 111-123, 148-151, 162, 164, 166, 168, 170-176, 178, 180-184, 186, 187, 189, 190, 192, 194, 196, and 198-204. In some embodiments, at least one of CAR polypeptides in the composition described herein consists of an amino acid sequence of any one of SEQ ID NOs: 27-49, 111-123, 148-151, 162, 164, 166, 168, 170-176, 178, 180-184, 186, 187, 189, 190, 192, 194, 196, and 198-204.

In some embodiments, the composition described herein is capable of activating a target cell expressing the first and the second CAR polypeptides. In some embodiments, the target cell is an immune cell. In some embodiments, the immune cell is a T cell, a regulatory T cell (Treg), a natural killer (NK) cell, a macrophage, a monocyte, a stem cell, a gamma delta T cell, a natural killer T (NKT) cell, or an induced pluripotent stem cell (iPSC)-derived T cell. In some embodiments, the target cell is a non-immune cell.

In some embodiments, the composition described herein is capable of reducing T cell exhaustion. For example, T cell exhaustion may be reduced to 90%, 80%, 70%, 60%, 50%, 40%, 30%, 20%, 10%, 5%, 1%, or less than a control T cell not treated with the composition or not expressing both of the first and the second CAR polypeptides in the composition, or a control T cell expressing a CAR molecule containing CD3zeta. The levels of T cell exhaustion may be represented by any methods known in the art, such as the levels of cytokine production by the T cell and/or the cytotoxicity of the T cell. In some embodiments, reducing T cell exhaustion by the composition described herein results in an increase of cytokine production by the T cell and/or the cytotoxicity of the T cell, and/or a prolonged timeframe for the T cell to maintain certain levels of cytokine production and/or cytotoxicity.

In another aspect, some embodiments of the disclosure provide a composition containing a first polynucleotide molecule and a second polynucleotide molecule. In some embodiments, the first polynucleotide molecule encodes the first chimeric antigen receptor (CAR) polypeptide in the composition described herein, and the second polynucleotide molecule encodes the second chimeric antigen receptor (CAR) polypeptide in the composition described herein. In some embodiments, the first and the second polynucleotide molecules are conjugated together. In some embodiments, the first and the second polynucleotide molecules are different parts on a same polynucleotide.

In another aspect, some embodiments of the disclosure provide a composition containing a first polynucleotide molecule, a second polynucleotide molecule, and a third polynucleotide molecule. In some embodiments, a first polynucleotide molecule encodes a first chimeric antigen receptor (CAR) polypeptide in the composition described herein, a second polynucleotide molecule encodes a second chimeric antigen receptor (CAR) polypeptide in the composition described herein, and a third polynucleotide molecule encodes a third chimeric antigen receptor (CAR) polypeptide in the composition described herein. In some embodiments, the first, the second and the third polynucleotide molecules are conjugated together. In some embodiments, the first, the second and the third polynucleotide molecules are different parts on a same polynucleotide.

In another aspect, some embodiments of the disclosure provide a first expression vector and a second expression vector, wherein the first expression vector has the first polynucleotide molecule and the second expression vector has the second polynucleotide molecule. In another aspect, some embodiments of the disclosure provide a first expression vector, a second expression vector and a third expression vector, wherein the first expression vector has the first polynucleotide molecule, the second expression vector has the second polynucleotide molecule and the third expression vector has the third polynucleotide molecule.

In another aspect, some embodiments of the disclosure provide a cell expressing the composition described herein. In some embodiments, the cell is an immune cell. In some embodiments, the immune cell is a T cell, a regulatory T cell (Treg), a natural killer (NK) cell, a macrophage, a monocyte, a stem cell, a natural killer T (NKT) cell, a gamma delta T cell, an induced pluripotent stem cell (iPSC)-derived NK cell, or an induced pluripotent stem cell (iPSC)-derived T cell. In some embodiments, the cell is a non-immune cell.

In another aspect, provided herein is a pharmaceutical composition including the composition containing the first and the second CAR polypeptides described herein, a composition containing the first polynucleotide encoding the first CAR polypeptide and the second polynucleotide encoding the second CAR polypeptide, an expression vector, a cell, and/or a composition as described herein, plus a pharmaceutically acceptable carrier. In some embodiments, the cell is a regulatory T cell (Treg).

In another aspect, provided herein is a method of preparing or producing the first and the second CAR polypeptides in the composition described herein. For example, the method may include introducing a composition containing the first and the second polynucleotides or an expression vector described herein into a cultured cell and inducing expression of the CAR polypeptide under a condition. In some embodiments, the cultured cell is a regulatory T cell (Treg).

In another aspect, provided herein is a method for selectively activating a cell including contacting the cell with first ligand and a second ligand, wherein the cell expresses a composition containing a first and a second CAR polypeptides described herein, wherein binding of the first and the second ligands to the first and the second extracellular ligand-binding domains activates the first and the second intracellular signaling domains, respectively, thereby activating the cell, wherein activation of only one of the first and the second intracellular signaling domains does not activate the cell. In some embodiments, the cell is a regulatory T cell (Treg).

In another aspect, provided herein is a method of antagonizing or killing a target cell including contacting the target cell with a cell expressing a composition containing a first and a second CAR polypeptides described herein, wherein the target cell expresses or specifically recognizes both the first ligand and the second ligand, wherein binding of the first ligand to the first extracellular ligand-binding domain and binding of the second ligand to the second extracellular ligand-binding domain activate the cell expressing the composition to antagonize or kill the target cell. In some embodiments, the target cell is correlated to a disease or disorder. Exemplary diseases or disorders may include, e.g., proliferative diseases (e.g., cancers), hematological malignancies, solid tumors, autoimmune diseases, inflammations, allergic diseases, infections, senescence/aging, etc. In some embodiments, the cell expressing the composition is a regulatory T cell (Treg).

In another aspect, provided herein is a method of treating a subject having a disease or disorder, including administering to the subject a pharmaceutically effective amount of cells expressing a first and a second CAR polypeptides in a composition described herein, wherein a target cell correlated to the disease or disorder in the subject expresses or specifically recognizes both the first ligand and the second ligand, wherein the binding of the first ligand to the first extracellular ligand-binding domain and binding of the second ligand to the second extracellular ligand-binding domain activate the cells expressing the composition to antagonize or kill the target cells. Exemplary diseases or disorders may include, e.g., proliferative diseases (e.g., cancers), hematological malignancies, solid tumors, autoimmune diseases, inflammations, allergic diseases, infections, senescence/aging, etc. In some embodiments, the cell expressing the composition is a regulatory T cell (Treg).

In another aspect, some embodiments of the disclosure provide various kits for the practice of the methods disclosed herein. Some embodiments relate to kits for methods of the diagnosis, prevention, and/or treatment of a condition in a subject in need thereof, wherein the kits include one or more of: a CAR polypeptide of the disclosure; a recombinant nucleic acid of the disclosure; a recombinant cell of the disclosure, and a pharmaceutical composition of the disclosure.

In another aspect, provided herein is the use of one or more of: a CAR polypeptide of the disclosure, a composition of a first and a second CAR polypeptides (or polynucleotides encoding such polypeptides) of the disclosure, a recombinant nucleic acid of the disclosure, a recombinant cell of the disclosure, and a pharmaceutical composition, for the diagnosis, prevention, and/or treatment of a condition. Exemplary conditions may include, e.g., proliferative diseases (e.g., cancers), hematological malignancies, solid tumors, autoimmune diseases, inflammations, allergic diseases, infections, senescence/aging, etc. In some embodiments, the condition is a proliferative disease. In some embodiments, the proliferative disease is a cancer.

In another aspect, provided herein is the use of one or more of the following: a CAR polypeptide of the disclosure, a composition of a first and a second CAR polypeptides (or polynucleotides encoding such polypeptides) of the disclosure, a recombinant nucleic acid of the disclosure, a recombinant cell of the disclosure, or a pharmaceutical composition of the disclosure, in the manufacture of a medicament for the prevention and/or treatment of a health condition. Exemplary conditions may include, e.g., proliferative diseases (e.g., cancers), hematological malignancies, solid tumors, autoimmune diseases, inflammations, allergic diseases, infections, senescence/aging, etc. In some embodiments, the condition is a proliferative disease. In some embodiments, the proliferative disease is a cancer.

In some embodiments, the cell expressing the CAR construct(s) described herein, useful for preparing a composition, or for use in the method described herein, is a regulatory T cell (Treg).

The foregoing summary is illustrative only and is not intended to be in any way limiting. In addition to the illustrative embodiments and features described herein, further aspects, embodiments, objects and features of the disclosure can become fully apparent from the drawings and the detailed description and the claims.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 shows histograms of FACS analyses for expressions of CD19-targeting CAR constructs containing various downstream TCR pathway signaling molecules.

FIG. 2 shows a graph illustrating lack of interleukin-2 (IL-2) generation by most (LCK, FYN, SLP-76, and LAT) TCR signaling molecule constructs in response to antigen exposure.

FIG. 3 shows histograms of FACS analyses for expressions of HER2-targeting CAR constructs containing either a full-length ZAP70 or ZAP70 fragments, demonstrating that only a fragment containing segments from both the interdomain B domain and the kinase domain from ZAP70 (i.e., ZAP70²⁵⁵⁻⁶⁰⁰), but not a full-length ZAP70 or a fragment containing only the kinase domain from ZAP70 (i.e., ZAP70^{kinase}), will express in the HER2-targeting CAR construct in the cell.

FIG. 4 shows a graph illustrating cell activation (detected by interleukin-2 (IL-2) generation) by CAR constructs containing either ZAP70 (“ZAP70²⁵⁵⁻⁶⁰⁰”) or PLC-gamma (“PLCG1”) signaling molecules, but not by CAR constructs containing other proximal signaling molecules, in response to antigen (i.e., CD19 expressed on CD19+HER2+ tumor cells) exposure. For each condition, tested constructs contain, from left to right, LAT, SLP-76, LcK, Fyn, ZAP70²⁵⁵⁻⁶⁰⁰, PLCG1, and mock as control.

FIGS. 5A-5C show several graphs comparing cell activation by different CAR molecules, including CAR molecules containing a truncated-ZAP70 fragment (ZAP70²⁵⁵⁻⁶⁰⁰) as the cytosolic signaling domain, recognizing HER2 (FIG. 5A), B7-H3 (FIG. 5B), or GD2 (FIG. 5C), and traditional CAR molecules containing either CD28-zeta (CD28ζ) or 4-1BB-zeta (4-1BBζ) as the cytosolic signaling domain. The left panel of FIG. 5A shows cytotoxicity index for a HER2-expressing Nalm6 leukemia tumor cell line mixed with T cells expressing these CAR molecules. The right panel of FIG. 5A is a bar chart comparing IL-2 production by T cells expressing these CAR molecules (containing, from left to right in each condition, CD28ζ, 4-1BBζ, or ZAP70²⁵⁵⁻⁶⁰⁰) when exposing to the HER2-expression Nalm6 tumor cell line. FIGS. 5B-5C show in vitro functionality of GD2/B7-H3-targeting CAR T cells bearing CARs with either the 4-1BB-zeta or the ZAP-70²⁵⁵⁻⁶⁰⁰ fragment endodomains. FIG. 5B shows cytokine production by B7-H3-targeting 4-1BB-zeta or ZAP-70²⁵⁵⁻⁶⁰⁰ fragment CAR T cells when co-cultured with tumor cells (left panel) and killing of CHLA-255 neuroblastoma cells by said CAR T cells (right panel). FIG. 5C shows cytokine production (left panel) and killing of neuroblastoma cells (CHLA-255) (right panel) by CAR T cells expressing GD2-targeting 4-1BB-zeta or ZAP-70²⁵⁵⁻⁶⁰⁰ fragment, when co-cultured with tumor cells.

FIGS. 6A-6B show graphs illustrating the expression and function of exemplary CAR constructs containing PLCG1. FIG. 6A shows a cartoon representation of a HER2−28H/TM-PLCG1 CAR construct (top) and graphs illustrating the expression of this construct in T cells (the histogram in the left panel at the bottom), the cytotoxicity of the T cells expressing the HER2−28H/TM-PLCG1 CAR against HER2-expressing tumor cells (bottom, the middle panel), and cytokine (IL-2) generation (bottom, the right panel) by the T cells, demonstrating an activity of this CAR to recognize and kill target cells and to produce cytokine in response to antigen exposure. A similar CAR construct containing PLCG1 and anti-CD19 scFv was prepared and tested, as shown in FIG. 6B, in which the top panel shows the expression of such CAR construct in T cells and the bottom panels show the capacity to induce IL-2 cytokine production upon stimulation with Nalm6 cells [bottom left; the y-axis represents the levels of IL-2 (pg/ml)] and the cytotoxicity (by measuring killing of Nalm6 tumor cells at 2:1 effector:target ratio; bottom right) of such CAR construct expressed on T cells.

FIGS. 7A-7C show an abbreviated depiction of the T cell proximal signaling cascade. FIG. 7A shows that LCK is responsible for phosphorylation of CD3zeta. FIG. 7B shows that ZAP70 docks on phosphorylated CD3zeta and is activated to phosphorylate SLP-76 and LAT. FIG. 7C shows that, once phosphorylated, SLP-76 and LAT come together to form a scaffold for activation of PLCG1, which propagates many downstream effector functions, such as T cell activation.

FIGS. 8A-8B show that a T cell expressing both SLP-76 and LAT CARs, which have different antigen specificities (i.e., Antigen A and Antigen B, respectively), generates a robust response to tumor cells expressing both target antigens. FIG. 8A shows a depiction of the CAR receptor structures. FIG. 8B shows a bar graph comparing the amount of IL-2 produced by T cells expressing either the LAT or SLP-76 CAR construct alone or in combination (“LAT+SLP-76”), when exposed to tumor cells expressing both antigens for the LAT and the SLP-76 CAR constructs.

FIG. 9 shows FACS data for cells expressing several different combinations of signaling molecule CAR constructs. As shown in FIG. 2, each of the constructs alone does not promote IL-2 generation in response to antigen exposure.

FIG. 10 is a bar graph comparing the amount of IL-2 produced by T cells expressing different combinations of CAR molecules as in FIG. 9, when exposed to both antigens for the CAR molecules in combinations.

FIG. 11 shows that LAT and SLP-76 CARs function as an AND-gate to induce T cell activation (i.e., activating cells only when both antigen A and antigen B are engaged) (left). Also shown are cartoon representations of the LAT and SLP-76 CAR constructs (right).

FIG. 12 shows graphs comparing the activity of T cells expressing both LAT and SLP-76 CARs against tumor lines expressing different antigens. In the left panel, a bar graph compares the IL-2 expression levels for T cells expressing both a CD19−CD28H/TM-LAT CAR molecule and a HER2−CD28H/TM-SLP-76 molecule and being exposed to tumor cell lines expressing only CD19 (“CD19+HER2−”), only HER2 (“CD19−HER2+”), or both CD19 and HER2 (“CD19+HER2+”) antigens. Histograms of FACS data are used to confirm the expression of the CAR molecules (the middle panel and the right-side upper and bottom panels).

FIG. 13 shows graphs comparing the IL-2 generation activity of T cells expressing both LAT and SLP-76 CARs when exposed to tumor cell lines expressing different antigens, illustrating that when a LAT CAR and a SLP-76 CAR share a same hinge/transmembrane domain, the system had some background activity against single antigen positive cells. The upper panel shows FACS data for exemplary T cells expressing LAT and SLP-76 CAR molecules with a same CD8 or CD28 hinge/transmembrane domain. The bottom panel is a bar graph comparing IL-2 production of the exemplary T cells exposed to tumor cells expressing no antigen (“CD19−HER2−”), CD19 antigen only (“CD19+HER2−”), HER2 antigen only (“CD19−HER2+”), or both antigens (“CD19+HER2+”). The exemplary T cells had some background activation in response to a single antigen of either CD19 or HER2, which was less than the activation when both antigens were present (“CD19+ER2+”) (the bottom panel).

FIG. 14 is a set of graphs showing expression of several LAT and SLP-76 CAR construct combinations with shared or alternate hinge-transmembrane domains between the constructs on primary human T cells.

FIG. 15 is a graph showing the generation of IL-2 by the T cells in FIG. 14 when exposed to tumor cells expressing no antigen (“CD19−HER2−”), CD19 antigen only (“CD19+HER2−”), HER2 antigen only (“CD19−HER2+”), or both antigens (“CD19+HER2+”). This data demonstrate that there is less background T cell activation in response to single antigen exposure when the LAT and SLP-76 CAR constructs do not share the same transmembrane domain.

FIG. 16 is a set of graphs illustrating cytotoxicity of T cells expressing different LAT CAR molecules alone, including the construct used previously with a CD28 hinge/TM domain and constructs with the H/TM domain swapped with the TM domain from CD4 and the H (hinge) domain from IgG4 or CD4 (see the lower panel on the right for a cartoon of the construct structure). The expression of each LAT CAR molecule was confirmed by histogram of FACS data (upper right panel), demonstrating that the CAR construct containing both CD4 hinge and transmembrane domains did not express well. The cytotoxicity of T cells expressing each of the CD19-targeting CARs containing LAT and being mixed with CD19+HER2− tumor cells at a ratio of 1:1 or 1:2 were plotted through the time (upper left and bottom left panels). In both cytotoxicity plots, the top line represents using the CD19−CD4H/TM-LAT construct, the middle line represents using CD19-IgG4H/CD4TM-LAT, and the bottom line represents using CD19−CD28H/TM-LAT.

FIG. 17 is a set of FACS histograms comparing expression of LAT CARs and SLP-76 CARs with different hinge/TM combinations.

FIG. 18 is a set of graphs illustrating cytotoxicity by the T cells expressing LAT and SLP-76 CARs combinations with different Hinge/TM combinations in FIG. 17. Tumor cells expressing CD19 only (“CD19+HER2−”, the left panel), HER2 only (“CD19−HER2+”, the middle panel), or both CD19 and HER2 (“CD19+HER2+”, the right panel) were used to present antigen to activate T cells. Only the combination containing a LAT CAR containing an IgG4-hinge/CD4 TM (paired with a SLP-76 CAR containing a CD8 hinge/TM domain) showed no background killing activity against single antigen positive cells (the left panel) but a good killing capacity against double antigen positive cells (the right panel).

FIG. 19 is a bar chart illustrating IL-2 production by the T cells in FIG. 18, when exposed to tumor cells not expressing CD19 or HER2 (“19−HER2−”), or tumor cells expressing CD19 only (“19+HER2−”), HER2 only (“19−HER2+”) or both CD19 and HER2 (“19+HER2+”).

FIGS. 20A-20C are a set of graphs showing CAR expression and IL-2 production for each of the LAT/SLP-76 CAR combinations. FIG. 20A is a set of FACS histograms to show CAR expression in T cells. FIG. 20B is a set of histograms illustrating the expressed CAR combinations, including a negative control without expressing CAR combinations (top trace), CD19−CD28H/TM-LAT and HER2−CD8H/TM-SLP-76 (second trace from top), CD19−IgG4H/CD4TM-LAT and HER2−CD8H/TM-SLP-76 (third trace from top), and CD19−IgG4H/CD4TM-LAT and HER2−CD28H/TM-SLP-76 (bottom trace). FIG. 20C is a bar chart comparing IL-2 production by T cells expressing the listed CAR combinations in response to no antigen, single antigen of CD19 or HER2, or CD19 and HER2 antigens. The combination of the LAT CAR with an IgG4-hinge/CD4TM domain and the SLP-76 CAR with a CD28 hinge/TM domain results in no activity against single antigen positive cells but high activation and IL-2 production against double antigen positive cells.

FIG. 21 is a set of graphs illustrating cytotoxicity of T cells expressing the listed LAT and SLP-76 CAR combinations in response to either single or both antigens. The combination of the LAT CAR with an IgG4-hinge/CD4TM domain and the SLP-76 CAR with a CD28 hinge/TM domain results in no activity against single antigen positive cells but strong killing activity against double antigen positive cells.

FIGS. 22A-22C are graphs illustrating T cell activation in response to single or double antigens. FIG. 22A are histograms showing expressed CAR combinations, including CD19−IgG4H/CD4TM-LAT+HER2−CD28H/TM-SLP-76 (the second trace from top), CD19−IgG4H/CD4TM-LAT+HER2−CD28H/TM-SLP-76^{K30R} (in which SLP-76 is mutated at the lysine at position 30 to decrease ubiquitination) (the bottom trace), and a negative control without CAR combinations (“Mock”, the top trace). FIG. 22B are FACS histograms showing the T cells expressing any one of the CAR combinations. FIG. 22C is a bar graph comparing IL-2 production by the T cells in FIG. 22B, demonstrating that the K30R mutation in the SLP-76 CAR construct results in enhanced activity against double antigen positive tumor without increasing activity against tumor cells expressing a single antigen.

FIGS. 23A-23C are graphs illustrating T cell activation in response to single or double antigens. FIG. 23A are histograms showing expressed CAR combinations, including CD19−IgG4H/CD4TM-LAT+HER2−CD28H/TM-SLP-76 (the second trace from top), CD19−IgG4H/CD4TM-LAT^{G160D}+HER2−CD28H/TM-SLP-76 (in which LAT is mutated at the Glycine at position 160 to increase activation) (the bottom trace), and a negative control without CAR combinations (“Mock”, the top trace). FIG. 23B are FACS histograms showing the T cells expressing any one of the CAR combinations. FIG. 23C is a bar graph comparing IL-2 production by T cells in this figure, demonstrating that the G160D mutation in the LAT construct results in enhanced activity against double antigen positive tumor with a minimal increase in activity against tumor cells expressing a single antigen CD19.

FIGS. 24A-24C are a set of graphs comparing T cell activation capacity of various CAR combinations. FIG. 24A is a set of FACS histograms illustrating T cells expressing the listed CAR combinations (e.g., a CAR construct containing LAT and a CAR construct containing CD5, CD6, FcγR1, or 4-1BB). FIG. 24B is a graph showing cytokine production induced by these CAR combinations against single or double antigen positive tumor cells. FIG. 24C is a series of graphs comparing killing of single or double antigen positive tumor cells by these CAR combinations.

FIGS. 25A-25C are a set of graphs comparing T cell activation capacity of various CAR combinations (e.g., a CD2 CAR combined with a LAT CAR with various hinge-transmembrane domains). FIG. 25A is a set of FACS histograms illustrating T cells expressing the listed CAR combinations. FIG. 25B is a graph showing cytokine production induced by these CAR combinations against single or double antigen positive tumor cells. FIG. 25C is a series of graphs showing killing of single or double antigen positive tumor cells by the listed CAR combinations.

FIGS. 26A-26C are a set of graphs comparing T cell activation capacity of various CAR combinations (e.g., a CD28 CAR combined with a LAT CAR with various hinge-transmembrane domains). FIG. 26A is a set of FACS histograms illustrating T cells expressing the listed CAR combinations. FIG. 26B is a graph showing cytokine production induced by the listed CAR combinations against single or double antigen positive tumor cells. FIG. 26C is a series of graphs showing killing of single or double antigen positive tumor cells by the listed CAR combinations.

FIGS. 27A and 27B are a set of graphs comparing T cell activation capacity of various CAR combinations, including a SLP-76 CAR with a CD8 hinge-transmembrane domain combined with either a LAT CAR with a CD28 hinge-transmembrane domain or a LAT CAR with a CD28 hinge-transmembrane domain in which the two cysteine residues have been mutated (2CA). FIG. 27A is a set of FACS histograms illustrating expression of T cells expressing the listed CAR combinations. FIG. 27B is a graph showing cytokine production induced by the listed CAR combinations against single or double antigen positive tumor cells.

FIGS. 28A and 28B are a set of graphs comparing T cell activation capacity of various CAR combinations, including a CAR molecule containing a wild-type LAT or a LAT mutant with mutations in its GADS binding site (“LAT^{2YF}”, containing mutations of Y200F and Y220F), combined with a CAR molecule containing a wild-type SLP-76 or a SLP-76 mutant in which the GADS binding site was deleted (“SLP76^{224-244 del}”) FIG. 28A is a set of FACS histograms illustrating expression of T cells expressing the listed CAR combinations. FIG. 28B is a graph showing cytokine production induced by the listed CAR combinations against single or double antigen positive tumor cells.

FIGS. 29A and 29B are a set of graphs comparing T cell activation capacity of various CAR combinations, including a CAR molecule containing a wild-type LAT or a LAT mutant with a deletion of its GADS and GRB2 binding sites (“LAT^{200-262 del}” with a deletion of amino acids from position 200 to position 262), combined with a CAR molecule containing a wild-type SLP-76 or a SLP-76 mutant in which the GADS binding site was deleted (“SLP76^{224-244del}”). FIG. 29A is a set of FACS histograms illustrating expression of T cells expressing the listed CAR combinations. FIG. 29B is a graph showing cytokine production induced by the listed CAR combinations against single or double antigen positive tumor cells.

FIGS. 30A-30C are a set of graphs comparing T cell activation capacity of various CAR combinations, including a wild-type LAT CAR construct combined with a wild-type SLP-76 CAR construct, or a CAR construct containing a LAT mutant with mutations in its GADS binding site (“LAT^{2YF}”, containing mutations of Y200F and Y220F) combined with a CAR construct containing a SLP-76 mutant in which the GADS binding site was deleted (“SLP76^{224-244del}”). FIG. 30A is a set of FACS histograms illustrating expression of T cells expressing the listed CAR combinations. FIG. 30B is a graph showing cytokine production induced by the listed CAR combinations against single or double antigen positive tumor cells. FIG. 30C is a series of graphs showing killing of single or double antigen positive tumor cells by the listed CAR combinations.

FIGS. 31A-31C are a set of graphs comparing T cell activation capacity of various CAR combinations, including a LAT CAR construct containing a CD28 hinge-transmembrane domain in which the two cysteine residues have been mutated (2CA), combined with a wild-type SLP-76 CAR construct, or a LAT CAR construct containing both the 2CA mutations and the 2YF (i.e., Y200F and Y220F) mutations in its GADS binding sites, combined with a CAR construct containing a SLP-76 mutant in which the GADS binding site was deleted (“SLP76^{224-244 del}”). FIG. 31A is a set of FACS histograms illustrating expression of T cells expressing the listed CAR combinations. FIG. 31B is a series of graphs showing cytokine production induced by the listed CAR combinations against single or double antigen positive tumor cells. FIG. 31C is a series of graphs showing killing of single or double antigen positive tumor cells by the listed CAR combinations. The bottom line in the left panel and the top line in the right panel represent the combination of CD19−28H/TM(2CA)-LAT+HER2−8H/TM-SLP-76.

FIGS. 32A and 32B are a set of graphs comparing T cell activation by variant CAR combinations. FIG. 32A is a set of FACS histograms illustrating T cells expressing CAR combinations, including a CAR construct containing a wild-type SLP-76 or a SLP-76 mutant with a deleted GADS binding site (“SLP76^{224-244 del}”), combined with a CAR construct containing a wild-type LAT or a LAT with mutations in the GADS and GRB2 binding sites (“LAT^{3YF}”, containing Y200F, Y220F and Y252F mutations). FIG. 32B is a graph showing cytokine production induced by the listed CAR combinations against single or double antigen positive tumor cells.

FIGS. 33A-33C are graphs showing that CAR constructs containing a truncated ZAP70 (“ZAP70²⁵⁵⁻⁶⁰⁰”) confer an advantage over traditional CARs when using scFvs prone to tonic signaling. In FIG. 33A, traditional CARs targeting GD2 with a high affinity binder (HA) (top panels) or B7-H3 (the bottom panels) were used, containing either a CD28zeta or a 4-1BBzeta domain. The “CAR” panels show T cell expression of each CAR molecule. The “CD39”/“LAG-3”/“PD-1”/“TIM-3” panels show surface expression of exhaustion markers on CAR-T cells, indicative of T cell exhaustion. In FIG. 33B, a similar experiment was perform as in FIG. 33A, while the used traditional CAR molecules recognize CD19, B7-H3 or GD2 and contain a 4-1BBzeta domain. FIG. 33C exhibits the proportions of LAG-3±, TIM-3±, PD-1±populations in CAR T cells targeting B7-H3 (the top panels) or GD2 (the bottom panels), containing corresponding B7-H3/GD2 CAR constructs with 4-1BB-zeta or ZAP-70²⁵⁵⁻⁶⁰⁰ endodomains. Outside each pie figure, from closer to farther from the pie, curves are used to show cell populations for LAG-3+, TIM-3+ and PD-1+, respectively.

FIGS. 34A-34E are graphs showing the anti-tumor function of T cells expressing different CAR constructs. FIG. 34A shows a histogram showing the expression of B7-H3-targeting CARs, including a traditional CAR construct bearing 4-1BB-zeta endodomains and a CAR construct containing the ZAP70²⁵⁵⁻⁶⁰⁰ fragment. FIG. 34B shows tumor measurements from mice inoculated with luciferase expressing neuroblastoma xenografts (CHLA255) and treated with T cells expressing the traditional B7-H3-4-1BB-zeta CAR or the B7-H3-truncated-ZAP70 CAR. FIG. 34C exhibits tumor measurements (top) and survival (bottom) from mice as treated in FIG. 34B in a longer time frame. FIG. 34D shows the amount of T cells bearing B7-H3-4-1BB-zeta CARs or B7-H3-truncated-ZAP70 CARs harvested from the spleens of mice inoculated with luciferase expressing CHLA-255. FIG. 34E shows the amount of T cells bearing B7-H3-4-1BB-zeta CARs or B7-H3-truncated-ZAP70 CARs harvested from the bone marrow of mice inoculated with luciferase-expressing CHLA-255.

FIG. 35 is a set of graphs showing cytokine generation by T cells expressing different CAR constructs. The top panel is a histogram showing the expression of B7-H3-targeting CARs containing the ZAP70²⁵⁵⁻⁶⁰⁰ fragment with or without further mutations that enhance its efficacy (e.g., Y292F, Y492F, K544R, Y597F, and Y598F). The bottom panel is a bar graph showing IL-2 generation by T cells expressing these CAR constructs in response to antigen encounter with Nalm6 leukemia tumor cells or 143B osteosarcoma cells. The bars for each condition refer to, from left to right, mock, B7-H3-28htm-ZAP-70²⁵⁵⁻⁶⁰⁰, B7-H3-28htm-ZAP-70^{255-600 Y292F}, B7-H3-28htm-ZAP-70^{255-600 Y492F}, B7-H3-28htm-ZAP-70^{255-600 K544R}, and B7-H3-28htm-ZAP-70^{255-600 Y597A Y598A}.

FIG. 36 is a set of graphs showing cytokine generation by T cells expressing different CAR constructs. The top panel is a histogram showing the expression of B7-H3-targeting CARs containing the ZAP70²⁵⁵⁻⁶⁰⁰ fragment with or without endowed costimulatory molecules (4-1BB or CD28). The bottom panel is a bar graph showing IL-2 generation by T cells expressing the CAR constructs in response to antigen encounter with tumor cells. The bars for each condition refer to, from left to right, mock, B7-H3-28htm-ZAP-70²⁵⁵⁻⁶⁰⁰, B7-H3-28htm-4-1BB-ZAP-70²⁵⁵⁻⁶⁰⁰, and B7-H3-28htm-CD28-ZAP-70²⁵⁵⁻⁶⁰⁰.

FIG. 37 is a set of graphs illustrating that the function of CAR constructs containing truncated ZAP70 does not depend on endogenous CD3zeta. The top panel shows expression of CAR and CD3zeta before (the third trace from top) or after (the bottom trace) deleting the endogenous CD3zeta using CRISPR-Cas9. The bottom panels show cytokine production by truncated-ZAP70 CARs with or without TCR (CD3zeta) knockout, demonstrating that the activity of the ZAP70 CAR construct is independent from the endogenous TCR in cells. The bars for each condition refer to, from left to right, HER2−28htm-ZAP-70²⁵⁵⁻⁶⁰⁰, HER2−28htm-ZAP-70²⁵⁵⁻⁶⁰⁰ TRAC KO, mock, and mock TRAC KO.

FIGS. 38A-38C are a set of graphs comparing T cell activation capacity of various CAR combinations, including a CAR construct containing a LAT containing a CD28 hinge-transmembrane domain in which the two cysteine residues have been mutated (2CA), combined with a wild-type SLP-76 CAR construct with a CD8 hinge-transmembrane, or a CAR construct containing a LAT mutant with both the 2CA mutation in the CD28 hinge-transmembrane region and a truncation to remove its GADS binding sites (LAT^{220-262 del}) combined with a CAR construct containing a SLP-76 mutant in which the GADS binding site was deleted (“SLP76^{224-244del}”) on a CD8 hinge-transmembrane domain. FIG. 38A is a set of FACS histograms illustrating expression of T cells expressing the listed CAR combinations. FIG. 38B is a graph showing cytokine production induced by the listed CAR combinations against single or double antigen positive tumor cells. FIG. 38C is a series of graphs showing killing of single or double antigen positive tumor cells by the listed CAR combinations. The bottom line in the left panel represents the combination of CD19−28H/TM(2CA)-LAT+HER2−8H/TM-SLP-76.

FIG. 39 is a set of graphs showing the anti-tumor function of T cells expressing different CAR constructs. The top panel exhibits graphs showing the fold change of bioluminescent tumor measurements from mice inoculated with luciferase expressing diffuse intrinsic pontine glioma 6 xenografts (DIPG-6) and treated with T cells expressing the traditional B7-H3-4-1BB-zeta CAR or the B7-H3-truncated-ZAP70 (ZAP-70²⁵⁵⁻⁶⁰⁰ fragment) CAR. The bottom panel shows the amount of T cells bearing B7-H3-4-1BB-zeta CARs or B7-H3-truncated-ZAP70 CARs harvested from the spleens of mice inoculated with luciferase-expressing DIPG-6.

FIG. 40 is a set of graphs showing the anti-tumor function of T cells expressing different CAR constructs. The top panel exhibits graphs showing tumor measurements from mice inoculated with luciferase expressing diffuse intrinsic pontine glioma 6 xenografts (DIPG-6) and treated with T cells expressing the traditional GD2-4-1BB-zeta CAR or the GD2-truncated-ZAP70 (ZAP-70²⁵⁵⁻⁶⁰⁰ fragment) CAR. The bottom panel shows the amount of T cells bearing GD2-4-1BB-zeta CARs or GD2-truncated-ZAP70 CARs harvested from the spleens of mice inoculated with luciferase-expressing DIPG-6.

FIG. 41 is a set of graphs showing the anti-tumor function of T cells expressing different CAR constructs. The top panel exhibits graphs showing tumor measurements from mice inoculated with GD2 and luciferase expressing leukemia xenografts (GD2⁺-Nalm6) and treated with T cells expressing the traditional GD2-4-1BB-zeta CAR or the GD2-truncated-ZAP70 (ZAP-70²⁵⁵⁻⁶⁰⁰ fragment) CAR. The bottom panel shows a histogram with expression of the CAR constructs (left) and the amount of T cells bearing GD2-4-1BB-zeta CARs or GD2-truncated-ZAP70 CARs harvested from the spleens of mice inoculated with luciferase-expressing GD2⁺-Nalm6.

FIG. 42 is a set of graphs that mutations capable of increasing kinase activity of ZAP-70 can be incorporated into CARs containing ZAP-70²⁵⁵⁻⁶⁰⁰ fragment endodomains. Similar to FIG. 35, the top panel is a histogram showing the expression of B7-H3-targeting CARs containing the ZAP70²⁵⁵⁻⁶⁰⁰ fragment with or without further mutations that enhance its efficacy (e.g. V314A, D327P, R360P, and K362E). The bottom panel is a graph showing cytokine production by T cells expressing these CAR constructs in response to antigen encounter with tumor cells.

FIG. 43 is a set of graphs showing that costimulatory domains and enhancing mutations can be combined to improve the potency of T cells bearing CARs containing ZAP-70²⁵⁵⁻⁶⁰⁰ fragment endodomains. The top panel is a histogram showing the expression of B7-H3-targeting ZAP-70²⁵⁵⁻⁶⁰⁰ fragment CARs with 4-1BB costimulatory domains and/or a Y292F enhancing mutation on T cells. The bottom panel shows IL-2 cytokine production by the indicated CAR T cells in response to antigen encounter with neuroblastoma CHLA-255 (left) or osteosarcoma 143B (right) tumor cells. In both panels, bars represent the constructs for, from left to right, mock control, B7-H3-28htm-ZAP-70²⁵⁵⁻⁶⁰⁰, B7-H3-28htm-4-1BB-ZAP-70²⁵⁵⁻⁶⁰⁰, B7-H3-28htm-ZAP-70^{255-600 Y292F}, and B7-H3-28htm-4-1BB-ZAP-70^{255-600 Y292F}, respectively.

FIG. 44 is a set of graphs showing that costimulatory domains and enhancing mutations can be added to improve the potency of T cells bearing CARs containing ZAP-70²⁵⁵⁻⁶⁰⁰ fragment endodomains for scFvs without tonic signaling such as anti-CD19 scFvs described herein. The top panel shows IL-2 (left) and IFNγ (right) cytokine production by CAR T cells combining ZAP-70²⁵⁵⁻⁶⁰⁰ fragments with CD28 costimulatory domains and/or a Y292F enhancing mutation. The bottom panel shows IL-2 (left) and IFNγ (right) cytokine production by CAR T cells combining ZAP-70²⁵⁵⁻⁶⁰⁰ fragments with 4-1BB costimulatory domains and/or a Y292F enhancing mutation in response to antigen encounter with Nalm6 tumor cells.

FIG. 45 is a set of graphs showing that the interdomain B portion of the ZAP-70²⁵⁵⁻⁶⁰⁰ fragment CAR can be further truncated to decrease packaging size of the delivery vector without compromising CAR T cell efficacy. The top panel is a histogram showing the expression of B7-H3-targeting CARs containing various truncated ZAP-70 endodomains (ZAP-70²⁸⁰⁻⁶⁰⁰ and ZAP-70³⁰⁸⁻⁶⁰⁰) in addition to the ZAP70²⁵⁵⁻⁶⁰⁰ fragment. The middle panel is a graph showing cytokine production by T cells expressing these CAR constructs in response to antigen encounter with B7-H3⁺Nalm6 tumor cells. The bottom panel is a series of graphs showing killing of B7-H3⁺Nalm6 tumor cells by T cells expressing these CAR constructs.

FIG. 46 is a set of graphs illustrating that T cells with combined LAT and SLP-76 endodomain CARs bearing B7-H3/GD2 scFvs show less exhausted phenotypes compared to traditional B7-H3/GD2-4-1BBzeta CAR T cells. The top panel exhibits histograms showing expression of the CAR constructs as well as PD-1, LAG-3, and TIM-3 exhaustion markers on CART cells with a B7H3-targeting 4-1BB-zeta construct or combinations of a B7-H3/CD19-targeting LAT−CD28 hinge-transmembrane domain CAR with a CD19/B7-H3-targeting SLP-76 CD8 hinge-transmembrane domain CAR. The bottom panel exhibits histograms showing expression of the CAR constructs as well as PD-1, LAG-3, and TIM-3 exhaustion markers on CART cells with a GD2-targeting 4-1BB-zeta construct or combinations of a GD2/CD19-targeting LAT−CD28 hinge-transmembrane domain CAR with a CD19/GD2-targeting SLP-76 CD8 hinge-transmembrane domain CAR.

FIGS. 47A-47C are graphs illustrating that CD19-targeting LAT−CD28 hinge-transmembrane CARs in which the GADS binding region has been deleted (LAT^{200-262del}) can be further truncated to decrease packaging size of the delivery vector without compromising CAR T cell efficacy, when paired with a HER2-targeting SLP-76 CAR construct. FIG. 47A shows flow cytometric plots exhibiting CAR expression of the listed combos in which additional amino acids have been removed from LAT (LAT^{28-90del} or LAT^{28-130del} in addition to the LAT^{200-262del}). FIG. 47B shows cytokine production by CAR T cells expressing the listed CAR molecule combinations in response to antigen encounter with no tumor control or CD19+HER2−, CD19−HER2+, or CD19+HER2+ leukemia cells (Nalm6). FIG. 47C shows graphs exhibiting killing of the CD19+HER2+ Nalm6 tumor cells by CAR T cells expressing the LAT/SLP-76 CAR molecule combinations as shown in FIG. 47A and FIG. 47B.

FIGS. 48A-48C are graphs showing that HER2-targeting SLP-76-CD8 hinge-transmembrane CARs in which the GADS binding region has been deleted (SLP-76_{224-244del}) can be further truncated to decrease packaging size of the delivery vector without compromising CAR T cell efficacy, when paired with a CD19-targeting LAT CAR construct. FIG. 48A shows flow cytometric plots exhibiting CAR expression of the listed combos in which additional amino acids have been removed from SLP-76 (SLP-76^{1-81del, 224-244del}, SLP-76^{224-265del}, or SLP-76^{224-300del}). FIG. 48B shows cytokine production by CAR T cells listed in FIG. 48A in response to antigen encounter with no tumor control or CD19+HER2−, CD19−HER2+, or CD19+HER2+ leukemia cells (Nalm6). FIG. 48C shows graphs exhibiting killing of the CD19+HER2+ Nalm6 tumor cells by CAR T cells listed in FIG. 48A and FIG. 48B.

FIGS. 49A-49B are graphs showing the expression of endogenous ROR1 on human and murine tissues. FIG. 49A is a human lung single-cell dataset from Travaglini et al. 2020 showing ROR1 expression most prominently on alveolar type I cells (AGER+, EMP2+) and to a lesser extent alveolar type II cells (SFTPC+), adventitial fibroblasts and alveolar fibroblasts (PDGFRA+), pericytes (HIGD1B+, PTN+), and vascular smooth muscle cells (ACTA2 high). Markers used to identify cells are indicated below the UMAP plot. FIG. 49B shows human (left) and mouse (right) ROR1 expression analyzed from single-cell RNA datasets. Violin plots (bottom) were generated by sampling 500 cells within each dataset and plotting ROR1 expression for all cells and the cluster with the highest mean expression of ROR1.

FIG. 50 is a schematic illustrating the potential for on-target/off-tumor toxicity when targeting ROR1 with CAR T cell therapy in a murine model.

FIGS. 51A-51B are graphs showing that various CAR combinations can be engineered to target tumor cells bearing ROR1 and CD19 antigens in vitro, including a ROR1−CD28-zeta CAR and CARs featuring a wild-type SLP-76 CAR with a CD8 hinge-transmembrane domain or a mutant in which the GADS binding region has been truncated (SLP-76^{224-244del}) combined with a wild-type LAT CAR with a CD28 hinge-transmembrane domain or a LAT CAR in which two cysteine residues in the CD28 hinge-transmembrane domain have been mutated (2CA) and/or the GADS binding region has been truncated (LAT^{200-262del}). FIG. 51A exhibits flow cytometry plots exhibiting the expression of the listed CAR combinations. For each panel, the x-axis represents the levels of CD19−SLP-76 CAR construct (DL 488), while they-axis represents the levels of ROR1-LAT CAR construct (DL 650). FIG. 51B exhibits killing of ROR1+CD19−, ROR1−CD19+, and ROR1+CD19+ leukemia lines (Nalm6) by T cells bearing the listed CAR combinations. For each panel, the x-axis represents the time (hours), while they-axis represents the cytotoxicity index.

FIGS. 52A-52C are graphs showing that various LAT & SLP-76 CAR combinations can be engineered to safely target ROR1/CD19-bearing leukemia cells (Nalm6) in a murine in vivo model, as opposed to a ROR1−CD28-zeta CAR which causes lethal on-target, off-tumor toxicity. These CARs include a CD19-targeting wild-type SLP-76 CAR with a CD8 hinge-transmembrane domain or a mutant in which the GADS binding region has been truncated (SLP-76^{224-244del}) combined with a ROR1-targeting wild-type LAT CAR with a CD28 hinge-transmembrane domain or a LAT CAR in which two cysteine residues in the CD28 hinge-transmembrane domain have been mutated (2CA) and/or the GADS binding region has been truncated (LAT^{200-262del}). FIG. 52A exhibits graphs showing percent weight changes from mice inoculated with luciferase expressing leukemia xenografts (ROR1⁺Nalm6) and treated with T cells bearing the listed CAR combinations. FIG. 52B exhibits graphs showing tumor measurements of the mice following CAR T cell treatment.

FIG. 52C compares probability of animal survival after each CAR combination treatment. Lines representing survival after the treatment refer to, in the order of increased survival, ROR1-28ζ, ROR1-28htm-LAT+CD19−8htm-SLP-76, mock, ROR1-28htm^{2CA}-LAT+CD19−8htm-SLP-76, ROR1-28htm-LAT^{200-262del}+CD19−8htm-SLP-76^{224-244del} and ROR1-28htm^{2CA}-LAT^{200-262del}+CD19−8htm-SLP-76^{224-244del}.

FIGS. 53A-53B are graphs showing additional LAT & SLP-76 CAR combinations can be engineered to safely target ROR1/CD19-bearing leukemia cells (Nalm6) in a murine in vivo model, as opposed to a ROR1−CD28-zeta CAR which causes lethal on-target, off-tumor toxicity. These CARs include a ROR1-targeting wild-type SLP-76 CAR with a CD8 hinge-transmembrane domain or a mutant in which the GADS binding region has been truncated (SLP-76^{224-244del}) combined with a CD19-targeting wild-type LAT CAR with a CD28 hinge-transmembrane domain or a LAT CAR in which two cysteine residues in the CD28 hinge-transmembrane domain have been mutated (2CA) and the GADS binding region has been truncated (LAT^{200-262del}). FIG. 53A exhibits graphs showing percent weight changes (top panel) from mice inoculated with luciferase expressing leukemia xenografts (ROR1⁺Nalm6) and treated with T cells bearing the listed CAR combinations, and graphs showing tumor measurements (bottom panel) of the mice following CAR T cell treatment. FIG. 53B shows survival of the mice following CAR T cell treatments. Lines representing survival after the treatment refer to, in the order of increased survival, ROR1-28ζ, CD19−28htm-LAT+ROR1-8htm-SLP-76, and CD19−28htm^{2CA}-LAT^{200-262del}+ROR1-8htm-SLP-76^{224-244del}.

FIGS. 54A-54C are graphs showing that a LAT/SLP-76 CAR combination can be engineered to safely target ROR1/CD19-bearing leukemia cells (Nalm6) in a murine in vivo model, as opposed to a ROR1−CD28-zeta CAR or a combination of CD19-SynNotch and ROR1−CD28-zeta CARs (SynNotch), all of which cause lethal on-target, off-tumor toxicity. A combination of ROR1−CD8 hinge/transmembrane domain-zeta and CD19−CD28 CARs (SPLIT CAR) has no antitumor activity. The safe and effective LAT/SLP-76 CAR combination includes a CD19-targeting mutant SLP-76 CAR with a CD8 hinge-transmembrane domain in which the GADS binding region has been truncated (SLP-76^{224-244del}) combined with a ROR1-targeting mutant LAT CAR with a CD28 hinge-transmembrane domain in which two CD28 hinge-transmembrane domain cysteine residues have bene mutated (2CA) and the GADS binding region has been truncated (LAT^{200-262del}). FIG. 54A exhibits graphs showing percent weight changes from mice inoculated with luciferase expressing leukemia xenografts (ROR1+Nalm6) and treated with T cells bearing the listed CAR combinations. FIG. 54B shows tumor measurements of the mice following CAR T cell treatments. FIG. 54C exhibits graphs showing survival of the mice following CAR T cell treatments. Lines representing survival after the treatment refer to, in the order of increased survival, ROR1-28ζ, CD19-SynNotch→ROR1-28ζ, ROR1-8htm-ζ+CD19−28htm-CD28, Mock, and ROR1-28htm^{2CA}-LAT^{200-262del}+CD19−8htm-SLP-76^{224-244del}.

FIG. 55 is a set of graphs showing lower in vitro baseline cytokine (IFNγ) production by tonic-signaling GD2/B7-H3-targeting CAR T cells bearing CARs with ZAP-70²⁵⁵⁻⁶⁰⁰ fragment endodomains, as opposed to CAR T cells with traditional 4-1BB-zeta endodomains. The top panel shows cytokine production by B7-H3-targeting 4-1BB-zeta or ZAP-70²⁵⁵⁻⁶⁰⁰ fragment CAR T cells when unchallenged by tumor cells. The bottom panel shows cytokine production by GD2-targeting 4-1BB-zeta or ZAP-70²⁵⁵⁻⁶⁰⁰ fragment CAR T cells when unchallenged by tumor cells.

FIG. 56 is a graph showing an exemplary LINK T-reg system. Regulatory T cells (T-regs) are transduced with one or several vectors that drive expression of SLP-76 and LAT CARs described herein. The SLP-76 CAR and LAT CAR target different antigens (antigen 1 and antigen 2, respectively) that specifically expressed on an organ-site of autoimmunity, leading to activation of both intracellular signaling domains (labeled as “SLP-76 ICD” and “LAT−ICD”), forming an AND Gate signaling system, and eventually activation of the T-regs. The activated T-regs suppress self-reactive T cells and other self-reactive immune cells harmful to the organ site, thus preventing or treating autoimmunity.

FIG. 57 is a graph comparing cytotoxicity of T cells expressing CAR constructs containing different LAT domains [i.e., wild type LAT vs. LAT with its GADS binding region deleted (LAT^{200-262del})] towards Nalm6 tumor cells at 2:1 effector:target ratio. CD19-targeting LAT−CD28 hinge-transmembrane CARs in which the GADS binding region has been deleted (LAT^{200-262del}) and the cysteine residues in the CD28-hinge-transmembrane domain are either unmutated or mutated into alanine residues (2CA) do not kill tumor cells (Nalm6) upon antigen engagement, unlike a WT LAT−CD28 hinge-transmembrane CAR.

### DETAILED DESCRIPTION OF THE DISCLOSURE

The present disclosure relates generally to, inter alia, chimeric antigen receptor (CAR) polypeptides that can be used, alone or in combination for logic gatings, to activate cells (e.g., immune cells) expressing such CAR polypeptides and/or to target, antagonize, and/or eliminate specific cells (e.g., cancer cells) when multiple antigens are present. Furthermore, the CARs and systems of CARs described herein allow for reduction of T cell exhaustion in CAR T cells while maintaining their efficacy and potency. In addition, CAR T cell activity can be enhanced when antigen density is limiting. In some embodiments, the CARs or CAR combinations have an intracellular signaling domain without an immune receptor tyrosine based activation motif (ITAM). In some embodiments, the CARs or CAR combinations have an intracellular signaling domain without a CD3zeta (CD3ζ) domain. The disclosure also provides compositions and methods useful for making such polypeptides and CARs, as well as methods for the detection and treatment of conditions, such as diseases (e.g., cancers, hematological malignancies, solid tumors, autoimmune diseases, inflammations, allergic diseases, infections, senescence/aging, etc.)

Chimeric antigen receptors are recombinant receptor constructs which, in their usual format, graft the specificity of an antibody to the effector function of a T cell. Complete response rates of up to 90% for CD-19 CAR T cells in patients with leukemia illustrate the enormous potential of these therapeutics to revolutionize care of solid tumors if they could be unleashed safely (Majzner et al. Nat Med. 2019; 25:1341-1355). However, the lack of truly tumor-specific cell surface antigens has hampered development of CARs for solid tumors due to concerns about on-target killing of normal tissues that share the target antigen (known as ‘on-target, off tumor toxicity’), as has been observed in early phase clinical trials (Lamers et al. Mol Ther. 2013; 21:904-912) and in preclinical models (Srivastava et al. Cancer Cell. 2019; 35:489-503.e8; Smith et al. Mol Ther. 2016; 24:1987-1999). CAR T cells cannot discriminate between cancer and normal tissue if both normal and cancerous tissue express the target antigen. To date, no developed system can effectively overcome this intractable problem, greatly limiting the number of potential therapeutic targets and diseases that can be treated. Though adoptive cell therapy relies on a plethora of technological advances, CARs employed in the clinic today are relatively crude receptors that are similar to the first iterations developed more than twenty years ago (Gross et al. Proc Natl Acad Sci USA. 1989; 86(24):10024-10028). A system that controls CAR T cells similar to Boolean logic gates (e.g. AND) which integrates signals based on the presence of multiple antigens would drastically increase their safety, revolutionizing the field and greatly benefiting patients with cancer.

Within a chimeric antigen receptor (CAR), the intracellular signaling domain (a.k.a., cytosolic signaling domain) generally refers to a cytoplasmic domain that transmits an activation signal to the cell expressing the CAR molecule, following binding of the extracellular domain to the corresponding ligand or antigen. In some cases, the intracellular signaling domain includes a functional signaling domain derived from a stimulatory molecule. Traditional intracellular signaling domains almost always include CD3zeta, the prototypical “master switch” that elicits T cell activity (Irving and Weiss Cell 1991; 64:891-901; Letourneur and Klausner Science 1992; 255:79-82), as well as various optional costimulatory domains to enhance potency and persistence.

As described in greater detail below, CAR molecules have been designed and tested for their activities, as a substitute for traditional CAR molecules. In some embodiments, the intracellular signaling domain of these CAR molecules does not include an immune receptor tyrosine based activation motif (ITAM). For example, these CAR molecules do not have a CD3zeta (CD3ζ) domain. Instead, proteins involved in downstream TCR signaling (e.g., ZAP70 and PLCG1) were identified and were used as intracellular signaling domains for these CAR molecules, enabling the CAR molecules to sense extracellular signals and activate cells without the traditional CD3zeta signal domain.

Furthermore, based on the identification of some CAR molecules without an ITAM (e.g., without CD3zeta) in their intracellular signaling domains, CAR molecules can be combined to sense a combination of extracellular signals (compared to one or limited number of signals for each of traditional CAR molecules) to activate cells, which enables more precise ligand/antigen selections to target specific cells (e.g., cancer/tumor cells) without a least “off-tumor” toxicity.

The experimental results presented herein demonstrate that CAR molecules, such as those without an ITAM (e.g., without CD3zeta), were capable of activating the corresponding CAR T cells, resulting in increased antitumor efficacy (e.g., enhanced cytokine production and cytotoxicity). Further, combinations of these CARs similar to Boolean logic gates (e.g. AND gates) integrate signals based on the presence of multiple antigens, which would drastically increase their safety. Domain swap and mutations have been used to engineer CAR molecules or CAR molecule combinations with enhanced specificity and/or potency. Other advantages may include, e.g., reducing T cell exhaustion in CAR T cells expressing the CAR molecules or CAR combinations, while maintaining their efficacy and potency, and/or enhancing CAR T cell activity when antigen density is limiting.

Nucleic acid molecules encoding these polypeptides are also provided. The disclosure also provides compositions and methods useful for producing such CAR polypeptides, as well as methods for the prevention and/or treatment of conditions, such as cancer.

All publications and patent applications mentioned in this disclosure are herein incorporated by reference to the same extent as if each individual publication or patent application was specifically and individually indicated to be incorporated by reference.

### General Experimental Procedures

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference. As appropriate, procedures involving the use of commercially available kits and reagents are generally carried out in accordance with manufacturer defined protocols and/or parameters unless otherwise noted.

### Definition

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “derived from” refers to the origin or source of a molecule, and may include naturally occurring, recombinant, unpurified, or purified molecules. Nucleic acid or polypeptide molecules are considered “derived from” when they include portions or elements assembled in such a way that they produce a functional unit. The portions or elements can be assembled from multiple sources provided that they retain evolutionarily conserved function. In some embodiments, the derivative nucleic acid or polypeptide molecules include substantially the same sequence as the source nucleic acid or polypeptide molecule. For example, the derivative nucleic acid or polypeptide molecules of the present disclosure may have at least 80%, 85%, 90%, 95%, 98%, 99%, or 100% sequence identity to the source nucleic acid or polypeptide molecule.

The term “recombinant” nucleic acid molecule, polypeptide, and/or cell, as used in the instant application, refers to a nucleic acid molecule, polypeptide, and/or cell that has been altered through human intervention. As non-limiting examples, a recombinant nucleic acid molecule can be one which: 1) has been synthesized or modified in vitro, for example, using chemical or enzymatic techniques, or recombination of nucleic acid molecules; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. A non-limiting example of a recombinant protein is a chimeric antigen receptor as provided herein.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, a “host cell” refers to a cell for introduction of a nucleic acid and/or a polypeptide (e.g., the CAR molecules) described herein and/or a cell for expressing a nucleic acid or a polypeptide described herein. Host cells can be either untransformed cells or cells that have already been introduced with at least one nucleic acid molecule (e.g., the CAR molecules) described herein. A “recombinant cell” refers to a cell having genetic modifications and/or having introduced nucleic acids and/or polypeptides described herein.

As used herein, a “subject” or an “individual” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

The term “vector” is used herein to refer to a nucleic acid molecule or sequence capable of transferring or transporting another nucleic acid molecule. For example, a vector can be used as a gene delivery vehicle to transfer a gene into a cell. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

As can be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As can also be understood by one skilled in the art all language such as “up to”, “at least”, “greater than”, “less than”, and the like include the number recited and refer to ranges which can be subsequently broken down into sub-ranges as discussed above. Finally, as can be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Compositions of the Disclosure

As described in greater detail below, the invention provides, inter alia, compositions of CARs and combinations of CARs that display enhanced efficacy and/or can be used for logic gating to target and eliminate specific cells (e.g., cancer cells) when multiple antigens are present. Furthermore, the CARs and system of CARs described herein allow for reduction of T cell exhaustion in CAR T cells while maintaining their efficacy and potency. In addition, CAR T cell activity can be enhanced when antigen density is limiting. In some embodiments, the invention provides chimeric antigen receptors (CARs) that do not have an immune receptor tyrosine based activation motif (ITAM) in their intracellular signaling domains. In some embodiments, the CARs of the disclosure do not have a CD3zeta (CD3ζ) domain. Also provided are combinations of these chimeric antigen receptors (CARs) (e.g., forming logic AND gates) that only activate cells expressing these CAR combinations when each and every CAR molecule in the combination is activated or binds to the corresponding extracellular ligand or antigen. Also provided are recombinant nucleic acids encoding such CARs or CAR combinations, as well as recombinant cells that have been engineered to express a CAR polypeptide or a combination of CAR polypeptides as disclosed herein and are directed against a cell of interest such as a cancer cell.

### Chimeric Antigen Receptors (Cars)

As described above, the CAR polypeptides of the present disclosure include (i) an extracellular ligand-binding domain (a.k.a., extracellular antigen-binding domain, or ECD) having a binding affinity for a ligand (or an antigen); (ii) a transmembrane domain (TMD); and (iii) an intracellular signaling domain (a.k.a., cytosolic signaling domain). In some embodiments, binding of the ligand/antigen to the extracellular ligand-binding domain activates the intracellular signaling domain of a CAR polypeptide, either alone or in a combination of CAR polypeptides (e.g., AND gates). In some embodiments, binding of the ligand/antigen to the extracellular ligand-binding domain does not activate the intracellular signaling domain of a CAR polypeptide, when in a combination of CAR polypeptides (e.g., AND gates). However, such binding may be required to activate a cell expressing the combination of CAR polypeptides. For example, a cell expressing two CAR molecules as an AND gate combination may be only activated when the extracellular ligand-binding domains of both CAR molecules bind to their specific ligands. In some embodiments, the CAR polypeptides described herein do not have an immune receptor tyrosine based activation motif (ITAM) in their intracellular signaling domains. In some embodiments, the disclosed CARs have the above listed domains in (i)-(iii) in a N-terminal to C-terminal direction. In some embodiments, the disclosed CARs are described in a format of X—Y—Z, wherein X represents the ligand/antigen recognizable by the extracellular ligand-binding domain, Y represents the hinge/transmembrane (H/TM) domain, and Z represents the intracellular signaling domain. For example, “CD19−CD28H/TM-LAT” refers to a CAR molecule having an extracellular ligand-binding domain which specifically binds to CD19, a CD28 hinge/transmembrane domain, and a LAT intracellular signaling domain. Combinations of CAR molecules follow the same format. For example, “CD19−CD28H/TM-LAT+HER2-CD28H/TM-SLP-76” refers to a CAR combination having two CAR molecules, including one CAR as described above, and another CAR having an extracellular ligand-binding domain which specifically binds to HER2, a CD28 hinge/transmembrane domain, and a SLP-76 intracellular signaling domain.

In some embodiments, the disclosed CARs further include one or more hinge domains and/or costimulatory domains. Description of these corresponding CAR constructs follows the same format as above, adding the optional hinge and/or costimulatory domain in the correspond place (in N-terminal to C-terminal direction). For example, “HER2-CD28H/TM-CD28-SLP-76” refers to a CAR molecule having an extracellular ligand-binding domain which specifically binds to HER2, a CD28 hinge/transmembrane domain, a CD28 costimulatory domain, and a SLP-76 intracellular signaling domain.

In some embodiments, the disclosed CARs contain at least one intracellular (i.e., cytosolic) signaling domain described herein, including, but not limiting to, ZAP70, PLCG1, PKC, ITK, NCK, VAV1, GRB2, GADS, SOS1, ADAP, SYK, LYN, PI3K, BLNK, or a biologically active fragment, mutant, or variant thereof. In some embodiments, the disclosed CARs contain more than one intracellular (i.e., cytosolic) signaling domain described herein, including, but not limiting to, ZAP70, PLCG1, PKC, ITK, NCK, VAV1, GRB2, GADS, SOS1, ADAP, SYK, LYN, PI3K, BLNK, or a biologically active fragment, mutant, or variant thereof. Sequences of exemplary intracellular signaling domains are disclosed herein, such as in Tables 1-2 and sequence listing.

### Extracellular Ligand (Antigen)-Binding Domains (ECD)

A CAR molecule described herein has at least one ECD which has a binding affinity for one or more target ligands (or antigens, which are used interchangeably in the instant application). In some embodiments, the target ligand is expressed on a cell surface, or is otherwise anchored, immobilized, or restrained on a cell surface. Non-limiting examples of suitable ligand types include cell surface receptors, adhesion proteins, carbohydrates, lipids, glycolipids, lipoproteins, and lipopolysaccharides that are surface-bound, integrins, mucins, and lectins. In some embodiments, the ligand is a protein. In some embodiments, the ligand is a carbohydrate. In some embodiments, the ligand is expressed by a target cell (e.g., a cancer/tumor cell). In some embodiments, the ligand is an adaptor molecule specifically recognizing a target cell (e.g., a cancer/tumor cell). In some embodiments, the ligand is a biomarker for a specific disease, disorder, or condition (e.g., a cancer/tumor). Non-limiting examples of suitable ligand include CD19, HER2, ROR1, B7-H3 (CD276), GD2, influenza hemagglutinin (HA), CD22, CD2, CD5, CD6, 4-1BB, FcγR1, and integrins, as well as those described in the below section titled “antigens”.

In some embodiments, the ECD of the CAR polypeptides disclosed herein includes an antigen-binding moiety that binds to one or more target antigens. In some embodiments, the antigen-binding moiety includes one or more antigen-binding determinants of an antibody or a functional antigen-binding fragment thereof, including, at least, a ligand-binding domain of an antibody, an antigen-binding fragment, an antibody mimetic, a receptor, or a ligand for a targeted receptor. One skilled in the art upon reading the present disclosure can readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a monoclonal antibody, an antigen-binding fragment (Fab), a nanobody, a diabody, a triabody, a minibody, an F(ab′)₂ fragment, an F(ab)v fragment, a single chain variable fragment (scFv), a single domain antibody (sdAb), a V_{H} domain, a V_{L} domain, an Fv fragment, a VNAR domain, and a VHH domain, or a functional fragment thereof. In some embodiments, the antigen-binding moiety includes a heavy chain variable region and a light chain variable region. In some embodiments, the antigen-binding moiety includes a scFv. In some embodiments, the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof. In some embodiments, the receptor is NKG2D or a biologically active fragment thereof. In some embodiments, the ligand for a targeted receptor is an IL-13 polypeptide, an IL-13 mutein, chlorotoxin, or a biologically active fragment thereof.

The antigen-binding moiety can include naturally-occurring amino acid sequences or can be engineered, designed, or modified so as to provide desired and/or improved properties, e.g., binding affinity. Generally, the binding affinity of an antibody or an antigen-binding moiety for a target antigen (e.g., CD19 antigen or HER2 antigen) can be calculated by the Scatchard method described by Frankel et al., Mol. Immunol, 16: 101-106, 1979. In some embodiments, binding affinity can be measured by an antigen/antibody dissociation rate. In some embodiments, a high binding affinity can be measured by a competition radioimmunoassay. In some embodiments, binding affinity can be measured by ELISA. In some embodiments, antibody affinity can be measured by flow cytometry. An antibody that “selectively binds” a target antigen (such as CD19 or HER2) is an antibody that binds the target antigen with high affinity and does not significantly bind other unrelated antigens but binds the antigen with high affinity, e.g., with an equilibrium constant (K_{D}) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a CAR polypeptide of the present disclosure. For example, a CAR polypeptide with an ECD including an antibody specific for a HER2 antigen can target cells to HER2-expressing breast cancer cells. In some embodiments, the ECD of the CAR polypeptides disclosed herein is capable of binding a tumor-associated antigen (TAA) or a tumor-specific antigen (TSA). A skilled artisan can understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

### Antigens

In the instant application, the terms “ligand(s)” and “antigen(s)” are used interchangeably to mean a target molecule(s) specifically recognized by an extracellular antigen-binding domain of a CAR molecule or a CAR molecule combination described herein. In principle, there are no particular limitations with regard to suitable target antigens. In some embodiments of the disclosure, the antigen-binding moiety of the ECD is specific for an epitope present in an antigen expressed or recognized by a target cell. In some embodiments, the target cell is correlated to a disease or disorder. Exemplary diseases or disorders may include, e.g., proliferative diseases (e.g., cancers), hematological malignancies, solid tumors, autoimmune diseases, inflammations, allergic diseases, infections, senescence/aging, etc. In some embodiments of the disclosure, the antigen-binding moiety of the ECD is specific for an epitope present in an antigen that is expressed by a tumor cell, i.e., a tumor-associated antigen. The tumor-associated antigen can be an antigen associated with, e.g., a leukemia cell, a neublastoma cell, an osteosarcoma cell, a pancreatic cancer cell, a colon cancer cell, an ovarian cancer cell, a prostate cancer cell, a lung cancer cell, mesothelioma cell, a breast cancer cell, a urothelial cancer cell, a liver cancer cell, a head and neck cancer cell, a sarcoma cell, a cervical cancer cell, a stomach cancer cell, a gastric cancer cell, a melanoma cell, a uveal melanoma cell, a cholangiocarcinoma cell, a multiple myeloma cell, a lymphoma cell, a glioblastoma cell, or other cancer cells described in the present disclosure. In some embodiments, the antigen-binding moiety is specific for an epitope present in a tissue-specific antigen. In some embodiments, the antigen-binding moiety is specific for an epitope present in a disease-associated antigen. Tumors often refers to a subgroup of cancers when an uncontrolled growth of cells occurs in solid tissue such as an organ, muscle, or bone. In the instant application, the terms “tumors” and “cancers” are generally used interchangeably to mean cells having an uncontrolled growth, unless specified otherwise.

In some embodiments, the antigen is selected from the group consisting of CD19, HER2, ROR1, B7-H3 (CD276), influenza hemagglutinin (HA), CD22, CD2, CD5, CD6, 4-1BB, FcγR1, and integrins. In some embodiments, the antigen is selected from the group consisting of CD19, HER2, ROR1, B7-H3 (CD276), influenza hemagglutinin (HA), CD22, CD2, CD5, CD6, 4-1BB, FcγR1, GD2, CD22, CD10, CD20, GPC2, GD3, GM2, GM3, and integrins.

Non-limiting examples of suitable target antigens also include Glypican 2 (GPC2), human epidermal growth factor receptor 2 (Her2/neu), CD276 (B7-H3), PSMA, IL-13-receptor alpha 1, IL-13-receptor alpha 2, alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, CD138, FolR1, LeY, MCSP, and TYRP1.

Additional antigens that can be suitable for the CARs disclosed herein include, but are not limited to, the dimeric form of the pyruvate kinase isoenzyme type M2 (tumor M2-PK), CD19, CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3(CD61), galactin, K-Ras (V-Ki-ras2 Kirsten rat sarcoma viral oncogene), and Ral-B. In some embodiments, the antigen is CD19, human epidermal growth factor receptor 2 (Her2/neu), CD276 (B7-H3), or HA.

Antigens that can be suitable for the CARs disclosed herein include, but are not limited to, one, or any combination thereof, of: CD1a, CD1b, CD1c, CD2, CD3, CD4, CD5, CD6, CD7, CD8, CD9, CD10, CD11a, CD11b, CD11c, CD12, CD13, CD14, CD15 (SSEA-1), CD16 (FcγRIII), CD17, CD18, CD19, CD20, CD21, CD22, CD23, CD24, CD25, CD26, CD27, CD28, CD29, CD30, CD31, CD32 (FcγRII), CD33, CD34, CD35, CD36, CD37, CD38, CD39, CD40, CD41, CD43, CD44, CD44V6, CD45, CD45R/B220, CD45RO, CD49b, CD49d, CD49f, CD52, CD53, CD54, CD56 (NCAM), CD57, CD61 (integrin β3), CD62L, CD63, CD64, CD66b, CD68, CD69, CD70, CD73, CD74, CD79a (Igα), CD79b (Igβ), CD80, CD83, CD85k (ILT3), CD86, CD88, CD93 (C1Rqp), CD94, CD95, CD99, CD103, CD105 (Endoglin), CD107a, CD107b, CD114 (G-CSFR), CD115, CD117, CD122, CD123, CD129, CD133, CD134, CD138 (Syndecan-1), CD141, CD146, CD152 (CTLA-4), CD158 (Kir), CD161 (NK-1.1), CD163, CD183, CD191, CD193 (CCR3), CD194 (CCR4), CD195 (CCR5), CD197 (CCR7), CD203c, CD205 (DEC-205), CD207 (Langerin), CD209 (DC-SIGN), CD223, CD235, CD244 (2B4), CD252 (OX40L), CD267, CD268 (BAFF-R), CD273 (B7-DC, PD-L2), CD276 (B7-H3), CD279 (PD1), CD282 (TLR2), CD284 (TLR4), CD294, CD304 (Neuropilin-1), CD305, CD314 (NKG2D), CD319 (CRACC), CD326, CD328 (Siglec-7), CD335 (NKp46), fetal acetylcholine receptor (AChR), ADGRE2, alpha-fetoprotein (AFP), ALK, BCMA, BDCA3, C3AR, Lewis A (CA19.9), carbonic anhydrase IX (CA1X), calretinin, cancer antigen-125 (CA-125), CCR1, CCR4, CDS, carcinoembryonic antigen (CEA), chromogranin, CLEC12A, an antigen of a cytomegalovirus (CMV) infected cell (e.g., a cell surface antigen), CS-1, CSPG4, cytokeratin, desmin, DLK1, DLL3, EGFRvIII (epidermal growth factor variant III), EGFR and isovariants thereof, epithelial cell adhesion molecule (EpCAM), epithelial glycoprotein2 (EGP 2), epithelial glycoprotein-40 (EGP-40), epithelial membrane protein (EMA), ERBB, epithelial tumor antigen (ETA), FAP, folate-binding protein (FBP), FcγR1, FcεRIα, FITC, FLT3, FOLR1, FOLR3, galactin, ganlgiosides, gross cystic disease fluid protein (GCDFP-15), GD2 (ganglioside G2), GD3, GM2, GM3, glial fibrillary acidic protein (GFAP), gpA33, glycopeptides, Glypican 2 (GPC2), oncofetal antigen (h5T4), influenza hemagglutinin (HA), human epidermal growth factor receptor 2 (Her2/neu), HLA-DR, HM1.24, HMB-45 antigen, HPV E6, HPV E7, ICAM-1, IgG, IgD, IgE, IgM, IL-13-receptor alpha 1, integrins, Integrin B7, Interleukin-13 receptor subunit alpha-2 (IL-13Rα2), Kappa light chain, kinase insert domain receptor (KDR), Lamba light chain, LILRB2, Lewis Y (LeY), LGR5, Ly49, Ly108, L1 cell adhesion molecule (L1-CAM), melanoma-associated antigen (MAGE), melanoma antigen family A 1 (MAGE-A1), protein melan-A (melanoma antigen recognized by T lymphocytes; MART-1), MCSP, c-Met, MICA/B, mesothelin, muscle-specific actin (MSA), Mesothelin (MSLN), the dimeric form of the pyruvate kinase isoenzyme type M2 (tumor M2-PK), Mucin 1 (Muc-1), Mucin 16 (Muc-16), myo-D1, Necl-2, neurofilament, NKCSI, NKG2D, neuron-specific enolase (NSE), NY-ESO, cancer-testis antigen NY-ESO-1, an abnormal p53 protein, PAP (prostatic acid phosphatase), PAMA, P-cadherin, placental alkaline phosphatase, PRAIVIE, prostein, prostate stem cell antigen (PSCA), prostate-specific membrane antigen (PSMA), Ral-B, K-Ras (V-Ki-ras2 Kirsten rat sarcoma viral oncogene), an abnormal ras protein, ROR1, SLAMF7/CS1, receptor tyrosine-protein kinases erb-B2,3,4, sperm protein 17 (Sp17), STEAP1 (six-transmembrane epithelial antigen of the prostate 1), synaptophysin, tumor-associated glycoprotein 72 (TAG-72), TALLA-1, TARP (T cell receptor gamma alternate reading frame protein), TEM-8, human telomerase reverse transcriptase (hTERT), TIM-3, TLR4, TRBC1, TRBC2, Trp-p8, thyroglobulin, thyroid transcription factor-1, TYRP1, tyrosinase, vascular endothelial growth factor R2 (VEGF-R2), Vα24, Wilms tumor protein (WT-1), and various pathogen antigen known in the art. In some embodiments, an antigen described herein is a tumor-specific antigen (TSA) or a tumor-associated antigen (TAA).

In some embodiments, the CAR polypeptides disclosed herein include an ECD including an antigen-binding moiety that binds CD19. In some embodiments, the CAR polypeptides disclosed herein include an ECD including an antigen-binding moiety that binds HER2. In some embodiments, the CAR polypeptides disclosed herein include an ECD including an antigen-binding moiety that binds B7-H3. In some embodiments, the CAR polypeptides disclosed herein include an ECD including an antigen-binding moiety that binds HA. In some embodiments, the CAR polypeptides disclosed herein include an ECD including an antigen-binding moiety that binds CD22. In some embodiments, the CAR polypeptides disclosed herein include an ECD including an antigen-binding moiety that binds CD2. In some embodiments, the CAR polypeptides disclosed herein include an ECD including an antigen-binding moiety that binds CD5. In some embodiments, the CAR polypeptides disclosed herein include an ECD including an antigen-binding moiety that binds CD6. In some embodiments, the CAR polypeptides disclosed herein include an ECD including an antigen-binding moiety that binds 4-1BB. In some embodiments, the CAR polypeptides disclosed herein include an ECD including an antigen-binding moiety that binds FcγR1. In some embodiments, the CAR polypeptides disclosed herein include an ECD including an antigen-binding moiety having an amino acid sequence exhibiting at least 50% sequence identity to any one of SEQ ID NOs: 1-5, 161 and 197. In some embodiments, the antigen-binding moiety has an amino acid sequence exhibiting at least 50%, at least 60%, at least 70%, at least 80%, at least 85%, at least 90%, at least 95%, at least 96%, at least 97%, at least 98%, at least 99%, or 100% sequence identity to any one of SEQ ID NOs: 1-5, 161 and 197. The percent identity as used herein refers to two or more sequences or subsequences that are the same or have a specified percentage of nucleotides or amino acids that are the same over a specified region. The two or more sequences or subsequences may be compared and aligned for maximum correspondence over a comparison window or designated region, as measured by, e.g., a BLAST or BLAST 2.0 sequence comparison algorithms, or by manual alignment and visual inspection. See e.g., the NCBI web site at ncbi.nlm.nih.gov/BLAST. This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. The amino acid substitution(s) may be a conservative amino acid substitution, for example at a non-essential amino acid residue in the CDR sequence(s). A “conservative amino acid substitution” is understood to be one in which the original amino acid residue is substituted with an amino acid residue having a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Sequences of exemplary ECDs are disclosed herein, such as in Tables 1-2 and sequence listing.

### Hinge Domains

As described above, the CAR polypeptides described herein may have an optional hinge domain. Within a chimeric antigen receptor, the term “hinge domain” generally refers to a flexible polypeptide connector region disposed between the targeting moiety (ECD) and the TMD. These sequences are generally derived from IgG subclasses (such as IgG1 and IgG4), IgD and CD8 domains, of which IgG1 has been most extensively used.

In some embodiments, the hinge domain provides structural flexibility to flanking polypeptide regions. The hinge domain may consist of natural or synthetic polypeptides. In recent years, several studies of the hinge domain mainly focused on the following aspects: (1) reducing binding affinity to the Fcγ receptor, thereby eliminating certain types of off-target activation; (2) enhancing the single-chain variable fragment (scFv) flexibility, thereby relieving the spatial constraints between particular epitopes targeted on tumor antigens and the CAR's antigen-targeting moiety; (3) reducing the distance between an scFv and the target epitope(s); and (4) facilitating the detection of CAR expression using anti-Fc reagents. Nevertheless, the influences of the hinge domain on CAR T cell physiology are not well understood.

It can be appreciated by those skilled in the art that hinge domains may improve the function of the CAR polypeptides described herein by promoting optimal positioning of the antigen-binding moiety in relationship to the portion of the antigen recognized by the same. It can be appreciated that, in some embodiments, the hinge domain may not be required for optimal CAR activity. In some embodiments, a beneficial hinge domain having a short sequence of amino acids promotes CAR activity by facilitating antigen binding by, e.g., relieving any steric constraints that may otherwise alter antibody binding kinetics. The sequence encoding the hinge domain may be positioned between the antigen recognition moiety and the TMD. In some embodiments, the hinge domain is operably linked downstream of the antigen-binding moiety and upstream of the TMD. In case a CAR polypeptide described herein has an optional hinge domain, the format to describe the hinge domain and the TMD domain may be “H/TM” or “H-TM”. For example, CD19-CD28H/TM-LAT or CD19−CD28H-TM-LAT represents the same CAR molecule having a CD28 hinge domain and a CD28 transmembrane domain (TMD), while IgG4H/CD4TM represents a CAR molecule having a hinge domain derived from IgG4 or including an IgG4 hinge domain.

The hinge sequence can generally be any moiety or sequence derived or obtained from any suitable molecule. For example, in some embodiments, the hinge sequence can be derived from the human CD8 molecule or a CD28 molecule and any other receptors that provide a similar function in providing flexibility to flanking regions. The hinge domain can have a length of from about 4 amino acid (aa) to about 50 aa, e.g., from about 4 aa to about 10 aa, from about 10 aa to about 15 aa, from about aa to about 20 aa, from about 20 aa to about 25 aa, from about 25 aa to about 30 aa, from about 30 aa to about 40 aa, or from about 40 aa to about 50 aa. Suitable hinge domains can be readily selected and can be of any of a number of suitable lengths, such as from 1 amino acid (e.g., Gly) to 20 aa, from 2 aa to 15 aa, from 3 aa to 12 aa, including 4 aa to 10 aa, 5 aa to 9 aa, 6 aa to 8 aa, or 7 aa to 8 aa, and can be 1, 2, 3, 4, 5, 6, or 7 aa. Non-limiting examples of suitable hinge domains include a CD8 hinge domain, a CD28 hinge domain, a CD4 hinge domain, a PD-1 hinge domain, a CD2 hinge domain, a CTLA4 hinge domain, or an IgG4 hinge domain. In some embodiments, the hinge domain can include regions derived from a human CD8α (a.k.a. CD8a) molecule or a CD28 molecule and any other receptors that provide a similar function in providing flexibility to flanking regions. Additional exemplary hinge domains derive from or include hinge domains of LFA-1 (CD11a/CD18), CD5, CD27 (TNFRSF7), CD70, 4-1BB, OX40 (CD134), ICOS (CD278), IgG1 Fc region, IgG2 Fc region, IgG3 Fc region, IgG4 Fc region, IgE Fc region, IgM Fc region, IgA Fc region, or a combination thereof. In some embodiments, the CAR disclosed herein includes a hinge domain derived from a CD8 hinge domain. In some embodiments, the hinge domain can include one or more copies of the CD8 hinge domain. In some embodiments, the CAR disclosed herein includes a hinge domain derived from a CD28 hinge domain. In some embodiments, the hinge domain can include one or more copies of the CD28 hinge domain. In some embodiments, the CAR disclosed herein includes a hinge domain derived from a CD4 hinge domain. In some embodiments, the hinge domain includes one or more copies of the CD4 hinge domain. In some embodiments, the CAR disclosed herein includes a hinge domain derived from an IgG4 hinge domain. In some embodiments, the hinge domain can include one or more copies of the IgG4 hinge domain.

### Costimulatory Domains

As described above, the CAR polypeptides described herein may have an optional costimulatory domain. Generally, the costimulatory domain suitable for the CAR polypeptides disclosed herein can be any one of the costimulatory domains known in the art. Examples of suitable costimulatory domains that can enhance cytokine production and include, but are not limited to, costimulatory polypeptide sequences derived from 4-1BB (CD137), CD27, CD28, OX40 (CD134), and costimulatory inducible T-cell co-stimulator (ICOS) polypeptide sequences. Additional exemplary costimulatory polypeptide sequences may be or be derived from a costimulatory domain of: CD28, ICOS (CD278), CD27, 4-1BB (CD137), OX40 (CD134), CD2, CD4, CD5, CD7, CD8, CD8α, CD8β, CD11a, CD11b, CD11c, CD11d, CD18, CD19, CD19a, CD29, CD30, CD30L, CD40, CD40L (CD154), CD48, CD49a, CD49D, CD49f, CD58, CD53, ICAM-1 (CD54), CD69, CD70, CD80 (B7-1), CD82, CD83, CD84, CD86 (B7-2), CD90, CD96, CD100, CD103, CD122, CD132, CD150 (SLAMF1), CD160 (BY55), CD162 (DNAM1), CD223 (LAG3), CD226, CD229, CD244, CD270 (HVEM), CD273 (PD-L2), CD274 (PD-L1), CD278, LAT, lymphocyte function-associated antigen-1 (LFA-1), LIGHT, NKG2C, NKG2D, NKp30, NKp44, NKp46, NKp80 (KLRF1), B7-H2, B7-H3, CD83 ligand, PD-1, SLP-76, Toll-like receptors (TLRs, such as TLR2), DAP10, DAP12, LAG-3, 2B4, CARD1, CTLA-4 (CD152), TRIM, ZAP70, FcERIγ, 4-1BBL, BAFF, GADS, GITR, GITR-L, BAFF-R, HVEM, CD27L, OX40L, TAC1, BLAME, CRACC, CD2F-10, NTB-A, integrin α4, integrin α4β1, integrin α4β7, IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LTBR, PAG/Cbp, PSGL1, SLAMF6 (NTB-A, Ly108), SLAMF7, SLP-76, TNFR2, TRANCE/RANKL, VLA1, VLA-6, BTLA, ikaros, LAG-3, LMIR, CEACAM1, CRTAM, TCL1A, DAP12, TIM-1, Dectin-1, PDCD6, PD-1, TIM-4, TSLP, EphB6, TSLP-R, HLA-DR, or any combination thereof.

Accordingly, in some embodiments, the costimulatory domain of the CARs disclosed herein is selected from the group consisting of a costimulatory 4-1BB (CD137) polypeptide sequence, a costimulatory CD27 polypeptide sequence, a costimulatory CD28 polypeptide sequence, a costimulatory OX40 (CD134) polypeptide sequence, and a costimulatory inducible T-cell co-stimulator (ICOS) polypeptide sequence. In some embodiments, the CARs disclosed herein include a costimulatory domain derived from a costimulatory 4-1BB (CD137) polypeptide sequence. In some embodiments, the CARs disclosed herein include a costimulatory 4-1BB (CD137) polypeptide sequence. In some embodiments, the CARs disclosed herein include a costimulatory domain derived from a costimulatory CD28 polypeptide sequence. In some embodiments, the CARs disclosed herein include a costimulatory CD28 polypeptide sequence. In some embodiments, the CARs disclosed herein include a costimulatory domain having an amino acid sequence exhibiting at least 50% sequence identity to the sequence of SEQ ID NO: 146 or 147. In some embodiments, the costimulatory domain has an amino acid sequence exhibiting at least 50%, at least 60%, at least 70%, at least 80%, at least 85%, at least 90%, at least 95%, at least 96%, at least 97%, at least 98%, at least 99%, or 100% sequence identity to the sequence of SEQ ID NO: 146 or 147.

### Transmembrane Domains (TMD)

Generally, the transmembrane domain (also referred to as transmembrane region) suitable for the CAR polypeptides disclosed herein can be any one of the TMDs known in the art. Without being bound to theory, it is believed that the TMD traverses the cell membrane, anchors the CAR to the cell surface, and connects the ECD to the intracellular signaling domain, thus impacting expression of the CAR on the cell surface. Examples of suitable TMDs include, but are not limited to, a CD28 TMD, a CD8 TMD, a CD4 TMD, a CD3 TMD, a CTLA-4 TMD, an OX40 TMD, a 4-1BB TMD, a CD2 TMD, and a PD-1 TMD. Additional exemplary TMDs include TMDs from CD3D, CD3E, CD3G, CD3zeta, CD8a, CD8b, CD16, CD25, CD27, CD40, CD79A, CD79B, CD80, CD84, CD86, CD95, CD150 (SLAMF1), CD166, CD200R, CD223 (LAG3), CD270 (HVEM), CD272 (BTLA), CD273 (PD-L2), CD274 (PD-L1), CD278 (ICOS), CD300, CD357 (GITR), A2aR, ICAM-1, 2B4, BTLA, DAP10, FcRα, FcRβ, Fyn, GAL9, IL7, IL12, IL15, KIR, KIR2DL4, KIR2DS1, LAG-3, Lck, LAT, LPA5, LRP, NKp30, NKp44, NKp46, NKG2C, NKG2D, NOTCH1, NOTCH2, NOTCH3, NOTCH4, PTCH2, ROR2, Ryk, SLP-76, SIRPα, pTα, T cell receptor polypeptides (e.g., TCRα and TCRβ), TIM3, TRIM, and ZAP70. Accordingly, in some embodiments, the TMD is derived from a CD28 TMD, a CD8 TMD, a CD4 TMD, a CD3 TMD, a CTLA4 TMD, an OX40 TMD, a 4-1BB TMD, a CD2 TMD, and a PD-1 TMD. In some embodiments, the TMD includes a CD28 TMD, a CD4 TMD, a CD8 TMD, a CD3 TMD, a CTLA4 TMD, an OX40 TMD, a 4-1BB TMD, a CD2 TMD, and a PD-1 TMD. In some embodiments, the CAR disclosed herein include a TMD derived from a CD8. In some embodiments, the CAR polypeptides disclosed herein include a CD8 TMD. In some embodiments, the CAR disclosed herein include a TMD derived from a CD28. In some embodiments, the CAR disclosed herein include a CD28 TMD. In some embodiments, the CAR disclosed herein include a TMD derived from a CD4. In some embodiments, the CAR disclosed herein include a CD4 TMD.

Exemplary CAR molecules as described herein contain a hinge domain and a TMD domain adjacent to each other. Sequences are disclosed herein for exemplary hinge/transmembrane (H/TM or hinge/TM) domains for various CAR molecules. In some embodiments, a CAR molecule disclosed herein includes an H/TM domain having an amino acid sequence exhibiting at least 50% sequence identity to any one of SEQ ID NOs: 6-10 and 102. In some embodiments, a CAR molecule disclosed herein includes an H/TM domain having an amino acid sequence exhibiting at least 50%, at least 60%, at least 70%, at least 80%, at least 85%, at least 90%, at least 95%, at least 96%, at least 97%, at least 98%, at least 99%, or 100% sequence identity to any one of SEQ ID NOs: 6-10 and 102.

### Extracellular Spacer

The CARs disclosed herein may further include an optional extracellular spacer domain including one or more intervening amino acid residues that are positioned between the ECD and an optional hinge domain. In some embodiments, the extracellular spacer domain is operably linked downstream to the ECD and upstream to an optional hinge domain. In principle, there are no particular limitations to the length and/or amino acid composition of the extracellular spacer. In some embodiments, any arbitrary single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as an extracellular spacer. In some embodiments, the extracellular spacer includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the extracellular spacer includes about 1 to 10, about 5 to 15, about 10 to 20, about 15 to 25, about 20 to 40, about 30 to 50, about 40 to 60, about 50 to 70 amino acid residues. In some embodiments, the extracellular spacer includes about 40 to 70, about 50 to 80, about 60 to 80, about 70 to 90, or about 80 to 100 amino acid residues. In some embodiments, the extracellular spacer includes about 1 to 10, about 5 to 15, about 10 to 20, about 15 to 25 amino acid residues. In some embodiments, the length and amino acid composition of the extracellular spacer can be optimized to vary the orientation and/or proximity of the ECD and an optional hinge domain to one another to achieve a desired activity of the CARs. In some embodiments, the orientation and/or proximity of the ECD and an optional hinge domain to one another can be varied and/or optimized as a “tuning” tool or effect that would enhance or reduce the efficacy of the CARs. In some embodiments, the orientation and/or proximity of the ECD and an optional hinge domain to one another can be varied and/or optimized to create fully functional or partially functional versions of the CARs. In some embodiments, the extracellular spacer domain includes an amino acid sequence corresponding to an IgG4 hinge domain and an IgG4 CH2-CH3 domain. Additional exemplary extracellular spacer domains may derive from or include an immunoglobulin hinge region (e.g., IgG1, IgG2, IgG3, IgG4, IgA, IgD), all or a portion of an immunoglobulin Fc domain (e.g., a CH1 domain, a CH2 domain, a CH3 domain, or combinations thereof), a stalk region of a type II C-lectin (the extracellular domain located between the C-type lectin domain and the transmembrane domain). Type II C-lectins include, e.g., CD23, CD69, CD72, CD94, NKG2A, and NKG2D. In yet further embodiments, an extracellular spacer domain may be derived from or include a toll-like receptor (TLR) juxtamembrane domain. A TLR juxtamembrane domain contains acidic amino acids lying between the leucine rich repeats (LRRs) and the transmembrane domain of a TLR. In certain embodiments, a TLR juxtamembrane domain is a TLR1, TLR2, TLR3, TLR4, TLR5, TLR6, TLR7, TLR8, or TLR9 juxtamembrane domain.

In some embodiments, the CAR polypeptide has an amino acid sequence having at least 50% sequence identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, the CAR polypeptide has an amino acid sequence having at least 50%, at least 60%, at least 70%, at least 80%, at least 85%, at least 90%, at least 95% sequence identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, the CAR polypeptide has an amino acid sequence having at least 95%, at least 96%, at least 97%, at least 98%, at least 99% sequence identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, the CAR polypeptide has an amino acid sequence having 100% sequence identity to any one of SEQ ID NOs: 27-49, 111-123, 148-151, 162, 164, 166, 168, 170-176, 178, 180-184, 186, 187, 189, 190, 192, 194, 196, and 198-204.

One skilled in the art can appreciate that the complete amino acid sequence of a CAR polypeptide of the disclosure can be used to construct a back-translated gene. For example, a DNA oligomer containing a nucleotide sequence coding for a given CAR can be synthesized. For example, several small oligonucleotides coding for portions of the desired CAR or antibody can be synthesized and then ligated. The individual oligonucleotides typically contain 5′ or 3′ overhangs for complementary assembly.

In addition to generating desired CARs via expression of nucleic acid molecules that have been altered by recombinant molecular biological techniques, a subject CAR in accordance with the present disclosure can be chemically synthesized. Chemically synthesized polypeptides are routinely generated by those of skill in the art.

Once assembled (by synthesis, recombinant methodologies, site-directed mutagenesis or other suitable techniques), the DNA sequences encoding a CAR as disclosed herein can be inserted into an expression vector and operably linked to an expression control sequence appropriate for expression of the CAR in the desired transformed host. Proper assembly can be confirmed by nucleotide sequencing, restriction mapping, and expression of a biologically active polypeptide in a suitable host. As is known in the art, in order to obtain high expression levels of a transfected gene in a host, take should be taken to ensure that the gene is operably linked to transcriptional and translational expression control sequences that are functional in the chosen expression host.

### Combinations of Chimeric Antigen Receptors (CARS) (Logic “And” Gates)

As described above, the CAR polypeptides of the present disclosure can be combined to form Boolean logic gates (e.g. AND) to response to a combination of extracellular ligands/antigens. In one aspect, provide herein is a composition having at least two CAR polypeptides of the CAR polypeptides described herein. The CAR combinations described herein, e.g., forming logic gatings, are capable of activating cells (e.g., T cells) expressing the CAR combinations to, e.g., kill target cells and respond only when multiple target antigens are present. The CAR combinations described herein may also reduce T cell exhaustion in CAR T cells while maintaining efficacy/potency. Furthermore, the CAR combinations described herein may enhance activity of the cells (e.g., T cells) expressing the CAR combinations when antigen density is limiting. In some embodiments, at least one CAR molecule of the CAR combinations has an intracellular signaling domain without an ITAM (e.g., without a CD3zeta domain). For example, the at least two CAR polypeptides in a CAR combination may have: 1) a first chimeric antigen receptor (CAR) polypeptide having: a first extracellular ligand-binding domain having a binding affinity for a first ligand; a first transmembrane domain; and a first intracellular signaling domain, and 2) a second chimeric antigen receptor (CAR) polypeptide having: a second extracellular ligand-binding domain having a binding affinity for a second ligand different from the first ligand; a second transmembrane domain; and a second intracellular signaling domain, wherein a cell expressing both CAR polypeptides is activated only when the first and the second extracellular ligand-binding domains bind to the first and the second ligand, respectively, and wherein neither of the first and the second intracellular signaling domains has an ITAM (e.g., neither having CD3zeta).

To form an “AND” gate CAR molecule combination, each of the CAR molecules, when binding to the corresponding ligand/antigen, is not able to activate, by itself, the cell expressing the composition of the CAR combination. Instead, the cell can be activated only when each and every CAR molecule in the “AND” gate combination are activated or bind to their specific ligands. In some embodiments, the intracellular signaling domains of the CAR molecules in the “AND” gate combination, when activated, interact with each other to produce a signaling to activate the cell. An “AND” gate CAR molecule combination can be evaluated based on its potency and specificity. For example, potency represents the degree/level of cell activation induced by a CAR combination after binding to extracellular antigens (i.e., activation of each and every CAR molecule in the combination), while specificity represents the ratio of such degree/level of activation to a background degree/level of cell activation when some but not all of CAR molecules in the combination bind to the corresponding antigen(s). As illustrated in the Examples described herein, different CAR combinations are compared to find combinations with a highest potency in response to a full panel of antigens (e.g., able to activate each and every CAR molecule in the combination) and with a highest specificity (i.e., least “leaky” in response to less than the corresponding full panel of antigens so that not all CAR molecules are activated).

In some embodiments, at least one of the first and the second intracellular signaling domains is a full-length or biologically active fragment of a protein kinase, a G protein, a GTP-binding protein, an adaptor signaling protein, or a scaffold protein capable of inducing cell activation. In some embodiments, at least one of the first and the second intracellular signaling domains is selected from the group consisting of: LAT, SLP-76, CD28, CD2, 4-1BB, CD6, and a biologically active fragment, mutant or variant thereof. In some embodiments, at least one of the first and the second intracellular signaling domains is LAT or SLP-76, or a biologically active fragment, mutant or variant thereof. In some embodiments, the first intracellular signaling domain is LAT or a biologically active fragment, mutant or variant thereof and the second intracellular signaling domain is SLP-76 or a biologically active fragment, mutant or variant thereof. In some embodiments, the first intracellular signaling domain is LAT or a biologically active fragment, mutant or variant thereof and the second intracellular signaling domain is CD28 or a biologically active fragment, mutant or variant thereof.

In some embodiments, CARs in the “AND” gate combination have a same TMD domain, a same optional hinge domain, and/or a same optional costimulatory domain. In some embodiments, CARs in the “AND” gate combination have different TMD domains, optional hinge domains, and/or optional costimulatory domains. For example, different domains, as described herein, may be used for the CARs in the “AND” gate combination to reduce aggregation of the CARs, to modulate the potency and/or the specificity (i.e., the “leakiness”) of the CAR combination, and/or to reduce T cell exhaustion. Sequences of exemplary CARs with a same or different hinge/TM domains are disclosed herein, such as in Tables 1-2 and sequence listing.

### Mutations to Chimeric Antigen Receptors (CARS)

Multiple mutations can be introduced to the CAR polypeptides of the present disclosure, either used alone or in “AND” gate combinations. Mutations include, at least, substitution, deletion, insertion, or other methods known in the art. The purpose of mutations may include, for example, to enhance the potency of the CAR polypeptide (or the CAR polypeptide combination), to enhance the stability (e.g., half-life) of the CAR polypeptide, to enhance the expression of the CAR polypeptide, to enhance the solubility and/or to reduce aggregation of the CAR polypeptide, to manipulate modifications of the CAR polypeptide during expression, to manipulate binding of the CAR polypeptide to its binding partner(s), to enhance the purification of the CAR polypeptide, to reduce ubiquitination and/or degradation of the CAR polypeptide, to reduce the background activation levels of the cell (i.e., the “leakiness”) when some but not all CAR polypeptides in an “AND” gate combination are activated, to reduce aggregation of the multiple CAR polypeptides in an “AND” gate combination in absence of the ligand, etc.

Mutations and deletions may be introduced into at least one domain of the CAR polypeptide. For example, mutations and deletions may include at least one of
i) a mutation of G160D, Y200F, Y220F, Y252F, Y200F/Y220F, or Y200F/Y220F/Y252F, a deletion of amino acid residues at the C terminus (e.g., positions 200-262), a deletion of amino acid residues at positions 28-90 and at positions 200-262, or a deletion of amino acid residues at positions 28-130 and at positions 200-262, corresponding to the wild-type LAT sequence;
ii) a mutation of K30R, a deletion of amino acid residues (e.g., positions 224-244), a deletion of amino acid residues at positions 224-265, a deletion of amino acid residues at positions 224-300, or a deletion of amino acid residues at positions 1-81 and at positions 224-244, corresponding to the wild-type SLP-76 sequence;
iii) at least one mutation in a region on the first and/or the second CAR polypeptide (or each of the CAR polypeptide in an “AND” gate combination) capable of binding to GADS and/or GRB2. Exemplary mutations or deletions for reducing binding to GADS and/or GRB2 include Y200F/Y220F or Y200F/Y220F/Y252F on LAT or deletions such as 4(200-262) on LAT (i.e., a deletion of amino acid residues from position 200 to position 262 of LAT; also as LAT^{200-262 del}) or Δ(224-244) on SLP-76 (also as SLP-76^{224-244 del}); and
iv) at least one deletion of a region on the first and/or the second CAR polypeptide (or each of the CAR polypeptide in an “AND” gate combination) capable of reducing the resulting packaging size of the CAR polypeptide in, e.g., expression vectors (such as viral vectors) without significantly losing its capacity to activate cells expressing such CAR polypeptide. For example, some deletions may be introduced to ZAP70, LAT, and/or SLP-76 domains in the CAR constructs described herein, in order to reduce the construct size and the resulting packaging size into expression vectors (e.g., viral vectors), without a significant loss of activity. Some exemplary deletions include: a deletion of amino acid residues at positions 28-90 or 28-130 of LAT, a deletion of amino acid residues at positions 1-81, 224-265, and/or 224-300 of SLP-76, and a deletion of other portions of ZAP70, keeping only the amino acids at positions 280-600 or 308-600 of ZAP70 in the CAR constructs. Besides the intracellular signaling domain, various mutations may be introduced into other domains for a CAR molecule, such as the hinge/TM domain (e.g., as in an exemplary CD28H/TM^{2CA} mutation described herein). In some embodiments, a CAR polypeptide described herein contains at least one deletion which reduces the length of a domain of the CAR polypeptide or the full CAR polypeptide, and/or the final packaging size of the CAR polypeptide in an expression vector (e.g., a viral vector) to at most 95%, 94%, 93%, 92%, 91%, 90%, 85%, 80%, 75%, 70%, 65%, 60%, 55%, 50%, 45%, 40%, 35%, 30%, 25%, 20%, 15%, 10%, or less. In some embodiments, the reduction of packaging size for a CAR molecule may increase the transduction efficiency of the CAR molecule into cells at least 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 1000%, 1500%, 2000%, 3000%, 5000%, 10000%, or more than the CAR molecule without the at least one deletion or mutation. In some embodiments, a CAR polypeptide described herein contains at least one deletion or other mutation described herein and maintains at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of its activity (e.g., its capacity to activate cells upon antigen exposure). In some embodiments, a CAR polypeptide described herein contains at least one deletion or other mutation described herein and has at least 10%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 1000%, 1500%, 2000%, 3000%, 5000%, 10000%, or more activity (e.g., its capacity to activate cells upon antigen exposure) than the CAR molecule without the at least one deletion or mutation.

Additional exemplary mutations, such as those to CAR molecules containing a truncated ZAP70 fragment, may be found in the present disclosure, such as Examples and Tables 1-2 and sequence listing. Various mutations and deletions, as described herein, can be combined to prepare CAR constructs, performed by a skilled artisan based on the instant description and common knowledge in the art, exemplified by those sequences in Examples and Tables 1-2 and sequence listing.

### Cells Expressing the CAR Polypeptides

The CAR polypeptides (alone or in “AND” gate combinations), when expressed in a host cell, are capable of activating the cell upon binding to the corresponding ligand(s)/antigen(s). In principle, there are no particular limitations with regard to suitable cells for expressing and activation by the CAR polypeptides. In some embodiments, the cell is an immune cell. For example, the immune cell may include, at least, a T cell, a regulatory T cell (Treg), a natural killer (NK) cell, a stem cell, a monocyte, a gamma delta T cell, a monocyte, a macrophage, a natural killer T (NKT) cell, or an iPSC-derived T cell. In some embodiments, the cell is a non-immune cell. The cells may be any type of natural or artificial cells and/or of any origins. By expressing the CAR polypeptides (alone or in “AND” gate combinations) described herein in these cells, an exemplary manipulating mechanism is introduced so that the cells may be activated and such activation may be detected. Thus, any type of cells that people want to study the activation may be used for expressing the CAR polypeptide described herein.

### Nucleic Acid Molecules

In one aspect, provided herein are various nucleic acid molecules including nucleotide sequences encoding a CAR polypeptide of the disclosure, including expression cassettes, and expression vectors containing these nucleic acid molecules operably linked to heterologous nucleic acid sequences such as, for example, regulator sequences which allow in vivo expression of the CAR polypeptide in a host cell or ex-vivo cell-free expression system.

Nucleic acid molecules of the present disclosure can be nucleic acid molecules of any length, including nucleic acid molecules that are generally between about 0.5 Kb and about 50 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb. In some embodiments, the nucleic acid molecules of the disclosure are between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the recombinant nucleic acid includes a nucleic acid sequence encoding a CAR that includes an extracellular ligand-binding domain having a binding affinity for a ligand]; a transmembrane domain; and an intracellular signaling domain. In some embodiments, the CAR encoded by the nucleic acid sequence further includes an optional hinge domain and/or costimulatory domain.

In some embodiments, a composition has at least two recombinant nucleic acids, each including a nucleic acid sequence encoding a CAR polypeptide described herein to form an “AND” gate CAR combination. In some embodiments, these at least two recombinant nucleic acids are conjugated together. In some embodiments, these at least two recombinant nucleic acids are in a single chain of a recombinant nucleic acid.

In some embodiments, the recombinant nucleic acid includes a nucleic acid sequence having at least 50% sequence identity to a nucleic acid sequence selected from the group consisting of SEQ ID NOs: 76-98, 133-145, 157-160, 206, 208, 210, 212, 214-220, 222, 224-228, 230, 231, 233, 234, 236, 238, 240, and 242-248. In some embodiments, the recombinant nucleic acid includes a nucleic acid sequence having at least 50%, at least 60%, at least 70%, at least 80%, at least 85%, at least 90%, at least 95%, at least 96%, at least 97%, at least 98%, at least 99%, or 100% sequence identity sequence identity to a nucleic acid sequence selected from the group consisting of SEQ ID NOs: 76-98, 133-145, 157-160, 206, 208, 210, 212, 214-220, 222, 224-228, 230, 231, 233, 234, 236, 238, 240, and 242-248.

In some embodiments, the recombinant nucleic acid molecule is operably linked to a heterologous nucleic acid sequence.

In some embodiments, the recombinant nucleic acid molecule is further defined as an expression cassette or a vector. It can be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the CAR polypeptide as disclosed herein, which is operably linked to expression control elements, such as a promoter, and optionally, any other sequences or a combination of other nucleic acid sequences that affect the transcription or translation of the coding sequence.

In some embodiments, the nucleotide sequence is incorporated into an expression vector. It can be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As can be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the vector is a vector derived from a lentivirus, an adeno virus, an adeno-associated virus, a baculovirus, or a retrovirus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 50%, 60%, 70%, 80%, 90%, 95%, 96%, 97, 98%, 99%, or 100% sequence identity to a CAR polypeptide disclosed herein. In some embodiments, provided herein are nucleic acid molecules encoding a polypeptide with an amino acid sequence having at least about 50% sequence identity to any one of SEQ ID NOs: 27-49, 111-123, 148-151, 162, 164, 166, 168, 170-176, 178, 180-184, 186, 187, 189, 190, 192, 194, 196, and 198-204. In some embodiments, the nucleic acid molecules encode a polypeptide with an amino acid sequence having at least about at least 50%, at least 60%, at least 70%, at least 80%, at least 85%, 90%, 95%, 96%, 97, 98%, 99%, or 100% sequence identity to SEQ ID NOs: 27-49, 111-123, 148-151, 162, 164, 166, 168, 170-176, 178, 180-184, 186, 187, 189, 190, 192, 194, 196, and 198-204.

The nucleic acid sequences encoding the CAR polypeptides can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

The nucleic acid molecules provided can contain naturally occurring sequences, or sequences that differ from those that occur naturally, but, due to the degeneracy of the genetic code, encode the same polypeptide, e.g., antibody for the ECD. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies for the ECD); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid molecules of the present disclosure can be introduced into a cell (i.e., a host cell), such as a human T cell or cancer cell, to produce a recombinant cell containing the nucleic acid molecule. Accordingly, some embodiments of the disclosure relate to methods for making a recombinant cell, including (a) providing a host cell capable of protein expression; and transducing the provided host cell with a recombinant nucleic acid of the disclosure to produce a recombinant cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be introduced into a host cell by viral or non-viral delivery vehicles known in the art to produce an engineered cell. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using zinc-finger proteins (ZNF), guide RNA directed CRISPR/Cas9, DNA-guided endonuclease genome editing NgAgo (Natronobacterium gregoryi Argonaute), or TALEN genome editing (transcription activator-like effector nucleases).

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the CAR polypeptides of interest.

In some embodiments, the recombinant cell is a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a mouse cell. In some embodiments, the animal cell is a human cell. In some embodiments, the cell is a non-human primate cell. In some embodiments, the recombinant cell is an immune system cell, e.g., a B cell, a monocyte, a NK cell, a natural killer T (NKT) cell, a regulatory T cell (Treg), a basophil, an eosinophil, a neutrophil, a dendritic cell, a macrophage, a regulatory T cell, a helper T cell (T_{H}), a cytotoxic T cell (T_{CTL}), a memory T cell, a gamma delta (γδ) T cell, another T cell, a stem cell (e.g., a hematopoietic stem cell), a stem cell progenitor (e.g., a hematopoietic stem cell progenitor) an induced pluripotent stem cell (iPSC)-derived NK cell, or an induced pluripotent stem cell (iPSC)-derived T cell.

In some embodiments, the immune system cell is a lymphocyte. In some embodiments, the lymphocyte is a T lymphocyte. In some embodiments, the lymphocyte is a T lymphocyte progenitor. In some embodiments, the T lymphocyte is a CD4+ T cell or a CD8+ T cell. In some embodiments, the T lymphocyte is a CD8+ T cytotoxic lymphocyte cell. Non-limiting examples of CD8+ T cytotoxic lymphocyte cell suitable for the compositions and methods disclosed herein include naïve CD8+ T cells, central memory CD8+ T cells, effector memory CD8+ T cells, effector CD8+ T cells, CD8+ stem memory T cells, and bulk CD8+ T cells. In some embodiments, the T lymphocyte is a CD4+T helper lymphocyte cell. Suitable CD4+ T helper lymphocyte cells include, but are not limited to, naïve CD4+ T cells, central memory CD4+ T cells, effector memory CD4+ T cells, effector CD4+ T cells, CD4+ stem memory T cells, and bulk CD4+ T cells.

In some embodiments, the host cell described herein is a non-immune system cell. For example, the CAR molecules and/or the CAR molecule combinations described herein provide a method to modulate the activity of (e.g., to activate) a cell expressing such CAR molecules and/or combinations, upon recognizing the corresponding extracellular ligand(s)/antigen(s). In this sense, there are no particular limitations with regard to suitable host cell.

As outlined above, some embodiments of the disclosure relate to various methods for making a recombinant cell, including (a) providing a host cell capable of protein expression; and transducing the provided host cell with a recombinant nucleic acid of the disclosure to produce a recombinant cell. Non-limiting exemplary embodiments of the disclosed methods for making a recombinant cell can further include one or more of the following features. In some embodiments, the host cell is obtained by leukapheresis performed on a sample obtained from a subject, and the cell is transduced ex vivo. In some embodiments, the recombinant nucleic acid is encapsulated in a viral capsid or a lipid nanoparticle. In some embodiments, the methods further include isolating and/or purifying the produced cells. Accordingly, the recombinant cells produced by the methods disclosed herein are also within the scope of the disclosure.

Techniques for transforming a wide variety of the above-mentioned host cells and species are known in the art and described in the technical and scientific literature. For example, DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection. In some embodiments, the nucleic acid molecule is introduced into a host cell by a transduction procedure, electroporation procedure, or a biolistic procedure. Accordingly, cell cultures including at least one recombinant cell as disclosed herein are also within the scope of this application. Methods and systems suitable for generating and maintaining cell cultures are known in the art.

In some embodiments, the recombinant cell includes a nucleic acid molecule including a nucleic acid sequence encoding a CAR having at least 50%, 60%, 70%, 80%, 90%, 95%, 96%, 97, 98%, 99%, or 100% sequence identity to a CAR polypeptide disclosed herein. In some embodiments, the recombinant cell includes a nucleic acid molecule encoding a polypeptide with an amino acid sequence having at least about 50%, 60%, 70%, 80%, 90%, 95%, 96%, 97, 98%, 99%, or 100% sequence identity to any one of SEQ ID NOs: 27-49, 111-123, 148-151, 162, 164, 166, 168, 170-176, 178, 180-184, 186, 187, 189, 190, 192, 194, 196, and 198-204.

In a related aspect, some embodiments of the disclosure relate to cell cultures including at least one recombinant cell as disclosed herein, and a culture medium. Generally, the culture medium can be any one of suitable culture media for the cell cultures described herein. In some embodiments, the recombinant cell expresses a CAR described herein. Accordingly, cell cultures including at least one recombinant cell as disclosed herein are also within the scope of this application. Methods and systems suitable for generating and maintaining cell cultures are known in the art.

### Pharmaceutical Compositions

The CAR polypeptides, nucleic acids, recombinant cells, and/or cell cultures of the disclosure can be incorporated into compositions, including pharmaceutical compositions. Such compositions generally include the CARs, nucleic acids, recombinant cells, and/or cell cultures as described herein and a pharmaceutically acceptable carrier. Accordingly, in one aspect, some embodiments of the disclosure relate to pharmaceutical compositions for treating, preventing, ameliorating, reducing or delaying the onset of a health condition, for example a proliferative disease (e.g., cancer). Other exemplary health conditions include, e.g., hematological malignancies, solid tumors, autoimmune diseases, inflammations, allergic diseases, infections, senescence/aging, etc.

Accordingly, one aspect of the present disclosure relates to pharmaceutical compositions that include a pharmaceutically acceptable carrier and one or more of the following: (a) a CAR polypeptide of the disclosure; (b) a nucleic acid molecule of the disclosure; and/or (c) a recombinant cell of the disclosure. In some embodiments, the composition includes (a) a recombinant nucleic acid of the disclosure and (b) a pharmaceutically acceptable carrier. In some embodiments, the recombinant nucleic acid is encapsulated in a viral capsid or a lipid nanoparticle. In some embodiments, the composition includes (a) a recombinant cell of the disclosure and (b) a pharmaceutically acceptable carrier.

In certain embodiments, the pharmaceutical compositions in accordance with some embodiments disclosed herein include cell cultures that can be washed, treated, combined, supplemented, or otherwise altered prior to administration to an individual in need thereof. Furthermore, administration can be at varied doses, time intervals or in multiple administrations.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art can appreciate that the formulation should suit the mode of administration.

In some embodiments, the pharmaceutical compositions of the present disclosure are formulated to be suitable for the intended route of administration to an individual. For example, the pharmaceutical composition may be formulated to be suitable for parenteral, intraperitoneal, colorectal, intraperitoneal, and intratumoral administration. In some embodiments, the pharmaceutical composition may be formulated for intravenous, oral, intraperitoneal, intratracheal, subcutaneous, intramuscular, topical, or intratumoral administration.

### Use of the Compositions

The compositions described herein, e.g., CARs (alone or in combinations), nucleic acids, recombinant cells, cell cultures, and/or pharmaceutical compositions, can be used for various conditions, as a system in response to extracellular signals (e.g., ligands/antigens).

For example, the compositions described herein may be used as a detection system for detecting certain ligands/antigens. As described herein, in principle, there are no particular limitations with regard to suitable target antigens and suitable host cells. In some embodiments, targeting a specific ligand/antigen or a specific profile of ligands/antigens, the corresponding CAR molecule(s) is prepared to having an extracellular antigen-binding domain (ECD) to specifically binding to the ligand(s)/antigen(s). The corresponding intracellular signaling domain(s) can then activate, after the ligand/antigen binding, the host cell to produce a signal to be detected, such as expressing a gene for detection (e.g., by detecting the expressed DNA/RNA/protein or any luminescence/fluorescence from the expressed protein). In some embodiments, such detection system may be used for detecting certain biomarkers in a biological sample, such as used for diagnosis of any disease or disorder.

The compositions described herein may also be used as an activation system to manipulate cell functions in response to certain ligands/antigens. In some embodiments, by sensing a specific ligand/antigen or a specific profile of ligands/antigens with the ECD(s) of the CAR molecules or CAR molecule combinations, the host is activated. Such activation may enhance or inhibit the normal biological functions of the host cell, and/or provide an exogenous signaling function to manipulate the cell functions.

The compositions described herein may also be used to manipulate the functions of a target cell in response to certain ligands/antigens. In some embodiments, by sensing a specific ligand/antigen or a specific profile of ligands/antigens with the ECD(s) of the CAR molecules or CAR molecule combinations, the host cell is activated. Such activation may enhance or inhibit the functions of the host cell to change the function of a target cell, which expresses such ligand(s)/antigen(s) or is specifically recognized by certain ligand(s)/antigen(s). For example, a cancer cell expressing certain ligand(s)/antigen(s) may be recognized by the CAR molecule(s) or CAR molecule combination(s) described herein, either through the ECD binding to the ligand(s)/antigen(s) on the surface of the cancer cell or binding to some ligand(s)/antigen(s) which specifically binds to the ligand(s)/antigen(s) expressed on the cancer cell. The activated host cell can then manipulate the function of the cancer cell. For example, the compositions described herein may be used to activate the host cell (e.g., an immune system cell, such as a T cell) to antagonize or kill the cancer cells (e.g., by secreting cytokines or direct killing). Methods are provided herein for a CAR molecule or CAR molecule combination to detect the recognized target cells (e.g., cancer cells) and/or to activate the cytotoxicity of the host cell, such as to antagonize and/or kill the recognized target cells (e.g., cancer cells). In some embodiments, such target cells (e.g., cancer cells) are obtained from a biological sample form a subject. In some embodiments, such target cells (e.g., cancer cells) are in a biological environment (e.g., a cancer microenvironment) of a subject. In some embodiments, the target cell is correlated to a disease or disorder. Exemplary diseases or disorders may include, e.g., proliferative diseases (e.g., cancers), hematological malignancies, solid tumors, autoimmune diseases, inflammations, allergic diseases, infections, senescence/aging, etc.

### Methods of Treatment

Administration of any one of the therapeutic compositions described herein, e.g., CARs, nucleic acids, recombinant cells, cell cultures, and/or pharmaceutical compositions, can be used in the diagnosis, prevention, and/or treatment of relevant conditions, such as proliferative diseases (e.g., cancer), hematological malignancies, solid tumors, autoimmune diseases, inflammations, allergic diseases, infections, senescence/aging, etc. In some embodiments, the CARs, nucleic acids, recombinant cells, cell cultures, and/or pharmaceutical compositions as described herein can be incorporated into therapies and therapeutic agents for use in methods of preventing and/or treating an individual who has, who is suspected of having, or who may be at high risk for developing one or more health conditions, such as proliferative diseases (e.g., cancers, such as a leukemia, a neuroblastoma, or an osteosarcoma). In some embodiments, the individual is a patient under the care of a physician.

Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is pancreatic cancer, colon cancer, ovarian cancer, prostate cancer, lung cancer, mesothelioma, breast cancer, urothelial cancer, liver cancer, head and neck cancer, sarcoma, cervical cancer, stomach cancer, gastric cancer, melanoma, uveal melanoma, cholangiocarcinoma, multiple myeloma, leukemia, lymphoma, haematological cancer, bladder cancer, neuroblastoma, malignant pleural mesothelioma, sarcoma, and glioblastoma. Exemplary cancer types also include: Acute myeloid leukemia, Angioimmunoblastic T-cell lymphoma, B-cell acute lymphoblastic leukemia, Sweet Syndrome, T-cell Non-Hodgkins lymphoma (including natural killer/T-cell lymphoma, adult T-cell leukaemia/lymphoma, enteropathy type T-cell lymphoma, hepatosplenic T-cell lymphoma and cutaneous T-cell lymphoma), T-cell acute lymphoblastic leukemia, B-cell Non-Hodgkins lymphoma (including Burkitt lymphoma, diffuse large B-cell lymphoma, Follicular lymphoma, Mantle cell lymphoma, Marginal Zone lymphoma, etc.), Hairy Cell Leukemia, Hodgkin lymphoma, Lymphoblastic lymphoma, Lymphoplasmacytic lymphoma, Mucosa-associated lymphoid tissue lymphoma, Multiple myeloma, Myelodysplastic syndrome, Plasma cell myeloma, Primary mediastinal large B-cell lymphoma, chronic myeloproliferative disorders (such as chronic myeloid leukemia, primary myelofibrosis, essential thrombocytemia, polycytemia vera) and chronic lymphocytic leukemia. Exemplary cancer types also include: acute lymphoblastic leukemia (ALL), acute myeloid leukemia (AML), adrenocortical carcinoma, AIDS-related cancers, Kaposi sarcoma (soft tissue sarcoma), AIDS-related lymphoma, primary CNS lymphoma, anal cancer, appendix cancer, astrocytomas, childhood brain cancer, atypical teratoid/rhabdoid tumor, central nervous system cancer, skin cancer (e.g., basal cell carcinoma), bile duct cancer, bladder cancer, bone cancer (includes Ewing sarcoma, osteosarcoma and malignant fibrous histiocytoma), brain tumors, breast cancer, bronchial tumors, Burkitt lymphoma, non-Hodgkin lymphoma, carcinoid tumor, Cardiac (heat) tumors, medulloblastoma and other CNS embryonal tumors, germ cell tumor, Primary CNS Lymphoma, Cervical Cancer, Childhood Cancers, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Mycosis Fungoides, Sézary Syndrome, ductal carcinoma in situ (DCIS), Embryonal Tumors, Medulloblastoma, Endometrial Cancer (Uterine Cancer), Ependymoma, Esophageal Cancer, Esthesioneuroblastoma (Head and Neck Cancer), Ewing Sarcoma, Extracranial Germ Cell Tumor, Childhood Extragonadal Germ Cell Tumor, Eye Cancer, Intraocular Melanoma, Retinoblastoma, Fallopian Tube Cancer, Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Childhood Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kidney (Renal Cell) Cancer, Langerhans Cell Histiocytosis, Laryngeal Cancer, Leukemia, Lip and Oral Cavity Cancer, Liver Cancer, Lung Cancer (e.g., Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Intraocular Melanoma, Merkel Cell Carcinoma, Mesothelioma, Metastatic Cancer, Metastatic Squamous Neck Cancer with Occult Primary, Midline Tract Carcinoma, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma, Undifferentiated Pleomorphic Sarcoma, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer Prostate Cancer, Rectal Cancer, Recurrent Cancer, Retinoblastoma, Rhabdomyosarcoma, Salivary Gland Cancer, Sarcoma, Childhood Rhabdomyosarcoma, Childhood Vascular Tumors, Soft Tissue Sarcoma, Uterine Sarcoma, Sézary Syndrome, Skin Cancer, Small Cell Lung Cancer Small Intestine Cancer, Soft Tissue Sarcoma, Squamous Cell Carcinoma, Squamous Neck Cancer with Occult Primary, Stomach (Gastric) Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Tracheobronchial Tumors, Transitional Cell Cancer of the Renal Pelvis and Ureter, Carcinoma of Ureter and Renal Pelvis, Transitional Cell Cancer, Urethral Cancer, Uterine Cancer, Uterine Sarcoma, Vaginal Cancer, Vascular Tumors, Vulvar Cancer, Wilms Tumor, and other Childhood Kidney Tumors.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Accordingly, in one aspect, some embodiments of the disclosure relate to methods for the prevention and/or treatment of a condition in a subject in need thereof, wherein the methods include administering to the subject a composition including one or more of: a CAR polypeptide of the disclosure, a recombinant nucleic acid of the disclosure, a recombinant cell of the disclosure, and/or a pharmaceutical composition of the disclosure.

In some embodiments, the administered composition inhibits proliferation of a target cancer cell, and/or inhibits tumor growth of the cancer in the subject. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit the proliferation of the target cancer cell and/or inhibit tumor growth of a target cancer in the subject compared to the proliferation of the target cell and/or tumor growth of the target cancer in subjects who have not been administered with the recombinant cells. In some embodiments, the target cancer cell is a leukemia cancer cell, a cell derived from a leukemia cancer cell, or a cell in a microenvironment of a leukemia. In some embodiments, the target cancer cell is a neuroblastoma cell, a cell derived from a neuroblastoma cell, or a cell in a microenvironment of a neuroblastoma. In some embodiments, the target cancer cell is an osteosarcoma cell, a cell derived from an osteosarcoma cell, or a cell in a microenvironment of an osteosarcoma. In some embodiments, the target cancer cell is an autoimmune cell.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

Administration of the compositions described herein, e.g., CARs, nucleic acids, recombinant cells, cell cultures, and/or pharmaceutical compositions, can be used in the stimulation of an immune response. In some embodiments, CARs, nucleic acids, recombinant cells, cell cultures, and/or pharmaceutical compositions as described herein are administered to an individual after induction of remission of cancer with chemotherapy, or after autologous or allogeneic hematopoietic stem cell transplantation. In some embodiments, compositions described herein are administered to an individual in need of increasing the production of interferon gamma (IFNγ), TNF-α, and/or interleukin-2 (IL-2) in the treated subject relative to the production of these molecules in subjects who have not been administered one of the therapeutic compositions disclosed herein.

An effective amount of the compositions described herein, e.g., CARs, nucleic acids, recombinant cells, cell cultures, and/or pharmaceutical compositions, is determined based on the intended goal, for example tumor regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each individual. Frequency of administration could range from 1-2 days, to 2-6 hours, to 6-10 hours, to 1-2 weeks or longer depending on the judgment of the practitioner.

In some embodiments, administration is by bolus injection. In some embodiments, administration is by intravenous infusion. In some embodiments, a composition is administered is administered in a dosage of about 100 ng/kg of body weight per day to about 100 mg/kg of body weight per day. In some embodiments, a composition as disclosed herein is administered in a dosage of about 0.001 mg/kg to 100 mg/kg of body weight per day. In some embodiments, the therapeutic agents are administered in a single administration. In some embodiments, therapeutic agents are administered in multiple administrations, (e.g., once or more per week for one or more weeks).

One of ordinary skill in the art would be familiar with techniques for administering compositions of the disclosure to an individual. Furthermore, one of ordinary skill in the art would be familiar with techniques and pharmaceutical reagents necessary for preparation of these compositions prior to administration to an individual.

In certain embodiments of the present disclosure, the composition of the disclosure contains an aqueous composition that includes one or more of the CARs, nucleic acids, recombinant cells, cell cultures, and/or pharmaceutical compositions as described herein. Aqueous compositions of the present disclosure contain an effective amount of a composition disclosed herein in a pharmaceutically acceptable carrier or aqueous medium. Thus, the “pharmaceutical preparation” or “pharmaceutical composition” of the disclosure can include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the recombinant cells disclosed herein, its use in the manufacture of the pharmaceutical compositions is contemplated. Supplementary active ingredients can also be incorporated into the compositions. For human administration, preparations should meet sterility, pyrogenicity, general safety, and purity standards as required by the FDA Center for Biologics.

One of ordinary skill in the art would appreciate that biological materials should be extensively dialyzed to remove undesired small molecular weight molecules and/or lyophilized for more ready formulation into a desired vehicle, where appropriate. The compositions described herein, e.g., CARs, nucleic acids, recombinant cells, cell cultures, and/or pharmaceutical compositions, can then generally be formulated for administration by any known route, such as parenteral administration. Determination of the amount of compositions to be administered can be made by one of skill in the art, and can in part be dependent on the extent and severity of cancer, and whether the recombinant cells are being administered for treatment of existing cancer or prevention of cancer. The preparation of the compositions containing the CARs, nucleic acids, recombinant cells, cell cultures, and/or pharmaceutical compositions of the disclosure can be known to those of skill in the art in light of the present disclosure.

Upon formulation, the compositions of the disclosure can be administered in a manner compatible with the dosage formulation and in such amount as is therapeutically effective. The compositions can be administered in a variety of dosage forms, such as the type of injectable solutions described above.

In some embodiments, CARs, nucleic acids, recombinant cells, cell cultures, and/or pharmaceutical compositions described herein can be used to reduce T cell exhaustion in the corresponding T cells or in the treated subject relative to a subject who has not been administered one of the therapeutic compositions disclosed herein. In some embodiments, CARs, nucleic acids, recombinant cells, cell cultures, and/or pharmaceutical compositions described herein can be used to stimulate proliferation and/or killing capacity of CAR T-cells in the treated subject relative to the production of these molecules in subjects who have not been administered one of the therapeutic compositions disclosed herein. The production of interferon gamma (IFNγ), TNF-α, and/or interleukin-2 (IL-2) can be stimulated to produce up to about 20 fold, such as any of about 2 fold, 3 fold, 4 fold, 5 fold, 6 fold, 7 fold, 8 fold, 9 fold, 10 fold, 11 fold, 12 fold, 13 fold, 14 fold, 15 fold 16 fold, 17 fold, 18 fold, 19 fold, or 20 fold or higher compared to the production of interferon gamma (IFNγ), TNF-α, and/or interleukin-2 (IL-2) in subjects who have not been administered one of the therapeutic compositions disclosed herein.

### Administration of Recombinant Cells to a Subject

In some embodiments, the methods of the disclosure involve administering an effective amount or number of the recombinants cells provided here to a subject in need thereof. This administering step can be accomplished using any method of implantation delivery in the art. For example, the recombinant cells can be infused directly in the subject's bloodstream or otherwise administered to the subject.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, i.e., long-term engraftment.

In some embodiments, the delivery of a recombinant cell composition (e.g., a composition including a plurality of recombinant cells according to any of the cells described herein) into a subject by a method or route results in at least partial localization of the cell composition at a desired site. Modes of administration include, e.g., injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection.

### Kits

Also provided herein are various kits for the practice of a method described herein. In particular, some embodiments of the disclosure provide kits for the diagnosis of a condition in a subject. Some other embodiments relate to kits for the prevention of a condition in a subject in need thereof. Some other embodiments relate to kits for methods of treating a condition in a subject in need thereof. For example, provided herein, in some embodiments, are kits that include one or more of the CAR polypeptides, recombinant nucleic acids, engineered cells, or pharmaceutical compositions as provided and described herein, as well as written instructions for making and using the same.

In some embodiments, the kits of the disclosure further include one or more means useful for the administration of any one of the provided CAR polypeptides, recombinant nucleic acids, engineered cells, or pharmaceutical compositions to an individual. For example, in some embodiments, the kits of the disclosure further include one or more syringes (including pre-filled syringes) and/or catheters (including pre-filled syringes) used to administer any one of the provided CAR polypeptides, recombinant nucleic acids, engineered cells, or pharmaceutical compositions to an individual. In some embodiments, a kit can have one or more additional therapeutic agents that can be administered simultaneously or sequentially with the other kit components for a desired purpose, e.g., for diagnosing, preventing, or treating a condition in a subject in need thereof.

In some embodiments, a kit can further include instructions for using the components of the kit to practice the methods disclosed herein.

No admission is made that any reference cited herein constitutes prior art. The discussion of the references states what their authors assert, and the inventors reserve the right to challenge the accuracy and pertinence of the cited documents. It can be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives can be apparent to those of skill in the art upon review of this disclosure, and are to be included within the spirit and purview of this application.

### EXAMPLES

Additional embodiments are disclosed in further detail in the following examples, which are provided by way of illustration and are not in any way intended to limit the scope of this disclosure or the claims.

### Example 1

### Identifying Downstream T Cell Response Signaling Molecules for Use in CAR Constructs

This Example describes experiments performed to illustrate the identification of essential downstream T cell response (TCR) signaling molecules. Exemplary CAR molecules were engineered by replacing traditional signaling domains with alternative molecules from the proximal T cell signaling cascade. The engineered CAR molecules were then tested for their functions to activate cells expressing these molecules, such as the function to elicit T cell anti-tumor activity.

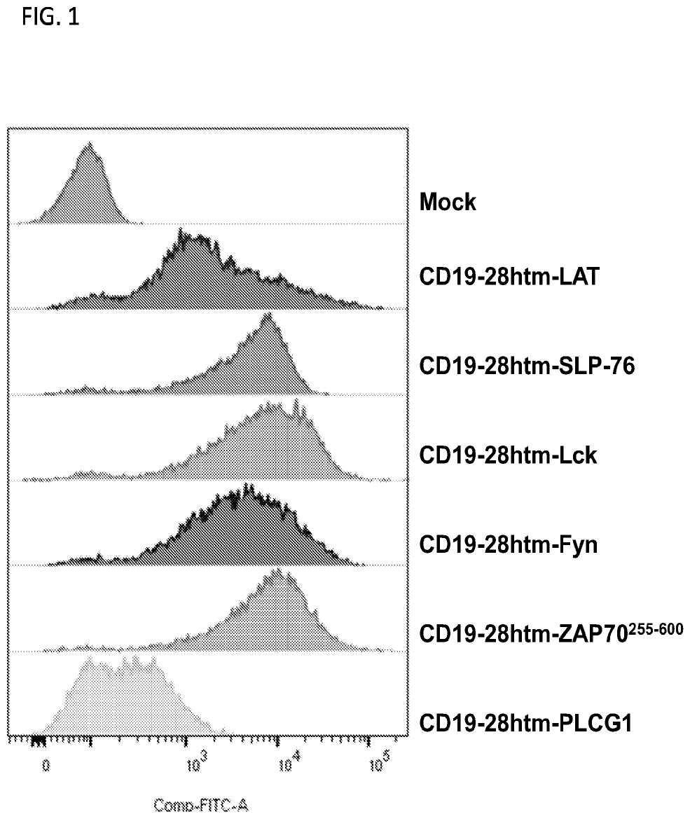

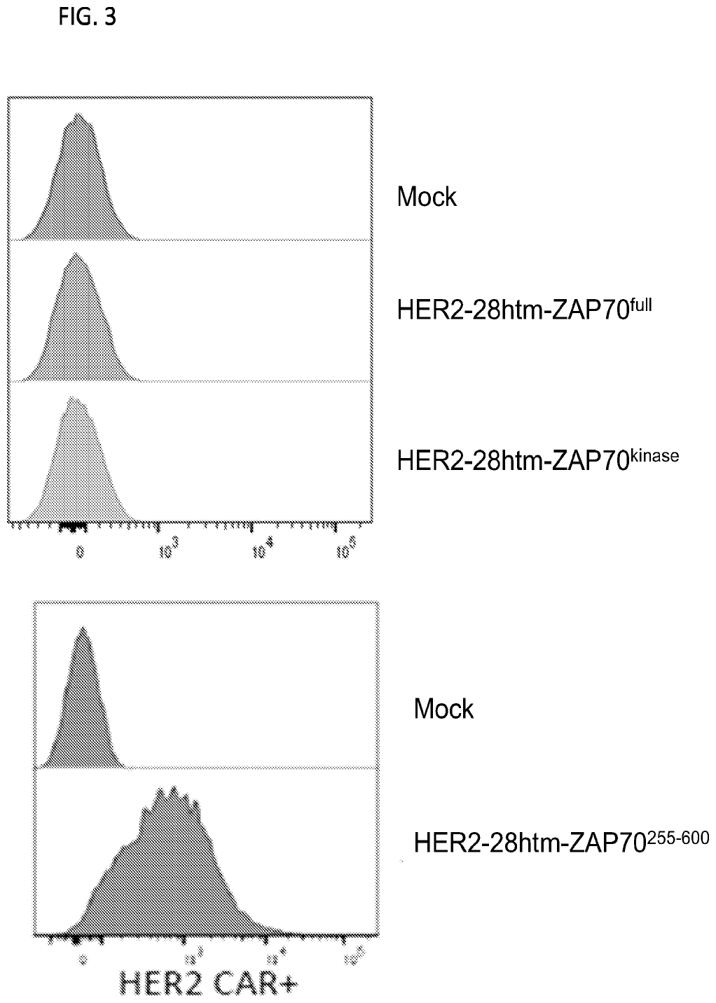

Proximal signaling molecules were tested to see if any of them is sufficient to propagate a T cell signal. Since most proximal signaling molecules are cytosolic and do not have a TM domain, CAR constructs were prepared by linking an scFv (e.g., recognizing CD19 or HER2) and a CD28 hinge/transmembrane (28H/TM) domain directly to each proximal signaling molecule. FIG. 1 shows FACS histograms illustrating each of several downstream TCR signaling molecule constructs. Exemplary constructs contain LCK, FYN, ZAP70, LAT, SLP-76, or PLC-gamma (PLCG1). The truncated ZAP70 construct ZAP70²⁵⁵⁻⁶⁰⁰ was used because the CAR construct having either the full-length ZAP70 or only the kinase domain of ZAP70 did not express on the surface of T cells, while the truncated ZAP70²⁵⁵⁻⁶⁰⁰ version did express (FIG. 3). The truncated ZAP70²⁵⁵⁻⁶⁰⁰ contains the intramolecular linker, interdomain B and a segment of the kinase domain of ZAP70. These differences in expression patterns were found in both HER2-recognizing ZAP70 CAR constructs (FIG. 3) and CD19-recognizing ZAP70 CAR constructs.

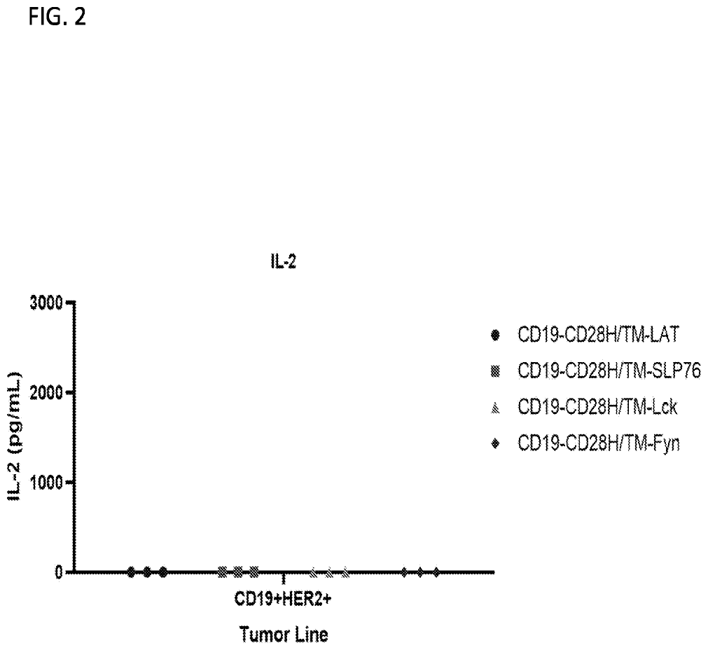

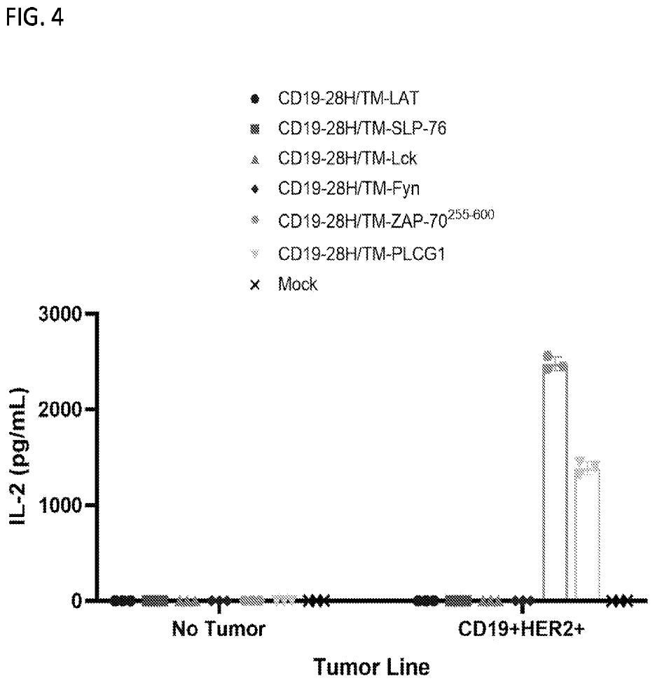

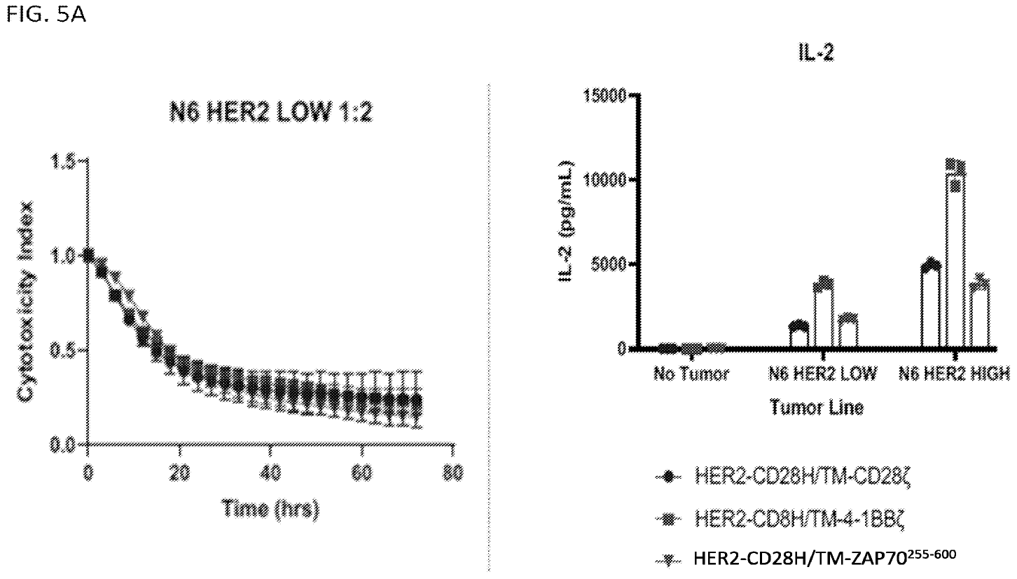

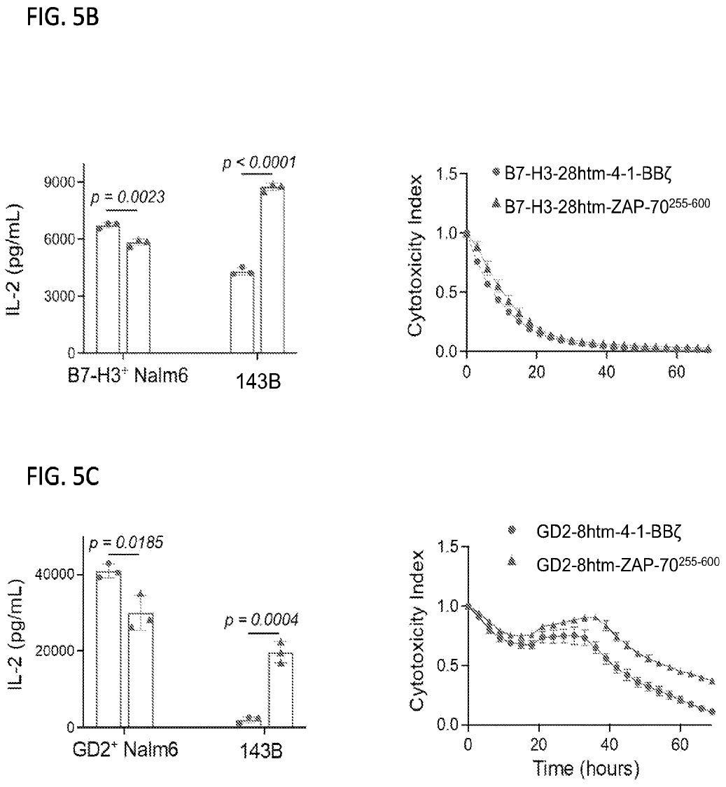

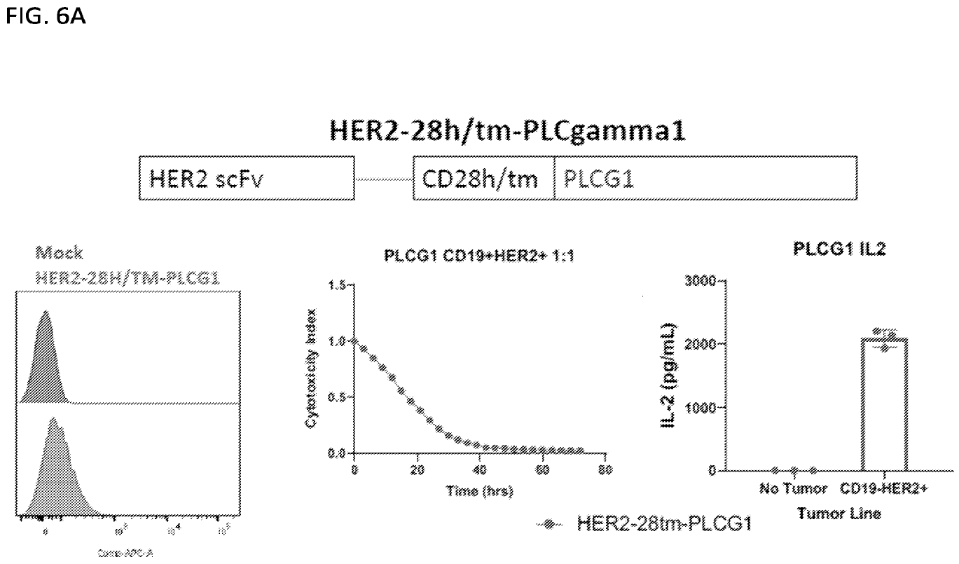

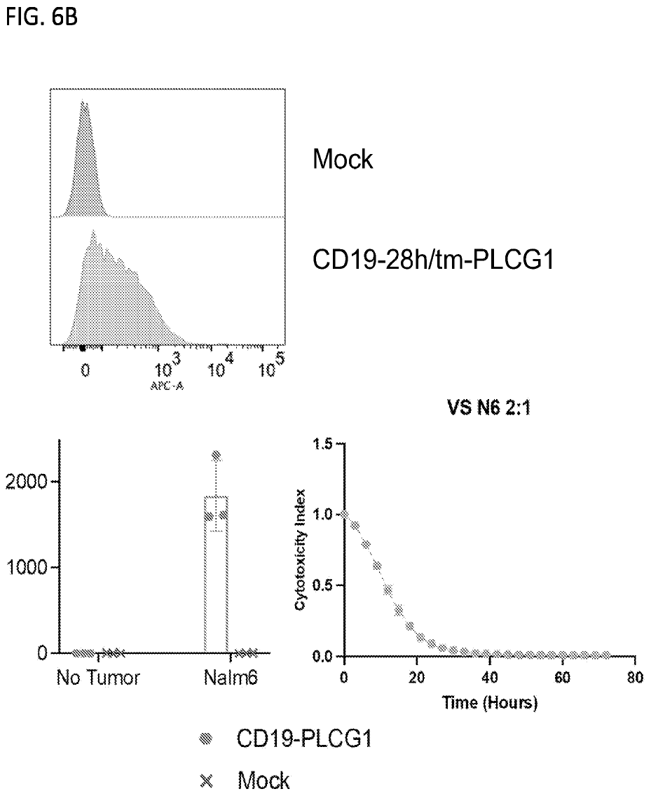

The TCR signaling molecule constructs were tested for their abilities to induce cytokine (e.g., IL-2) generation in response to antigen exposure. Exemplary constructs listed above were expressed in primary T cells and the cytokine production by the T cells in the presence of tumor cell lines expressing CD19 and HER2 antigens were measured and compared. As shown in FIGS. 2 and 4, LAT, SLP-76, LCK, and FYN CARs did not promote T cells to produce IL-2 in response to antigen (CD19). Thus, each of these downstream TCR signaling molecules, as in the CAR construct, is not sufficient for inducing T cell activity. Furthermore, CAR molecules with a truncated ZAP70²⁵⁵⁻⁶⁰⁰ fragment were tested for their abilities to induce the T cell cytotoxic activity to tumor cells expressing the antigen (e.g., CD19 or HER2). As shown in FIGS. 4 and 5A-5C, the ZAP70²⁵⁵⁻⁶⁰⁰ CARs targeting HER2, B7-H3, or GD2, similar to traditional CD28-zeta (CD28) and 4-1BB-zeta (4-1BB CARs, were able to promote T cells to kill tumor cells and to generate cytokines (e.g., IL-2) in response to tumor exposure. A comparison of all these CAR molecules for their capacities of inducing IL-2 expression by T cells is shown in FIG. 4. As shown in FIGS. 4 and 6A-6B, similar PLCG1 (i.e., PLCgamma1 or PLCγ1) CAR constructs also induced the cytotoxic and/or cytokine (IL-2) production activity of T cells in response to tumor exposure. As a summary of these experiments, CAR molecules having endodomains consisting of either the truncated form of ZAP70 (i.e., ZAP70²⁵⁵⁻⁶⁰⁰) or PLCG1 activated T cells, measured by cytokine production and tumor cytotoxicity of the T cells, while others tested CAR constructs (LAT, LCK, FYN, or SLP-76) did not activate T cells to produce IL-2. These findings demonstrate that T cell activity is not dependent on CD3zeta, but can be elicited, in an antigen specific manner, by CARs containing specific proximal signaling molecules as well. These also demonstrate that cytosolic molecules that appear downstream in signaling cascades can be used in CAR constructs and can be sufficient to initiate and drive cell activation.

### Example 2

### Engineering Boolean Logic Gated CAR Constructs

This Example describes experiments performed to generate a molecular toolbox to develop effective Boolean logic gated CAR T cells (e.g., a CAR T cell expressing two CAR molecules in which the cell is activated only when both CAR molecules bind to their specific ligands and activate their cytosolic signaling domains, a.k.a., an “AND” gate). The generated CAR T cells are capable of discriminating between diseased cells and normal tissues.

It is important to note that CD3zeta alone within a CAR molecule is sufficient to trigger cytolytic activity in the presence of antigen. Therefore, for solid tumors and myeloid malignancies, where most antigen targets are shared with normal and vital tissues, CARs capable of inducing an anti-tumor response are also capable of mediating serious and life threatening on-target, off-tumor toxicity. Reliance on incorporating CD3zeta within CARs has severely limited the ability to develop complex Boolean gated CARs that conditionally control CAR T cell function. For example, multiple groups have attempted to design an AND-gate CAR which can trigger an anti-tumor response only when two target antigens are both present. All have major limitations that preclude their widespread use in the clinic, largely based on their reliance on CD3zeta. In a first attempt, the CD3zeta and costimulatory domain were separated onto two different CARs with distinct specificities (“Split CAR”,) (Kloss et al. Nat Biotechnol. 2013; 31:71-75). While this led to attenuation of CAR activity when only one antigen was engaged, CARs containing only CD3zeta without a costimulatory domain are still active and capable of mediating on-target, off-tumor toxicity in patients (Lamers et al. Mol Ther. 2013; 21:904-912). A technically more sophisticated system, SynNotch, relied on transcriptional control of a CD3zeta containing CAR in response to antigen encounter (Roybal et al. Cell. 2016; 164:770-779). Upon encountering antigen A, the SynNotch receptor drove expression of a fully enabled CD3zeta containing CAR against antigen B. Studies have demonstrated that this system does not function as a true AND gate because once the CD3zeta containing CAR is expressed, T cells are cable of killing both tumor cells and normal bystander cells that express only the second antigen. In fact, mice treated with a ROR1-specific CAR using this system demonstrated on-target, off-tumor toxicity that resulted in death (Srivastava et al. Cancer Cell. 2019; 35:489-503.e8).

In this Example, by linking cytosolic molecules from the T cell signaling cascade to a CAR, a molecular toolbox was generated to develop exemplary truly effective Boolean logic gated CAR T cells. An exemplary system described herein, not relying on CD3zeta, is capable of rapidly and reversibly activating T cells only when encountering two antigens simultaneously. By escaping the dominant paradigm that CD3zeta is required for CAR T cell activation, complete control of CAR T cell activity is achieved by a Boolean logic gated system, greatly expanding the CAR reach in the clinic.

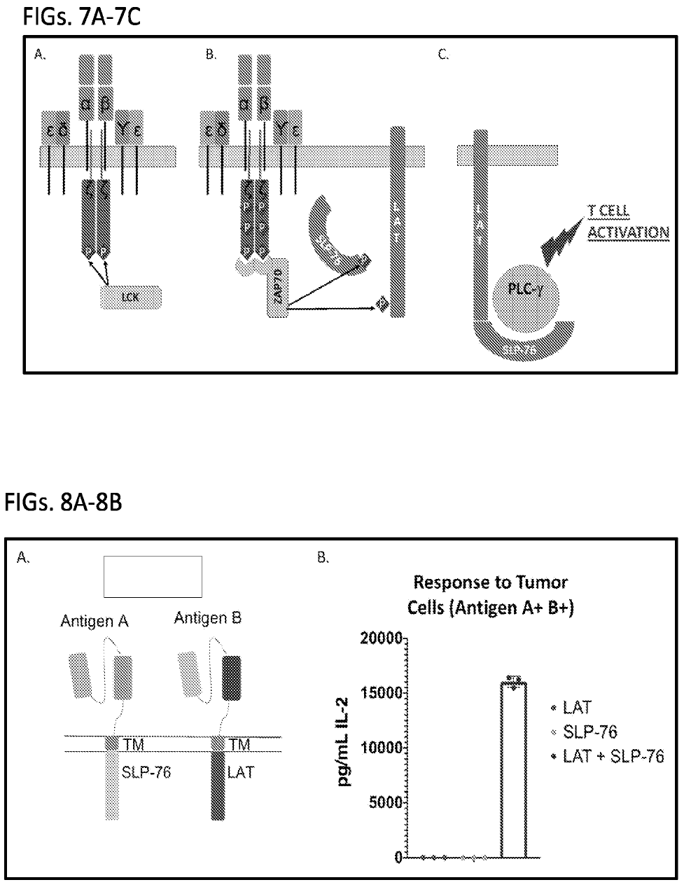

In a detailed illustration of the T cell signaling cascade, CD3zeta is phosphorylated by LCK and/or FYN (FIG. 7A) and then serves as a docking site for ZAP70, resulting in its activation (FIG. 7B). ZAP70 then phosphorylates several downstream adapter and scaffold proteins, the most important of which are LAT and SLP-76. Once phosphorylated, LAT and SLP-76 come together to form a scaffold for PLCG1 and other molecules that are capable of mediating downstream effector functions (FIG. 7C).

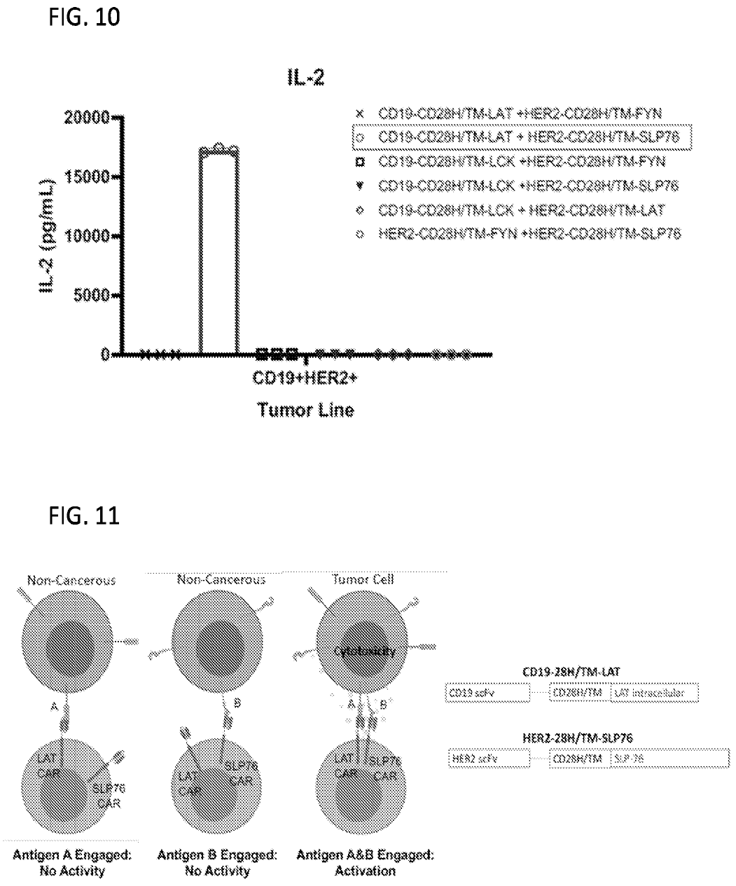

An exemplary AND gate for CAR signaling was designed by expressing separate CARs to bring together LAT and SLP-76, the targets of ZAP70 that form a binding site for PLCG1 (FIG. 8A). Either a LAT CAR or a SLP-76 CAR, prepared as described in Example 1, was not sufficient to activate T cells exposed to target antigens to produce cytokine (IL-2) (FIG. 8B). However, when the two CARs (targeting different antigens) were co-expressed on a single T cell, which was exposed to tumor cells expressing both target antigens, robust T cell activation was achieved (FIGS. 8B and 10).

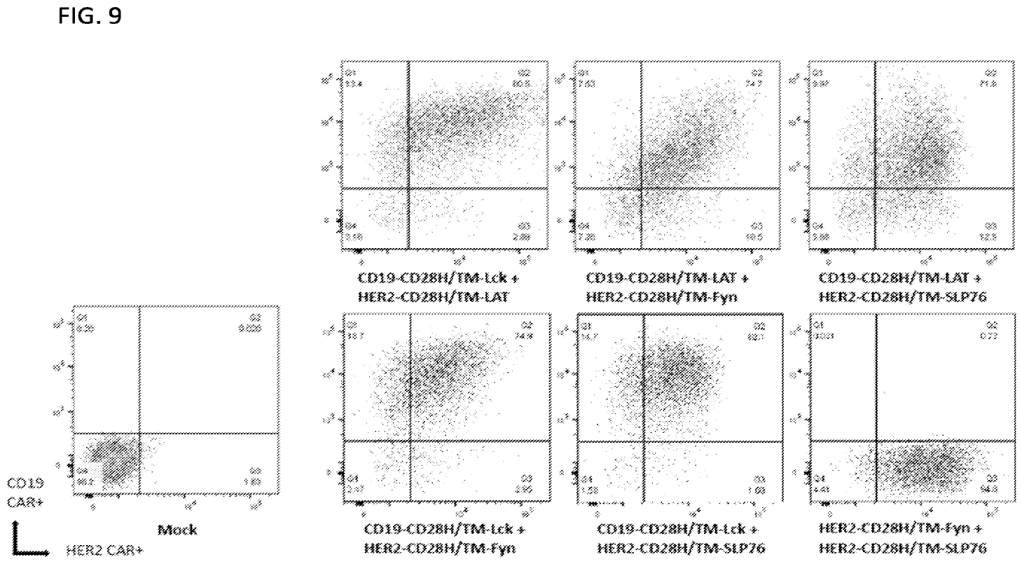

CAR molecules were prepared in a construct framework having an extracellular antigen-binding domain specifically recognizing CD19 or HER2 antigens, a CD28 hinge/transmembrane domain, and a cytosolic signaling domain having each of the proximal signaling molecules. FACS data for several combinations of signaling molecules is shown in FIG. 9. Other combinations of signaling molecules in similar CAR constructs were tested for comparison. As shown in FIG. 10, only a CAR construct containing LAT and a CAR construct containing SLP-76, when co-expressed on T cells, induced cytokine (IL-2) expression by the T cells. By contrast, CAR combinations containing other proximal signaling molecules (e.g. LCK plus LAT, SLP-76 plus FYN, or other combinations shown in FIG. 10) had no activity. Thus, the combination of LAT and SLP-76 CARs was designed to co-opt the T cell signaling cascade and functions as a specific AND gate for signaling (FIGS. 8B and 11). A cartoon of T cells expressing this AND gate system, which specifically targets tumor cells expressing both antigens, is shown in FIG. 11. In short, a synthetic dependency for CAR T cell signaling was created, generating a system where T cell activation is dependent on two separate antigen specific inputs.

### Example 3

### Optimization of AND-Gate CAR Activity

This Example describes experiments performed to introduce modifications, mutations, and deletions to either LAT or SLP-76 CAR molecule to enhance the potency of the AND gate system only in response to dual antigens, without a substantial background activation in response to a single antigen.

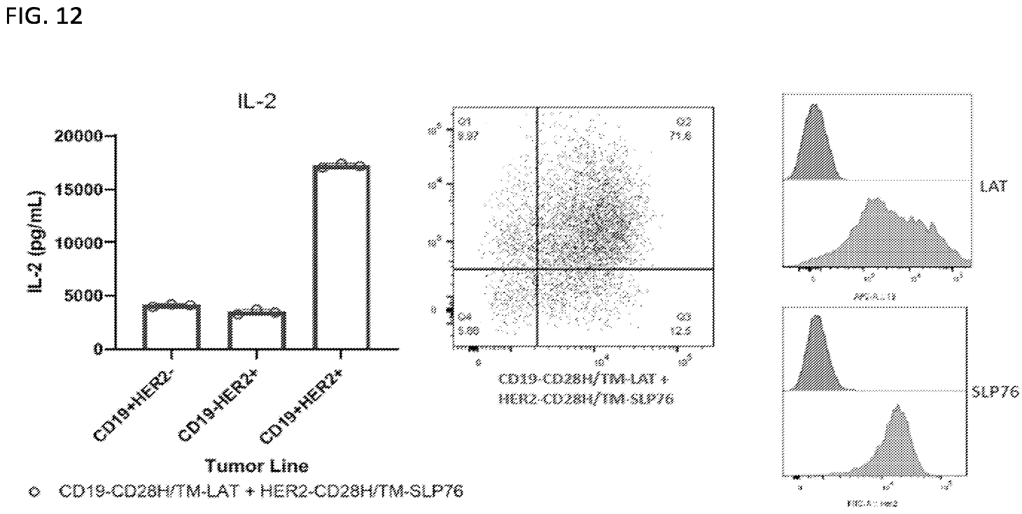

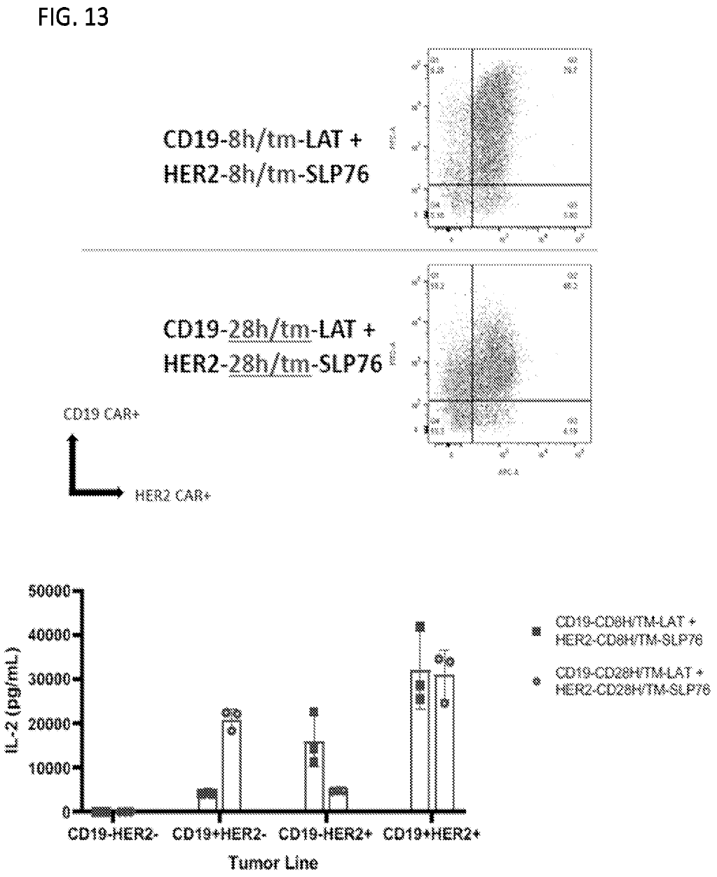

The first iteration of the AND-gate CAR, described in the previous Example, demonstrated a potent cell activation after encounter of tumor cells expressing both target antigens. The system, however, still resulted in some background T-cell activation in response to a single antigen (termed “leakiness”, FIG. 12). The initial signaling molecule CARs contained a TM domain from CD28, which may bring the LAT and SLP-76 CARs together, by a homodimerization between their TM domains, for signaling in the absence of both antigens/absence of dual ligation. An exemplary system having a CD19−CD28H/TM-LAT and a HER2−CD28H/TM-SLP-76 CAR molecules was found to have a detectable background activation of T cells in response to either one of CD19 and HER2 antigens (FIG. 12). Similarly, this leakiness was found in other AND-gate CAR combinations containing a same TM domain in both CAR molecules, such as a pair of LAT/SLP-76 CAR molecules with a same CD8 or CD28 hinge/TM domain (FIG. 13). Such background activation induced IL-2 production by the T cells exposed to only a single antigen (FIG. 13, the bottom panel). T cells expressing the CAR molecules sharing a CD8 hinge/TM domain were leakier when exposed to HER2 than to CD19, while the cells expressing the CAR molecules sharing a CD28 hinge/TM domain were leakier when exposed to CD19 (FIG. 13, the bottom panel).

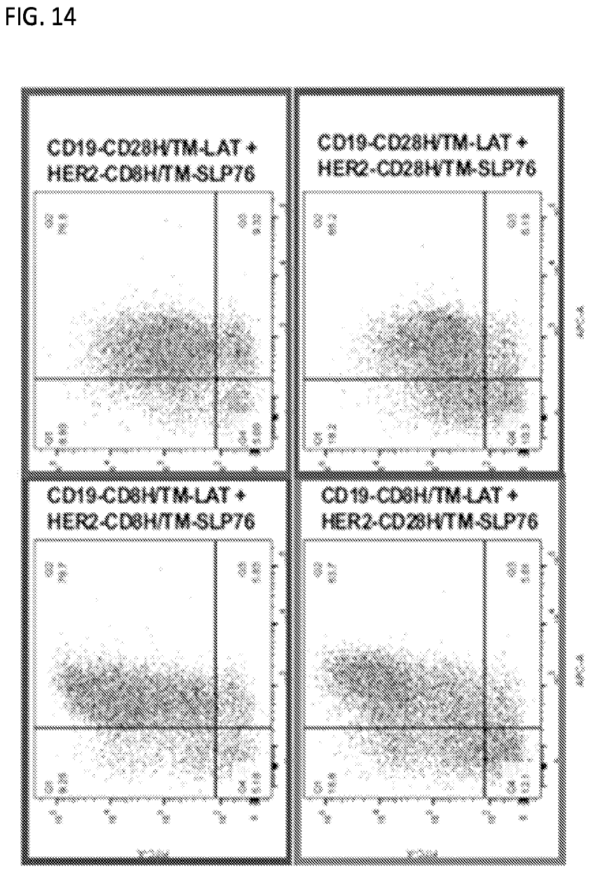

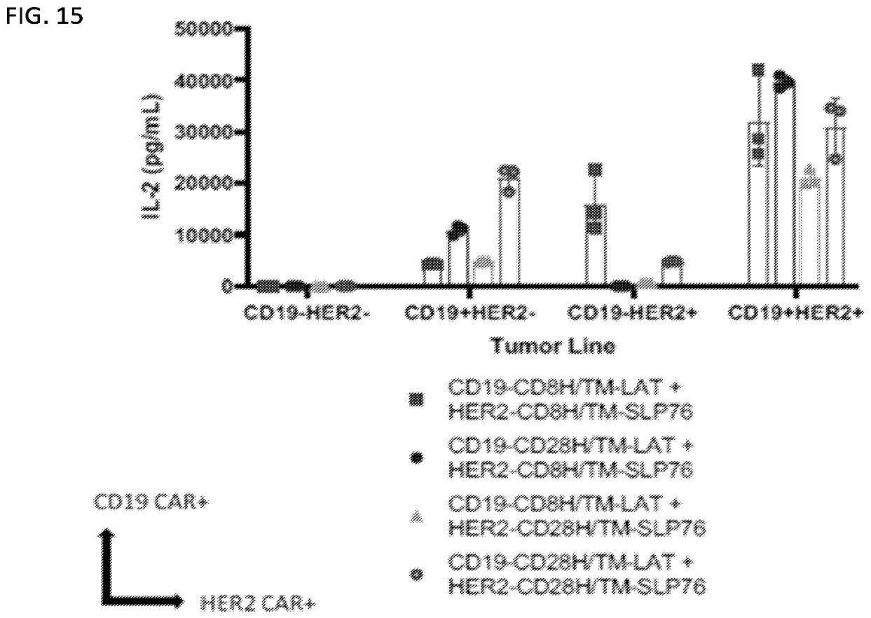

To produce improved AND-gate CAR systems which strongly recognize double antigen positive tumor cells with minimal leakiness, different TMs were introduced to the LAT and SLP-76 CARs. For example, each of CD8 hinge/TM and CD28 hinge/TM domains was used for constructing one of the AND-gate CAR molecules, resulting in a pair of exemplary CAR molecules of CD19−CD28H/TM-LAT and HER2−CD8H/TM-SLP-76, or CD19−CD8H/TM-LAT and HER2−CD28H/TM-SLP-76 (FIG. 14). T cells expressing CAR molecules having these mixed hinge/TM domains had a reduced leakiness in activation (as evidenced by IL-2 production), particularly when exposed to the HER2 antigen (the target of the SLP-76 CAR) (FIG. 15).

The CD28H/TM-LAT+CD8H/TM-SLP-76 CAR molecule combination was a first candidate for optimizing the AND gate. T cells expressing this candidate showed good cytotoxicity and cytokine production against double antigen positive cell lines, and no activity against HER2 single positive cells. However, some cytokine production against CD19 single positive cells was observed in vitro (FIG. 15). To address these issues, a number of mutations and hinge/transmembrane domain alterations were engineered.

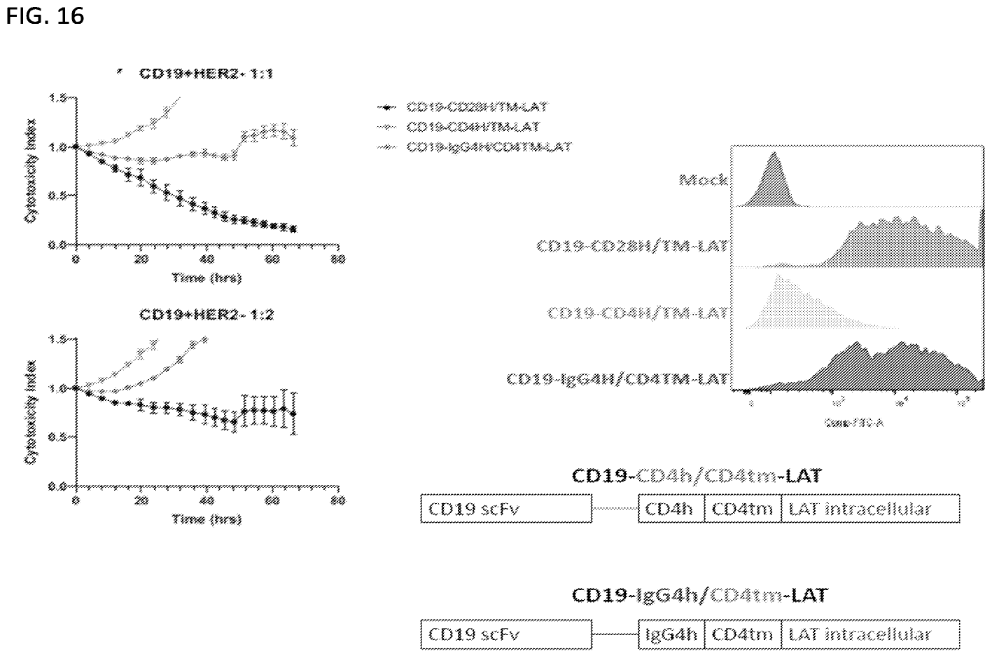

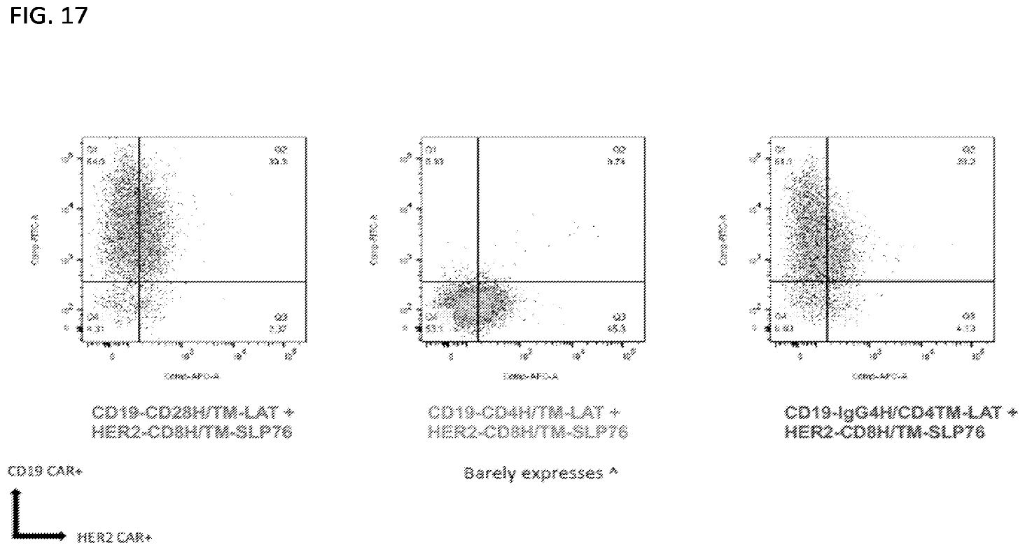

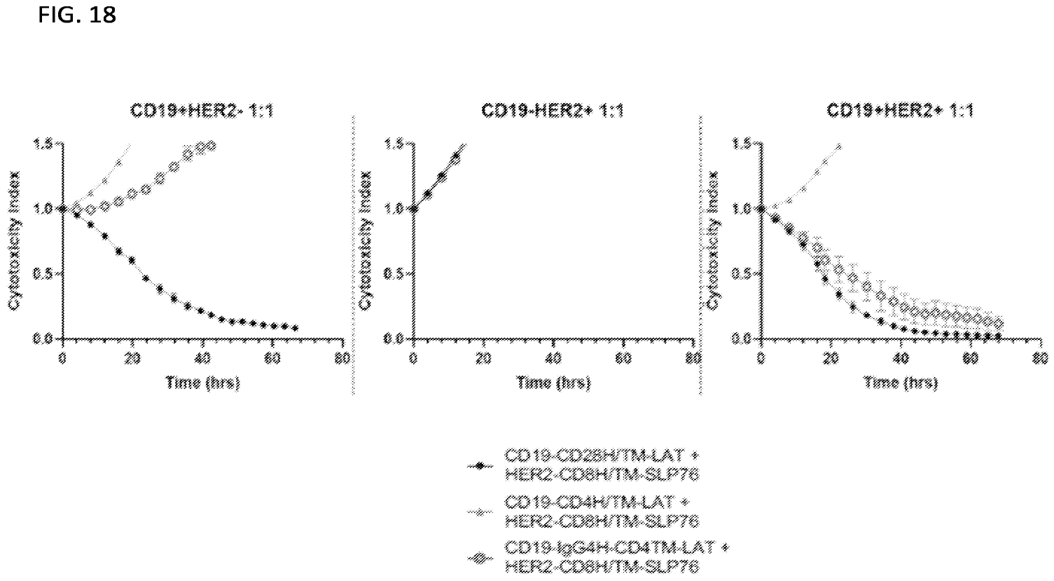

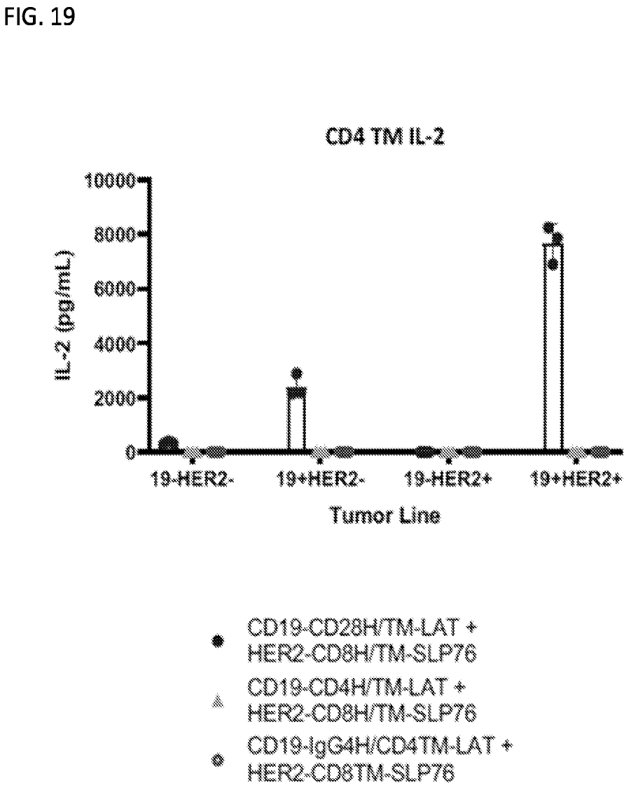

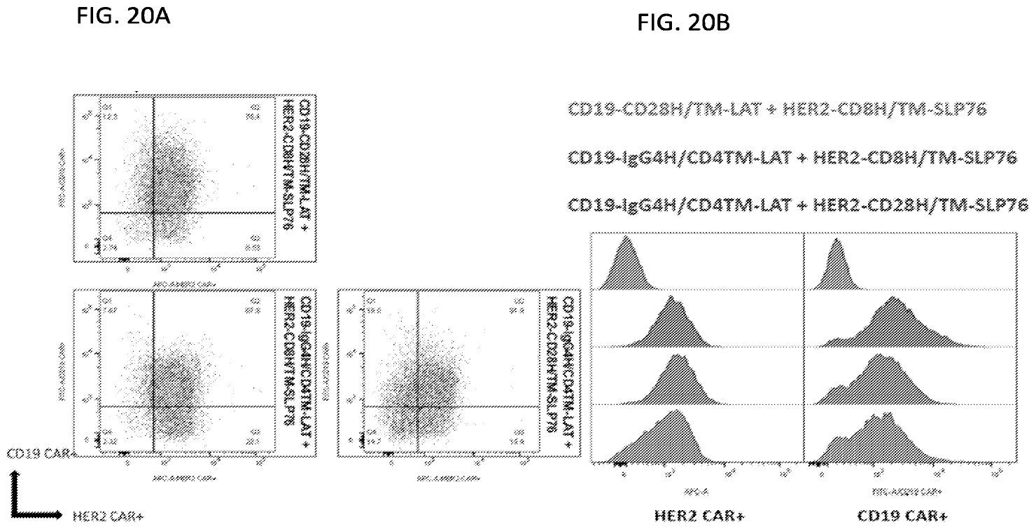

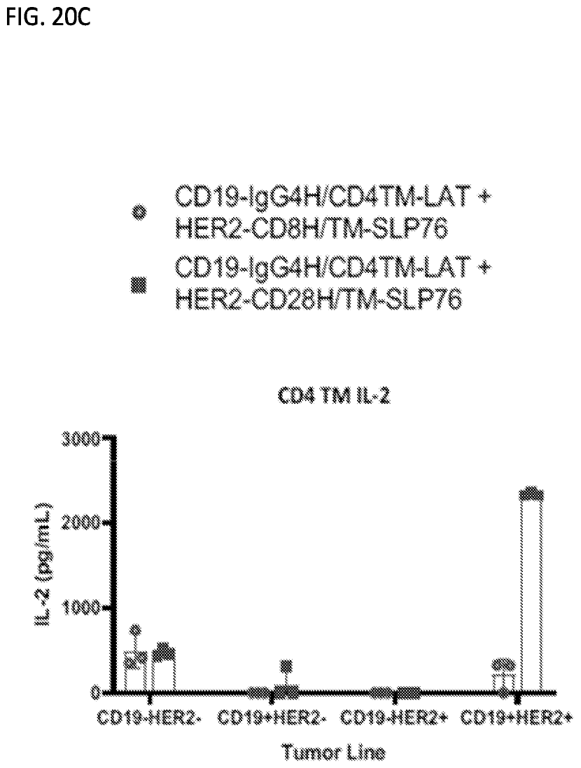

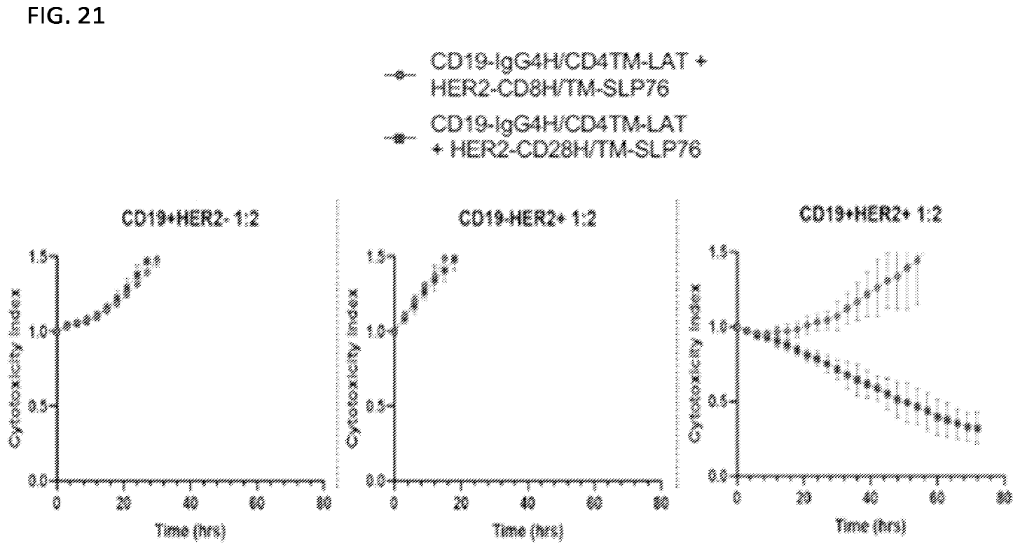

Since CAR efficacy is largely dependent on the hinge/TM domain, mutations or alterations to the hinge/TM domain were prepared for their activity to modulate CAR functions. For example, CD4 hinge/TM or IgG4H/CD4TM were used to substitute the CD28 hinge/TM domain used in the CAR molecules described in previous Examples. T cell cytotoxicity experiments were performed for cells expressing CAR constructs for CD19-CD28H/TM-LAT, CD19−CD4H/TM-LAT, and CD19−IgG4H/CD4TM-LAT. As shown in FIG. 16, a LAT CAR with a CD4H/TM did not express, but a LAT CAR with a IgG4H/CD4TM did express and it reduced cell cytotoxicity, either when expressed alone (FIG. 16) or together with a SLP-76 CAR molecule to form an AND gate (FIG. 18). However, although this H/TM domain swap method reduced background cytotoxicity of LAT CAR molecules, these swapped constructs abrogated cytokine (e.g., IL-2) production by the T cells (FIG. 19). This loss of activity may be due to the fact that the CD28 hinge/TM domain is more effective for most CARs. Therefore, to compensate for this loss of activity with a controlled background activation, exemplary SLP-76 CAR molecules were engineered to, e.g., contain the CD28 hinge/TM domain. The CAR combinations of CD19−CD28H/TM-LAT and HER2−CD8H/TM-SLP-76, CD19−IgG4H/CD4TM-LAT and HER2−CD8H/TM-SLP-76, or CD19−IgG4H/CD4TM-LAT and HER2−CD28H/TM-SLP-76 were expressed in T cells (FIGS. 20A and 20B). IL-2 production by T cells expressing the last two CAR combinations were compared to show that the SLP-76 CAR molecule with a CD28H/TM domain, when co-expressed with the LAT CAR with a IgG4H/CD4TM domain, led to a low background T cell activation in response to single antigen (either CD19 or HER2) but a significant activation in response to CD19+HER2+ antigens (FIG. 20C). In cytotoxicity experiments, T cells expressing the CD19−IgG4H/CD4TM-LAT+HER2−CD28H/TM-SLP-76 combination showed almost no killing in response to either CD19 or HER2 single antigen, but an improved cytotoxicity to tumor cells expressing both antigens, compared to T cells expressing a CAR combination in which a SLP-76 CAR has a CD8 hinge/TM domain (FIG. 21).

As described above, a second AND gate combination candidate, CD19-IgG4H/CD4TM-LAT+HER2−CD28H/TM-SLP-76 was much less leaky to CD19 single positive tumor cells, but produced less IL-2 compared to the combinations in which a LAT CAR contained a CD28 H/TM domain. Thus, the system was further engineered to strengthen its activity in response to both antigens without compromising leakiness to a single antigen.

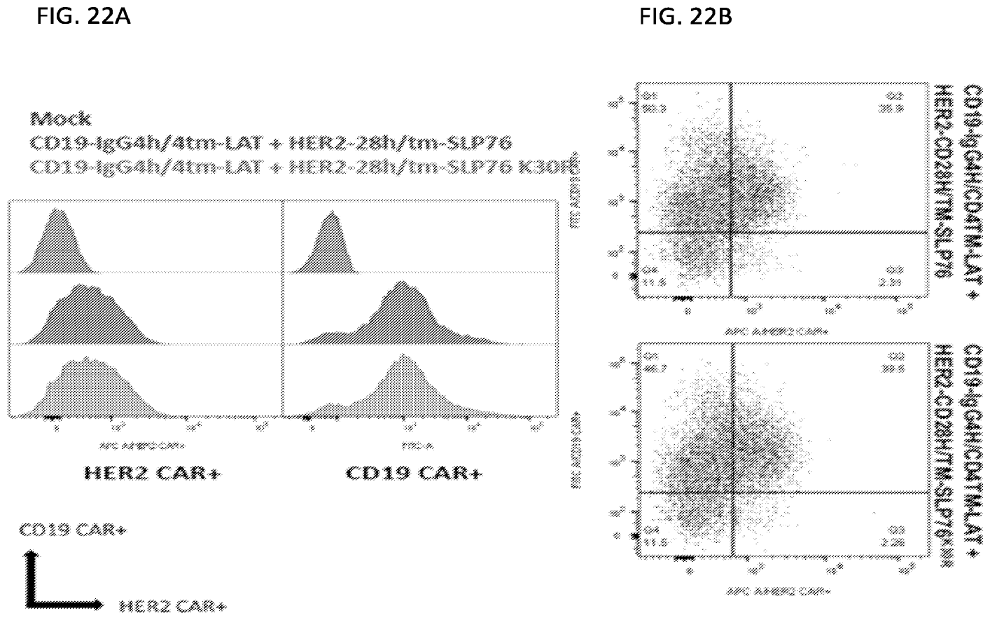

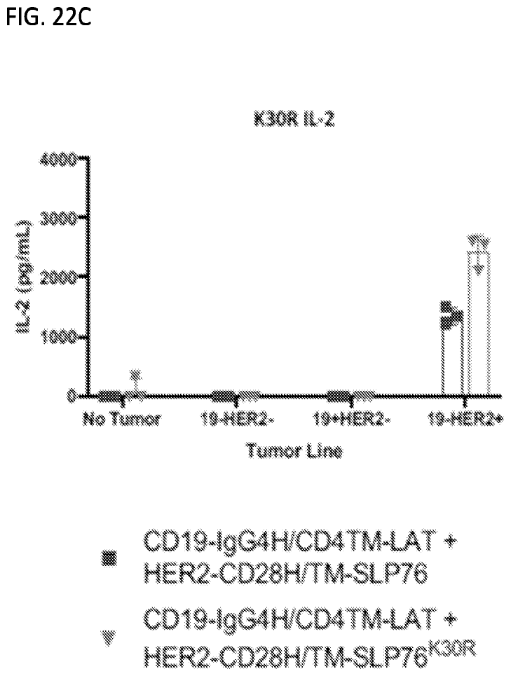

For strengthening SLP-76, a K30R mutation that was previously reported to reduce ubiquitination of SLP-76 is capable of enhancing downstream T cell activation. An exemplary SLP-76 CAR construct was prepared to contain the K30R mutation, resulting in a AND gate combination of CD19−IgG4H/CD4TM-LAT+HER2−CD28H/TM-SLP-76^{K30R} (FIGS. 22A and 22B). The single mutation K30R improved T cell activation (measured by IL-2 production in FIG. 22C) in response to double antigens and maintained no leakiness in response to single antigen.

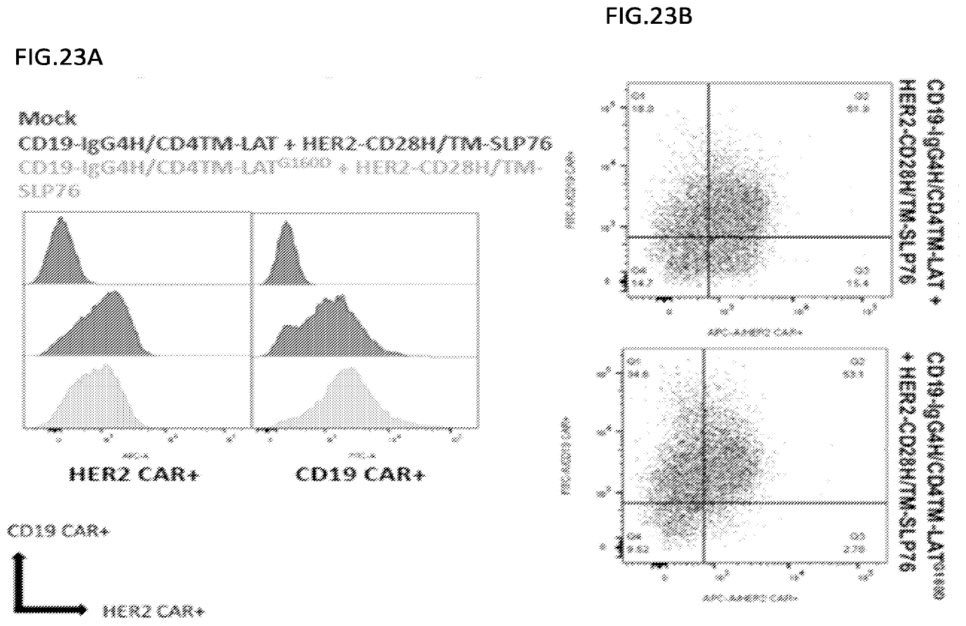

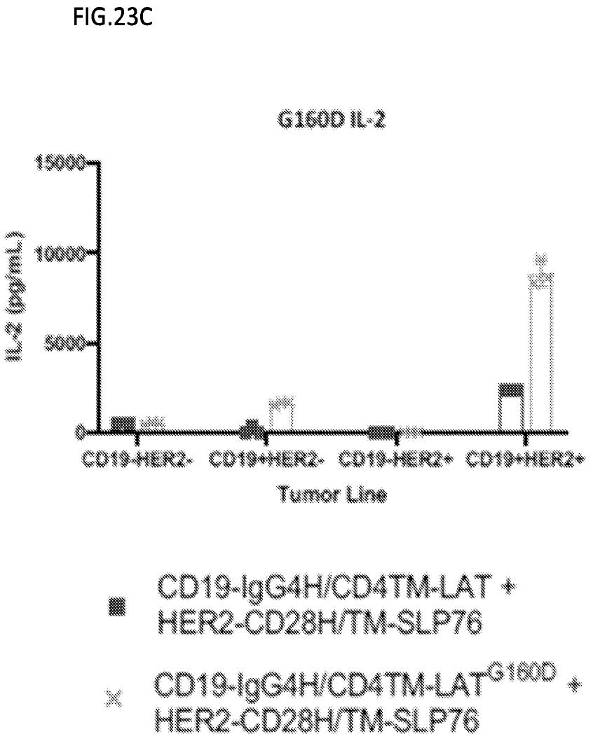

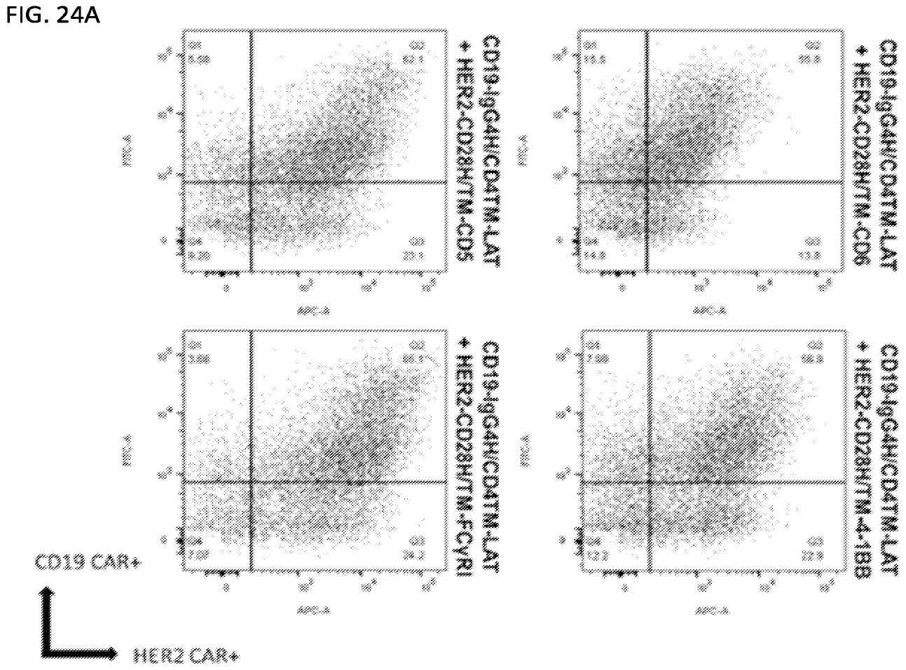

On the LAT CAR side, strengthening LAT is not ideal because LAT CARs may sometimes kill cells on its own. However, a G160D mutation, known to enhance PLCG1 activation, was shown to enhance the AND-gate. An exemplary LAT CAR construct was prepared to contain the G160D mutation, resulting in an AND gate CAR combination of CD19−IgG4H/CD4TM-LAT^{G160D}+HER2−CD28H/TM-SLP-76 (FIGS. 23A and 23B). As shown in FIG. 23C, this G160D mutation greatly enhanced cytokine production against double positive cells, with limited leakiness against single antigen positive cells. In fact, with such enhancement, this combination has a specific activity comparable to the initial combination.

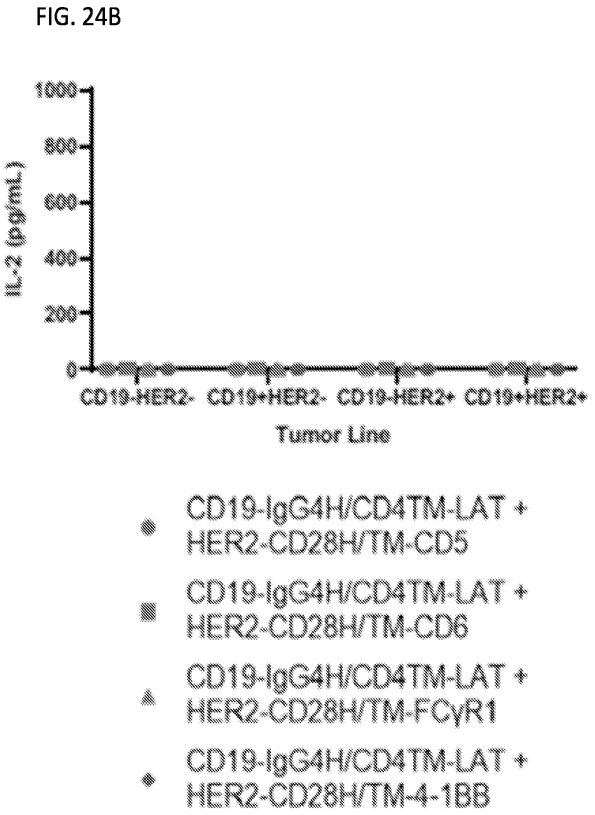

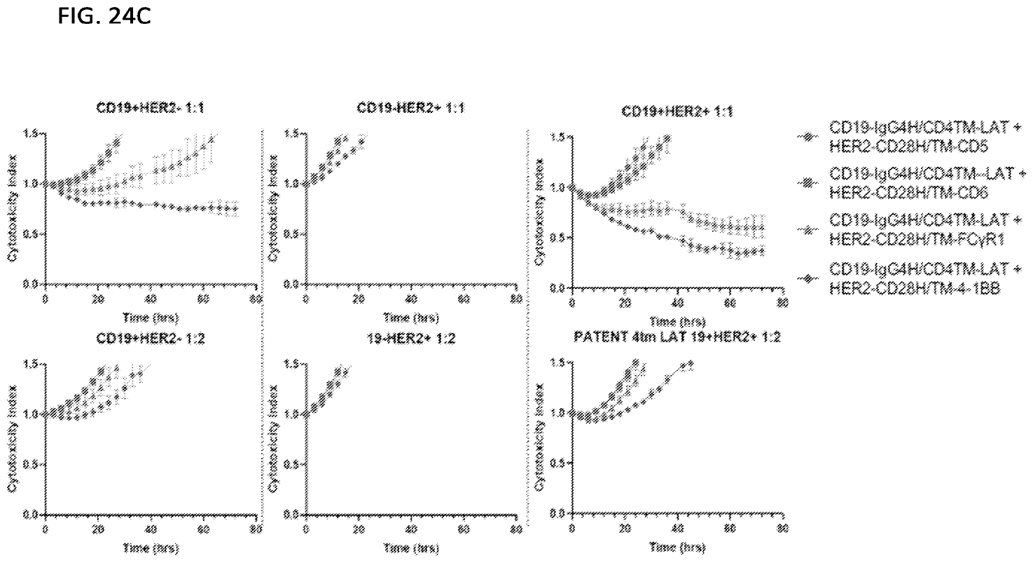

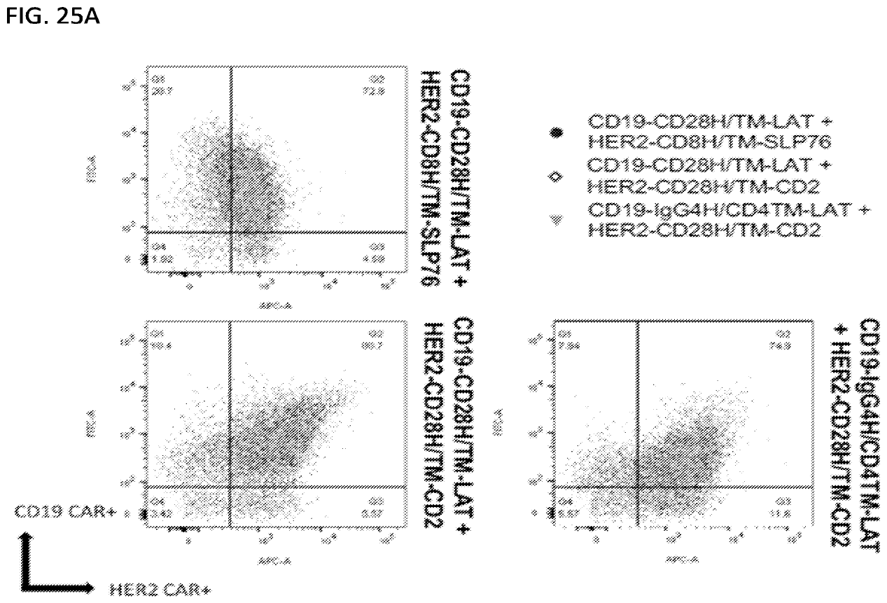

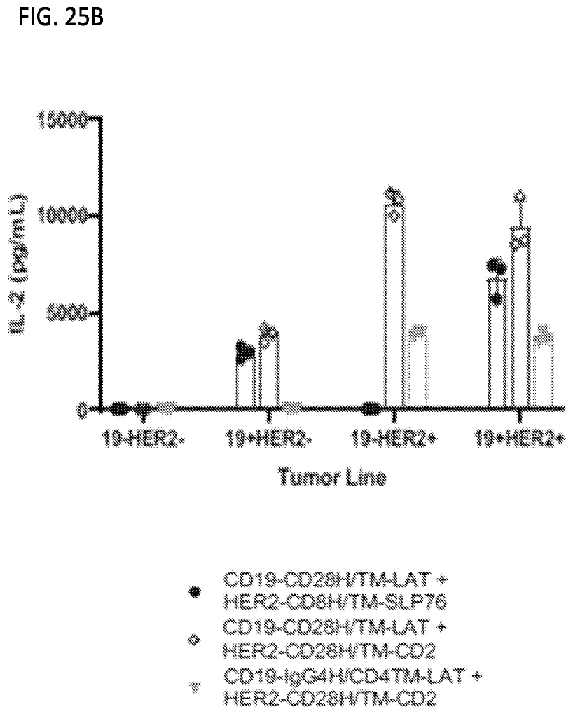

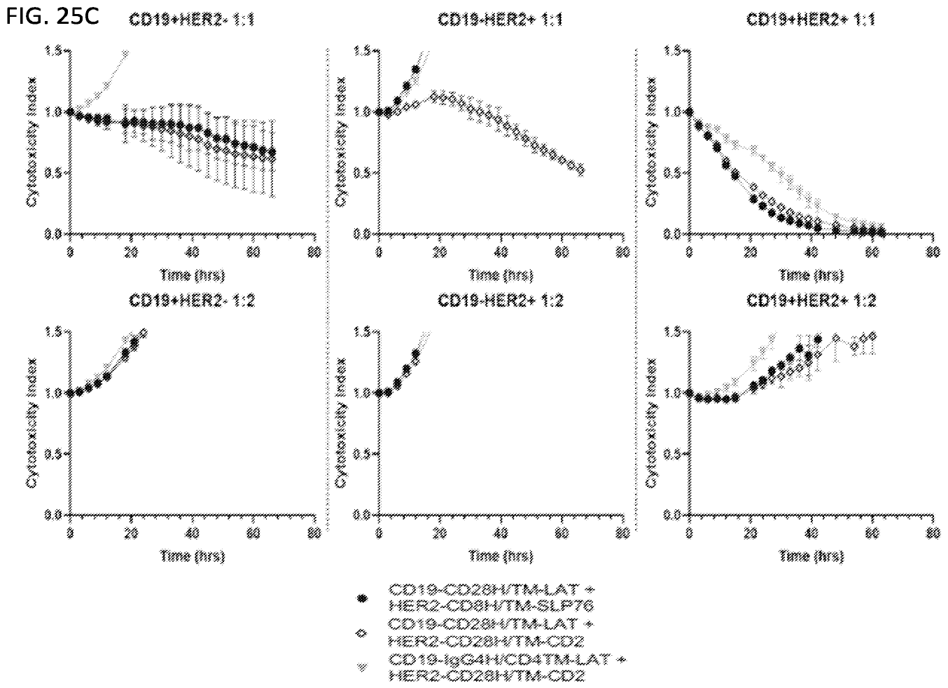

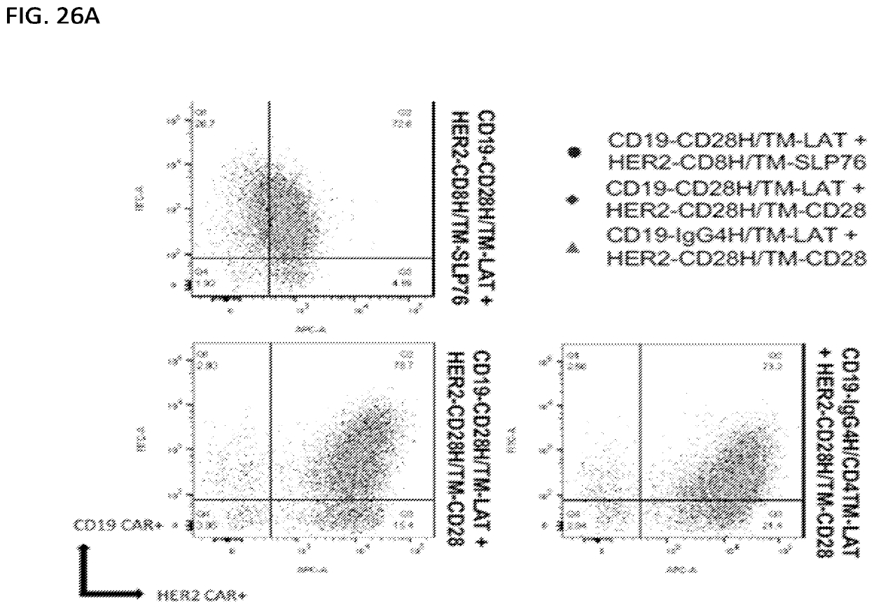

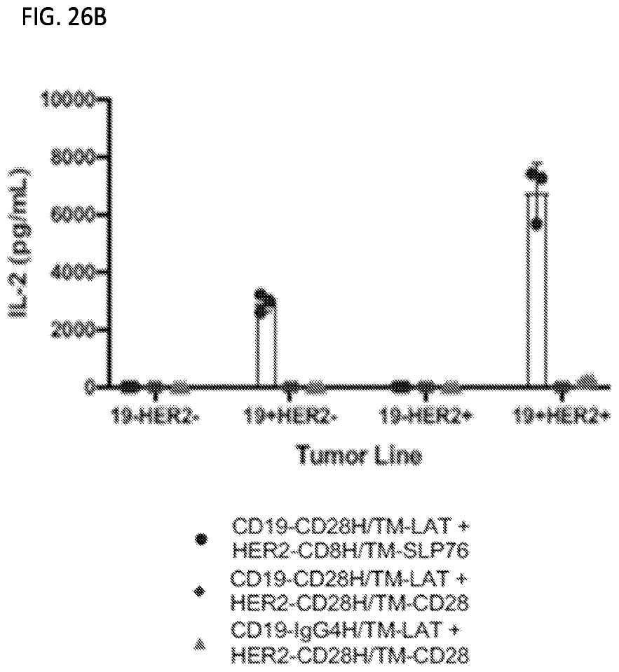

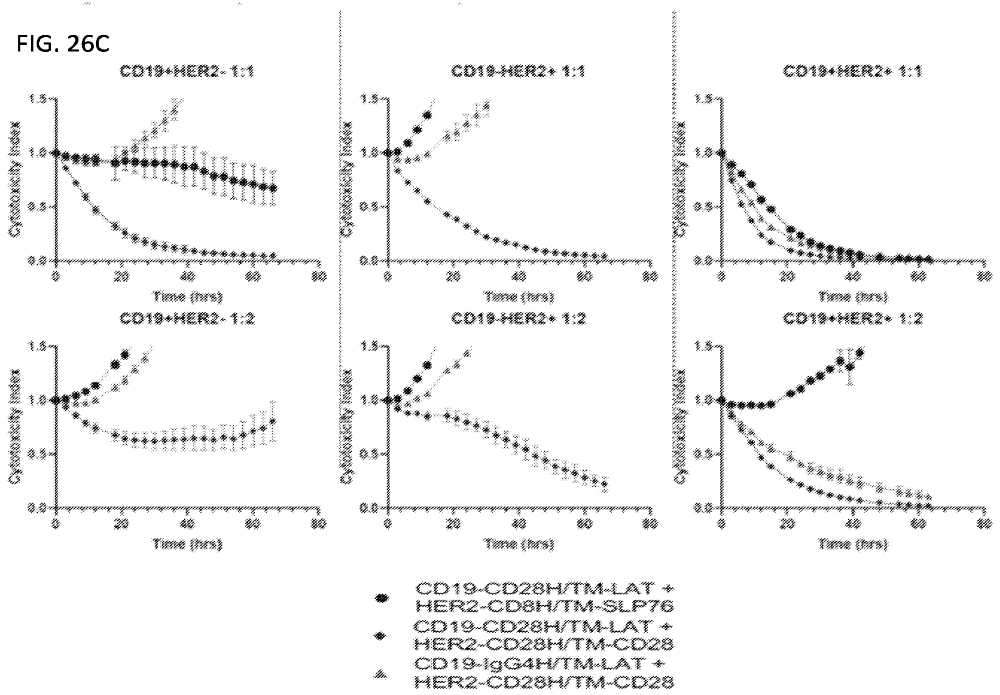

As described above, CD19−IgG4H/CD4TM-LAT^{G160D}+HER2−CD28H/TM-SLP-76 had excellent activity against double positive cell lines, and a small amount of leakiness on LAT antigen. When using LAT and SLP-76 constructs, AND-gates were leaky against CD19+ cells because LAT is presumably interacting with SLP-76 in some degree. Further engineering was performed to replace SLP-76 with costimulation molecules (CD28, CD2, FCgammaR1, CD5, CD6, or 4-1BB) known to associate with TCR signaling molecules and recruit SLP-76. LAT+CD5/CD6/4-1BB/FcgammaR1 combinations did not activate T cells to produce cytokine (FIGS. 24A and 24B) or to sufficiently kill tumor cells expressing the corresponding antigens (FIG. 24C). LAT+CD2 CAR combinations showed a high background activation in response to single antigen for the CD2 CAR molecule (FIGS. 25A and 25B) and did not promote T cells to sufficiently kill tumor cells expressing both antigens (FIG. 25C). By contrast, a combination having a CD28 CAR molecule substituting for the SLP-76 CAR and an IgG4H/CD4TM-LAT CAR molecule (FIG. 26A) showed minimal leakiness with good cytotoxicity on double positive cell lines (FIGS. 26B and 26C).

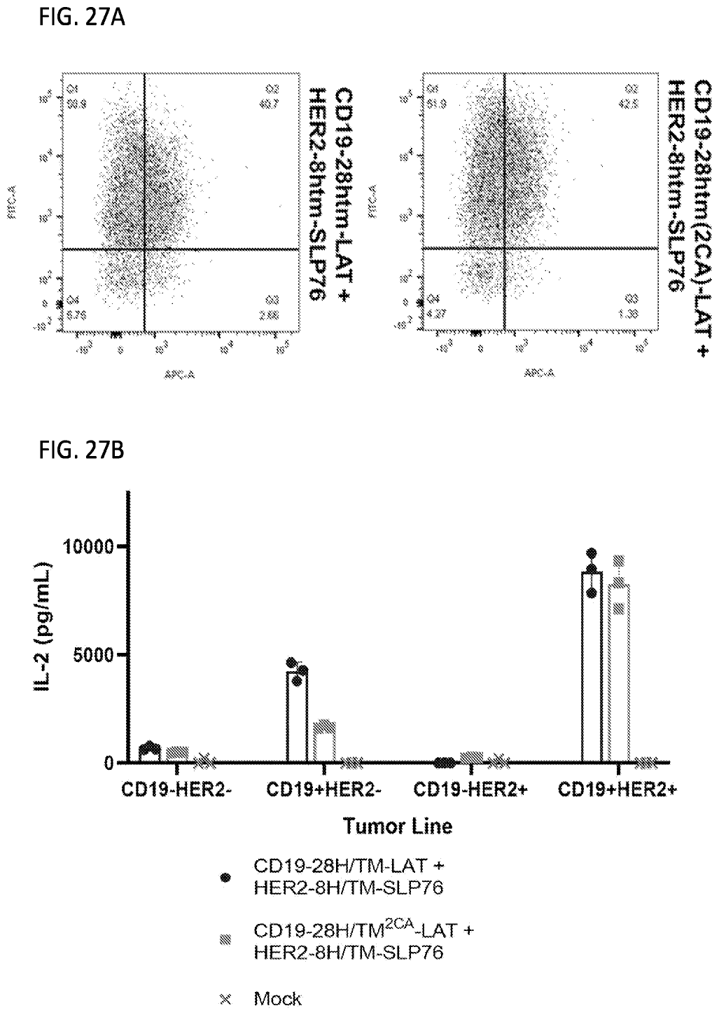

CARs incorporating a CD28-H/T demonstrate a more stable and efficient immunologic synapse (Majzner et. al, Cancer Discovery, 2020; 10:702-723). As an alternative to using an IgG4H/CD4TM domain on a LAT CAR, a CD28 H/TM domain was further mutated to substitute two cysteine residues to alanine residues (CD28H/TM^{2CA}). An AND-gate CAR combination containing a CD28H/TM^{2CA}-LAT had less leakiness when exposed to single antigen positive tumor cells but maintained good efficacy to double antigen positive cells (FIGS. 27A and 27B).

### Example 4

### Mechanistic Exploration to Further Optimize AND Gate CAR Activity

This Example describes experiments performed to introduce further modifications, mutations, and deletions to either LAT or SLP-76 CAR molecule to enhance the potency of the AND gate system only in response to dual antigens, without a substantial background activation in response to a single antigen.

The exemplary AND gate described in this application was rationally designed based on the role of SLP-76 and LAT in generating a scaffold from which T cell activity is directed. When expressed individually on T cells, the LAT CAR and SLP-76 CAR generate no cytokine response to target antigen encounter. Therefore, the source of leakiness in the system, as described in previous Examples, is related to the interaction of the two molecules when they are co-expressed on a single cell. LAT and SLP-76 interact with one another through adapter proteins such as GRB2 and GADS. Targeted mutations were prepared in each molecule that interrupt these interactions, which reduces the baseline aggregation of the CARs (in the absence of antigen) and thus reduces any leakiness in the system.

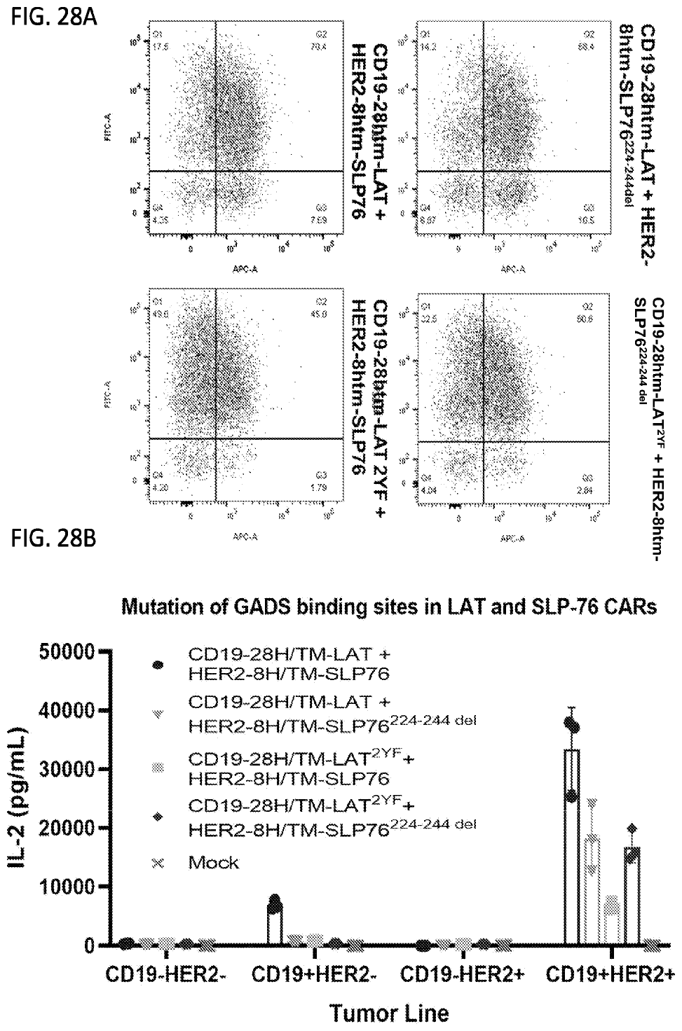

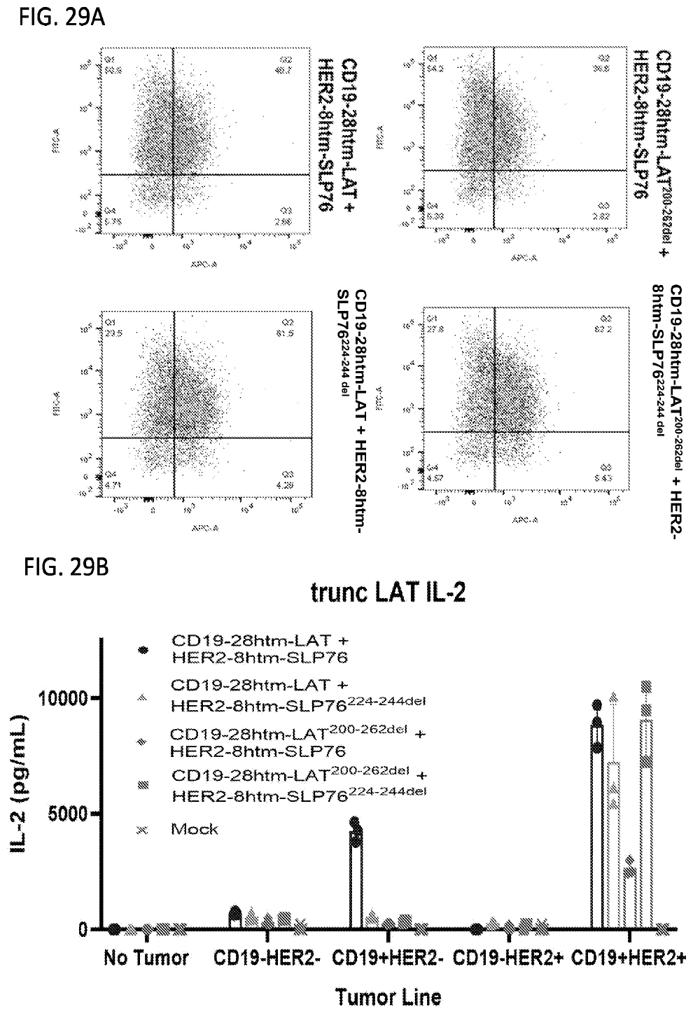

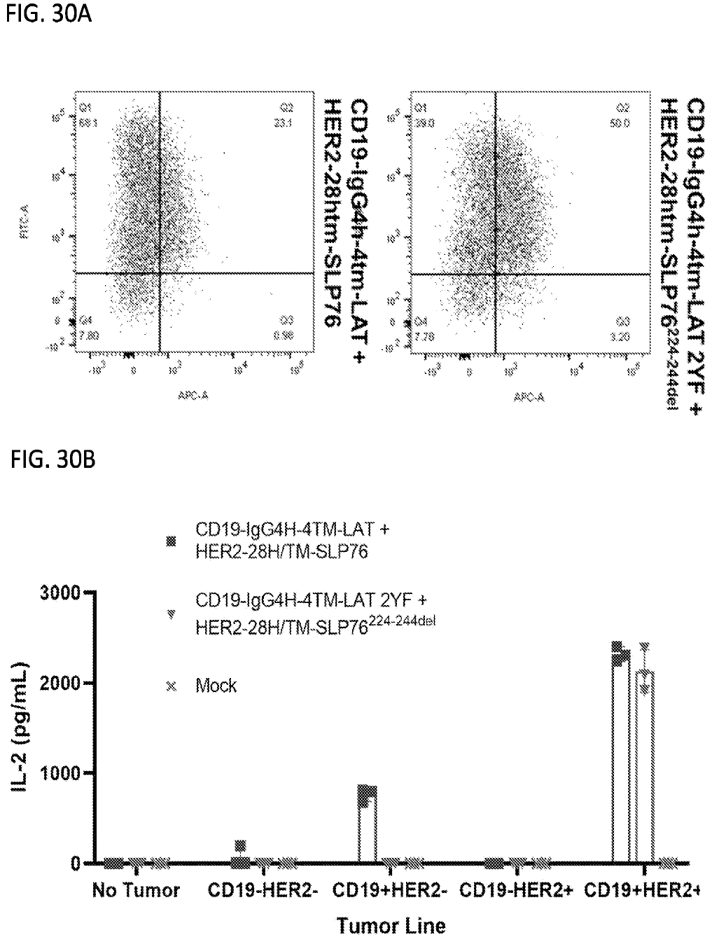

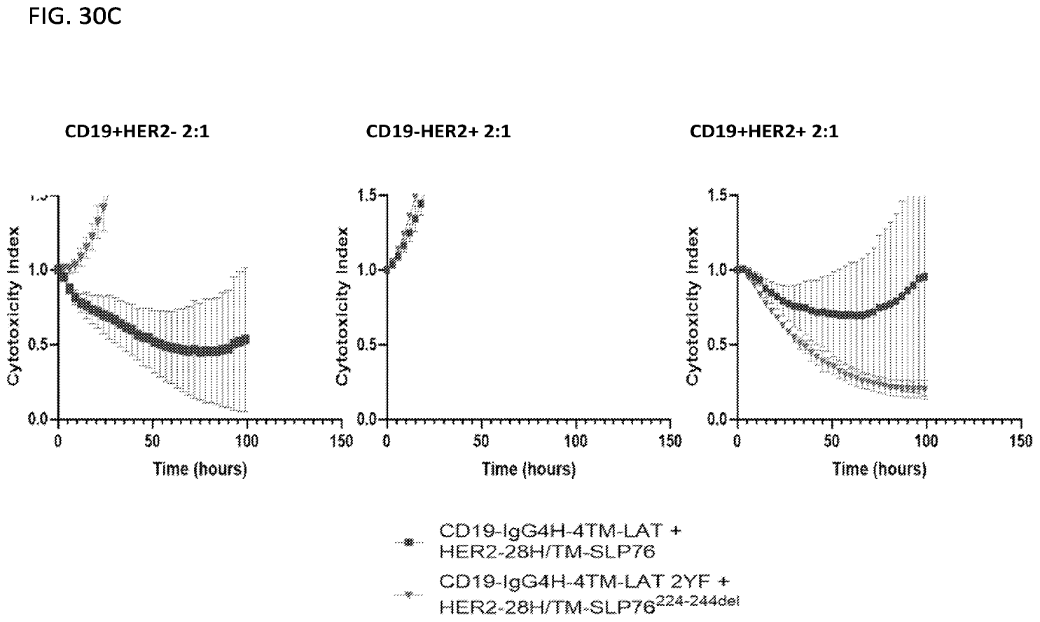

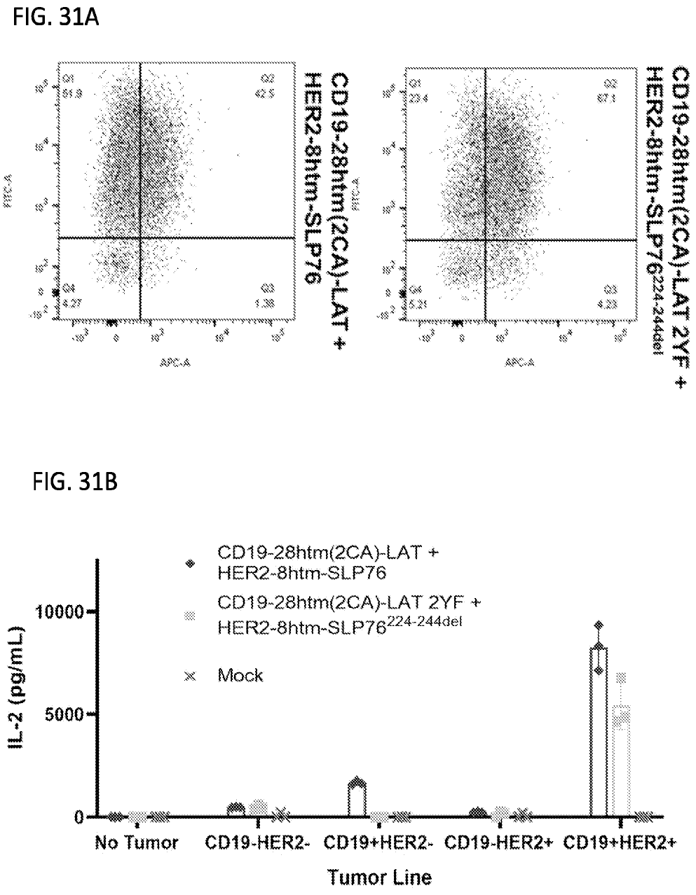

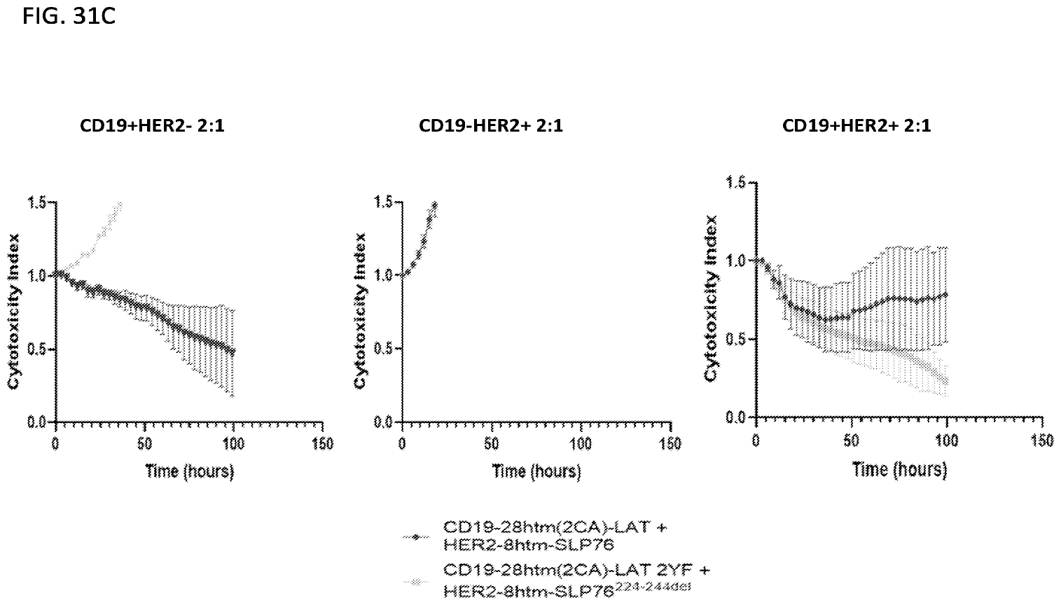

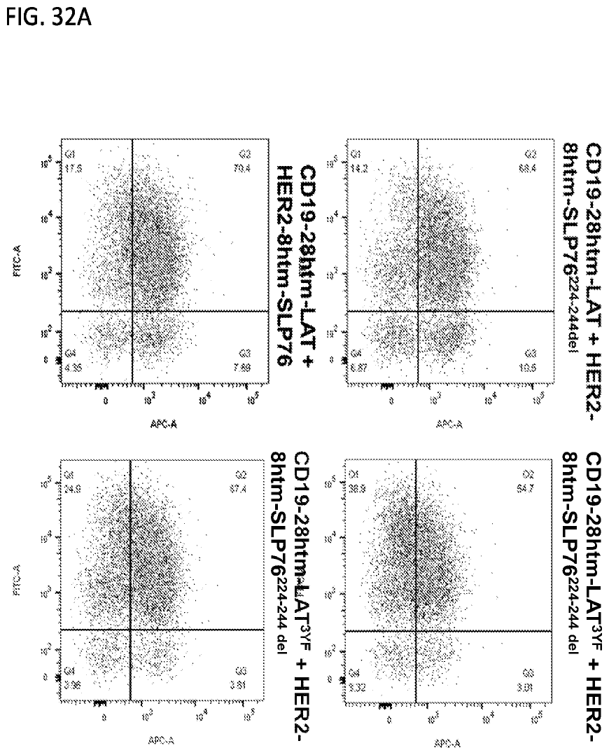

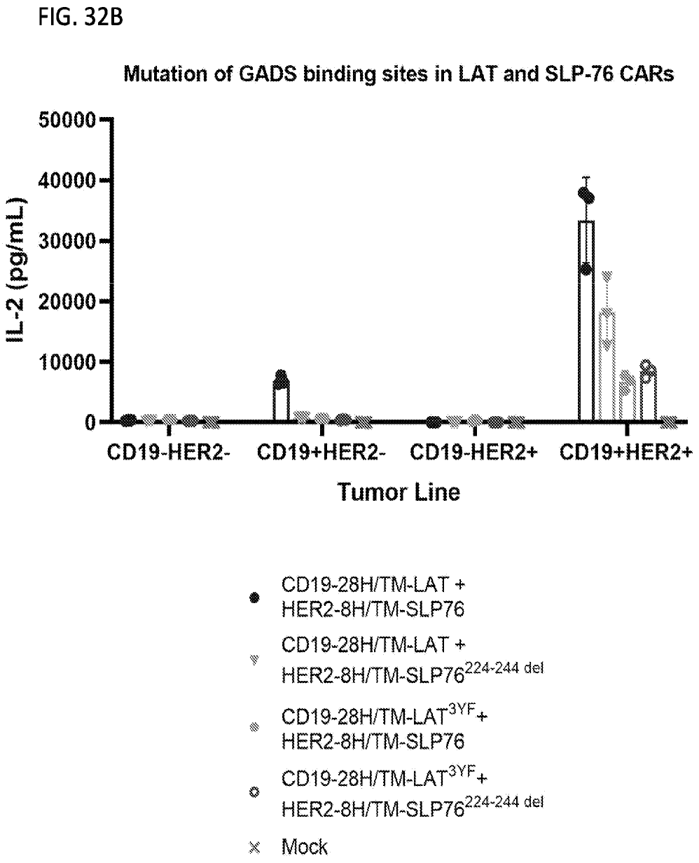

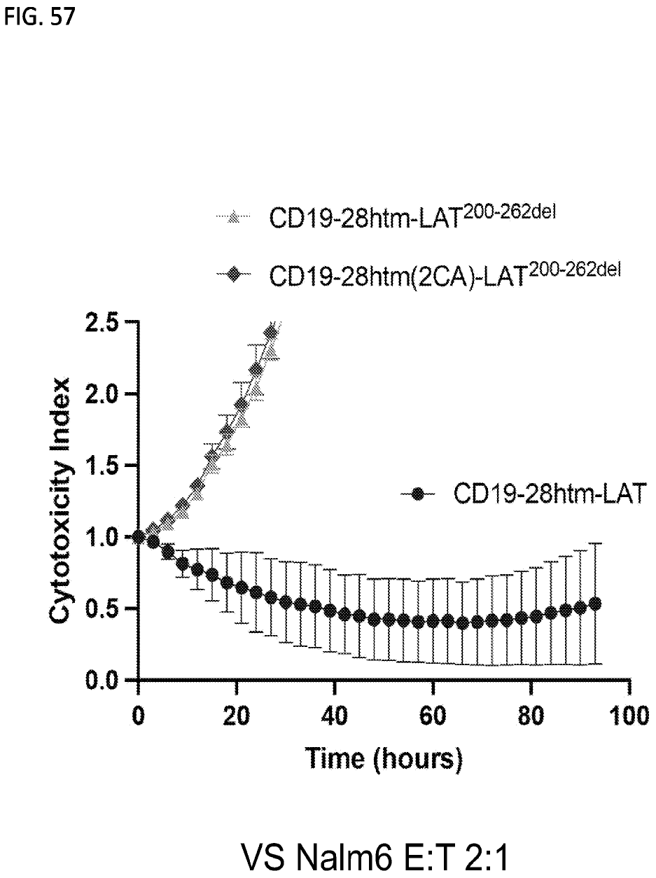

When mutations and deletions that abrogate GADS and GRB2 binding were introduced into the LAT and SLP-76 CAR constructs, leakiness or single antigen activity was eliminated. In particular, mutations such as Y200F/Y220F (LAT^{2YF}) or Y200F/Y220F/Y252F (LAT^{3YF}) on LAT or deletions such as Δ(200-262) on LAT (i.e., a deletion of amino acid residues from position 200 to position 262 of LAT; also as LAT^{200-262 del}) or Δ(224-244) on SLP-76 (also as SLP-76^{224-244 del}) greatly reduce the single antigen activity of the system, but still retain strong activity against double antigen positive tumor cells (see, e.g., FIGS. 28A and 28B for a SLP-76^{224-244 del} CAR alone or in combination with a LAT^{2YF} CAR; FIGS. 29A and 29B for a LAT^{200-262 del} CAR alone or in combination with a SLP-76^{224-244 del} CAR; FIGS. 30A-30C for a LAT^{2YF} (i.e., Y200F/Y220F) with a IgG4H/CD4TM CAR combined with a SLP-76^{224-244 del} CAR; FIGS. 31A-31C for a CD28H/TM^{2CA}-LAT^{2YF} CAR combined with a SLP-76^{224-244 del} CAR; and FIGS. 32A and 32B for a LAT 3YF (i.e., Y200F/Y220F/Y252F) CAR combined with a SLP-76^{224-244 del} CAR). In particular, when mutations that abrogate GADS binding are made on both LAT and SLP-76 CAR constructs, leakiness is minimized. This is demonstrated by both tumor cell killing and cytokine production by T cells. Additionally, unlike a LAT CAR with a CD28 H/TM domain, a LAT CAR with the GADS binding site deleted (LAT^{200-262 del}) with either a CD28H/TM or CD28H/TM^{2CA} does not demonstrate any degree of killing of tumor cells on its own (FIG. 57).

When mutations that abrogate GADS binding were combined with a hinge/transmembrane combination that minimized leakiness as well (using a LAT molecule with either a CD28 H/TM domain with mutated cysteine residues (FIGS. 31A-31C) or a IgG4H-CD4TM domain (FIGS. 30A-30C)), a system was achieved that demonstrated minimal to no background activity against one antigen (minimal ‘leakiness’), but maximal activity when both antigens are encountered.

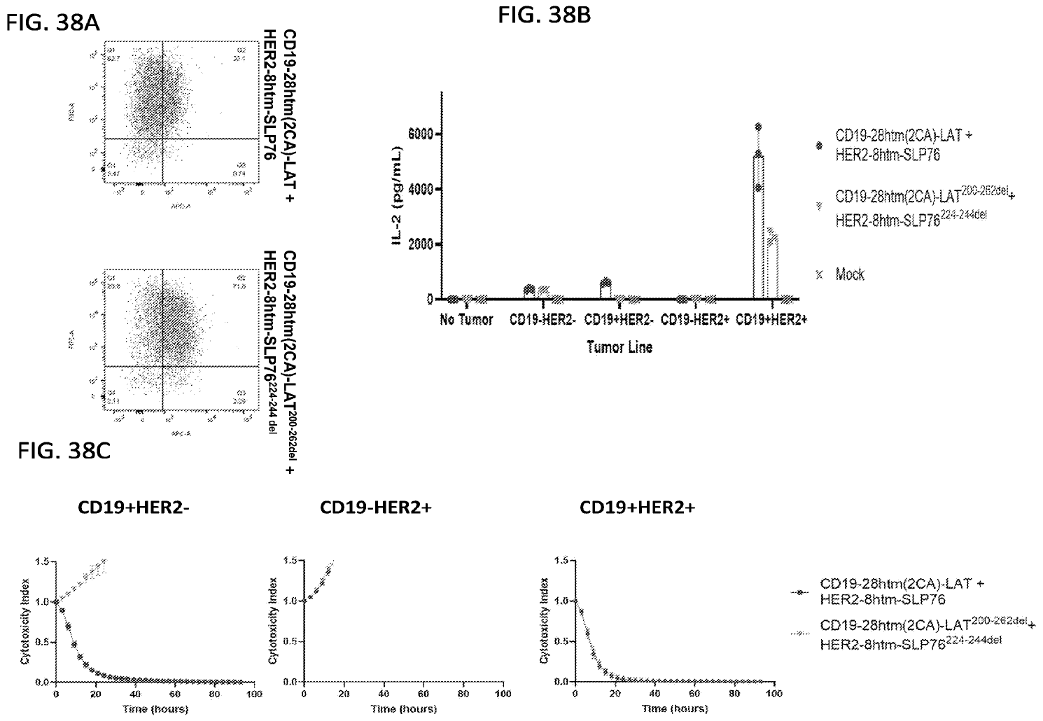

An exemplary system with maximal potency against double antigen positive tumor cells and minimal activity against single antigen was achieved when combining truncations/deletions in LAT and SLP-76 that abrogate GADS binding with a hinge/transmembrane combination that minimizes leakiness by using a CD28H/TM^{2CA} on one molecule and a CD8H/TM on the other, as shown in FIGS. 38A-38C.

### Example 5

### Exemplary ZAP70 CAR Constructs with Different Extracellular Ligand/Antigen-Binding Domains, Costimulatory Domains, or Enhancing Mutations

This Example describes experiments performed to show further engineering to the ZAP70 CAR described in the instant application.

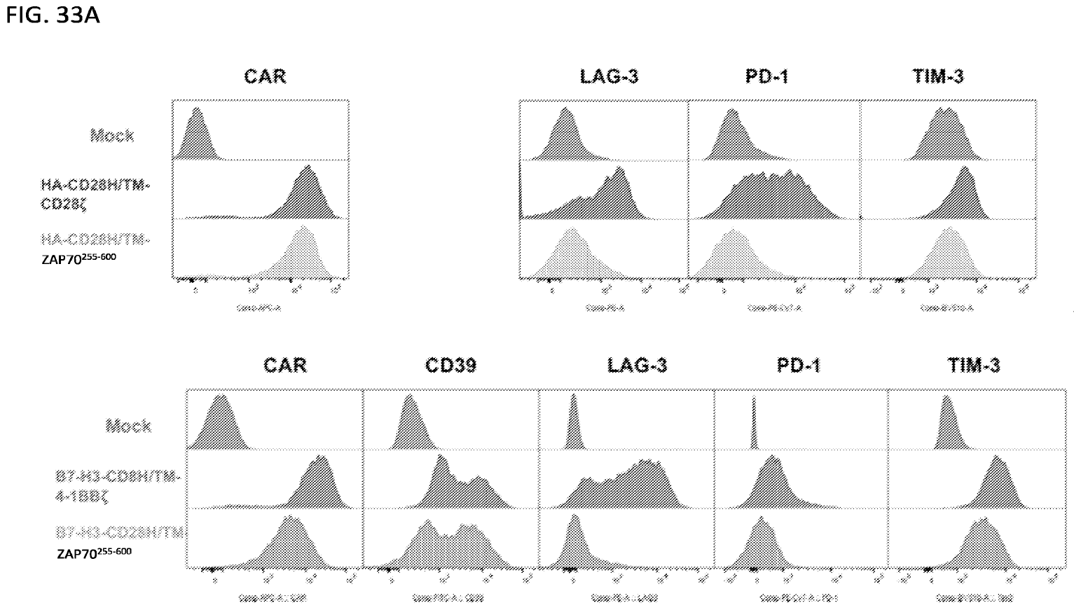

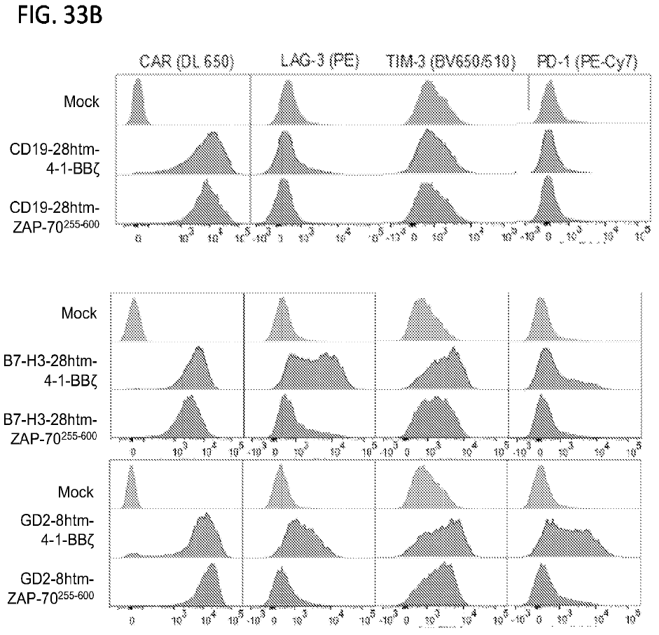

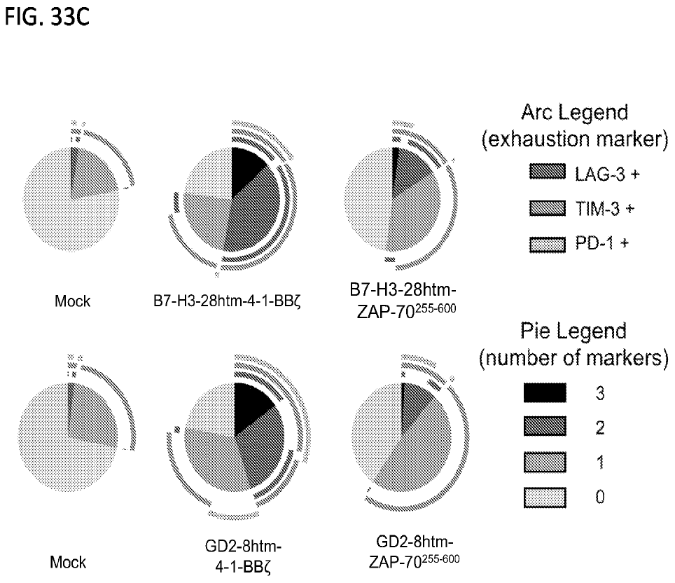

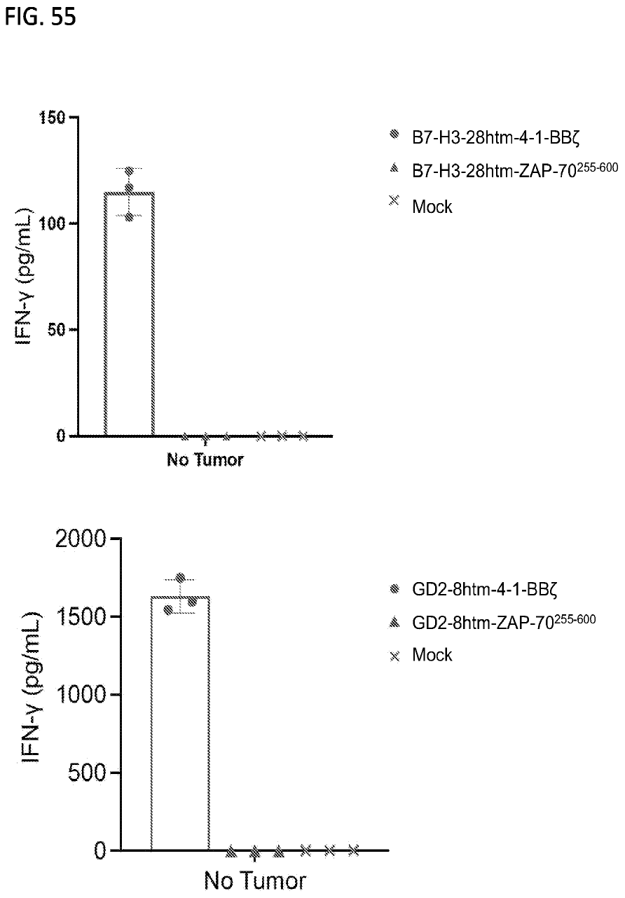

Different extracellular antigen-binding domains were engineered for the CAR molecules described herein. For example, ECDs specific for B7-H3 (CD276) or GD2 (HA=high affinity GD2 binder) may be used to construct a CAR molecule containing ZAP70. B7-H3 and GD2 specific (HA or standard GD2) ZAP70²⁵⁵⁻⁶⁰⁰ CAR molecules showed a reduced T cell exhaustion phenotype compared to traditional CARs containing CD3zeta and costimulatory molecule endodomains (FIGS. 33A-33C). FIG. 55 further shows that tonic-signaling GD2 or B7-H3-targeting CAR T cells bearing CARs with ZAP-70²⁵⁵⁻⁶⁰⁰ fragment endodomains produce lower baseline cytokine (IFNγ) in vitro, compared to CAR T cells with traditional 4-1BB-zeta endodomains, indicative of reduced effects of tonic signaling, a state that is usually detrimental to T cell function. On the other hand, for an ECD specific for CD19 that does not drive tonic signaling, there is no decrease in exhaustion markers by utilizing a ZAP70²⁵⁵⁻⁶⁰⁰ CAR because the traditional CARs containing CD3zeta and costimulatory molecule endodomains do not manifest signs of T cell exhaustion.

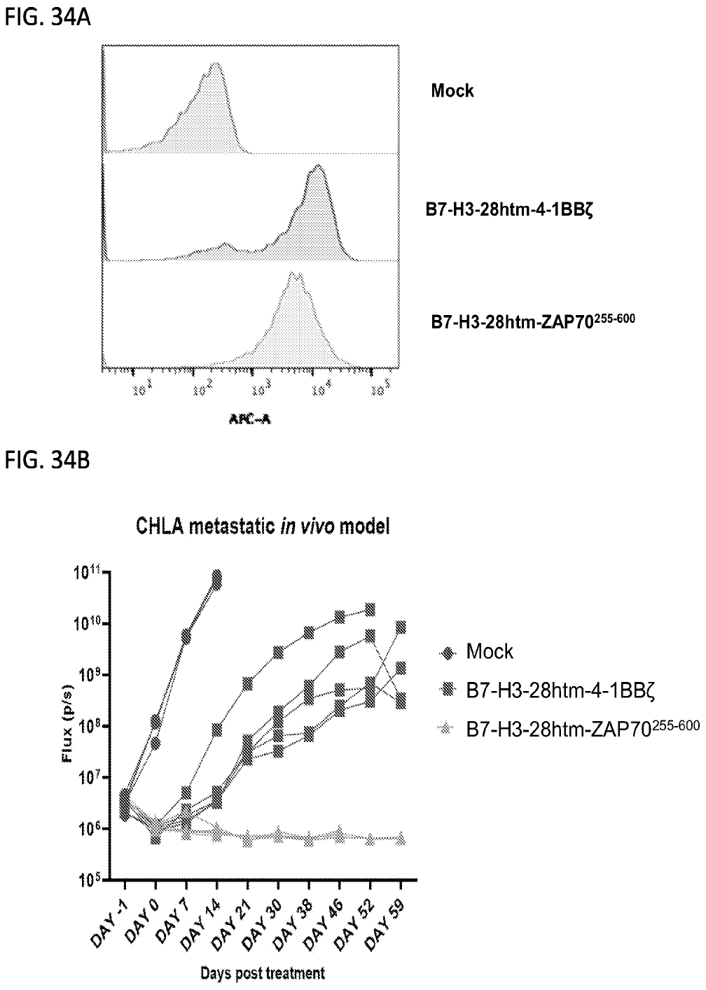

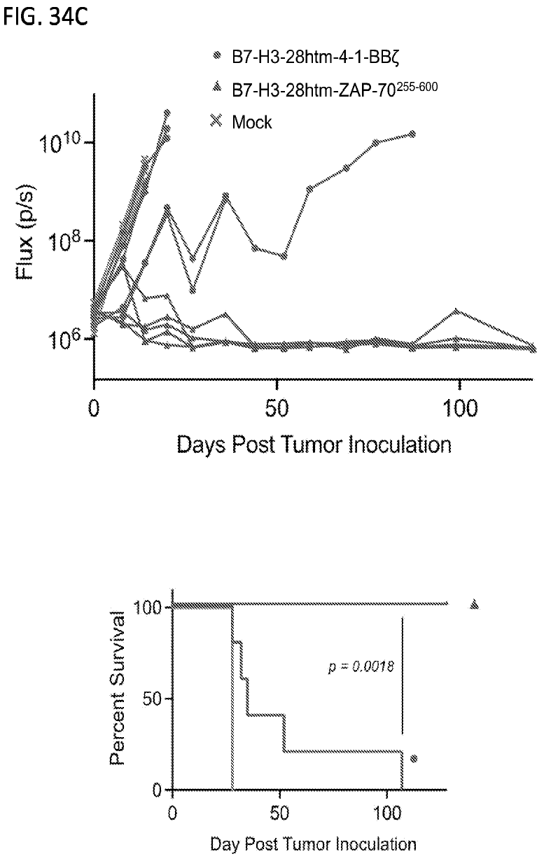

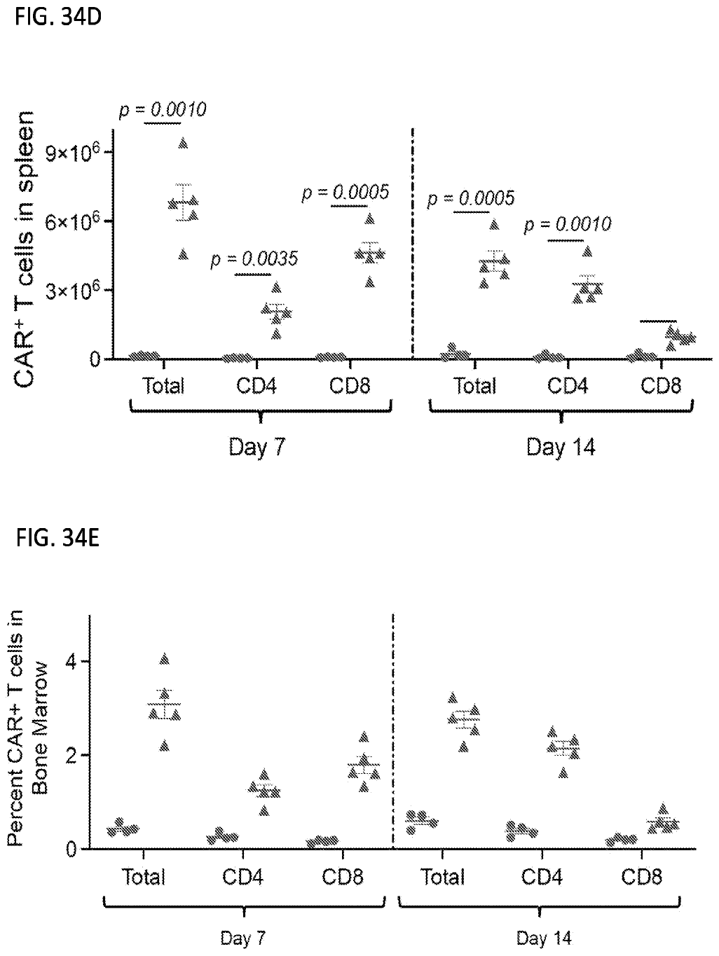

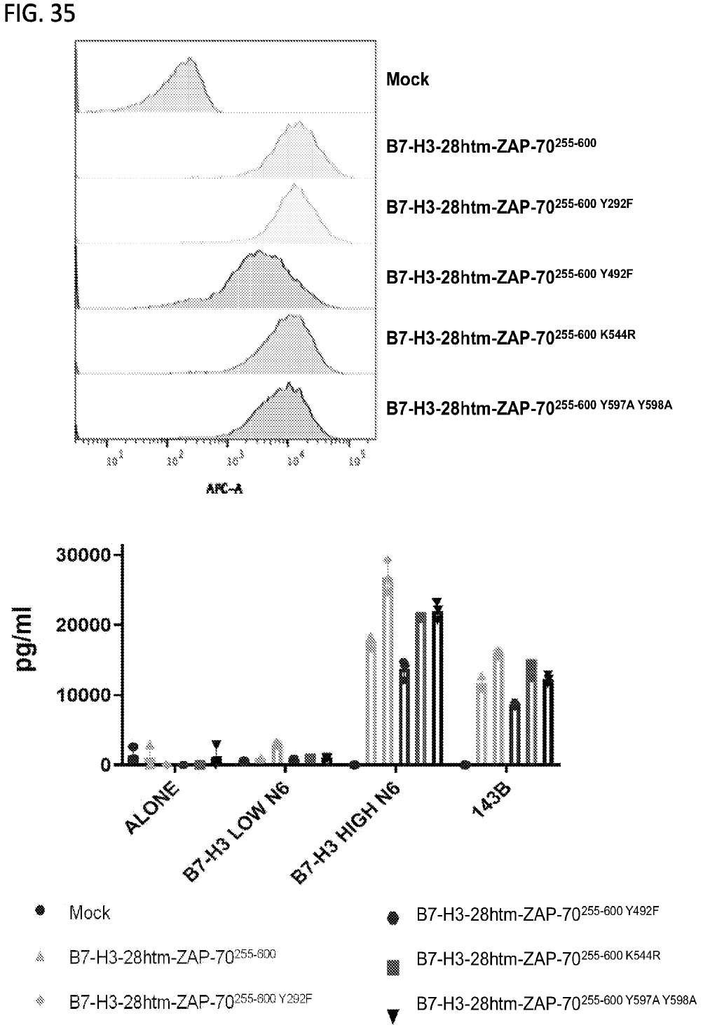

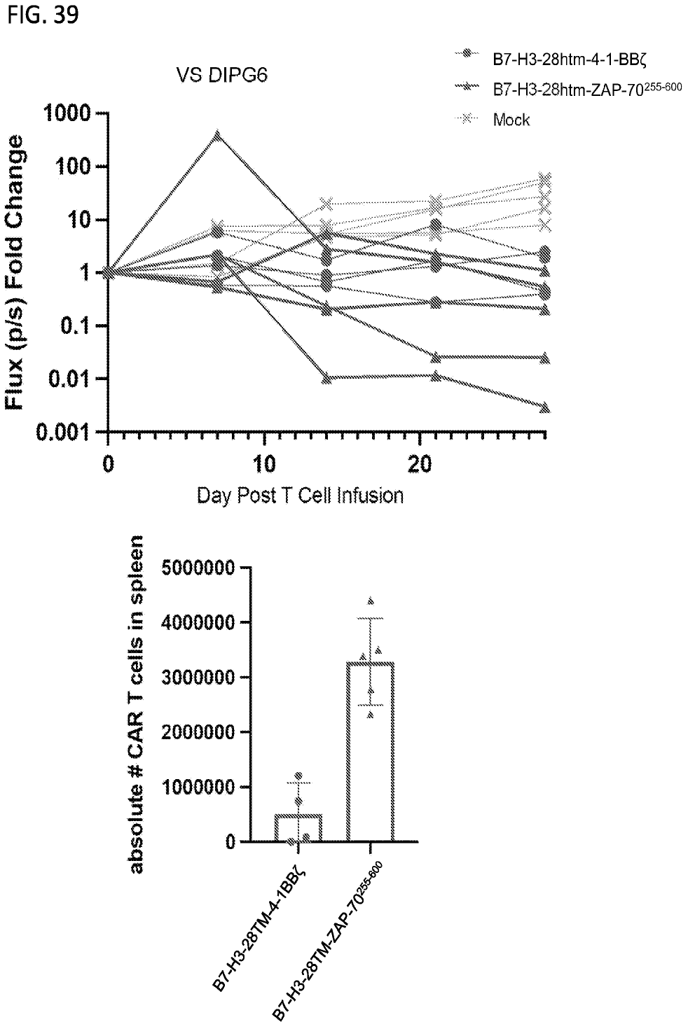

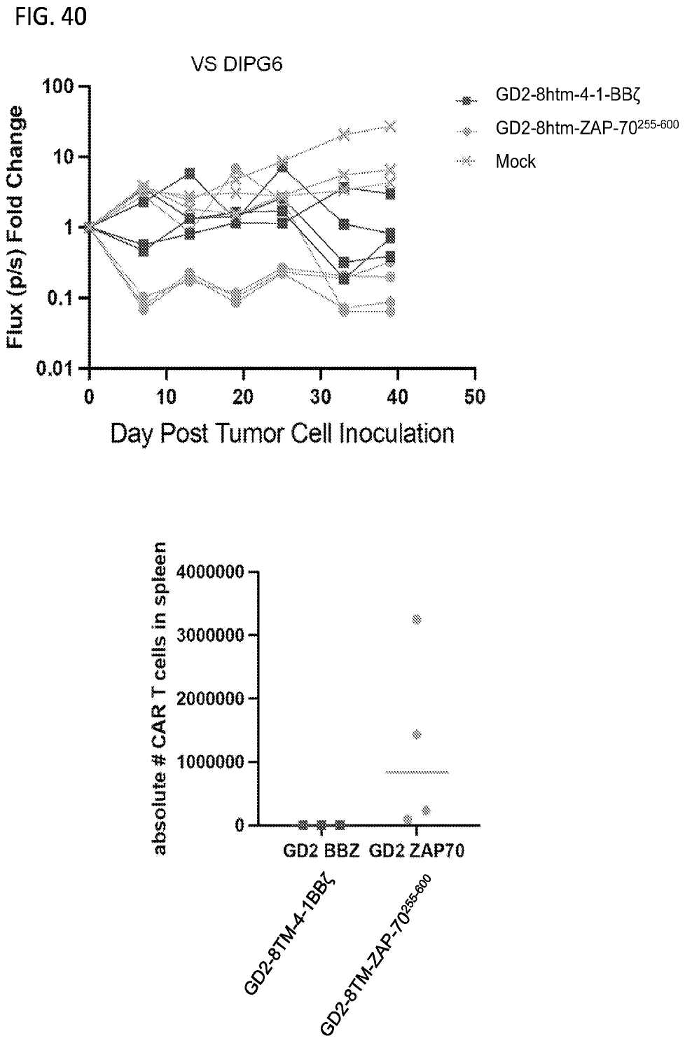

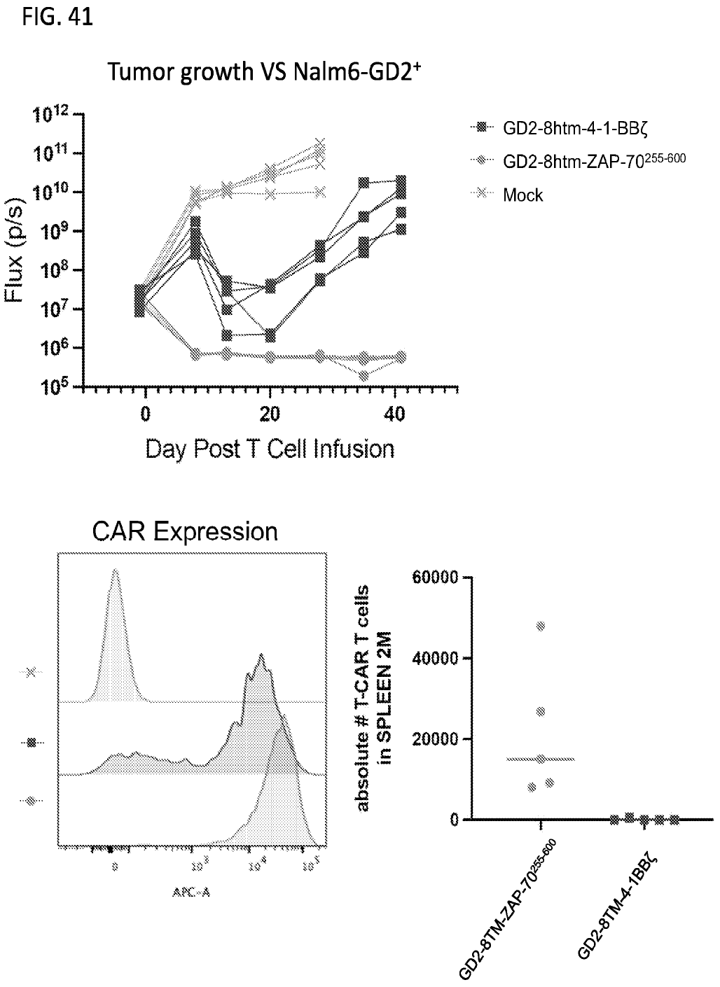

In a mouse model of xenograft neuroblastoma, B7-H3-ZAP70²⁵⁵⁻⁶⁰⁰ CARs outperformed traditional B7-H3-4-1BB-zeta CARs in controlling and eliminating tumor. Here, mice were injected with tumor cells intravenously in a metastatic model and then treated with MOCK (control) or T cells expressing B7-H3-4-1BBζ or B7-H3-ZAP70²⁵⁵⁻⁶⁰⁰ CAR constructs. As shown in FIGS. 34A-34E, B7-H3-truncated ZAP70 CAR-expressing T cells outperformed traditional CARs in tumor eradication. Similar advantage over 4-1BB-zeta CARs were identified for ZAP-70²⁵⁵⁻⁶⁰⁰ CARs, when using scFvs prone to tonic signaling (B7-H3 and GD2), in animal models involving diffuse intrinsic pontine glioma 6 xenografts (DIPG-6) (FIG. 39 and FIG. 40), and an animal model involving leukemia xenografts (GD2⁺-Nalm6) (FIG. 41). In all of these models, T cells recovered from mice at endpoint or specific timepoints demonstrated enhanced expansion and persistence of CAR T cells containing ZAP-70²⁵⁵⁻⁶⁰⁰ endodomains.

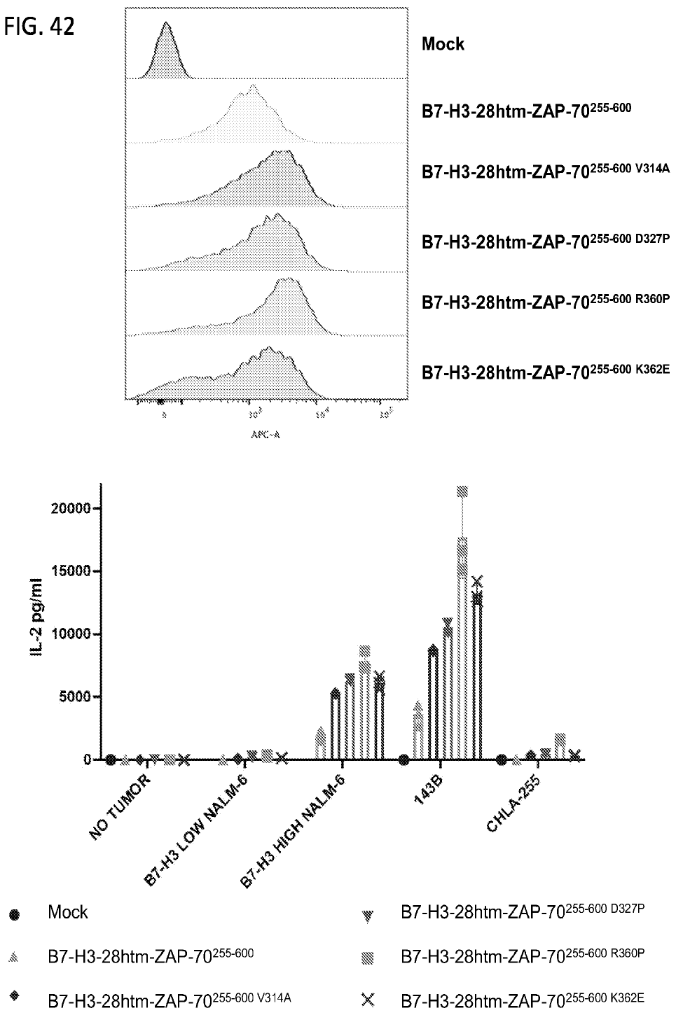

ZAP70 CAR-expressing T cells can be further enhanced through targeted mutations. In one instance, mutations such as Y292F, Y492F, K544R, and Y597F+Y598F can be introduced in the ZAP70 interdomain B, resulting in enhanced cytokine production in response to tumor cells (FIG. 35). Similarly, mutations such as V314A, D327P, R360P, and K362E also enhanced cytokine production in response to tumor cells (FIG. 42).

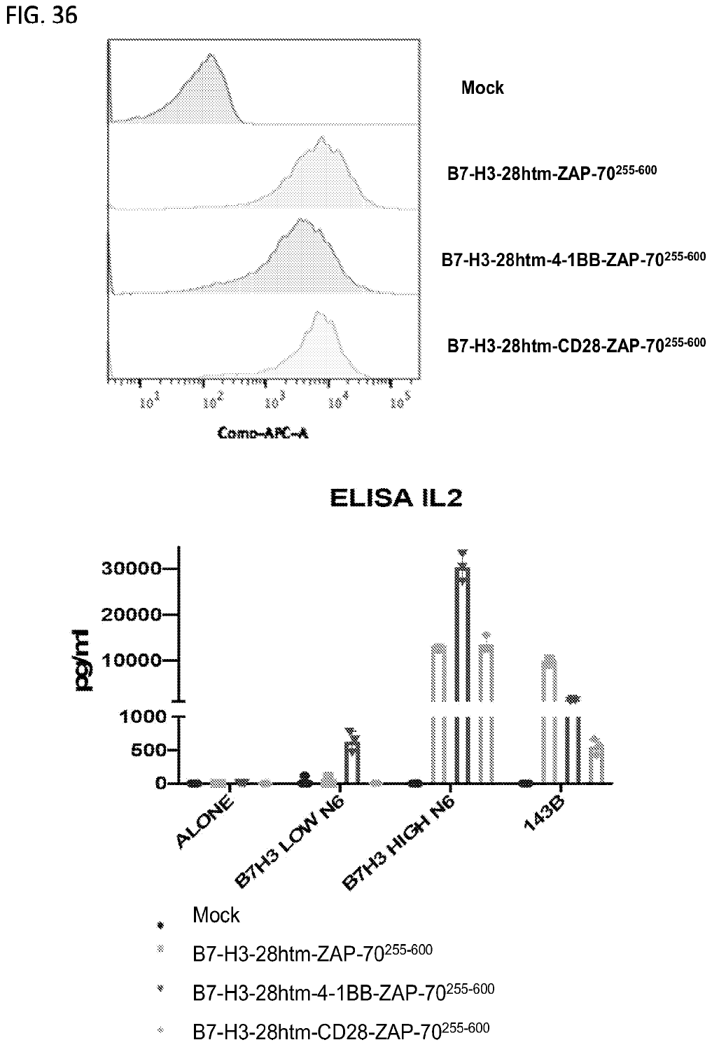

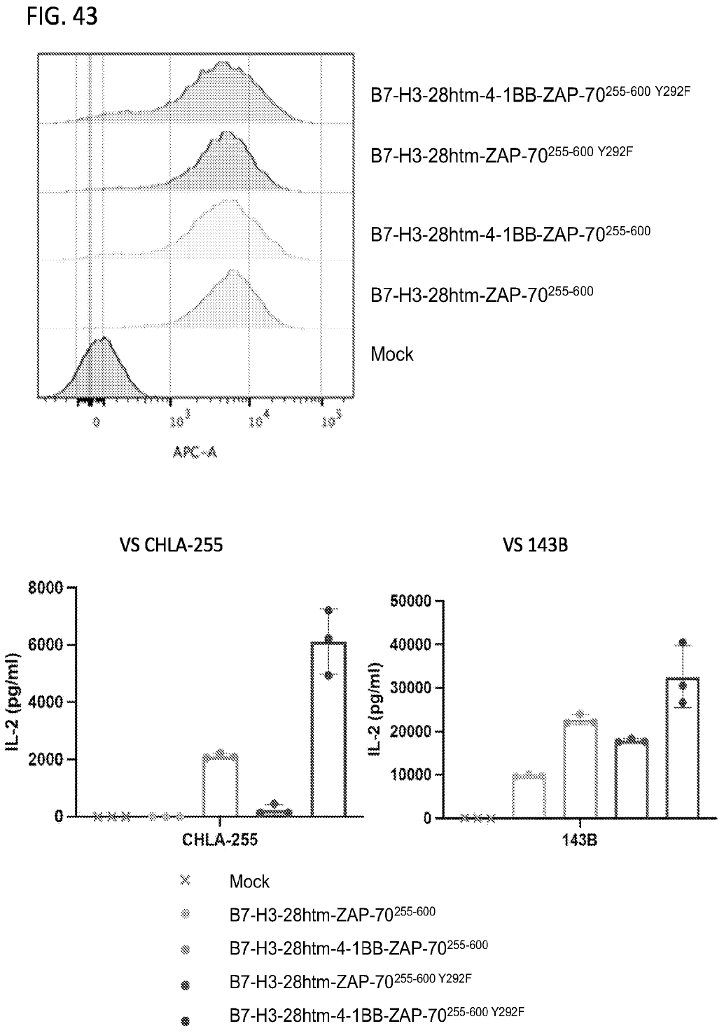

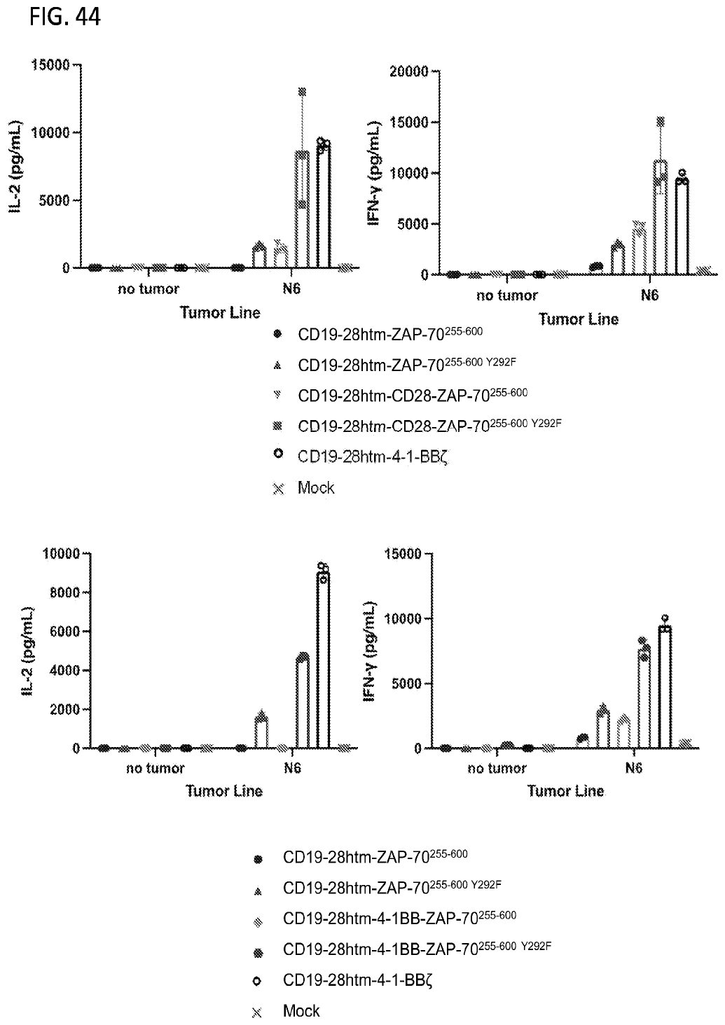

Additionally, ZAP70 CAR T cells can be further enhanced through integration of costimulatory domains. For instance, a 4-1BB costimulatory domain was introduced into a CAR construct, resulting in enhanced cytokine production in response to tumor cells (FIG. 36). In addition, combining enhancing mutations (e.g., Y292F) with costimulatory domains further enhanced the anti-tumor activity (e.g., measured by cytokine production levels) of the ZAP70 CAR molecules (FIG. 43). Costimulatory domains and enhancing mutations can be added to improve the potency of T cells bearing CARs containing ZAP-70²⁵⁵⁻⁶⁰⁰ fragment endodomains and non-tonic signaling scFvs to achieve enhanced potency (FIG. 44).

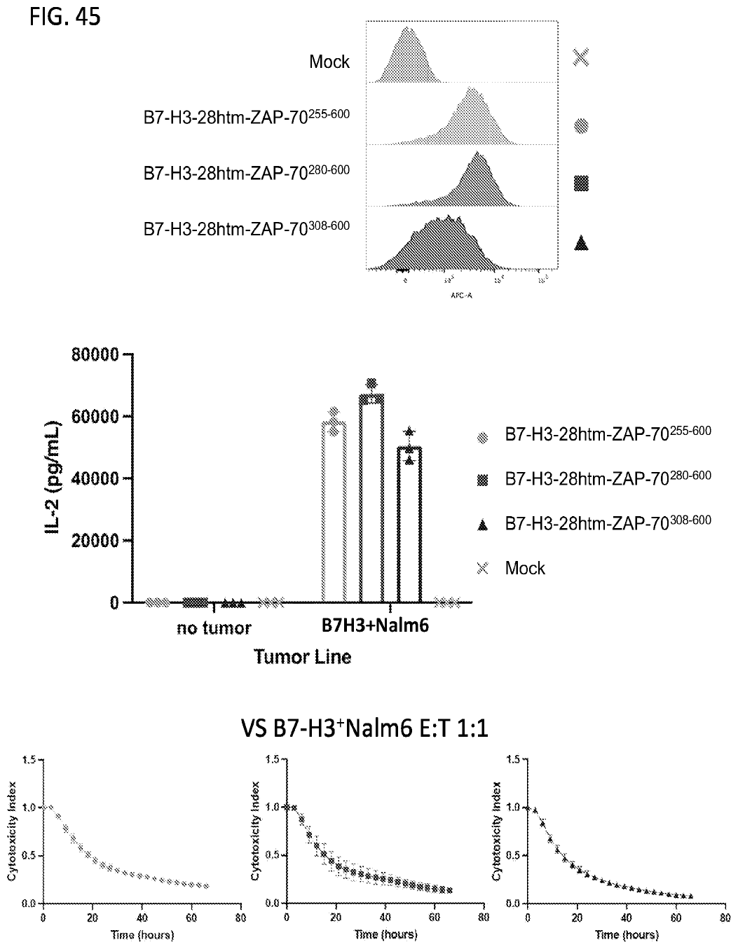

Different ZAP70 fragments were engineered for the CAR molecules described herein. For example, compared to ZAP70²⁵⁵⁻⁶⁰⁰ fragment endodomains illustrated in previous Examples, further truncated ZAP70 fragments were used in the CAR molecules. One exemplary advantage of these further truncated CAR molecules is their decreased packing size, without compromising efficacy. Exemplary CAR molecules comprising shorter ZAP70 fragments (e.g., ZAP-70²⁸⁰⁻⁶⁰⁰ and ZAP-70³⁰⁸⁻⁶⁰⁰) showed comparable activities to the ZAP-70²⁵⁵⁻⁶⁰⁰ CAR molecule in promoting cytokine production and cytotoxicity (FIG. 45).

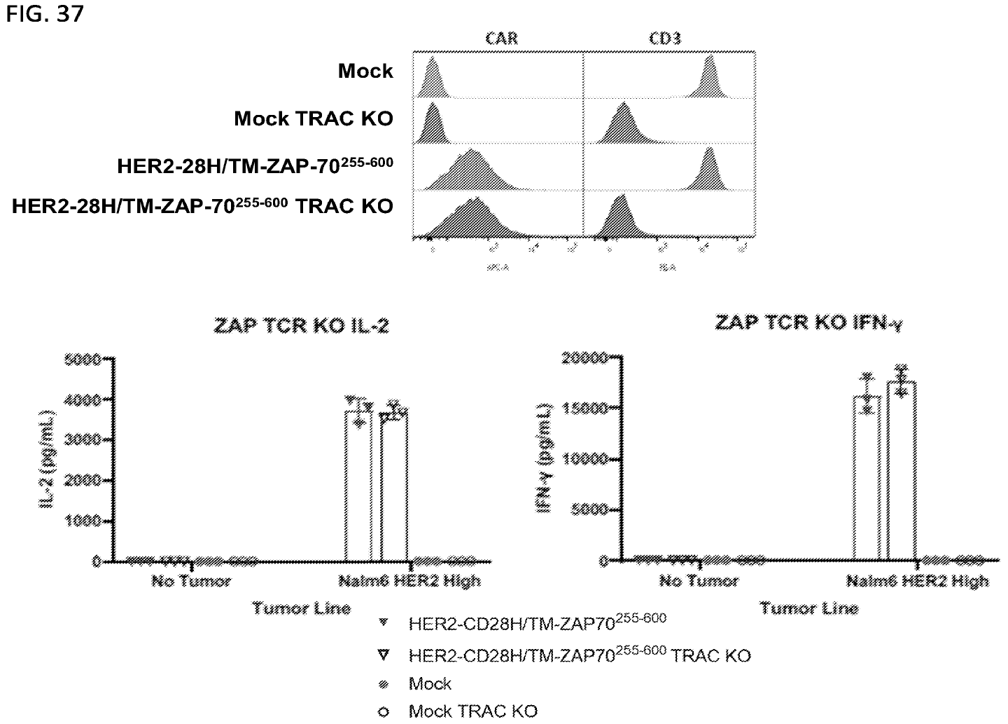

Activity of ZAP70 CARs does not depend on endogenous CD3zeta, as demonstrated after using CRISPR-Cas9 to knock out the native TCR in T cells bearing ZAP70 CARs, which performed equivalently to unedited T cells (FIG. 37).

### Example 6

### Discussion

A major advance of the discovery described herein is that it opens a landscape of potential antigens that may be shared with normal tissues for targeting by CARs. To date, there has been insufficient exploration of novel targets for solid tumor CARs and many current CAR targets are those that were already known as potential targets for antibody therapeutics (e.g. HER2, GD2, EGFR, B7-H3 etc.). With a system capable of discriminating normal tissue from tumor cells through recognition of antigen combinations, the work of what constitutes a tumor target antigen needs to be revisited.

The proximal signaling molecule CARs described herein (ZAP70, PLCG1, LAT/SLP-76, etc.) challenge the dominant paradigm that CAR T cell activity can only be initiated with CD3zeta. This work reorients the field and demonstrates the continued need to expand the CAR toolbox to include additional molecules that can activate immune cell effector functions. The pursuit of signaling molecules for CARs not only allows for more advanced Boolean logic receptors, but also has many additional potential benefits. For instance, incorporation of different signaling domains may help alleviate T cell exhaustion, a state of dysfunction that can develop with certain CARs. While a B7-H3 CAR with a traditional architecture (containing 4-1BB and CD3zeta signaling domains) expresses high levels of T cell exhaustion markers in the absence of antigen, a B7-H3-ZAP70 CAR expresses much lower levels of exhaustion markers (FIG. 33) and mediates improved anti-tumor control in a xenograft models of neuroblastoma, diffuse intrinsic pontine glioma, and leukemia (FIG. 34).

Additionally, use of different signaling molecules may help preventing major toxicities of CAR T cell therapies. The current generation of CAR T cells secretes large quantities of inflammatory cytokines that cause toxicity (called cytokine release syndrome) in patients. Alternative signaling pathways may restrict activity of CAR T cells to tumor cell killing without a production of high levels of cytokines, presenting a potential advantage. For instance, T cells expressing CAR constructs described herein may produce less cytokine in response to tumor than traditional CARs containing CD3zeta domains, but maintain their in vivo cytotoxicity activity.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### Example 7

### Further Characterization of Exemplary AND Gate CAR Constructs

This Example describes experiments performed to show further engineering to the AND Gate CAR constructs described in the instant application.

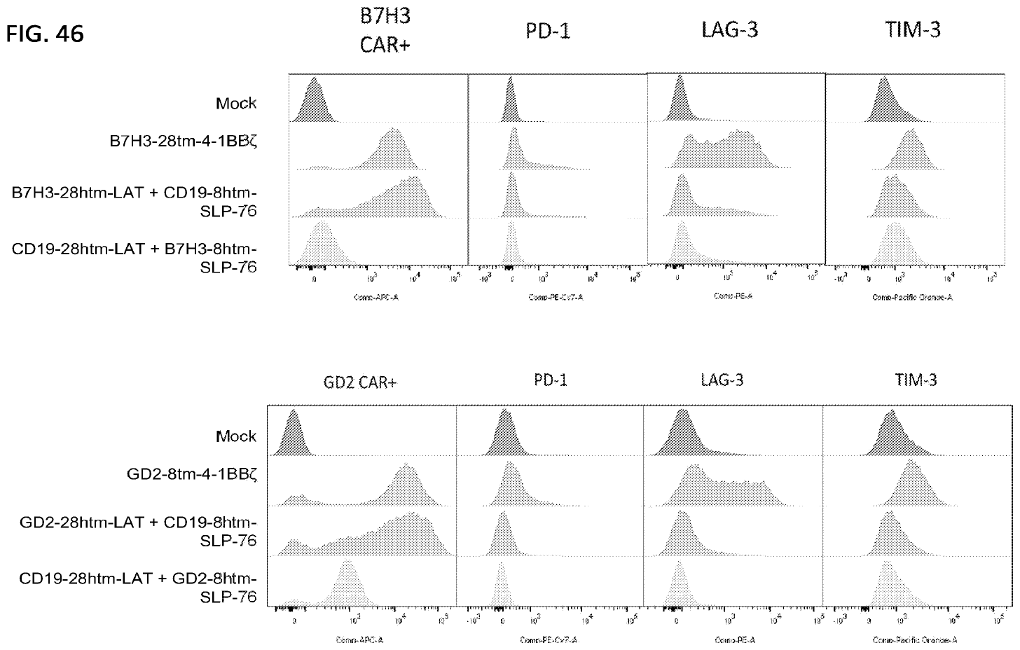

LINK CARs bearing one tonic-signaling scFv, described herein, do not show phenotypical characteristics of exhaustion. For example, T cells with LAT/SLP-76 CARs bearing B7-H3 or GD2 scFvs show less exhausted phenotypes compared to traditional B7-H3 or GD2-4-1BBzeta CAR T cells (FIG. 46).

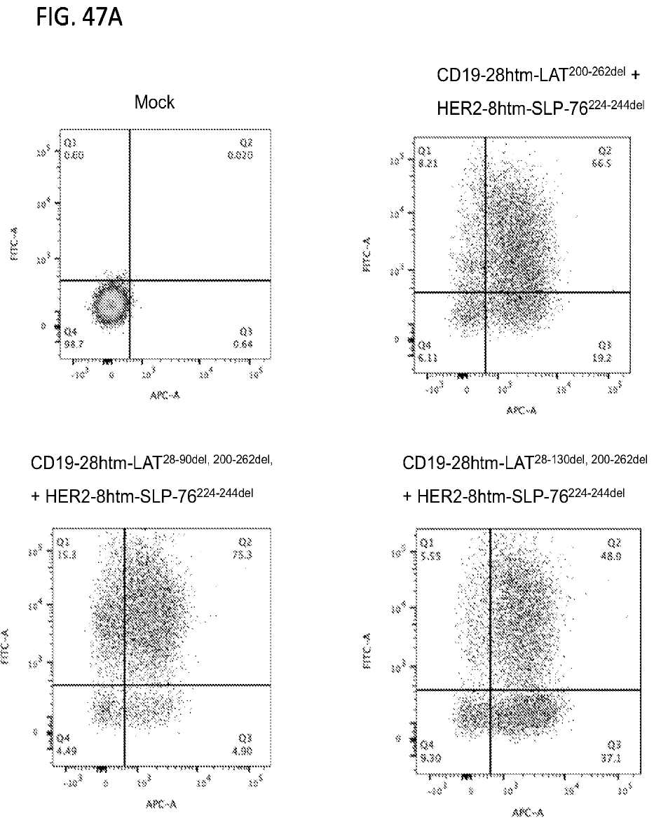

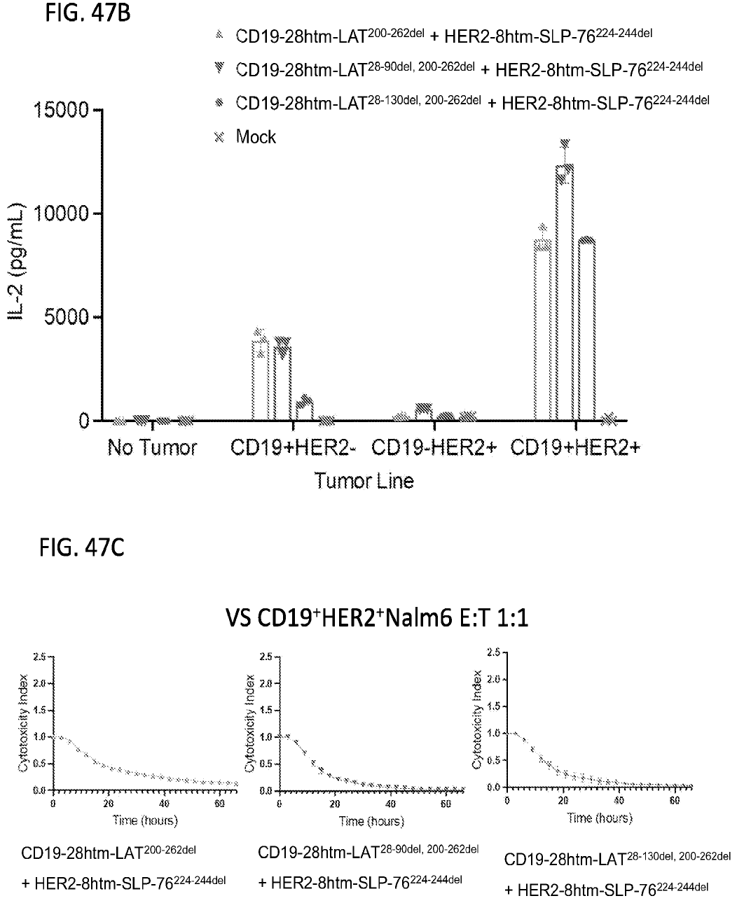

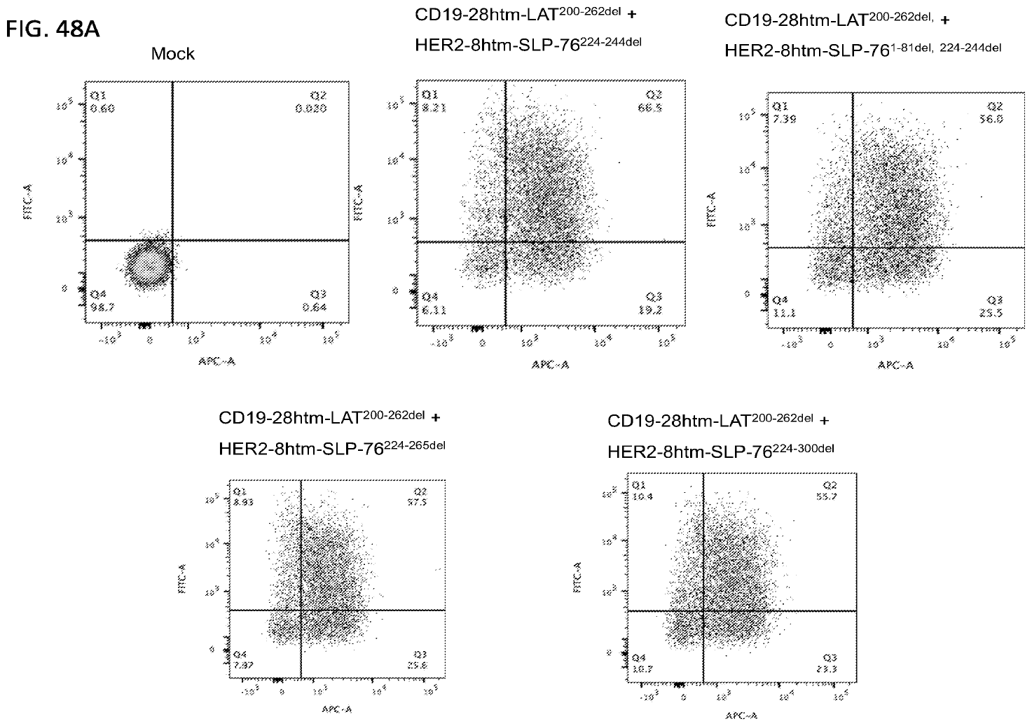

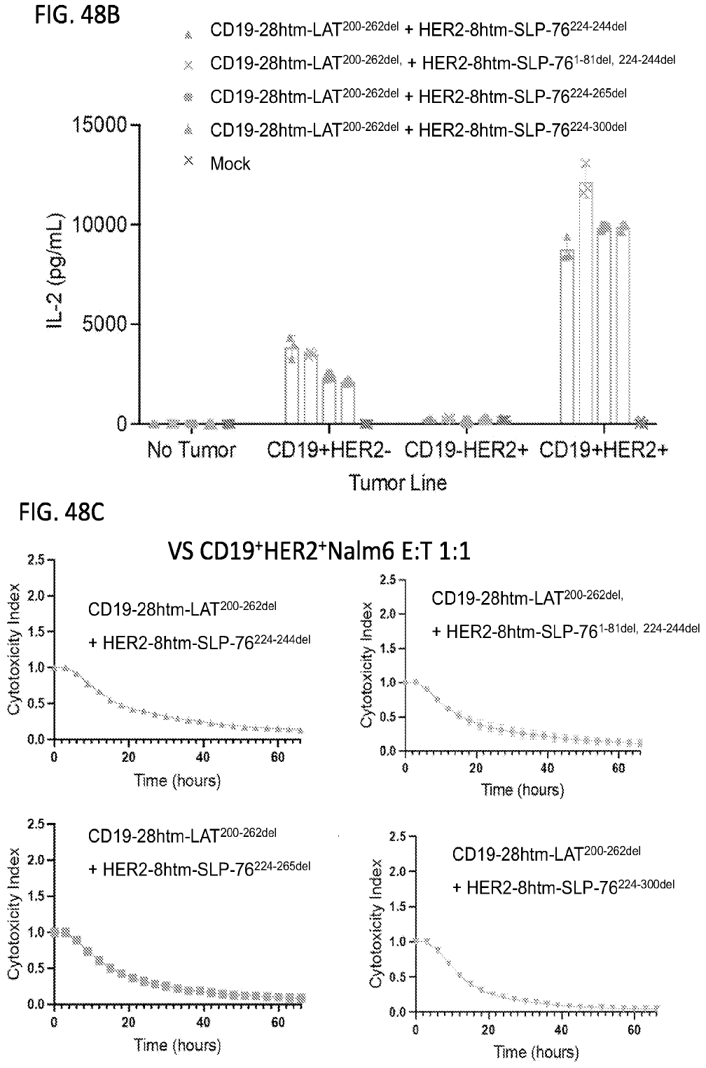

Either LAT CAR or SLP-76 CAR molecules of the AND Gate CAR system described herein can be further modified for optimization. For example, the LAT CAR (FIGS. 47A-47C) and the SLP-76 CAR (FIGS. 48A-48C) can be further truncated to reduce the packaging size, without negatively effecting efficacy. Exemplary shorter LAT domains include LAT^{28-90del, 200-262del} and LAT^{28-130del, 200-262del}. Exemplary shorter SLP-76 domains include SLP-76^{1-81del, 224-244del}, SLP-76^{224-265del}, and SLP-76^{224-300del}.

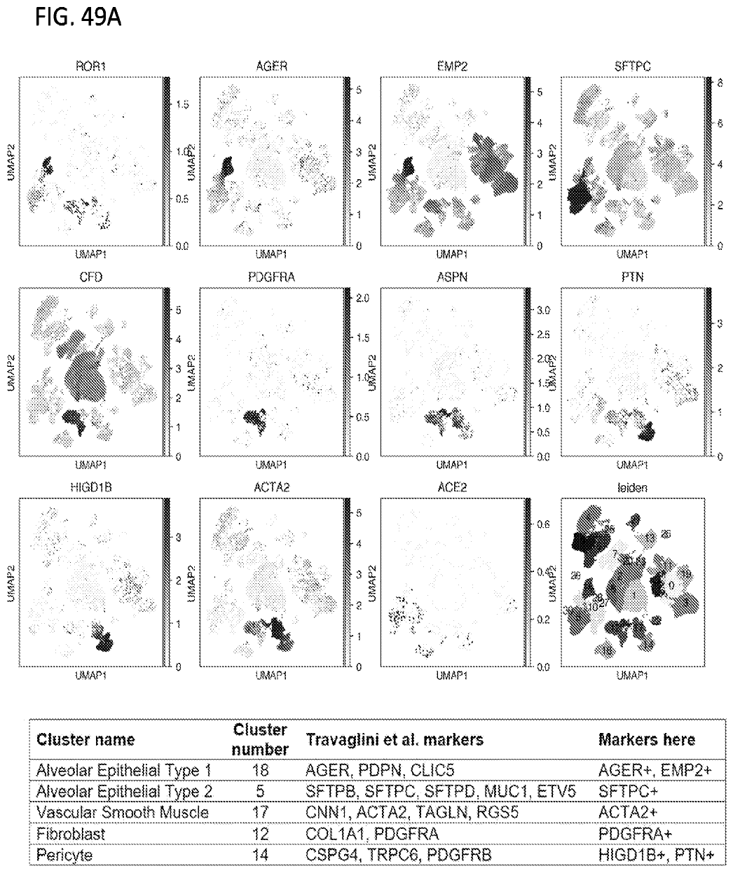

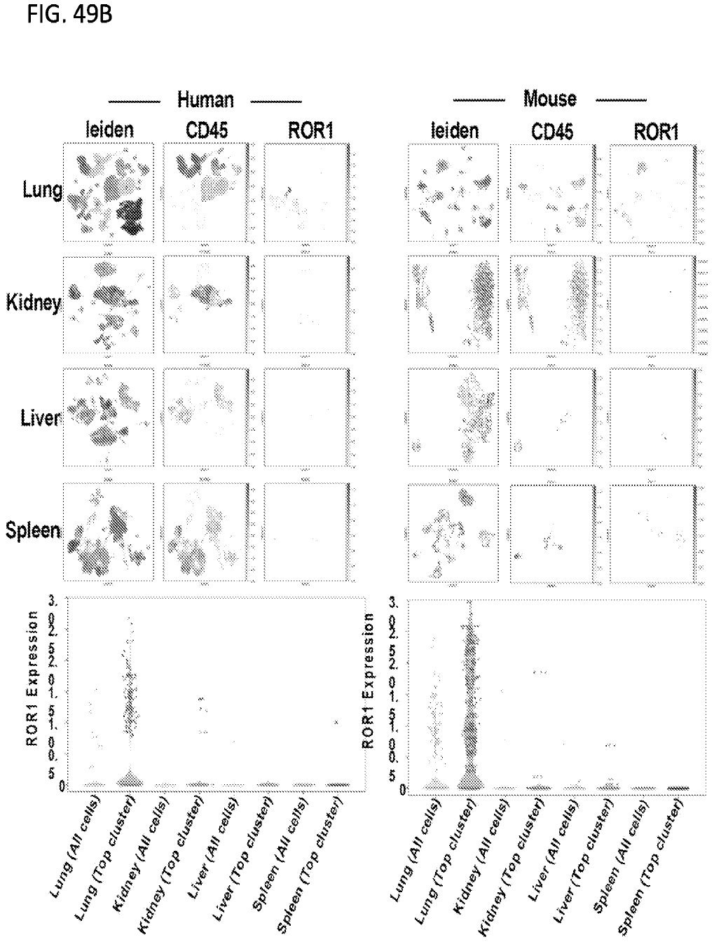

The AND Gate CAR systems described herein can be used to treat multiple diseases, such as cancers. Exemplary cancers may include ROR1+/CD19+ tumor cells. Expression of endogenous ROR1 on human and murine tissues is illustrated in FIGS. 49A-49B. This analysis revealed that the lungs are the most prominent site of ROR1 expression in these datasets. The mouse lung single-cell dataset from Raredon et al. Sci. Adv. (2019). doi:10.1126/sciadv.aaw3851 was also queried through the online web tool at World Wide Web site lungconnectome.net, which showed ROR1 expression on alveolar type II cells, pericytes (Peri), smooth muscle cells (SMCs), Col14a1+ fibroblasts (Fib_col14a1+), Col13a1+ fibroblasts (Fib_col13a1+), and mesothelial stromal cells (Meso) (from Raredon et al., 2019).

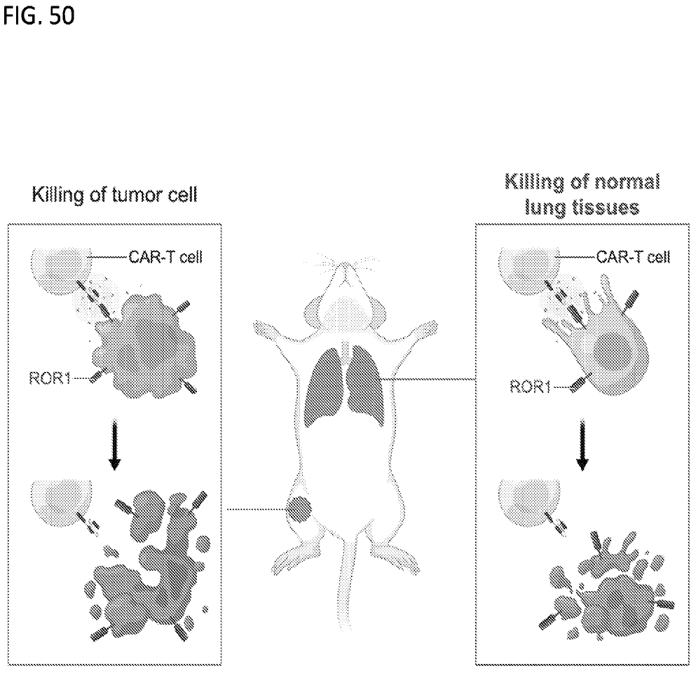

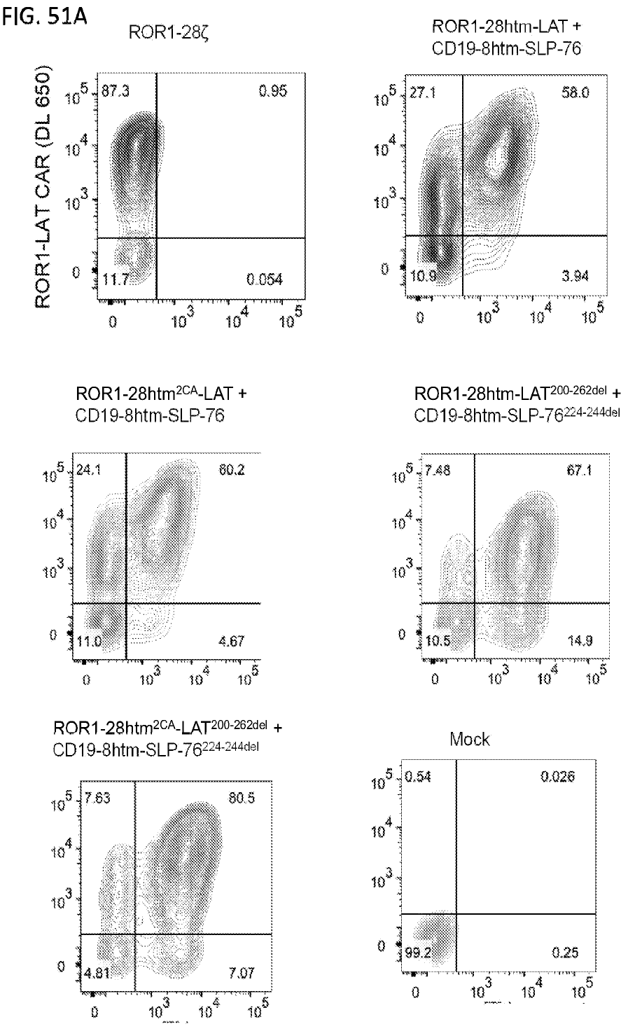

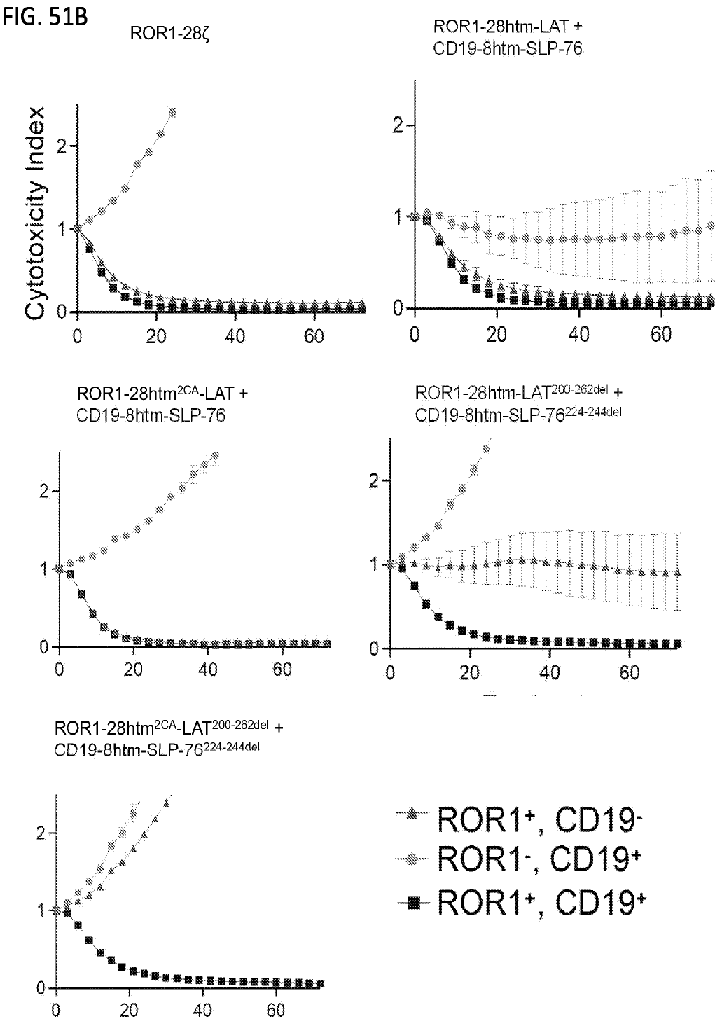

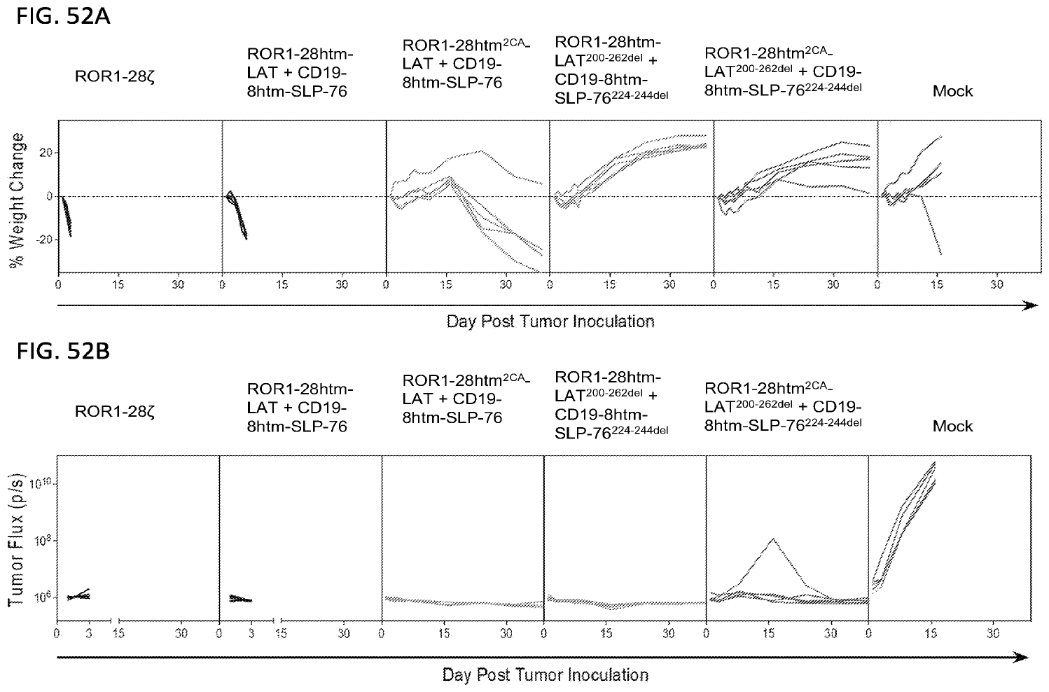

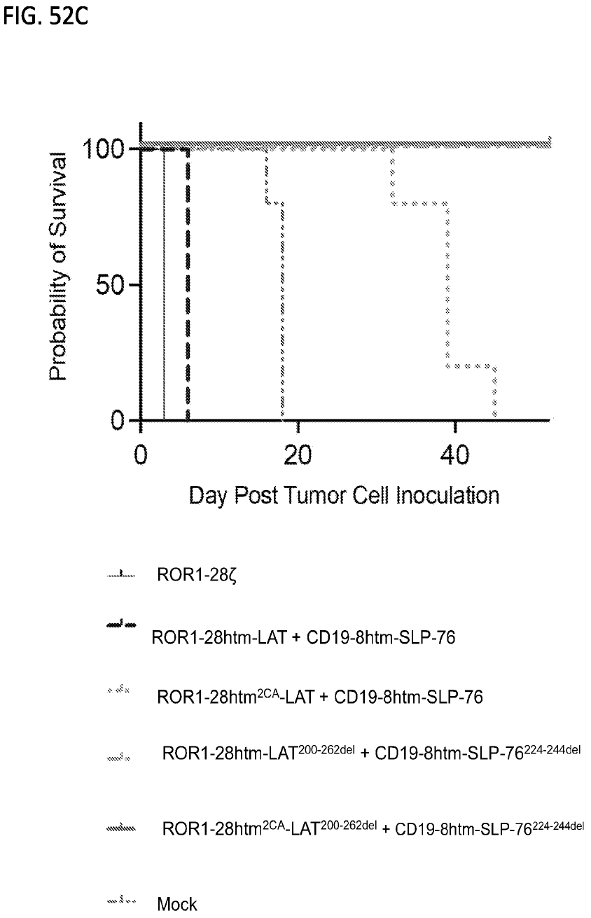

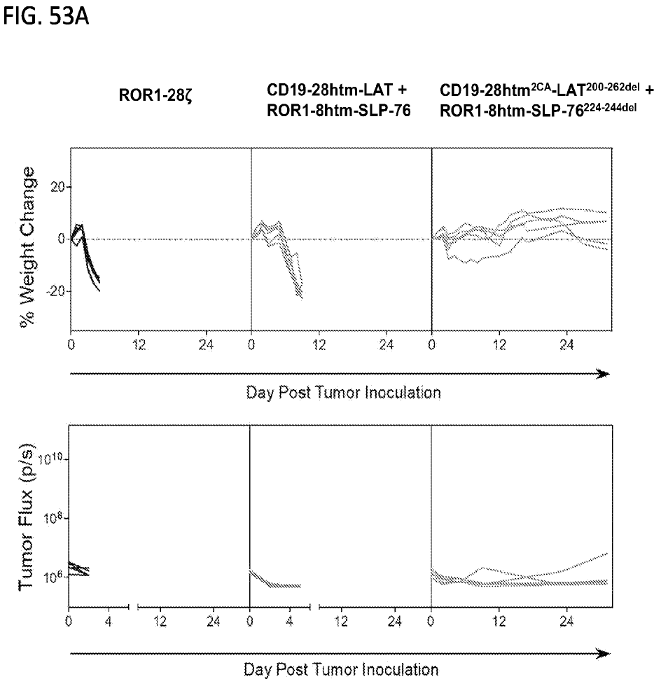

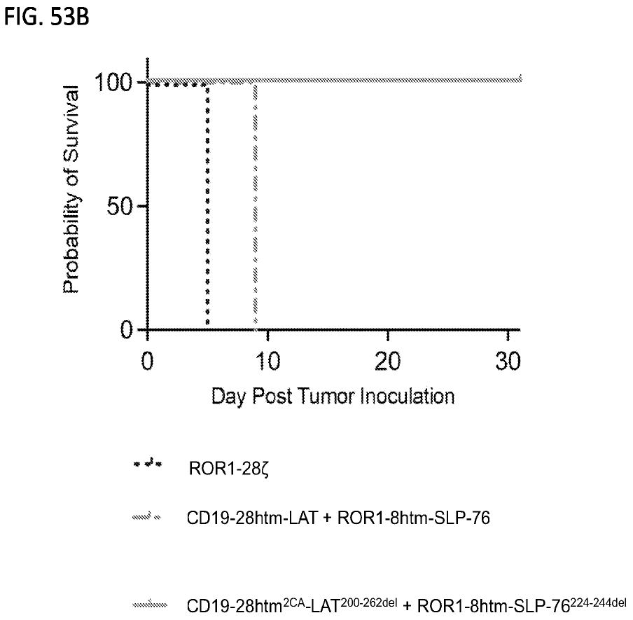

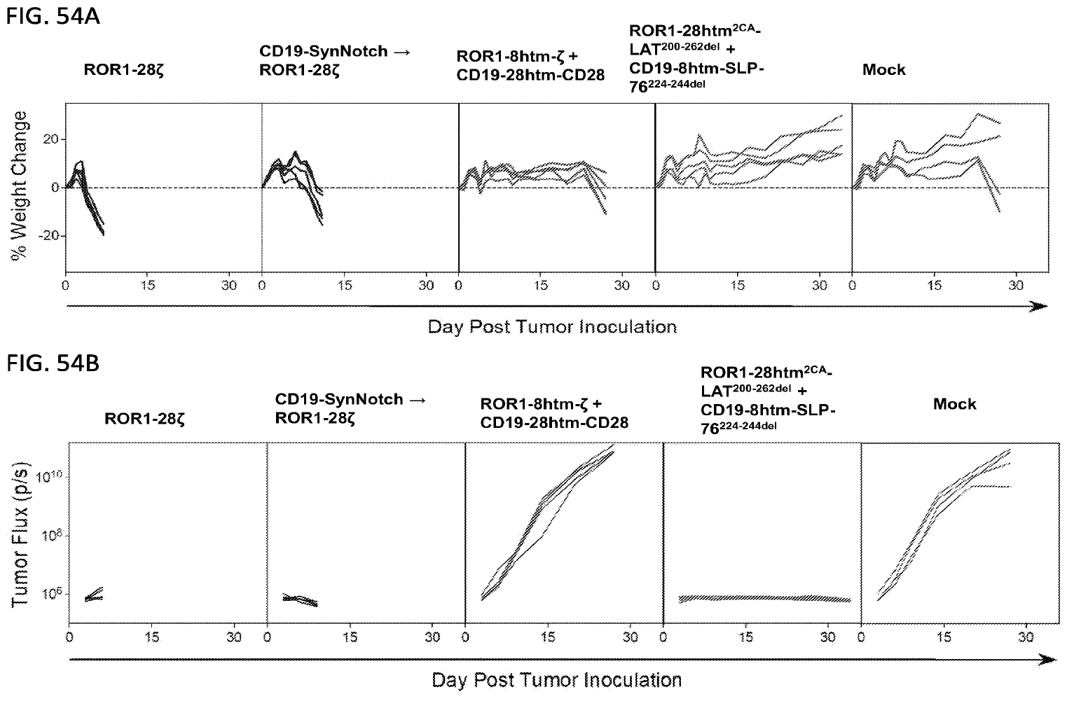

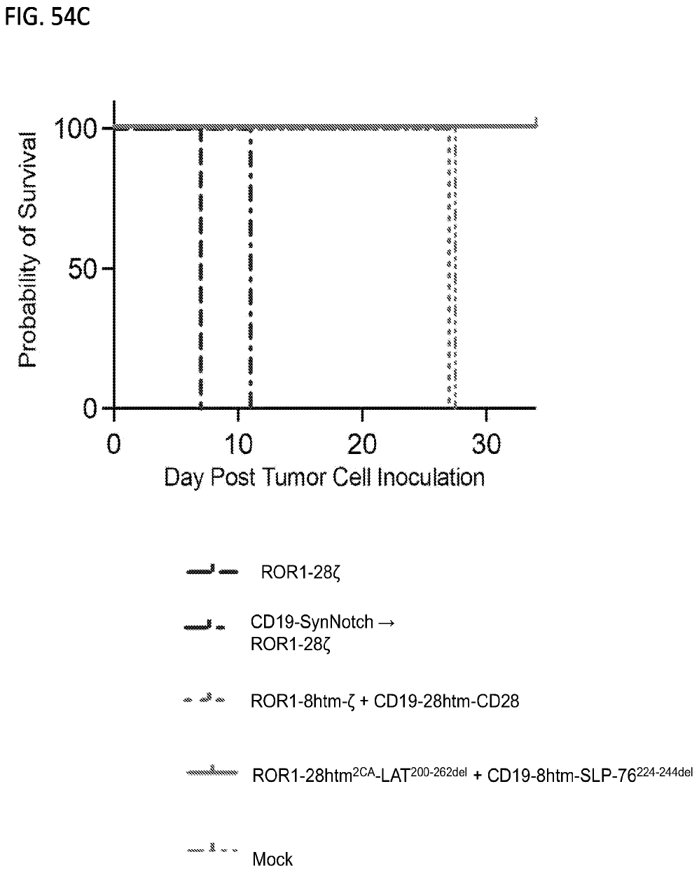

FIG. 50 shows an exemplary investigation of the capability of the LAT/SLP-76 AND Gate CAR molecules described herein to target ROR1⁺/CD19⁺ tumor cells in a murine model. CAR T cells primed to recognize ROR1 on ROR1+ tumor cells can also bind ROR1 on normal, endogenous tissues, resulting in killing of non-tumor cells and potential adverse side effects. As shown in FIGS. 51A-51B, LAT/SLP-76 CARs can selectively target ROR1⁺/CD19⁺ tumor cells without on-target, off-tumor leakiness. In mice, on-target, off-tumor toxicity due to ROR1 recognition of ROR1 on normal mouse tissues is manifested by weight loss. FIGS. 52A-52C show that different ROR1/CD-19-targeting LAT/SLP-76 CARs have different capacities of reducing tumor size and improving animal survival, while the traditional ROR1−CD28zeta CAR kills the animal quickly due to weight loss. Several combinations of ROR1/CD-19-targeting LAT/SLP-76 CAR mediate complete tumor clearance in the absence of any toxicity. Different ROR1 or CD-19-targeting LAT/SLP-76 CAR combinations were used in FIGS. 53A-53B, showing tumor eradication and improved survival with the LAT/SLP-76 CAR combinations compared to a standard ROR1−CD28zeta CAR. The ROR1/CD-19-targeting LAT/SLP-76 CAR was compared to several other AND gate systems (SPLIT CAR from Kloss et al. Nat Biotechnol. 2013; 31:71-75 and Syn-Notch from Roybal et al. Cell. 2016; 164:770-779. The SPLIT CAR system was ineffective in controlling tumor. The Syn-Notch system did not prevent on-target recognition of ROR1 on normal mouse tissues (and caused toxicity), while the ROR1/CD-19-targeting LAT/SLP-76 CAR mediated complete tumor clearance and no signs of toxicity (FIGS. 54A-54C).

### Example 8

### Treg Therapy Using CAR Construct and/or AND Gate CAR Constructs

This Example describes methods that can be used to produce regulatory T cells (Tregs) expressing the CAR molecules and/or the AND Gate CAR constructs described herein. Such CAR-Tregs can be used for immunotherapy for various diseases or disorder. However, one skilled in the art will appreciate that methods that deviate from these specific methods can also be used to successfully produce and/or use such CAR-Tregs for treatment.

Regulatory T cells (Tregs) are a subset of T cells that function to maintain homeostasis and prevent autoimmunity (1). Tregs make up 5-10% of the CD4+ T cell population (2) and are characterized by co-expression of CD4, CD25, the transcription factor Forkhead box protein 3 (FOXP3) and low levels of CD127. Tregs suppress the immune system by different mechanisms including contact-dependent mechanisms, through CTLA-4 engagement for example, and contact-independent, such as the release of cytokines e.g., IL-35 or IL-10. Given their proven role in preventing autoimmune diseases, Tregs may have potential in the promotion of tolerance. Although human Tregs constitute a small proportion of circulating CD4+ T cells, they are attractive candidates for immunotherapeutic purposes given that they can be isolated, manipulated and expanded in large numbers in vitro. Tregs can be applied in the treatment of autoimmune diseases and in the prevention of transplant rejection and graft vs. host disease (GvHD). For reviews on Treg therapy, see Zhang et al., Front. Immunol., 2018; 9:2359. doi: 10.3389/fimmu.2018.02359 and Mohseni et al., Front. Immunol. 2020; 11:1608. doi: 10.3389/fimmu.2020.01608.

CAR molecules, e.g., those described herein, may be introduced into Tregs to produce antigen-specific CAR-Treg cells (CAR-Tregs). Tregs expressing specific CAR molecules described herein, such as those in the sequence tables below and elsewhere in the specification (e.g., Examples) and/or figures, may be activated after specifically recognizing target cells (e.g., cancer/tumor cells) expressing antigen(s) recognizable by the ECD(s) of the CAR molecules or CAR molecule combinations (e.g., the AND Gate constructs). Activation of the CAR-Tregs may lead to production of cytokines and/or inhibit or kill target cells through cytotoxicity, with minimal off-target and/or on-target, off-tumor side effects and minimal T cell exhaustion.

In one exemplary experiment, Treg cells (e.g., collected by isolation and/or expansion from subject samples or healthy donors) can be genetically engineered (e.g., transduced or transformed or transfected) with, for example, a vector construct of the present application including for example, a viral vector or an expression vector for the expression of the CAR polypeptides of interest, using the methods described herein or in common knowledge in the art known by a skilled artisan.

In another exemplary experiment, animals carrying certain cells expressing specific antigen(s) are treated (e.g., injected intravenously or subcutaneously) by the CAR-Treg cells described herein. Compared to mock treatment (such as constructs that are designed not to work or have specificity toward irrelevant target), CAR-Tregs are capable to specifically inhibit immune cells at the organ site of disease that expresses specific antigen(s), with minimal off-target and/or on-target, off-tumor side effects and minimal T cell exhaustion.

In another exemplary experiment, a subject (e.g., a human having an autoimmune disease) is treated (e.g., by administering a retroviral vector) with the CAR-Treg cells described herein in a dosage regimen disclosed herein or determined by a physician. CAR-Tregs are capable to inhibit and/or kill the autoimmune cells located at an organ site expressing specific antigen(s) in the subject, with minimal off-target and/or on-target, off-tumor side effects and minimal T cell exhaustion, thus treating the subject and/or ameliorating at least one symptom of the autoimmune disease.

LINK T-REG Enables Specific Activity at Organ Sties of Auto-Immunity

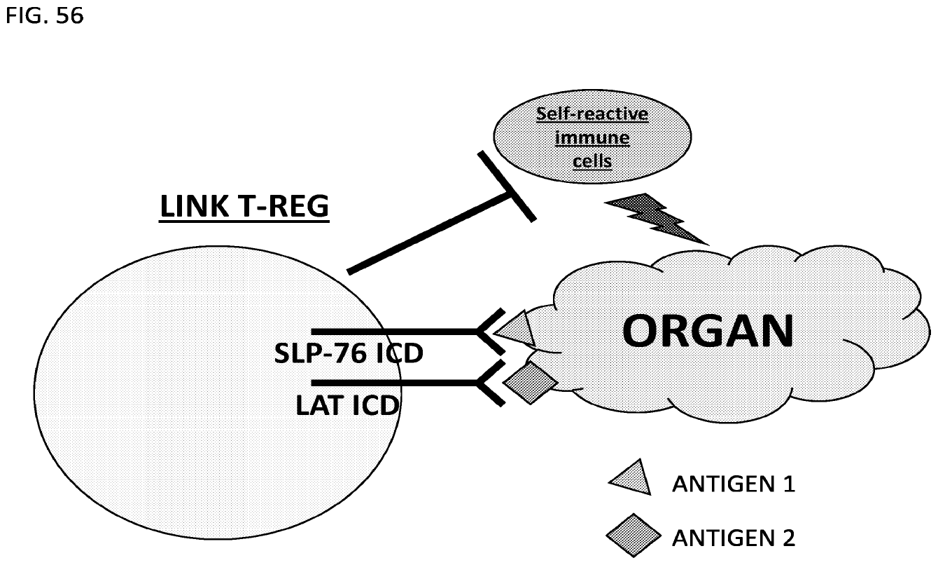

In another exemplary experiment, a system depicted as LINK T-REG is used to treat autoimmune diseases. Regulatory T cells are T cells that adapt a suppressive phenotype. T-regs can be redirected with a CAR to suppress self-reactive T cells and other self-reactive immune cells at an organ site of autoimmunity. This approach can treat autoimmune disorders. However, CAR Tregs have the potential to cause off-target immune suppression in organs that are not affected by the autoimmune condition because a CAR with a single antigen specificity may not be specific solely to the organ of involvement. Immune suppression by CAR T-regs outside the organ of interest can cause major infectious complications and unwanted immune suppression. This can be overcome by utilizing LINK T-REGs in which the LINK AND-gate system (e.g., the AND Gate system described in the present application) is deployed in a T-reg, as shown in FIG. 56. Specially, regulatory T cells are obtained from any method known to a skilled artisan, including, e.g., a) directly from patient blood or apheresis, b) using cytokines to differentiate peripherally obtained T cells to a T-reg phenotype, or c) reprogramming cells through overexpression of transcription factors such a FOXP3. These T-regs are transduced with one or several vectors that drive expression of SLP-76 and LAT CARs described herein. The LAT CAR and SLP-76 CAR target different antigens that specifically expressed on an organ-site of autoimmunity, thus leading to more specific activation of the T-regs than any single antigen can. This system is depicted as LINK T-REG. The combination of the two specificities (listed as Antigen 1 and Antigen 2) with LINK T-REG allows for specific targeting of the organ site of autoimmune disease and treatment of this autoimmune condition.

### Sequence Tables

The tables below lists exemplary sequences for CAR molecules described herein.

TABLE 1
Amino acid sequences for domains of and full-length exemplary CAR constructs

| | | | (Optional | |
|---|---|---|---|---|
| | | | costimulatory | |
| | | | domain amino | |
| | | | acid sequence) + | |
| | | | Intracellular | |
| | ECD domain | Hinge/TM | signaling | Full-length |
| | amino acid | domain amino | domain amino | construct amino |
| Construct name | sequence | acid sequence | acid sequence | acid sequence |
| HER2-CD28H/TM-SLP-76 | SEQ ID NO: 1 | SEQ ID NO: 6 | SEQ ID NO: 11 | SEQ ID NO: 27 |
| HER2-CD28H/TM-Fyn | SEQ ID NO: 1 | SEQ ID NO: 6 | SEQ ID NO: 12 | SEQ ID NO: 28 |
| CD19-CD28H/TM-LAT | SEQ ID NO: 2 | SEQ ID NO: 6 | SEQ ID NO: 13 | SEQ ID NO: 29 |
| CD19-CD28H/TM-Lck | SEQ ID NO: 2 | SEQ ID NO: 6 | SEQ ID NO: 14 | SEQ ID NO: 30 |
| HER2-CD28H/TM-ZAP70²⁵⁵⁻⁶⁰⁰ | SEQ ID NO: 1 | SEQ ID NO: 6 | SEQ ID NO: 15 | SEQ ID NO: 31 |
| HER2-CD28H/TM-PLCG1 | SEQ ID NO: 1 | SEQ ID NO: 6 | SEQ ID NO: 16 | SEQ ID NO: 32 |
| CD19-CD8H/TM-LAT | SEQ ID NO: 2 | SEQ ID NO: 7 | SEQ ID NO: 13 | SEQ ID NO: 33 |
| HER2-CD8H/TM-SLP76 | SEQ ID NO: 1 | SEQ ID NO: 7 | SEQ ID NO: 11 | SEQ ID NO: 34 |
| CD19-CD28H/TM-LAT^{Y161F} | SEQ ID NO: 2 | SEQ ID NO: 6 | SEQ ID NO: 17 | SEQ ID NO: 35 |
| CD19-CD28H/TM-LAT^{Y200F} | SEQ ID NO: 2 | SEQ ID NO: 6 | SEQ ID NO: 18 | SEQ ID NO: 36 |
| CD19- IgG4H/CD4TM-LAT^{G160D} | SEQ ID NO: 2 | SEQ ID NO: 8 | SEQ ID NO: 19 | SEQ ID NO: 37 |
| CD19-CD4H/TM-LAT | SEQ ID NO: 2 | SEQ ID NO: 9 | SEQ ID NO: 13 | SEQ ID NO: 38 |
| CD19-IgG4H/CD4TM-LAT | SEQ ID NO: 2 | SEQ ID NO: 8 | SEQ ID NO: 13 | SEQ ID NO: 39 |
| HER2-CD28H/TM-SLP-76^{K30R} | SEQ ID NO: 1 | SEQ ID NO: 6 | SEQ ID NO: 20 | SEQ ID NO: 40 |
| HER2-CD28H/TM-CD5 | SEQ ID NO: 1 | SEQ ID NO: 6 | SEQ ID NO: 21 | SEQ ID NO: 41 |
| HER2-CD28H/TM-CD6 | SEQ ID NO: 1 | SEQ ID NO: 6 | SEQ ID NO: 22 | SEQ ID NO: 42 |
| HER2-CD28H/TM-FcγR1 | SEQ ID NO: 1 | SEQ ID NO: 6 | SEQ ID NO: 23 | SEQ ID NO: 43 |
| HER2-CD28H/TM-4-1BB | SEQ ID NO: 1 | SEQ ID NO: 6 | SEQ ID NO: 24 | SEQ ID NO: 44 |
| HER2-CD28H/TM-CD28 | SEQ ID NO: 1 | SEQ ID NO: 6 | SEQ ID NO: 25 | SEQ ID NO: 45 |
| HER2-CD28H/TM-CD2 | SEQ ID NO: 1 | SEQ ID NO: 6 | SEQ ID NO: 26 | SEQ ID NO: 46 |
| HA-IgG1 long-CD28H/TM-ZAP70²⁵⁵⁻⁶⁰⁰ | SEQ ID NO: 3 | SEQ ID NO: 10 | SEQ ID NO: 15 | SEQ ID NO: 47 |
| B7-H3-CD28H/TM- ZAP70²⁵⁵⁻⁶⁰⁰ | SEQ ID NO: 4 | SEQ ID NO: 6 | SEQ ID NO: 15 | SEQ ID NO: 48 |
| CD22-CD8H/TM-LAT | SEQ ID NO: 5 | SEQ ID NO: 7 | SEQ ID NO: 13 | SEQ ID NO: 49 |
| CD19-CD28H/TM^{2CA}-LAT | SEQ ID NO: 2 | SEQ ID NO: 102 | SEQ ID NO: 13 | SEQ ID NO: 111 |
| CD19-CD28H/TM-LAT^{2YF} | SEQ ID NO: 2 | SEQ ID NO: 6 | SEQ ID NO: 103 | SEQ ID NO: 112 |
| CD19-IgG4H-CD4TM-LAT^{2YF} | SEQ ID NO: 2 | SEQ ID NO: 8 | SEQ ID NO: 103 | SEQ ID NO: 113 |
| CD19-CD28H/TM-LAT^{200-262 del} | SEQ ID NO: 2 | SEQ ID NO: 6 | SEQ ID NO: 104 | SEQ ID NO: 114 |
| CD19-CD28H/TM^{2CA}-LAT^{2YF} | SEQ ID NO: 2 | SEQ ID NO: 102 | SEQ ID NO: 103 | SEQ ID NO: 115 |
| CD19-CD28H/TM^{2CA}-LAT^{200-262 del} | SEQ ID NO: 2 | SEQ ID NO: 102 | SEQ ID NO: 104 | SEQ ID NO: 116 |
| CD19-CD28H/TM-LAT^{3YF} | SEQ ID NO: 2 | SEQ ID NO: 6 | SEQ ID NO: 105 | SEQ ID NO: 117 |
| HER2-CD8H/TM-SLP-76^{224-244 del} | SEQ ID NO: 1 | SEQ ID NO: 7 | SEQ ID NO: 106 | SEQ ID NO: 118 |
| HER2-CD28H/TM-SLP-76^{224-244 del} | SEQ ID NO: 1 | SEQ ID NO: 6 | SEQ ID NO: 106 | SEQ ID NO: 119 |
| B7-H3-CD28H/TM-ZAP70^{255-600 Y292F} | SEQ ID NO: 4 | SEQ ID NO: 6 | SEQ ID NO: 107 | SEQ ID NO: 120 |
| B7-H3-CD28H/TM-ZAP70^{255-600 Y492F} | SEQ ID NO: 4 | SEQ ID NO: 6 | SEQ ID NO: 108 | SEQ ID NO: 121 |
| B7-H3-CD28H/TM-ZAP70^{255-600 K544R} | SEQ ID NO: 4 | SEQ ID NO: 6 | SEQ ID NO: 109 | SEQ ID NO: 122 |
| B7-H3-CD28H/TM-ZAP70^{255-600 Y597F Y598F} | SEQ ID NO: 4 | SEQ ID NO: 6 | SEQ ID NO: 110 | SEQ ID NO: 123 |
| B7-H3-CD28H/TM-4-1BB-ZAP70²⁵⁵⁻⁶⁰⁰ | SEQ ID NO: 4 | SEQ ID NO: 6 | (SEQ ID NO: 146) + | SEQ ID NO: 148 |
| | | | SEQ ID NO: 15 | |
| B7-H3-CD28H/TM-CD28-ZAP70²⁵⁵⁻⁶⁰⁰ | SEQ ID NO: 4 | SEQ ID NO: 6 | (SEQ ID NO: 147) + | SEQ ID NO: 149 |
| | | | SEQ ID NO: 15 | |
| CD19-CD28H/TM-ZAP70²⁵⁵⁻⁶⁰⁰ | SEQ ID NO: 2 | SEQ ID NO: 6 | SEQ ID NO: 15 | SEQ ID NO: 150 |
| CD19-CD28H/TM-PLCG1 | SEQ ID NO: 2 | SEQ ID NO: 6 | SEQ ID NO: 16 | SEQ ID NO: 151 |
| GD2-8h/tm-ZAP70 (255-600) | SEQ ID NO: 161 | SEQ ID NO: 7 | SEQ ID NO: 15 | SEQ ID NO: 162 |
| B7-H3-28h/tm-ZAP70 (255-600 V314A) | SEQ ID NO: 4 | SEQ ID NO: 6 | SEQ ID NO: 163 | SEQ ID NO: 164 |
| B7-H3-28h/tm-ZAP70 (255-600 D327P) | SEQ ID NO: 4 | SEQ ID NO: 6 | SEQ ID NO: 165 | SEQ ID NO: 166 |
| B7-H3-28h/tm-ZAP70 (255-600 R360P) | SEQ ID NO: 4 | SEQ ID NO: 6 | SEQ ID NO: 167 | SEQ ID NO: 168 |
| B7-H3-28h/tm-ZAP70 (255-600 K362E) | SEQ ID NO: 4 | SEQ ID NO: 6 | SEQ ID NO: 169 | SEQ ID NO: 170 |
| B7-H3-28h/tm-4-1BB-ZAP70 (255-600 | SEQ ID NO: 4 | SEQ ID NO: 6 | (SEQ ID NO: 146) + | SEQ ID NO: 171 |
| Y292F) | | | SEQ ID NO: 107 | |
| CD19-28h/tm-ZAP70 (255-600 Y292F) | SEQ ID NO: 2 | SEQ ID NO: 6 | SEQ ID NO: 107 | SEQ ID NO: 172 |
| CD19-28h/tm-CD28-ZAP70 (255-600) | SEQ ID NO: 2 | SEQ ID NO: 6 | (SEQ ID NO: 147) + | SEQ ID NO: 173 |
| | | | SEQ ID NO: 15 | |
| CD19-28h/tm-4-1BB-ZAP70 (255-600) | SEQ ID NO: 2 | SEQ ID NO: 6 | (SEQ ID NO: 146) + | SEQ ID NO: 174 |
| | | | SEQ ID NO: 15 | |
| CD19-28h/tm-CD28-ZAP70 (255-600 | SEQ ID NO: 2 | SEQ ID NO: 6 | (SEQ ID NO: 147) + | SEQ ID NO: 175 |
| Y292F) | | | SEQ ID NO: 107 | |
| CD19-28h/tm-4-1BB-ZAP70 (255-600 | SEQ ID NO: 2 | SEQ ID NO: 6 | (SEQ ID NO: 146) + | SEQ ID NO: 176 |
| Y292F) | | | SEQ ID NO: 107 | |
| B7-H3-28h/tm-ZAP70 (280-600) | SEQ ID NO: 4 | SEQ ID NO: 6 | SEQ ID NO: 177 | SEQ ID NO: 178 |
| B7-H3-28h/tm-ZAP70 (308-600) | SEQ ID NO: 4 | SEQ ID NO: 6 | SEQ ID NO: 179 | SEQ ID NO: 180 |
| B7-H3-28h/tm-LAT | SEQ ID NO: 4 | SEQ ID NO: 6 | SEQ ID NO: 13 | SEQ ID NO: 181 |
| B7-H3-8h/tm-SLP-76 | SEQ ID NO: 4 | SEQ ID NO: 7 | SEQ ID NO: 11 | SEQ ID NO: 182 |
| GD2-28h/tm-LAT | SEQ ID NO: 161 | SEQ ID NO: 6 | SEQ ID NO: 13 | SEQ ID NO: 183 |
| GD2-8h/tm-SLP-76 | SEQ ID NO: 161 | SEQ ID NO: 7 | SEQ ID NO: 11 | SEQ ID NO: 184 |
| CD19-28h/tm-LAT (28-90del, 200-262del) | SEQ ID NO: 2 | SEQ ID NO: 6 | SEQ ID NO: 185 | SEQ ID NO: 186 |
| CD19-28h/tm(2CA)-LAT (28-90del, 200- | SEQ ID NO: 2 | SEQ ID NO: 102 | SEQ ID NO: 185 | SEQ ID NO: 187 |
| 262del) | | | | |
| CD19-28h/tm-LAT (28-130del, 200- | SEQ ID NO: 2 | SEQ ID NO: 6 | SEQ ID NO: 188 | SEQ ID NO: 189 |
| 262del) | | | | |
| CD19-28h/tm(2CA)-LAT (28-130del, 200- | SEQ ID NO: 2 | SEQ ID NO: 102 | SEQ ID NO: 188 | SEQ ID NO: 190 |
| 262del) | | | | |
| HER2-8h/tm-SLP-76 (1-81del, 224-244del) | SEQ ID NO: 1 | SEQ ID NO: 7 | SEQ ID NO: 191 | SEQ ID NO: 192 |
| HER2-8h/tm-SLP-76 (224-265del) | SEQ ID NO: 1 | SEQ ID NO: 7 | SEQ ID NO: 193 | SEQ ID NO: 194 |
| HER2-8h/tm-SLP-76 (1-81del, 224- | SEQ ID NO: 1 | SEQ ID NO: 7 | SEQ ID NO: 195 | SEQ ID NO: 196 |
| 300del)) | | | | |
| ROR1-28h/tm-LAT | SEQ ID NO: 197 | SEQ ID NO: 6 | SEQ ID NO: 13 | SEQ ID NO: 198 |
| ROR1-28h/tm(2CA)-LAT | SEQ ID NO: 197 | SEQ ID NO: 102 | SEQ ID NO: 13 | SEQ ID NO: 199 |
| ROR1-28h/tm-LAT (200-262del) | SEQ ID NO: 197 | SEQ ID NO: 6 | SEQ ID NO: 104 | SEQ ID NO: 200 |
| ROR1-28h/tm(2CA)-LAT (200-262del) | SEQ ID NO: 197 | SEQ ID NO: 102 | SEQ ID NO: 104 | SEQ ID NO: 201 |
| CD19-8h/tm-SLP-76 (224-244del) | SEQ ID NO: 2 | SEQ ID NO: 7 | SEQ ID NO: 106 | SEQ ID NO: 202 |
| ROR1-8h/tm-SLP-76 | SEQ ID NO: 197 | SEQ ID NO: 7 | SEQ ID NO: 11 | SEQ ID NO: 203 |
| ROR1-8h/tm-SLP-76 (224-244del) | SEQ ID NO: 197 | SEQ ID NO: 7 | SEQ ID NO: 106 | SEQ ID NO: 204 |

TABLE 2
Nucleic acid sequences for domains of and full-length exemplary CAR constructs

| | | | (Optional | |
|---|---|---|---|---|
| | | | costimulatory | |
| | | | domain nucleic | |
| | | | acid sequence) + | |
| | | | Intracellular | |
| | ECD domain | Hinge/TM | signaling | Full-length |
| | nucleic acid | domain nucleic | domain nucleic | nucleic acid |
| Construct name | sequence | acid sequence | acid sequence | sequence |
| HER2-CD28H/TM-SLP-76 | SEQ ID NO: 50 | SEQ ID NO: 55 | SEQ ID NO: 60 | SEQ ID NO: 76 |
| HER2-CD28H/TM-Fyn | SEQ ID NO: 50 | SEQ ID NO: 55 | SEQ ID NO: 61 | SEQ ID NO: 77 |
| CD19-CD28H/TM-LAT | SEQ ID NO: 51 | SEQ ID NO: 55 | SEQ ID NO: 62 | SEQ ID NO: 78 |
| CD19-CD28H/TM-Lck | SEQ ID NO: 51 | SEQ ID NO: 55 | SEQ ID NO: 63 | SEQ ID NO: 79 |
| HER2-CD28H/TM-ZAP70²⁵⁵⁻⁶⁰⁰ | SEQ ID NO: 50 | SEQ ID NO: 55 | SEQ ID NO: 64 | SEQ ID NO: 80 |
| HER2-CD28H/TM-PLCG1 | SEQ ID NO: 50 | SEQ ID NO: 99 | SEQ ID NO: 65 | SEQ ID NO: 81 |
| CD19-CD8H/TM-LAT | SEQ ID NO: 51 | SEQ ID NO: 56 | SEQ ID NO: 62 | SEQ ID NO: 82 |
| HER2-CD8H/TM-SLP76 | SEQ ID NO: 50 | SEQ ID NO: 56 | SEQ ID NO: 60 | SEQ ID NO: 83 |
| CD19-CD28H/TM-LAT^{Y161F} | SEQ ID NO: 51 | SEQ ID NO: 55 | SEQ ID NO: 66 | SEQ ID NO: 84 |
| CD19-CD28H/TM-LAT^{Y200F} | SEQ ID NO: 51 | SEQ ID NO: 55 | SEQ ID NO: 67 | SEQ ID NO: 85 |
| CD19- IgG4H/CD4TM-LAT^{G160D} | SEQ ID NO: 51 | SEQ ID NO: 57 | SEQ ID NO: 68 | SEQ ID NO: 86 |
| CD19-CD4H/TM-LAT | SEQ ID NO: 51 | SEQ ID NO: 58 | SEQ ID NO: 62 | SEQ ID NO: 87 |
| CD19-IgG4H/CD4TM-LAT | SEQ ID NO: 51 | SEQ ID NO: 57 | SEQ ID NO: 62 | SEQ ID NO: 88 |
| HER2-CD28H/TM-SLP-76^{K30R} | SEQ ID NO: 50 | SEQ ID NO: 55 | SEQ ID NO: 69 | SEQ ID NO: 89 |
| HER2-CD28H/TM-CD5 | SEQ ID NO: 50 | SEQ ID NO: 55 | SEQ ID NO: 70 | SEQ ID NO: 90 |
| HER2-CD28H/TM-CD6 | SEQ ID NO: 50 | SEQ ID NO: 55 | SEQ ID NO: 71 | SEQ ID NO: 91 |
| HER2-CD28H/TM-FcγR1 | SEQ ID NO: 50 | SEQ ID NO: 55 | SEQ ID NO: 72 | SEQ ID NO: 92 |
| HER2-CD28H/TM-4-1BB | SEQ ID NO: 50 | SEQ ID NO: 55 | SEQ ID NO: 73 | SEQ ID NO: 93 |
| HER2-CD28H/TM-CD28 | SEQ ID NO: 50 | SEQ ID NO: 100 | SEQ ID NO: 74 | SEQ ID NO: 94 |
| HER2-CD28H/TM-CD2 | SEQ ID NO: 50 | SEQ ID NO: 101 | SEQ ID NO: 75 | SEQ ID NO: 95 |
| HA-IgG1 long-CD28H/TM-ZAP70²⁵⁵⁻⁶⁰⁰ | SEQ ID NO: 52 | SEQ ID NO: 59 | SEQ ID NO: 64 | SEQ ID NO: 96 |
| B7-H3-CD28H/TM- ZAP70²⁵⁵⁻⁶⁰⁰ | SEQ ID NO: 53 | SEQ ID NO: 55 | SEQ ID NO: 64 | SEQ ID NO: 97 |
| CD22-CD8H/TM-LAT | SEQ ID NO: 54 | SEQ ID NO: 56 | SEQ ID NO: 62 | SEQ ID NO: 98 |
| CD19-CD28H/TM^{2CA}-LAT | SEQ ID NO: 51 | SEQ ID NO: 124 | SEQ ID NO: 62 | SEQ ID NO: 133 |
| CD19-CD28H/TM-LAT^{2YF} | SEQ ID NO: 51 | SEQ ID NO: 55 | SEQ ID NO: 125 | SEQ ID NO: 134 |
| CD19-IgG4H-CD4TM-LAT^{2YF} | SEQ ID NO: 51 | SEQ ID NO: 57 | SEQ ID NO: 125 | SEQ ID NO: 135 |
| CD19-CD28H/TM-LAT^{200-262 del} | SEQ ID NO: 51 | SEQ ID NO: 55 | SEQ ID NO: 126 | SEQ ID NO: 136 |
| CD19-CD28H/TM^{2CA}-LAT^{2YF} | SEQ ID NO: 51 | SEQ ID NO: 124 | SEQ ID NO: 125 | SEQ ID NO: 137 |
| CD19-CD28H/TM^{2CA}-LAT^{200-262 del} | SEQ ID NO: 51 | SEQ ID NO: 124 | SEQ ID NO: 126 | SEQ ID NO: 138 |
| CD19-CD28H/TM-LAT^{3YF} | SEQ ID NO: 51 | SEQ ID NO: 55 | SEQ ID NO: 127 | SEQ ID NO: 139 |
| HER2-CD8H/TM-SLP-76^{224-244 del} | SEQ ID NO: 50 | SEQ ID NO: 56 | SEQ ID NO: 128 | SEQ ID NO: 140 |
| HER2-CD28H/TM-SLP-76^{224-244 del} | SEQ ID NO: 50 | SEQ ID NO: 55 | SEQ ID NO: 128 | SEQ ID NO: 141 |
| B7-H3-CD28H/TM-ZAP70^{255-600 Y292F} | SEQ ID NO: 53 | SEQ ID NO: 55 | SEQ ID NO: 129 | SEQ ID NO: 142 |
| B7-H3-CD28H/TM-ZAP70^{255-600 Y492F} | SEQ ID NO: 53 | SEQ ID NO: 55 | SEQ ID NO: 130 | SEQ ID NO: 143 |
| B7-H3-CD28H/TM-ZAP70^{255-600 K544R} | SEQ ID NO: 53 | SEQ ID NO: 55 | SEQ ID NO: 131 | SEQ ID NO: 144 |
| B7-H3-CD28H/TM-ZAP70^{255-600 Y597F Y598F} | SEQ ID NO: 53 | SEQ ID NO: 55 | SEQ ID NO: 132 | SEQ ID NO: 145 |
| B7-H3-CD28H/TM-4-1BB-ZAP70²⁵⁵⁻⁶⁰⁰ | SEQ ID NO: 53 | SEQ ID NO: 153 | (SEQ ID NO: 154) + | SEQ ID NO: 157 |
| | | | SEQ ID NO: 156 | |
| B7-H3-CD28H/TM-CD28-ZAP70²⁵⁵⁻⁶⁰⁰ | SEQ ID NO: 53 | SEQ ID NO: 100 | (SEQ ID NO: 155) + | SEQ ID NO: 158 |
| | | | SEQ ID NO: 64 | |
| CD19-CD28H/TM-ZAP70²⁵⁵⁻⁶⁰⁰ | SEQ ID NO: 152 | SEQ ID NO: 55 | SEQ ID NO: 64 | SEQ ID NO: 159 |
| CD19-CD28H/TM-PLCG1 | SEQ ID NO: 152 | SEQ ID NO: 55 | SEQ ID NO: 65 | SEQ ID NO: 160 |
| GD2-8h/tm-ZAP70 (255-600) | SEQ ID NO: 205 | SEQ ID NO: 56 | SEQ ID NO: 64 | SEQ ID NO: 206 |
| B7-H3-28h/tm-ZAP70 (255-600 V314A) | SEQ ID NO: 53 | SEQ ID NO: 55 | SEQ ID NO: 207 | SEQ ID NO: 208 |
| B7-H3-28h/tm-ZAP70 (255-600 D327P) | SEQ ID NO: 53 | SEQ ID NO: 55 | SEQ ID NO: 209 | SEQ ID NO: 210 |
| B7-H3-28h/tm-ZAP70 (255-600 R360P) | SEQ ID NO: 53 | SEQ ID NO: 55 | SEQ ID NO: 211 | SEQ ID NO: 212 |
| B7-H3-28h/tm-ZAP70 (255-600 K362E) | SEQ ID NO: 53 | SEQ ID NO: 55 | SEQ ID NO: 213 | SEQ ID NO: 214 |
| B7-H3-28h/tm-4-1BB-ZAP70 (255-600 | SEQ ID NO: 53 | SEQ ID NO: 55 | (SEQ ID NO: 154) + | SEQ ID NO: 215 |
| Y292F) | | | SEQ ID NO: 129 | |
| CD19-28h/tm-ZAP70 (255-600 Y292F) | SEQ ID NO: 152 | SEQ ID NO: 55 | SEQ ID NO: 129 | SEQ ID NO: 216 |
| CD19-28h/tm-CD28-ZAP70 (255-600) | SEQ ID NO: 152 | SEQ ID NO: 55 | (SEQ ID NO: 155) + | SEQ ID NO: 217 |
| | | | SEQ ID NO: 64 | |
| CD19-28h/tm-4-1BB-ZAP70 (255-600) | SEQ ID NO: 152 | SEQ ID NO: 55 | (SEQ ID NO: 154) + | SEQ ID NO: 218 |
| | | | SEQ ID NO: 64 | |
| CD19-28h/tm-CD28-ZAP70 (255-600 | SEQ ID NO: 152 | SEQ ID NO: 55 | (SEQ ID NO: 155) + | SEQ ID NO: 219 |
| Y292F) | | | SEQ ID NO: 129 | |
| CD19-28h/tm-4-1BB-ZAP70 (255-600 | SEQ ID NO: 152 | SEQ ID NO: 55 | (SEQ ID NO: 154) + | SEQ ID NO: 220 |
| Y292F) | | | SEQ ID NO: 129 | |
| B7-H3-28h/tm-ZAP70 (280-600) | SEQ ID NO: 53 | SEQ ID NO: 55 | SEQ ID NO: 221 | SEQ ID NO: 222 |
| B7-H3-28h/tm-ZAP70 (308-600) | SEQ ID NO: 53 | SEQ ID NO: 55 | SEQ ID NO: 223 | SEQ ID NO: 224 |
| B7-H3-28h/tm-LAT | SEQ ID NO: 53 | SEQ ID NO: 55 | SEQ ID NO: 62 | SEQ ID NO: 225 |
| B7-H3-8h/tm-SLP-76 | SEQ ID NO: 53 | SEQ ID NO: 56 | SEQ ID NO: 60 | SEQ ID NO: 226 |
| GD2-28h/tm-LAT | SEQ ID NO: 205 | SEQ ID NO: 55 | SEQ ID NO: 62 | SEQ ID NO: 227 |
| GD2-8h/tm-SLP-76 | SEQ ID NO: 205 | SEQ ID NO: 56 | SEQ ID NO: 60 | SEQ ID NO: 228 |
| CD19-28h/tm-LAT (28-90del, 200-262del) | SEQ ID NO: 51 | SEQ ID NO: 55 | SEQ ID NO: 229 | SEQ ID NO: 230 |
| CD19-28h/tm(2CA)-LAT (28-90del, 200- | SEQ ID NO: 51 | SEQ ID NO: 124 | SEQ ID NO: 229 | SEQ ID NO: 231 |
| 262del) | | | | |
| CD19-28h/tm-LAT (28-130del, 200-262del) | SEQ ID NO: 51 | SEQ ID NO: 55 | SEQ ID NO: 232 | SEQ ID NO: 233 |
| CD19-28h/tm(2CA)-LAT (28-130del, 200- | SEQ ID NO: 51 | SEQ ID NO: 124 | SEQ ID NO: 232 | SEQ ID NO: 234 |
| 262del) | | | | |
| HER2-8h/tm-SLP-76 (1-81del, 224-244del) | SEQ ID NO: 50 | SEQ ID NO: 56 | SEQ ID NO: 235 | SEQ ID NO: 236 |
| HER2-8h/tm-SLP-76 (224-265del) | SEQ ID NO: 50 | SEQ ID NO: 56 | SEQ ID NO: 237 | SEQ ID NO: 238 |
| HER2-8h/tm-SLP-76 (1-81del, 224- | SEQ ID NO: 50 | SEQ ID NO: 56 | SEQ ID NO: 239 | SEQ ID NO: 240 |
| 300del)) | | | | |
| ROR1-28h/tm-LAT | SEQ ID NO: 241 | SEQ ID NO: 55 | SEQ ID NO: 62 | SEQ ID NO: 242 |
| ROR1-28h/tm(2CA)-LAT | SEQ ID NO: 241 | SEQ ID NO: 124 | SEQ ID NO: 62 | SEQ ID NO: 243 |
| ROR1-28h/tm-LAT (200-262del) | SEQ ID NO: 241 | SEQ ID NO: 55 | SEQ ID NO: 126 | SEQ ID NO: 244 |
| ROR1-28h/tm(2CA)-LAT (200-262del) | SEQ ID NO: 241 | SEQ ID NO: 124 | SEQ ID NO: 126 | SEQ ID NO: 245 |
| CD19-8h/tm-SLP-76 (224-244del) | SEQ ID NO: 51 | SEQ ID NO: 56 | SEQ ID NO: 128 | SEQ ID NO: 246 |
| ROR1-8h/tm-SLP-76 | SEQ ID NO: 241 | SEQ ID NO: 56 | SEQ ID NO: 60 | SEQ ID NO: 247 |
| ROR1-8h/tm-SLP-76 (224-244del) | SEQ ID NO: 241 | SEQ ID NO: 56 | SEQ ID NO: 128 | SEQ ID NO: 248 |

### REFERENCES

1. Majzner R G, Mackall C L. Clinical lessons learned from the first leg of the CAR T cell journey. Nat Med. 2019; 25(9):1341-55. Epub 2019/09/11. doi: 10.1038/s41591-019-0564-6. PubMed PMID: 31501612.
2. Lamers C H, Sleijfer S, van Steenbergen S, van Elzakker P, van Krimpen B, Groot C, Vulto A, den Bakker M, Oosterwijk E, Debets R, Gratama J W. Treatment of metastatic renal cell carcinoma with CAIX CAR-engineered T cells: clinical evaluation and management of on-target toxicity. Mol Ther. 2013; 21(4):904-12. Epub 2013/02/21. doi: 10.1038/mt.2013.17. PubMed PMID: 23423337; PMCID: PMC5189272.
3. Srivastava S, Salter A I, Liggitt D, Yechan-Gunja S, Sarvothama M, Cooper K, Smythe K S, Dudakov J A, Pierce R H, Rader C, Riddell S R. Logic-Gated ROR1 Chimeric Antigen Receptor Expression Rescues T Cell-Mediated Toxicity to Normal Tissues and Enables Selective Tumor Targeting. Cancer Cell. 2019; 35(3):489-503 e8. Epub 2019/03/20. doi: 10.1016/j.ccell.2019.02.003. PubMed PMID: 30889382; PMCID: PMC6450658.
4. Smith J B, Lanitis E, Dangaj D, Buza E, Poussin M, Stashwick C, Scholler N, Powell D J, Jr. Tumor Regression and Delayed Onset Toxicity Following B7-H4 CAR T Cell Therapy. Mol Ther. 2016; 24(11):1987-99. Epub 2016/07/22. doi: 10.1038/mt.2016.149. PubMed PMID: 27439899; PMCID: PMC5154474.
5. Gross G, Waks T, Eshhar Z. Expression of immunoglobulin-T-cell receptor chimeric molecules as functional receptors with antibody-type specificity. Proc Natl Acad Sci U S A. 1989; 86(24):10024-8. Epub 1989/12/01. doi: 10.1073/pnas.86.24.10024. PubMed PMID: 2513569; PMCID: PMC298636.
6. Irving B A, Weiss A. The cytoplasmic domain of the T cell receptor zeta chain is sufficient to couple to receptor-associated signal transduction pathways. Cell. 1991; 64(5):891-901. Epub 1991/03/08. doi: 10.1016/0092-8674(91)90314-o. PubMed PMID: 1705867.
7. Letourneur F, Klausner R D. Activation of T cells by a tyrosine kinase activation domain in the cytoplasmic tail of CD3 epsilon. Science. 1992; 255(5040):79-82. Epub 1992/01/03. doi: 10.1126/science.1532456. PubMed PMID: 1532456.
8. Kloss C C, Condomines M, Cartellieri M, Bachmann M, Sadelain M. Combinatorial antigen recognition with balanced signaling promotes selective tumor eradication by engineered T cells. Nat Biotechnol. 2013; 31(1):71-5. Epub 2012/12/18. doi: 10.1038/nbt.2459. PubMed PMID: 23242161; PMCID: PMC5505184.
9. Roybal K T, Rupp L J, Morsut L, Walker W J, McNally K A, Park J S, Lim W A. Precision Tumor Recognition by T Cells With Combinatorial Antigen-Sensing Circuits. Cell. 2016; 164(4):770-9. Epub 2016/02/03. doi: 10.1016/j.cell.2016.01.011. PubMed PMID: 26830879; PMCID: PMC4752902.
10. Majzner R G, Rietberg S P, Sotillo E, Dong R, Vachharajani V T, Labanieh L, Myklebust J H, Kadapakkam M, Weber E W, Tousley A M, Richards R M, Heitzeneder S, Nguyen S M, Wiebking V, Theruvath J, Lynn R C, Xu P, Dunn A R, Vale R D, Mackall C L. Tuning the Antigen Density Requirement for CAR T-cell Activity. Cancer Discov. 2020; 10(5):702-23. Epub 2020/03/21. doi: 10.1158/2159-8290.CD-19-0945. PubMed PMID: 32193224.
11. Lo W L, Shah N H, Rubin S A, Zhang W, Horkova V, Fallahee I R, Stepanek O, Zon L I, Kuriyan J, Weiss A. Slow phosphorylation of a tyrosine residue in LAT optimizes T cell ligand discrimination. Nat Immunol. 2019; 20(11):1481-93. Epub 2019/10/16. doi: 10.1038/s41590-019-0502-2. PubMed PMID: 31611699; PMCID: PMC6858552.
12. Wang X, Li J P, Chiu L L, Lan J L, Chen D Y, Boomer J, Tan T H. Attenuation of T cell receptor signaling by serine phosphorylation-mediated lysine 30 ubiquitination of SLP-76 protein. J Biol Chem. 2012; 287(41):34091-100. Epub 2012/08/21. doi: 10.1074/jbc.M112.371062. PubMed PMID: 22902619; PMCID: PMC3464518.
13. Currier N V, Ackerman S E, Kintzing J R, Chen R, Filsinger Interrante M, Steiner A, Sato A K, Cochran J R. Targeted Drug Delivery with an Integrin-Binding Knottin-Fc-MMAF Conjugate Produced by Cell-Free Protein Synthesis. Mol Cancer Ther. 2016; 15(6):1291-300. Epub 2016/05/20. doi: 10.1158/1535-7163.MCT-15-0881. PubMed PMID: 27197305.
14. Majzner R G, Theruvath J L, Nellan A, Heitzeneder S, Cui Y, Mount C W, Rietberg S P, Linde M H, Xu P, Rota C, Sotillo E, Labanieh L, Lee D W, Orentas R J, Dimitrov D S, Zhu Z, Croix B S, Delaidelli A, Sekunova A, Bonvini E, Mitra S S, Quezado M M, Majeti R, Monje M, Sorensen P H B, Maris J M, Mackall C L. CAR T Cells Targeting B7-H3, a Pan-Cancer Antigen, Demonstrate Potent Preclinical Activity Against Pediatric Solid Tumors and Brain Tumors. Clin Cancer Res. 2019. Epub 2019 Jan. 19. doi: 10.1158/1078-0432.CCR-18-0432. PubMed PMID: 30655315.
15. Travaglini K J, Nabhan A N, Penland L, Sinha R, Gillich A, Sit R V, Chang S, Conley S D, Mori Y, Seita J, Berry G J, Shrager J B, Metzger R J, Kuo C S, Neff N, Weissman I L, Quake S R, Krasnow M A. A molecular cell atlas of the human lung from single cell RNA sequencing. bioRxiv. 2020:742320. doi: 10.1101/742320.
16. Fedorov V D, Themeli M, Sadelain M. PD-1- and CTLA-4-based inhibitory chimeric antigen receptors (iCARs) divert off-target immunotherapy responses. Sci Transl Med. 2013; 5(215):215ra172. Epub 2013/12/18. doi: 10.1126/scitranslmed.3006597. PubMed PMID: 24337479; PMCID: PMC4238416.
17. Feucht J, Sun J, Eyquem J, Ho Y J, Zhao Z, Leibold J, Dobrin A, Cabriolu A, Hamieh M, Sadelain M. Calibration of CAR activation potential directs alternative T cell fates and therapeutic potency. Nat Med. 2019; 25(1):82-8. Epub 2018/12/19. doi: 10.1038/s41591-018-0290-5. PubMed PMID: 30559421; PMCID: PMC6532069.
18. Schultz L M, Muffly L S, Spiegel J Y, Ramakrishna S, Hossain N, Baggott C, Sahaf B, Patel S, Craig J, Yoon J, Kadapakkam M, Majzner R G, Frank M J, Erickson C, Marcy A C, Fujimoto M, Bhatia N, Meyer E H, Kong K A, Egeler E, Mavroukakis S, Qin H, Fry T J, Feldman S A, Miklos D B, Mackall C L, Davis K L. Phase I Trial Using CD19/CD22 Bispecific CAR T Cells in Pediatric and Adult Acute Lymphoblastic Leukemia (ALL). Blood. 2019; 134(Supplement_1):744-. doi: 10.1182/blood-2019-129411.

What is claimed is:

1. A chimeric antigen receptor (CAR) polypeptide comprising:
a) an extracellular ligand-binding domain having a binding affinity for a ligand;
b) a transmembrane domain; and
c) an intracellular signaling domain,
wherein binding of the ligand to the extracellular ligand-binding domain activates the intracellular signaling domain,
and wherein the intracellular signaling domain does not comprise an immune receptor tyrosine based activation motif (ITAM).

2. The CAR polypeptide of claim 1, wherein the intracellular signaling domain does not comprise CD3ζ.

3. The CAR polypeptide of claim 1 or 2, wherein the intracellular signaling domain comprises a full-length or biologically active fragment of a protein kinase, a G protein, a GTP-binding protein, an adaptor signaling protein, or a scaffold protein capable of inducing cell activation.

4. The CAR polypeptide any one of claims 1 to 3, wherein the intracellular signaling domain comprises ZAP70, PLCG1, PKC, ITK, NCK, VAV1, GRB2, GADS, SOS1, ADAP, SYK, LYN, PI3K, BLNK, or a biologically active fragment, mutant, or variant thereof.

5. The CAR polypeptide of any one of claims 1 to 4, wherein the intracellular signaling domain comprises ZAP70 or PLCG1, or a biologically active fragment, mutant, or variant thereof.

6. The CAR polypeptide of claim 5, wherein the biologically active fragment, mutant, or variant thereof comprises a fragment comprising Interdomain B and the kinase domain from ZAP70.

7. The CAR polypeptide of claim 5 or 6, wherein the biologically active fragment, mutant, or variant thereof comprises:
i) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment;
ii) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further comprising at least one of the mutations at the position of Y292, Y492, K544, Y597, Y598, V314, D327, R360, and K362;
iii) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further comprising at least one of the mutations of Y292F, Y492F, K544R, Y597F, Y598F, V314A, D327P, R360P, and K362E;
iv) a ZAP70^{255-600 Y292F} fragment;
v) a ZAP70^{255-600 Y492F} fragment;
vi) a ZAP70^{255-600 K544R} fragment;
vii) a ZAP70^{255-600 Y597F Y598F} fragment;
viii) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further comprising at least one costimulatory domain;
ix) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further comprising a 4-1BB costimulatory domain;
x) a ZAP70³⁰⁸⁻⁶⁰⁰ fragment, a ZAP70²⁸⁰⁻⁶⁰⁰ fragment, or a ZAP70²⁵⁵⁻⁶⁰⁰ fragment, further comprising a CD28 costimulatory domain;
xi) a ZAP70^{255-600 V314A} fragment;
xii) a ZAP70^{255-600 D327P} fragment;
xiii) a ZAP70^{255-600 R360P} fragment; or
xiv) a ZAP70^{255-600 K362E} fragment.

8. The CAR polypeptide of any one of claims 1 to 7, wherein the intracellular signaling domain comprises an amino acid sequence having at least 50%, 60%, 70%, 75%, 80%, 90%, 95%, 99%, or more identity to any one of SEQ ID NOs: 11-26, 103-110, 163, 165, 167, 169, 177, 179, 185, 188, 191, 193, and 195.

9. The CAR polypeptide of any one of claims 1 to 8, wherein the intracellular signaling domain comprises an amino acid sequence selected from SEQ ID NOs: 11-26, 103-110, 163, 165, 167, 169, 177, 179, 185, 188, 191, 193, and 195.

10. The CAR polypeptide of any one of claims 1 to 9, wherein the extracellular ligand-binding domain comprises a ligand binding domain of an antibody, an antigen-binding fragment, an antibody mimetic, a receptor, or a ligand for a targeted receptor.

11. The CAR polypeptide of claim 10, wherein the antibody or the antigen-binding fragment is selected from the group consisting of: a single chain variable fragment (scFv), a monoclonal antibody, an antigen-binding fragment (Fab), a nanobody, a diabody, a triabody, a minibody, an F(ab′)₂ fragment, an F(ab)v fragment, a single domain antibody (sdAb), a V_{H} domain, a V_{L} domain, an Fv fragment, a VNAR domain, and a VHH domain.

12. The CAR polypeptide of claim 10, wherein the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof.

13. The CAR polypeptide of any one of claims 1 to 12, wherein the extracellular ligand-binding domain is multivalent.

14. The CAR polypeptide of any one of claims 1 to 13, wherein the extracellular ligand-binding domain is multispecific.

15. The CAR polypeptide of any one of claims 1 to 14, wherein the ligand localizes on the surface of a cell.

16. The CAR polypeptide of claim 15, wherein the cell is a cancer cell.

17. The CAR polypeptide of any one of claims 1 to 16, wherein the ligand is an adaptor molecule specifically recognizing a cancer cell.

18. The CAR polypeptide of claim 16 or 17, wherein activation of the intracellular signaling domain promotes repression and/or killing of the cancer cell.

19. The CAR polypeptide of any one of claims 1 to 18, wherein the ligand is selected from the group consisting of: CD1a, CD1b, CD1c, CD2, CD3, CD4, CD5, CD6, CD7, CD8, CD9, CD10, CD11a, CD11b, CD11c, CD12, CD13, CD14, CD15 (SSEA-1), CD16 (FcγRIII), CD17, CD18, CD19, CD20, CD21, CD22, CD23, CD24, CD25, CD26, CD27, CD28, CD29, CD30, CD31, CD32 (FcγRII), CD33, CD34, CD35, CD36, CD37, CD38, CD39, CD40, CD41, CD43, CD44, CD44V6, CD45, CD45R/B220, CD45RO, CD49b, CD49d, CD49f, CD52, CD53, CD54, CD56 (NCAM), CD57, CD61 (integrin β3), CD62L, CD63, CD64, CD66b, CD68, CD69, CD70, CD73, CD74, CD79a (Igα), CD79b (Igβ), CD80, CD83, CD85k (ILT3), CD86, CD88, CD93 (C1Rqp), CD94, CD95, CD99, CD103, CD105 (Endoglin), CD107a, CD107b, CD114 (G-CSFR), CD115, CD117, CD122, CD123, CD129, CD133, CD134, CD138 (Syndecan-1), CD141, CD146, CD152 (CTLA-4), CD158 (Kir), CD161 (NK-1.1), CD163, CD183, CD191, CD193 (CCR3), CD194 (CCR4), CD195 (CCR5), CD197 (CCR7), CD203c, CD205 (DEC-205), CD207 (Langerin), CD209 (DC-SIGN), CD223, CD235, CD244 (2B4), CD252 (OX40L), CD267, CD268 (BAFF-R), CD273 (B7-DC, PD-L2), CD276 (B7-H3), CD279 (PD1), CD282 (TLR2), CD284 (TLR4), CD294, CD304 (Neuropilin-1), CD305, CD314 (NKG2D), CD319 (CRACC), CD326, CD328 (Siglec-7), CD335 (NKp46), fetal acetylcholine receptor (AChR), ADGRE2, alpha-fetoprotein (AFP), ALK, BCMA, BDCA3, C3AR, Lewis A (CA19.9), carbonic anhydrase IX (CA1X), calretinin, cancer antigen-125 (CA-125), CCR1, CCR4, CDS, carcinoembryonic antigen (CEA), chromogranin, CLEC12A, an antigen of a cytomegalovirus (CMV) infected cell (e.g., a cell surface antigen), CS-1, CSPG4, cytokeratin, desmin, DLK1, DLL3, EGFRvIII (epidermal growth factor variant III), EGFR and isovariants thereof, epithelial cell adhesion molecule (EpCAM), epithelial glycoprotein2 (EGP 2), epithelial glycoprotein-40 (EGP-40), epithelial membrane protein (EMA), ERBB, epithelial tumor antigen (ETA), FAP, folate-binding protein (FBP), FcγR1, FcεRIα, FITC, FLT3, FOLR1, FOLR3, galactin, ganlgiosides, gross cystic disease fluid protein (GCDFP-15), GD2 (ganglioside G2), GD3, GM2, GM3, glial fibrillary acidic protein (GFAP), gpA33, glycopeptides, Glypican 2 (GPC2), oncofetal antigen (h5T4), influenza hemagglutinin (HA), human epidermal growth factor receptor 2 (Her2/neu), HLA-DR, HM1.24, HMB-45 antigen, HPV E6, HPV E7, ICAM-1, IgG, IgD, IgE, IgM, IL-13-receptor alpha 1, integrins, Integrin B7, Interleukin-13 receptor subunit alpha-2 (IL-13Rα2), Kappa light chain, kinase insert domain receptor (KDR), Lamba light chain, LILRB2, Lewis Y (LeY), LGR5, Ly49, Ly108, L1 cell adhesion molecule (L1-CAM), melanoma-associated antigen (MAGE), melanoma antigen family A 1 (MAGE-A1), protein melan-A (melanoma antigen recognized by T lymphocytes; MART-1), MCSP, c-Met, MICA/B, mesothelin, muscle-specific actin (MSA), Mesothelin (MSLN), the dimeric form of the pyruvate kinase isoenzyme type M2 (tumor M2-PK), Mucin 1 (Muc-1), Mucin 16 (Muc-16), myo-D1, Necl-2, neurofilament, NKCSI, NKG2D, neuron-specific enolase (NSE), NY-ESO, cancer-testis antigen NY-ESO-1, an abnormal p53 protein, PAP (prostatic acid phosphatase), PAMA, P-cadherin, placental alkaline phosphatase, PRAIVIE, prostein, prostate stem cell antigen (PSCA), prostate-specific membrane antigen (PSMA), Ral-B, K-Ras (V-Ki-ras2 Kirsten rat sarcoma viral oncogene), an abnormal ras protein, ROR1, SLAMF7/CS1, receptor tyrosine-protein kinases erb-B2,3,4, sperm protein 17 (Sp17), STEAP1 (six-transmembrane epithelial antigen of the prostate 1), synaptophysin, tumor-associated glycoprotein 72 (TAG-72), TALLA-1, TARP (T cell receptor gamma alternate reading frame protein), TEM-8, human telomerase reverse transcriptase (hTERT), TIM-3, TLR4, TRBC1, TRBC2, Trp-p8, thyroglobulin, thyroid transcription factor-1, TYRP1, tyrosinase, vascular endothelial growth factor R2 (VEGF-R2), Vα24, Wilms tumor protein (WT-1), or any combination thereof.

20. The CAR polypeptide of any one of claims 1 to 19, wherein the transmembrane domain is derived from a transmembrane domain of CD4, CD8, CD28, PD-1, OX40, 4-1BB, CTLA-4, CD2, CD3D, CD3E, CD3G, CD3zeta, CD8a, CD8b, CD16, CD25, CD27, CD40, CD79A, CD79B, CD80, CD84, CD86, CD95, CD150 (SLAMF1), CD166, CD200R, CD223 (LAG3), CD270 (HVEM), CD272 (BTLA), CD273 (PD-L2), CD274 (PD-L1), CD278 (ICOS), CD300, CD357 (GITR), A2aR, ICAM-1, 2B4, BTLA, DAP10, FcRα, FcRβ, Fyn, GAL9, IL7, IL12, IL15, KIR, KIR2DL4, KIR2DS1, LAG-3, Lck, LAT, LPA5, LRP, NKp30, NKp44, NKp46, NKG2C, NKG2D, NOTCH1, NOTCH2, NOTCH3, NOTCH4, PTCH2, ROR2, Ryk, SLP-76, SIRPα, pTα, T cell receptor polypeptides (e.g., TCRα and TCRβ), TIM3, TRIM, ZAP70, or any combination thereof.

21. The CAR polypeptide of any one of claims 1 to 20, further comprising a hinge domain.

22. The CAR polypeptide of claim 21, wherein the hinge domain is derived from a hinge domain of CD8, CD28, CD4, IgG, PD-1, CTLA-4, CD2, LFA-1 (CD11a/CD18), CD5, CD27 (TNFRSF7), CD70, 4-1BB, OX40 (CD134), ICOS (CD278), IgG1 Fc region, IgG2 Fc region, IgG3 Fc region, IgG4 Fc region, IgE Fc region, IgM Fc region, IgA Fc region, or any combination thereof.

23. The CAR polypeptide of any one of claims 1 to 22, further comprising a costimulatory domain.

24. The CAR polypeptide of claim 23, wherein the costimulatory domain is derived from a costimulatory domain of CD28, ICOS (CD278), CD27, 4-1BB (CD137), OX40 (CD134), CD2, CD4, CD5, CD7, CD8, CD8α, CD8β, CD11a, CD11b, CD11c, CD11d, CD18, CD19, CD19a, CD29, CD30, CD30L, CD40, CD40L (CD154), CD48, CD49a, CD49D, CD49f, CD58, CD53, ICAM-1 (CD54), CD69, CD70, CD80 (B7-1), CD82, CD83, CD84, CD86 (B7-2), CD90, CD96, CD100, CD103, CD122, CD132, CD150 (SLAMF1), CD160 (BY55), CD162 (DNAM1), CD223 (LAG3), CD226, CD229, CD244, CD270 (HVEM), CD273 (PD-L2), CD274 (PD-L1), CD278, LAT, lymphocyte function-associated antigen-1 (LFA-1), LIGHT, NKG2C, NKG2D, NKp30, NKp44, NKp46, NKp80 (KLRF1), B7-H2, B7-H3, CD83 ligand, PD-1, SLP-76, Toll-like receptors (TLRs, such as TLR2), DAP10, DAP12, LAG-3, 2B4, CARD1, CTLA-4 (CD152), TRIM, ZAP70, FcERIγ, 4-1BBL, BAFF, GADS, GITR, GITR-L, BAFF-R, HVEM, CD27L, OX40L, TAC1, BLAME, CRACC, CD2F-10, NTB-A, integrin α4, integrin α4β1, integrin α4β7, IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LTBR, PAG/Cbp, PSGL1, SLAMF6 (NTB-A, Ly108), SLAMF7, SLP-76, TNFR2, TRANCE/RANKL, VLA1, VLA-6, BTLA, ikaros, LAG-3, LMIR, CEACAM1, CRTAM, TCL1A, DAP12, TIM-1, Dectin-1, PDCD6, PD-1, TIM-4, TSLP, EphB6, TSLP-R, HLA-DR, or any combination thereof.

25. The CAR polypeptide of any one of claims 1 to 24, comprising an amino acid sequence having at least 50%, 60%, 70%, 75%, 80%, 90%, 95%, 99%, or more identity to any one of SEQ ID NOs: 27-49, 111-123, 148-151, 162, 164, 166, 168, 170-176, 178, 180-184, 186, 187, 189, 190, 192, 194, 196, and 198-204.

26. The CAR polypeptide of any one of claims 1 to 25, comprising an amino acid sequence selected from SEQ ID NOs: 27-49, 111-123, 148-151, 162, 164, 166, 168, 170-176, 178, 180-184, 186, 187, 189, 190, 192, 194, 196, and 198-204.

27. The CAR polypeptide of any one of claims 1 to 26, capable of activating a cell expressing the CAR polypeptide.

28. The CAR polypeptide of claim 27, wherein the cell is an immune cell.

29. The CAR polypeptide of claim 28, wherein the immune cell is a T cell, a regulatory T cell (Treg), a natural killer (NK) cell, a macrophage, a monocyte, a gamma delta T cell, a stem cell, a natural killer T (NKT) cell, an induced pluripotent stem cell (iPSC)-derived NK cell, or an induced pluripotent stem cell (iPSC)-derived T cell.

30. The CAR polypeptide of claim 27, wherein the cell is a non-immune cell.

31. The CAR polypeptide of any one of claims 1 to 30, wherein activation of the intracellular signaling domain increases cytokine production in a T cell expressing the (CAR) polypeptide.

32. The CAR polypeptide of claim 31, wherein the cytokine comprises IL-2 and/or IFN-γ.

33. The CAR polypeptide of any one of claims 1 to 32, capable of reducing T cell exhaustion, compared to a CAR polypeptide comprising CD3zeta.

34. A polynucleotide encoding the chimeric antigen receptor (CAR) polypeptide of any one of claims 1 to 33.

35. An expression vector comprising the polynucleotide of claim 34.

36. A cell expressing the CAR polypeptide of any one of claims 1 to 33.

37. A composition comprising the CAR polypeptide of any one of claims 1 to 33.

38. A method of expressing a CAR polypeptide in a cell, comprising introducing a polynucleotide of claim 34 or an expression vector of claim 35 into the cell, and inducing expression of the CAR polypeptide under a condition.

39. A composition comprising
i) a first chimeric antigen receptor (CAR) polypeptide comprising:
a) a first extracellular ligand-binding domain having a binding affinity for a first ligand;
b) a first transmembrane domain; and
c) a first intracellular signaling domain,
and
ii) a second chimeric antigen receptor (CAR) polypeptide comprising:
a) a second extracellular ligand-binding domain having a binding affinity for a second ligand different from the first ligand;
b) a second transmembrane domain; and
c) a second intracellular signaling domain,
wherein a cell expressing both CAR polypeptides is activated only when the first extracellular ligand-binding domain binds to the first ligand and the second extracellular ligand-binding domain binds to the second ligand, and
wherein neither of the first and the second intracellular signaling domain comprises an ITAM.

40. The composition of claim 39, wherein neither of the first and the second intracellular signaling domains comprises CD3ζ.

41. The composition of claim 39 or 40, wherein at least one of the first and the second intracellular signaling domains comprises a full-length or biologically active fragment of a protein kinase, a G protein, a GTP-binding protein, an adaptor signaling protein, or a scaffold protein capable of inducing cell activation.

42. The composition of any one of claims 39 to 41, wherein at least one of the first and the second intracellular signaling domains is selected from the group consisting of: LAT, SLP-76, CD28, CD2, 4-1BB, CD6, and a biologically active fragment, mutant or variant thereof.

43. The composition of claim 42, wherein at least one of the first and the second intracellular signaling domains comprises LAT or SLP-76, or a biologically active fragment, mutant or variant thereof.

44. The composition of any one of claims 39 to 43, wherein the first intracellular signaling domain comprises LAT or a biologically active fragment, mutant or variant thereof and the second intracellular signaling domain comprises SLP-76 or a biologically active fragment, mutant or variant thereof.

45. The composition of any one of claims 39 to 44, wherein the first intracellular signaling domain comprises LAT or a biologically active fragment, mutant or variant thereof and the second intracellular signaling domain comprises CD28 or a biologically active fragment, mutant or variant thereof.

46. The composition of any one of claims 42 to 45, wherein the biologically active fragment, mutant, or variant thereof comprises a mutant of LAT, SLP-76, CD28, CD2, 4-1BB, or CD6, wherein the mutant comprises at least one mutation to the corresponding wild-type sequence.

47. The composition of claim 46, wherein the at least one mutation or deletion
i) enhances the potency of the composition;
ii) reduces the background activation levels of the cell when only one of the first and the second intracellular signaling domains is activated;
iii) reduces aggregation of the first and the second CAR polypeptides in absence of the ligand; and/or
iv) reduces ubiquitination and/or degradation of the first and/or the second CAR polypeptides.

48. The composition of claim 47, wherein the at least one mutation or deletion comprises
i) a mutation of G160D, Y200F, Y220F, Y252F, Y200F/Y220F, or Y200F/Y220F/Y252F, a deletion of amino acid residues at positions 200-262, a deletion of amino acid residues at positions 28-90, a deletion of amino acid residues at positions 28-130, deletions of amino acid residues at positions 28-90 and at positions 200-262, or deletions of amino acid residues at positions 28-130 and at positions 200-262, corresponding to the wild-type LAT sequence;
ii) a mutation of K30R, a deletion of amino acid residues at positions 224-244, a deletion of amino acid residues at positions 1-81, a deletion of amino acid residues at positions 224-265, a deletion of amino acid residues at positions 224-300, or deletions of amino acid residues at positions 1-81 and at positions 224-244, 224-265, or 224-300, corresponding to the wild-type SLP-76 sequence; and/or
iii) at least one mutation in a region on the first and/or the second CAR polypeptide capable of binding to GADS and/or GRB2.

49. The composition of any one of claims 39 to 48, wherein at least one of the first and the second intracellular signaling domains comprises an amino acid sequence having at least 50%, 60%, 70%, 75%, 80%, 90%, 95%, 99%, or more identity to any one of SEQ ID NOs: 11-26, 103-110, 163, 165, 167, 169, 177, 179, 185, 188, 191, 193, and 195.

50. The composition of any one of claims 39 to 49, wherein at least one of the first and the second intracellular signaling domains comprises an amino acid sequence selected from SEQ ID NOs: 11-26, 103-110, 163, 165, 167, 169, 177, 179, 185, 188, 191, 193, and 195.

51. The composition of any one of claims 39 to 50, wherein at least one of the first and the second extracellular ligand-binding domains comprises a ligand-binding domain of an antibody, an antigen-binding fragment, an antibody mimetic, a receptor, or a ligand for a targeted receptor.

52. The composition of claim 51, wherein the antibody or the antigen-binding fragment is selected from the group consisting of: a single chain variable fragment (scFv),a monoclonal antibody, an antigen-binding fragment (Fab), a nanobody, a diabody, a triabody, a minibody, an F(ab′)₂ fragment, an F(ab)v fragment, a single domain antibody (sdAb), a V_{H} domain, a V_{L} domain, an Fv fragment, a VNAR domain, and a VHH domain.

53. The composition of claim 51, wherein the antibody mimetic is selected from the group consisting of: Affibody molecules, Affilins, Affimers, Alphabodies, Avimers, DARPins, Fynomers, Kunitz domain peptides, Monobodies, nanoCLAMPs, and a biologically active fragment thereof.

54. The composition of claim 51, wherein the receptor comprises NKG2D, or a biologically active fragment thereof.

55. The composition of claim 51, wherein the ligand for a targeted receptor is an IL-13 polypeptide, an IL-13 mutein, chlorotoxin, or a biologically active fragment thereof.

56. The composition of any one of claims 39 to 55, wherein at least one of the first and the second extracellular ligand-binding domains is multivalent.

57. The composition of any one of claims 39 to 56, wherein at least one of the first and the second extracellular ligand-binding domains is multispecific.

58. The composition of any one of claims 39 to 57, wherein at least one of the first and the second ligands localizes on the surface of a cell.

59. The composition of claim 58, wherein the cell is a cancer cell.

60. The composition of any one of claims 39 to 57, wherein at least one of the first and the second ligands is an adaptor molecule specifically recognizing a cancer cell.

61. The composition of claim 59 or 60, wherein activation of both of the first and the second intracellular signaling domains promotes repression and/or killing of the cancer cell.

62. The composition of any one of claims 39 to 61, wherein at least one of the first and the second ligands is selected from the group consisting of: CD1a, CD1b, CD1c, CD2, CD3, CD4, CD5, CD6, CD7, CD8, CD9, CD10, CD11a, CD11b, CD11c, CD12, CD13, CD14, CD15 (SSEA-1), CD16 (FcγRIII), CD17, CD18, CD19, CD20, CD21, CD22, CD23, CD24, CD25, CD26, CD27, CD28, CD29, CD30, CD31, CD32 (FcγRII), CD33, CD34, CD35, CD36, CD37, CD38, CD39, CD40, CD41, CD43, CD44, CD44V6, CD45, CD45R/B220, CD45RO, CD49b, CD49d, CD49f, CD52, CD53, CD54, CD56 (NCAM), CD57, CD61 (integrin β3), CD62L, CD63, CD64, CD66b, CD68, CD69, CD70, CD73, CD74, CD79a (Igα), CD79b (Igβ), CD80, CD83, CD85k (ILT3), CD86, CD88, CD93 (C1Rqp), CD94, CD95, CD99, CD103, CD105 (Endoglin), CD107a, CD107b, CD114 (G-CSFR), CD115, CD117, CD122, CD123, CD129, CD133, CD134, CD138 (Syndecan-1), CD141, CD146, CD152 (CTLA-4), CD158 (Kir), CD161 (NK-1.1), CD163, CD183, CD191, CD193 (CCR3), CD194 (CCR4), CD195 (CCR5), CD197 (CCR7), CD203c, CD205 (DEC-205), CD207 (Langerin), CD209 (DC-SIGN), CD223, CD235, CD244 (2B4), CD252 (OX40L), CD267, CD268 (BAFF-R), CD273 (B7-DC, PD-L2), CD276 (B7-H3), CD279 (PD1), CD282 (TLR2), CD284 (TLR4), CD294, CD304 (Neuropilin-1), CD305, CD314 (NKG2D), CD319 (CRACC), CD326, CD328 (Siglec-7), CD335 (NKp46), fetal acetylcholine receptor (AChR), ADGRE2, alpha-fetoprotein (AFP), ALK, BCMA, BDCA3, C3AR, Lewis A (CA19.9), carbonic anhydrase IX (CA1X), calretinin, cancer antigen-125 (CA-125), CCR1, CCR4, CDS, carcinoembryonic antigen (CEA), chromogranin, CLEC12A, an antigen of a cytomegalovirus (CMV) infected cell (e.g., a cell surface antigen), CS-1, CSPG4, cytokeratin, desmin, DLK1, DLL3, EGFRvIII (epidermal growth factor variant III), EGFR and isovariants thereof, epithelial cell adhesion molecule (EpCAM), epithelial glycoprotein2 (EGP 2), epithelial glycoprotein-40 (EGP-40), epithelial membrane protein (EMA), ERBB, epithelial tumor antigen (ETA), FAP, folate-binding protein (FBP), FcγR1, FcεRIα, FITC, FLT3, FOLR1, FOLR3, galactin, ganlgiosides, gross cystic disease fluid protein (GCDFP-15), GD2 (ganglioside G2), GD3, GM2, GM3, glial fibrillary acidic protein (GFAP), gpA33, glycopeptides, Glypican 2 (GPC2), oncofetal antigen (h5T4), influenza hemagglutinin (HA), human epidermal growth factor receptor 2 (Her2/neu), HLA-DR, HM1.24, HMB-45 antigen, HPV E6, HPV E7, ICAM-1, IgG, IgD, IgE, IgM, IL-13-receptor alpha 1, integrins, Integrin B7, Interleukin-13 receptor subunit alpha-2 (IL-13Rα2), Kappa light chain, kinase insert domain receptor (KDR), Lamba light chain, LILRB2, Lewis Y (LeY), LGR5, Ly49, Ly108, L1 cell adhesion molecule (L1-CAM), melanoma-associated antigen (MAGE), melanoma antigen family A 1 (MAGE-A1), protein melan-A (melanoma antigen recognized by T lymphocytes; MART-1), MCSP, c-Met, MICA/B, mesothelin, muscle-specific actin (MSA), Mesothelin (MSLN), the dimeric form of the pyruvate kinase isoenzyme type M2 (tumor M2-PK), Mucin 1 (Muc-1), Mucin 16 (Muc-16), myo-D1, Necl-2, neurofilament, NKCSI, NKG2D, neuron-specific enolase (NSE), NY-ESO, cancer-testis antigen NY-ESO-1, an abnormal p53 protein, PAP (prostatic acid phosphatase), PAMA, P-cadherin, placental alkaline phosphatase, PRAIVIE, prostein, prostate stem cell antigen (PSCA), prostate-specific membrane antigen (PSMA), Ral-B, K-Ras (V-Ki-ras2 Kirsten rat sarcoma viral oncogene), an abnormal ras protein, ROR1, SLAMF7/CS1, receptor tyrosine-protein kinases erb-B2,3,4, sperm protein 17 (Sp17), STEAP1 (six-transmembrane epithelial antigen of the prostate 1), synaptophysin, tumor-associated glycoprotein 72 (TAG-72), TALLA-1, TARP (T cell receptor gamma alternate reading frame protein), TEM-8, human telomerase reverse transcriptase (hTERT), TIM-3, TLR4, TRBC1, TRBC2, Trp-p8, thyroglobulin, thyroid transcription factor-1, TYRP1, tyrosinase, vascular endothelial growth factor R2 (VEGF-R2), Vα24, Wilms tumor protein (WT-1), or any combination thereof.

63. The composition of any one of claims 39 to 62, wherein at least one of the first and the second transmembrane domains is derived from a transmembrane domain of: CD4, CD8, CD28, PD-1, OX40, 4-1BB, CTLA-4, CD2, CD3D, CD3E, CD3G, CD3zeta, CD8a, CD8b, CD16, CD25, CD27, CD40, CD79A, CD79B, CD80, CD84, CD86, CD95, CD150 (SLAMF1), CD166, CD200R, CD223 (LAG3), CD270 (HVEM), CD272 (BTLA), CD273 (PD-L2), CD274 (PD-L1), CD278 (ICOS), CD300, CD357 (GITR), A2aR, ICAM-1, 2B4, BTLA, DAP10, FcRα, FcRβ, Fyn, GAL9, IL7, IL12, IL15, KIR, KIR2DL4, KIR2DS1, LAG-3, Lck, LAT, LPA5, LRP, NKp30, NKp44, NKp46, NKG2C, NKG2D, NOTCH1, NOTCH2, NOTCH3, NOTCH4, PTCH2, ROR2, Ryk, SLP-76, SIRPα, pTα, T cell receptor polypeptides (e.g., TCRα and TCRβ), TIM3, TRIM, ZAP70, or any combination thereof.

64. The composition of any one of claims 39 to 63, wherein the first transmembrane domain and the second transmembrane domain are different.

65. The composition of claim 64, wherein using different transmembrane domains for the first and the second CAR polypeptides reduces background activation levels of the cell when only one of the first and the second intracellular signaling domains is activated.

66. The composition of any one of claims 39 to 65, wherein the first CAR polypeptide comprises a CD4 transmembrane domain and the second CAR polypeptide comprises a CD8 or CD28 hinge/transmembrane domain.

67. The composition of any one of claims 39 to 66, wherein at least one of the first and the second CAR polypeptides further comprises a hinge domain.

68. The composition of claim 67, wherein the hinge domain is derived from a hinge domain of CD8, CD28, CD4, IgG, PD-1, CTLA-4, CD2, LFA-1 (CD11a/CD18), CD5, CD27 (TNFRSF7), CD70, 4-1BB, OX40 (CD134), ICOS (CD278), IgG1 Fc region, IgG2 Fc region, IgG3 Fc region, IgG4 Fc region, IgE Fc region, IgM Fc region, IgA Fc region, or any combination thereof.

69. The composition of claim 67 or 68, wherein the first and the second CAR polypeptides further comprise a same hinge domain.

70. The composition of claim 67 or 68, wherein the first and the second CAR polypeptides further comprise different hinge domains.

71. The composition of claim 70, wherein a pair of the hinge domains of the first and the second CAR polypeptides is selected from the group consisting of: CD8 hinge domain and CD28 hinge domain, CD4 hinge domain and IgG4 hinge domain, CD8 hinge domain and IgG4 hinge domain, and CD28 hinge domain and IgG4 hinge domain.

72. The composition of claim 70 or 71, wherein using different hinge domains for the first and the second CAR polypeptides reduces background activation levels of the cell when only one of the first and the second intracellular signaling domains is activated.

73. The composition of any one of claims 68 to 72, wherein at least one of the first and the second CAR polypeptides comprises a mutation in the hinge/transmembrane domain.

74. The composition of claim 73, wherein the mutation reduces aggregation of the first and the second CAR polypeptides in absence of the ligand.

75. The composition of any one of claims 39 to 73, wherein at least one of the first and the second CAR polypeptides further comprises a costimulatory domain.

76. The composition of claim 75, wherein the costimulatory domain is derived from a costimulatory domain of: CD28, ICOS (CD278), CD27, 4-1BB (CD137), OX40 (CD134), CD2, CD4, CD5, CD7, CD8, CD8α, CD8β, CD11a, CD11b, CD11c, CD11d, CD18, CD19, CD19a, CD29, CD30, CD30L, CD40, CD40L (CD154), CD48, CD49a, CD49D, CD49f, CD58, CD53, ICAM-1 (CD54), CD69, CD70, CD80 (B7-1), CD82, CD83, CD84, CD86 (B7-2), CD90, CD96, CD100, CD103, CD122, CD132, CD150 (SLAMF1), CD160 (BY55), CD162 (DNAM1), CD223 (LAG3), CD226, CD229, CD244, CD270 (HVEM), CD273 (PD-L2), CD274 (PD-L1), CD278, LAT, lymphocyte function-associated antigen-1 (LFA-1), LIGHT, NKG2C, NKG2D, NKp30, NKp44, NKp46, NKp80 (KLRF1), B7-H2, B7-H3, CD83 ligand, PD-1, SLP-76, Toll-like receptors (TLRs, such as TLR2), DAP10, DAP12, LAG-3, 2B4, CARD1, CTLA-4 (CD152), TRIM, ZAP70, FcERIγ, 4-1BBL, BAFF, GADS, GITR, GITR-L, BAFF-R, HVEM, CD27L, OX40L, TAC1, BLAME, CRACC, CD2F-10, NTB-A, integrin α4, integrin α4β1, integrin α4β7, IA4, ICAM-1, IL2Rβ, IL2Rγ, IL7Rα, ITGA4, ITGA6, ITGAD, ITGAE, ITGAL, ITGAM, ITGAX, ITGB1, ITGB2, ITGB7, KIRDS2, LTBR, PAG/Cbp, PSGL1, SLAMF6 (NTB-A, Ly108), SLAMF7, SLP-76, TNFR2, TRANCE/RANKL, VLA1, VLA-6, BTLA, ikaros, LAG-3, LMIR, CEACAM1, CRTAM, TCL1A, DAP12, TIM-1, Dectin-1, PDCD6, PD-1, TIM-4, TSLP, EphB6, TSLP-R, HLA-DR, or any combination thereof.

77. The composition of any one of claims 39 to 76, further comprising a third CAR polypeptide comprising:
a) a third extracellular ligand-binding domain having a binding affinity for a third ligand different from the first ligand and the second ligand;
b) a third transmembrane domain; and
c) a third intracellular signaling domain,
wherein a cell expressing all three CAR polypeptides is activated only when the first extracellular ligand-binding domain binds to the first ligand and at least one of the second and the third extracellular ligand-binding domains binds to the second or the third ligand.

78. The composition of any one of claims 39 to 77, wherein at least one of the CAR polypeptides comprises an amino acid sequence having at least 50%, 60%, 70%, 75%, 80%, 90%, 95%, 99%, or more identity to any one of SEQ ID NOs: 27-49, 111-123, 148-151, 162, 164, 166, 168, 170-176, 178, 180-184, 186, 187, 189, 190, 192, 194, 196, and 198-204.

79. The composition of any one of claims 39 to 78, wherein at least one of the CAR polypeptides comprises an amino acid sequence selected from SEQ ID NOs: 27-49, 111-123, 148-151, 162, 164, 166, 168, 170-176, 178, 180-184, 186, 187, 189, 190, 192, 194, 196, and 198-204.

80. The composition of any one of claims 39 to 79, capable of activating a cell expressing the first and the second CAR polypeptides.

81. The composition of claim 80, wherein the cell is an immune cell.

82. The composition of claim 81, wherein the immune cell is a T cell, a natural killer (NK) cell, a macrophage, a monocyte, a stem cell, a gamma delta T cell, a natural killer T (NKT) cell, an induced pluripotent stem cell (iPSC)-derived NK cell, or an iPSC-derived T cell.

83. The composition of claim 80, wherein the cell is a non-immune cell.

84. The composition of any one of claims 39 to 83, wherein activation of both of the first and the second intracellular signaling domains increases cytokine production in a T cell expressing the first and the second CAR polypeptides.

85. The composition of claim 84, wherein the cytokine comprises IL-2 and/or IFN-γ.

86. The composition of any one of claims 39 to 85, capable of reducing T cell exhaustion.

87. A composition comprising a first polynucleotide molecule and a second polynucleotide molecule, wherein the first polynucleotide molecule encodes the first chimeric antigen receptor (CAR) polypeptide in the composition of any one of claims 39 to 86, and wherein the second polynucleotide molecule encodes the second chimeric antigen receptor (CAR) polypeptide in the composition of any one of claims 39 to 86.

88. The composition of claim 87, wherein the first and the second polynucleotide molecules are conjugated together.

89. A composition comprising a first expression vector and a second expression vector, wherein the first expression vector comprises the first polynucleotide molecule of claim 87, and wherein the second expression vector comprises the second polynucleotide molecule of claim 87.

90. An expression vector comprising the conjugated first and second polynucleotide molecules in the composition of claim 88.

91. A cell expressing the composition of any one of claims 39 to 86.

92. A method of expressing of the composition of any one of claims 39 to 86 in a cell, comprising introducing the composition of claim 87 or 88, the composition of claim 89, or the expression vector of claim 90 into the cell, and inducing expression of the CAR polypeptides under a condition.

93. A method for selectively activating a cell comprising contacting the cell with a ligand, wherein the cell expresses a CAR polypeptide of any one of claims 1 to 33, wherein the binding of the ligand to the extracellular ligand-binding domain activates the intracellular signaling domain of the CAR polypeptide, thereby activating the cell.

94. A method for selectively activating a cell comprising contacting the cell with a first ligand and a second ligand, wherein the cell expresses a composition of any one of claims 39 to 86, wherein binding of the first and the second ligands to the first and the second extracellular ligand-binding domains activates the first and the second intracellular signaling domains, respectively, thereby activating the cell, wherein activation of only one of the first and the second intracellular signaling domains does not activate the cell.

95. A method of antagonizing or killing a cancer cell comprising contacting the cancer cell with a cell expressing a CAR polypeptide of any one of claims 1 to 33, wherein the cancer cell expresses or specifically recognizes the ligand, wherein binding of the ligand to the extracellular ligand-binding domain activates the cell expressing the CAR polypeptide to antagonize or kill the cancer cell.

96. A method of antagonizing or killing a cancer cell comprising contacting the cancer cell with a cell expressing a composition of any one of claims 39 to 86, wherein the cancer cell expresses or specifically recognizes both the first ligand and the second ligand, wherein binding of the first ligand to the first extracellular ligand-binding domain and binding of the second ligand to the second extracellular ligand-binding domain activate the cell expressing the composition to antagonize or kill the cancer cell.

97. A method of treating a subject having a cancer, comprising administering to the subject a pharmaceutically effective amount of cells expressing a CAR polypeptide of any one of claims 1 to 33, wherein the cancer cells in the subject express the ligand on the surface, wherein the binding of the ligand to the extracellular ligand-binding domain activates the cell expressing the CAR polypeptide to antagonize or kill the cancer cells.

98. A method of treating a subject having a cancer, comprising administering to the subject a pharmaceutically effective amount of cells expressing or specifically recognizing a composition of any one of claims 39 to 86, wherein the cancer cells in the subject express both the first ligand and the second ligand, wherein the binding of the first ligand to the first extracellular ligand-binding domain and binding of the second ligand to the second extracellular ligand-binding domain activate the cells expressing the composition to antagonize or kill the cancer cells.

99. The cell of claim 36 or 91, wherein the cell is a regulatory T cell (Treg).

100. The method of any one of claims 38 and 92-98, wherein the cell is a regulatory T cell (Treg).

101. The composition of any one of claims 39 to 86, wherein the cell is a regulatory T cell (Treg).
